SEQUENCE LISTING (2 sequences)

SEQ ID NO: 1 - DNA, 37 bp, Artificial Sequence
  Description of Artificial Sequence Synthetic primer
gcctcactgtggccgtgatgaccttgaaggaggacac

SEQ ID NO: 2 - DNA, 37 bp, Artificial Sequence
  Description of Artificial Sequence Synthetic primer
gtgtcctccttcaaggtcatcacggccacagtgaggc

(19) United States
(12) Patent Application Publication
(10) Pub. No.: US 2020/0352914 A1
(43) Pub. Date: Nov. 12, 2020

(54) METHODS OF TREATING HYPERTENSION WITH ACTIVATORS OF TIE-2

(71) Applicant: Aerpio Pharmaceuticals, Inc., Cincinnati, OH (US)

(72) Inventor: Kevin PETERS, CINCINNATI, OH (US)

(21) Appl. No.: 16/850,613

(22) Filed: Apr. 16, 2020

Related U.S. Application Data
(60) Provisional application No. 62/840,655, filed on Apr. 30, 2019.
(60) Provisional application No. 62/835,626, filed on Apr. 18, 2019.

(51) Int. Cl. A61K 31/427; A61B 5/021; A61P 9/12; A61K 31/426

(52) U.S. Cl. CPC A61K 31/427; A61B 5/021; A61K 9/0019; A61K 31/426; A61P 9/12

(57) ABSTRACT

Disclosed herein are methods for treating hypertension, pulmonary hypertension, and associated conditions using activators of Tie-2 and inhibitors of HPTPβ. The methods include decreasing systolic blood pressure, decreasing diastolic blood pressure, decreasing mean arterial pressure, and modulating vascularization in the lungs.

### CROSS-REFERENCE

This application claims the benefit of U.S. Provisional Application No. 62/835,626, filed Apr. 18, 2019, and U.S. Provisional Application No. 62/840,655, filed Apr. 30, 2019, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 11, 2020, is named 45725727201_SL.txt and is 788 bytes in size.

### BACKGROUND

Hypertension is a condition that arises when the pressure exerted on vessel walls by the blood surpasses the normal range of pressure. Hypertension can be caused by a variety of factors, and is often asymptomatic. If left untreated over long periods of time then hypertension can strain the heart, damage blood vessels, and increase the risk of conditions such as heart attack, stroke, renal dysfunction, and vision loss due to diabetic retinopathy and diabetic macular edema (DME).

Pulmonary hypertension is characterized by elevated blood pressure in the lungs and right side of the heart. Elevated blood pressure in the pulmonary blood vessels can result from obstruction in or constriction of the arteries of the lung. Pulmonary hypertension can lead to a number of complications including heart failure, heart enlargement, blood clots, arrhythmia, pulmonary hemorrhage, and hemoptysis.

### SUMMARY

In some embodiments, the invention provides a method of modulating a blood pressure in a human in need thereof, the method comprising administering to the human a therapeutically effective amount of a Tie-2 activator, wherein the administration changes the blood pressure in the human by about 0.1 mmHg to about 100 mmHg.

In some embodiments, the invention provides a method of modulating blood pressure in a subject in need thereof, comprising administering to the subject a therapeutically effective amount of a Tie-2 activator, wherein: in a study of a human with hypertension, modulation in blood pressure in the human 90 minutes after administration of the Tie-2 activator to the human correlates to a baseline sitting blood pressure of the human as illustrated in the bottom panel of FIG. 22, and wherein the modulation in blood pressure in the human versus the baseline sitting blood pressure in the human has at most a 30% deviation from the regression line shown in the bottom panel of FIG. 22.

In some embodiments, the invention provides a method of modulating blood pressure in a subject in need thereof, comprising administering to the subject a therapeutically effective amount of a HPTPβ inhibitor, wherein: in a study of a human with hypertension, modulation in blood pressure in the human 90 minutes after administration of the HPTPβ inhibitor to the human correlates to a baseline sitting blood pressure of the human as illustrated in the bottom panel of FIG. 22.

In some embodiments, the invention provides a method of treating pulmonary hypertension in a subject in need thereof, the method comprising administering to the subject a therapeutically-effective amount of a Tie-activator, wherein the Tie-2 activator is a small organic molecule.

In some embodiments, the invention provides a method of treating hypertension in a subject in need thereof, comprising administering to the subject a therapeutically effective amount of a Tie-2 activator, wherein: in a study of a human with hypertension, modulation in blood pressure in the human 90 minutes after administration of the Tie-2 activator to the human correlates to a baseline sitting blood pressure of the human as illustrated in the bottom panel of FIG. 22, and wherein the modulation in blood pressure in the human versus the baseline sitting blood pressure in the human has at most a 30% deviation from the regression line shown in the bottom panel of FIG. 22.

In some embodiments, the invention provides a method of treating hypertension in a subject in need thereof, comprising administering to the subject a therapeutically effective amount of a HPTPβ inhibitor, wherein: in a study of a human with hypertension, modulation in blood pressure in the human 90 minutes after administration of the HPTPβ inhibitor to the human correlates to a baseline sitting blood pressure of the human as illustrated in the bottom panel of FIG. 22.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWINGS

FIG. 1 illustrates changes in VE-PTP expression in HUVECs cultured under hypoxic conditions.

FIG. 2 illustrates changes in Tie-2 phosphorylation in hypoxic HUVECs with Compound 1 treatment in the presence or absence of ANG-1 and ANG-2.

FIG. 3 illustrates changes in protein phosphorylation in hypoxic HUVECs with Compound 1 treatment in the presence or absence of ANG-1 and ANG-2.

FIG. 4 illustrates the heart rate of dogs at various time points after treatment with Compound 1 (treatment administered at time=0).

FIG. 5 illustrates the R wave-to-R wave (RR) interval of dogs at various time points after treatment with Compound 1 (treatment administered at time=0).

FIG. 6 illustrates the P wave-to-R wave interval (PR) interval of dogs at various time points after treatment with Compound 1 (treatment administered at time=0).

FIG. 7 illustrates the QRS duration in dogs at various time points after treatment with Compound 1 (treatment administered at time=0).

FIG. 8 illustrates the Q wave-to-T wave (QT) interval of dogs at various time points after treatment with Compound 1 (treatment administered at time=0).

FIG. 9 illustrates the corrected QT (QTc) interval of dogs at various time points after treatment with Compound 1 (treatment administered at time=0).

FIG. 10 illustrates the systolic blood pressure of dogs at various time points after treatment with Compound 1 (treatment administered at time=0).

FIG. 11 illustrates the diastolic blood pressure of dogs at various time points after treatment with Compound 1 (treatment administered at time=0).

FIG. 12 illustrates the mean arterial pressure of dogs at various time points after treatment with Compound 1 (treatment administered at time=0).

FIG. 13 illustrates the pulse pressure of dogs at various time points after treatment with Compound 1 (treatment administered at time=0).

FIG. 14 illustrates changes in systolic blood pressure from baseline with Compound 1 treatment in SHR and WKY rats.

FIG. 15 illustrates changes in mean plasma concentration of Compound 1 after subcutaneous administration of Compound 1 in human subjects with DME.

FIG. 16 illustrates changes in systolic blood pressure from baseline after subcutaneous administration of Compound 1 in human subjects with DME.

FIG. 17 illustrates the correlation between pre-dose systolic blood pressure and the change in systolic blood pressure after subcutaneous administration of Compound 1.

FIG. 18 illustrates the plasma concentration of Compound 1 in human subjects. EXAMPLE 8: subcutaneous Compound 1; EXAMPLE 9, Group 1: subcutaneous Compound 1+intravitreal sham; EXAMPLE 9, Group 2: subcutaneous Compound 1+intravitreal ranibizumab.

FIG. 19 illustrates changes in systolic pressure from baseline. Group 1: subcutaneous Compound 1+intravitreal sham; Group 2: subcutaneous Compound 1+intravitreal ranibizumab; Group 3: subcutaneous placebo+intravitreal ranibizumab.

FIG. 20 panel A illustrates changes in sitting systolic blood pressure (SBP) from baseline in subjects having a baseline sitting SBP≥140 mmHg. Panel B illustrates changes in sitting SBP from baseline in subjects having a baseline SBP<140 mmHg.

FIG. 21 illustrates the correlation between pre-dose systolic blood pressure and the change in systolic blood pressure 30 minutes after subcutaneous administration of Compound 1 with or without intravitreal ranibizumab administration.

FIG. 22 illustrates the correlation between pre-dose systolic blood pressure and the change in systolic blood pressure 90 minutes after subcutaneous administration of Compound 1 with or without intravitreal ranibizumab administration.

FIG. 23 illustrates the correlation between the change in heart rate and the change in sitting systolic (SYS) blood pressure (BP) with combinations of Compound 1 and ranibizumab treatment. Group 1 (denoted by “o”): subcutaneous Compound 1+intravitreal sham; Group 2 (denoted by “x”): subcutaneous Compound 1+intravitreal ranibizumab; Group 3 (denoted by “Δ”): subcutaneous placebo+intravitreal ranibizumab.

FIG. 24 panel A illustrates the effect of Compound 2 on the tone of endothelium-intact aortic rings. Panel B illustrates the effect of Compound 2 on the contractile response to phenylephrine (PE). Panel C illustrates the effect of Compound 2 on relaxation induced by sodium nitroprusside (SNP). Panel D illustrates the effect of Compound 2 on relaxation induced by acetylcholine (ACh).

FIG. 25 panel A illustrates the effect of Compound 2 on nitrite levels in the supernatant of human endothelial cells. Panel B illustrates the effect of Compound 2 on eNOS phosphorylation on Tyr81 (Y81; assessed in eNOS immunoprecipitates) and Ser1177 (S1177; assessed in whole cell lysates). Panel C illustrates the quantification of the changes in eNOS phosphorylation on Tyr81. Panel D illustrates the quantification of the changes in eNOS phosphorylation on Ser1177.

FIG. 26 panel A illustrates the effect of Compound 2 on eNOS phosphorylation on Tyr81, Ser1177, and Ser633, as well as Akt phosphorylation on Ser473. Panel B illustrates the quantification of the changes in eNOS and Akt phosphorylation.

FIG. 27 panel A illustrates the effect of Compound 2 on eNOS phosphorylation on Tyr81. Panel B illustrates the effect of ABL1 on eNOS phosphorylation on Tyr81. Panel C illustrates the effect of ABL1 downregulation on eNOS phosphorylation and activity. Panel D illustrates the effect on nitrite levels in the supernatant.

FIG. 28 panel A illustrates the interaction of VE-PTP with eNOS immunoprecipitated (IP) from cells treated with solvent or Yoda1. Panel B illustrates the results of an in vitro phosphatase assay using eNOS immunoprecipitated from Yoda1-stimulated cells and recombinant human VE-PTP.

FIG. 29 panel A illustrates the effects of Compound 2 on acetylcholine-induced relaxation of endothelium-intact aortic rings from WT and Akita mice. Panel B illustrates the effects of Compound 2 on phenylephrine-induced contraction of aortic rings from WT and Akita mice.

FIG. 30 panel A illustrates the effects of Compound 1 on systolic blood pressure in diabetic patients. Panel B illustrates the effects of Compound 1 on diastolic blood pressure in diabetic patients. Panel C illustrates the effects of Compound 1 on heart rate in diabetic patients. Panel D illustrates the effects of Compound 1 on systolic blood pressure in diabetic patients. Panel E illustrates the effects of Compound 1 on diastolic blood pressure in diabetic patients. Panel F illustrates the effects of Compound 1 on heart rate in diabetic patients.

FIG. 31 panel A illustrates the effects of Compound 1 on mean arterial blood pressure, heart rate, and right ventricular pressure. Panel B illustrates an assessment of right ventricular hypertrophy based on the Fulton index.

FIG. 32 illustrates the nexus between the endothelial cell layer of arteries, veins, and capillaries.

### DETAILED DESCRIPTION

Described herein are therapies using a Tie-2 activator for treatment of, for example, elevated blood pressure, hypertension, pulmonary hypertension, or an ongoing hypertensive crisis. A Tie-2 activator of the disclosure can activate Tie-2 signaling by promoting protein phosphorylation, such as phosphorylation of the Tie-2 protein.

### Tie-2 Activation and Blood Pressure.

Tie-2 (tyrosine kinase with immunoglobulin and epidermal growth factor homology domains 2) is a membrane receptor tyrosine kinase expressed primarily in vascular endothelial cells and a subset of hematopoietic stem cells (HSCs) and macrophages. Phosphorylation of Tie-2 leads to Tie-2 activation. Upstream factors regulate Tie-2 phosphorylation, which influences downstream signaling pathways. Non-limiting examples of factors regulating Tie-2 include angiopoietin 1 (Ang-1), angiopoietin 2 (Ang-2), and human protein tyrosine phosphatase beta (often abbreviated as HPTPβ or HPTP-beta).

Ang-1 is an agonist of Tie-2, and binding of Ang-1 to Tie-2 promotes receptor phosphorylation. Ang-2 is a Tie-2 ligand that acts in a context-dependent antagonistic or agonistic manner. Binding of Ang-1 to Tie-2 increases the level of endogenous Tie-2 receptor phosphorylation and initiates multiple pathways including downstream AKT signaling and the Ras/Raf/MEK/ERK pathway. This binding initiates a signaling cascade that can induce distinctive vascular remodeling through highly organized angiogenesis and tightening of the endothelial cell junctions (endothelium cell proximity). Within the vascular endothelium, Ang-1-Tie-2 signaling promotes endothelial cell proximity. In the HSC microenvironment, Ang-1-Tie-2 signaling contributes in a paracrine manner to the long-term repopulation of HSCs.

Under physiological conditions, the duration of Tie-2 phosphorylation is regulated by HPTPβ, which removes the phosphate group from the Tie-2 receptor. Inhibiting HPTPβ substantially increases Tie-2 phosphorylation levels, and restores proper cell proximity. A small molecule of the disclosure can activate Tie-2 downstream signaling by inhibiting HPTPβ/VE-PTP.

HPTPβ and vascular endothelial protein tyrosine phosphatase (VE-PTP; the mouse orthologue of HPTPβ) are expressed in vascular endothelial cells throughout development and in the adult vasculature. HPTPβ plays a functional role in endothelial cell proliferation, endothelial cell viability, endothelial cell differentiation, endothelial cell permeability, vasculogenesis, and angiogenesis. HPTPβ also modulates interactions with inflammatory and endothelial support cells, such as pericytes, podocytes, and smooth muscle cells. HPTPβ maintains the integrity of the endothelial barrier by regulating the phosphorylation of proteins within endothelial cell junctions, including Tie-2, the adherens junction components, VE-cadherin, plakoglobin, and vascular endothelial growth factor receptor 2 (VEGFR2).

Various proteins are important for the formation and maintenance of tight junctions. Tight junctions are areas of close proximity between cells whose exteriors together form a barrier to fluid. Tight junctions are joined together by sealing strands. A variety of proteins play functional roles in maintaining the homeostasis of tight junctions. VE-cadherin is a calcium dependent cell-cell adhesion glycoprotein that is required for maintaining a restrictive endothelial barrier. Claudins are a family of proteins that function as a physical barrier to control the flow of molecules in the intercellular space between the cells of an epithelium. Tight junction protein ZO-1 is involved in transducing a signal required for tight junction assembly. Platelet endothelial cell adhesion molecule 1 (PECAM1 or CD31) is another potential HPTPβ substrate involved in the regulation of junctional integrity and signaling.

The expression of HPTPβ is upregulated by hypoxia, diabetes, and renin-induced hypertension, which results in reduced Tie-2 signaling and the loss of endothelial cell barrier integrity. Thus, targeting HPTPβ can activate Tie-2 and restore downstream signaling in endothelial cells.

HPTPβ dephosphorylates Tie-2, PECAM1/CD31, VE-cadherin, and VEGFR2. PECAM1/CD31, VE-cadherin, and VEGFR2 together form a signal transduction complex that regulates the endothelial cellular response to fluid shear stress, including the activation of the endothelial nitric oxide (NO) synthase (eNOS) following the opening of the mechanosensitive cation channel, PIEZO1. Activation of Tie-2 can lead to a decrease in blood pressure through phosphorylation of eNOS. Activated Tie-2 can lead to PI3K signaling, which can lead to the phosphorylation and activation of Akt, and localization of Akt to the plasma membrane. Activated Akt can induce phosphorylation of eNOS at the plasma membrane, thereby activating eNOS. Activated eNOS can catalyze nitric oxide production in the vascular endothelium. Nitric oxide can diffuse from the vascular endothelium into vascular smooth muscle cells, where nitric oxide can activate guanylyl cyclase, and cause the dephosphorylation of guanosine triphosphate (GTP). GTP dephosphorylation can lead to the relaxation of vascular smooth muscle via multiple mechanisms including, for example, the inhibition of intracellular Ca²⁺ entry, activation of K⁺ channels, and activation of myosin light chain phosphatase. Relaxation of vascular smooth muscle can lead to a decrease in blood pressure. A method disclosed herein can decrease blood pressure by activating Tie-2 signaling.

HPTPβ/VE-PTP inhibition can lead to the phosphorylation of the receptor protein tyrosine kinase, Ephrin type-B receptor 4 (EphB4). Ephrin receptors and their ephrin ligands mediate numerous developmental processes, particularly in the nervous system. EphB4 plays a role in vascular stabilization and regression of pathologic neovascularization. EphB4 can form a ternary complex with VE-PTP and Tie-2 in endothelial cells, and VE-PTP controls the phosphorylation of both Tie-2 and EphB4.

Cdc42 GEF facio-genital dysplasia-5 (FGD5) and bone morphogenetic protein receptor type 2 (BMPR2) are also enriched upon inhibition of HPTPβ/VE-PTP. FGD5 is a FYVE, RhoGEF, and PH domain-containing protein 5. FGD5 regulates pro-angiogenic effects of VEGF in vascular endothelial cells, including network formation, directional movement, and proliferation. FGD5 mediates VEGF-induced Cdc42 activation at endothelial cell junctions. Tie-2 activation caused by HPTPβ/VE-PTP inhibition can lead to the phosphorylation of FGD5, which prevents translocation of FGD5 to cell-cell junctions. The inability of FGD5 to localize to cell-cell junctions can contribute to junction stabilization. Thus, FGD5 can be important factor for maintaining endothelial junction integrity caused by HPTPβ/VE-PTP inhibition.

BMPR2 is a member of the bone morphogenetic protein (BMP) receptor family of transmembrane serine/threonine kinases. BMPs are involved in endochondral bone formation and embryogenesis. BMPR2 plays a critical role in dampening inflammatory signals in the pulmonary vasculature. Mutations in BMPR2 have been associated with primary pulmonary hypertension, both familial and idiosyncratic pulmonary hypertension, and pulmonary venoocclusive disease. The vascular endothelium provides a barrier between blood and tissues, thus preventing underlying stromal cells from exposure to growth factors present in the blood. In pulmonary arterial hypertension, loss of endothelial-barrier integrity can result in the abnormal exposure of the underlying smooth muscle cells to growth factors, leading to uncontrolled proliferation. BMPR2 can maintain the barrier function of the pulmonary artery endothelial monolayer by suppressing leukocyte transmigration. Loss of BMPR2 in the endothelial layer of the pulmonary vasculature can lead to increased susceptibility to inflammation by promoting the extravasation of leukocytes into the pulmonary artery wall. Thus, mutations in BMPR2 in the presence of inflammatory stimuli can lead to the development of pulmonary arterial hypertension.

### Elevated Blood Pressure, Hypertension, and Hypertensive Crisis.

A therapy of the present disclosure can be used to treat, for example, elevated blood pressure, hypertension, or an ongoing hypertensive crisis. Each of the foregoing is a medical condition that arises when the pressure exerted by the blood on the vessel walls is persistently elevated. The indications disclosed herein can be diagnosed by measuring the blood pressure of a subject using a sphygmomanometer, or blood pressure meter. The sphygmomanometer reading provides two measurements of pressure: systolic and diastolic. Systolic blood pressure (SBP) is the maximum pressure exerted on the vessel wall during a heartbeat, and occurs during the contraction of heart muscles. The normal range for systolic blood pressure in a healthy human adult is, for example, between about 90 mmHg to about 120 mmHg. Diastolic blood pressure (DBP) is the minimum pressure exerted on the vessel wall between two heart beats, and occurs while the heart fills with blood. The normal range for diastolic blood pressure in a healthy human adult is, for example, between about 60 mmHg to about 80 mmHg.

From measurements of systolic and diastolic blood pressure, further measures can be calculated or approximated such as, for example, pulse pressure (PP) and mean arterial pressure. Pulse pressure is the difference between systolic and diastolic blood pressure. The normal range for pulse pressure in a healthy human adult is, for example, about 30 mmHg to about 60 mmHg. Mean arterial pressure is the average blood pressure during a cardiac cycle, and can be approximated by the equation (2DBP+SBP)/3, where DPB is diastolic blood pressure and SBP is systolic blood pressure. The normal range for mean arterial pressure in a healthy human adult is, for example, between about 70 mmHg to about 100 mmHg.

Elevated blood pressure can occur when systolic pressure in a subject consistently ranges from about 120 mmHg to about 129 mmHg, and diastolic pressure is less than about 80 mmHg. Stage 1 hypertension can occur when systolic pressure in a subject is about 130 mmHg to about 139 mmHg, or diastolic pressure is about 80 mmHg to about 89 mmHg. Stage 2 hypertension can occur when systolic pressure in a subject is about 140 mmHg or greater, or diastolic pressure is about 90 mmHg or greater. A hypertensive crisis can occur when the systolic pressure in a subject is greater than about 180 mmHg, or diastolic pressure is greater than about 120 mmHg. In some embodiments, a compound disclosed herein is used for treatment of elevated blood pressure. In some embodiments, a compound disclosed herein is used for the treatment of stage 1 hypertension. In some embodiments, a compound disclosed herein is used for the treatment of stage 2 hypertension. In some embodiments, a compound disclosed herein is used for the treatment of hypertensive crisis.

Hypertension can be classified as either primary (also known as essential or idiopathic) or secondary hypertension. Primary hypertension can be caused by nonspecific genetic and lifestyle factors. Non-limiting examples of lifestyle factors that can be risk factors for primary hypertension include age, race, obesity, dietary choices, tobacco use, alcohol consumption, and high stress levels. Secondary hypertension can be due to identifiable causes including, for example, chronic kidney disease or the use of certain medications.

### Symptoms of Hypertension and Related Indications.

Subjects with hypertension can be asymptomatic. However, symptoms of hypertension can include, for example, headaches, nosebleeds, and shortness of breath. Even in the absence of symptoms, hypertension can damage blood vessels and lead to other comorbidities including, for example, cardiovascular diseases. Persistent hypertension can be a risk factor for many cardiovascular disorders including, for example, atherosclerosis, coronary artery disease, left ventricular hypertrophy, heart failure, coronary microvascular disease, and cardiac arrhythmias.

Atherosclerosis is characterized by the hardening and narrowing of arteries due to the build-up of plaque on the arterial wall. Hypertension can increase a subject's risk for atherosclerosis as the added force placed on the arterial walls due to increased blood pressure can make the walls more susceptible to plaque build-up and narrowing. In early stages, atherosclerosis can by asymptomatic; however, over time, symptoms such as chest pain, numbness and/weakness in arms or legs, leg pain, temporary loss of vision in one eye, drooping muscles in the face, and difficulty speaking can occur. Atherosclerosis can be treated using, for example, statins such as atorvastatin, simvastatin, pravastatin, and lovastatin; blood thinners such as aspirin; and cholesterol medication such as gemfibrozil, ezetimibe, or fenofibrate.

As atherosclerosis progresses, the vessel narrowing and plaque build-up can occur in the arteries that supply blood to the heart muscle. This narrowing and plaque build-up deprives the heart of oxygen, resulting in coronary artery disease. Coronary artery disease can present with symptoms including, for example, pain the chest, neck, arm, or back, chest tightness, shortness of breath, fatigue, and nausea. As coronary artery disease worsens, a complete blockage of an artery can cause a heart attack. Treatments for coronary artery disease include, for example, blood thinners such as clopidogrel, and aspirin; statins such as atorvastatin, simvastatin, pravastatin, and lovastatin; beta blockers such as atenolol and metoprolol; heart medications such as nitroglycerin and isosorbide; and calcium channel blockers such as amlodipine.

Hypertension can also lead to left ventricular hypertrophy. In a healthy subject, the ventricular walls stretch when filled with blood, and contract to pump blood out of the heart. In a hypertensive subject, the muscles of the ventricle must work harder to pump blood due to increased pressure in blood vessels. The increased workload caused by the increased blood pressure causes the ventricular walls to thicken and become less flexible, resulting in ventricular hypertrophy. Left ventricular hypertrophy can be asymptomatic, or symptoms such as shortness of breath, fatigue, chest pain, heart palpitations, dizziness, and fainting can occur.

As left ventricular hypertrophy progresses, the hypertrophy can cause the heart to lose the elasticity necessary to provide enough force to pump blood throughout the body effectively. The inability of the heart to pump the necessary amount of blood results in heart failure. Heart failure can present with symptoms such as shortness of breath, fatigue, and swelling of the legs. Several treatment strategies for managing left ventricular hypertrophy and/or heart failure exist, including, for example, surgical procedures, life style changes, and treatment with angiotensin-converting enzyme (ACE) inhibitors, angiotensin II receptor blockers, calcium channel blockers, diuretics, beta blockers, vasodilator agents, positive inotropes, and aldosterone antagonists.

Another non-limiting example of a condition that can be caused by hypertension is coronary microvascular disease. In coronary microvascular disease, the walls of small arteries in the heart are damaged. This damage can lead to symptoms including, for example, chest pain, discomfort in the arm, jaw, neck, back or abdomen, shortness of breath, and fatigue. Coronary artery disease can be treated with medications including cholesterol medications such as gemfibrozil, ezetimibe, and fenofibrate; statins such as atorvastatin, simvastatin, pravastatin, and lovastatin; aspirin; and nitroglycerin.

Cardiac arrhythmias are another non-limiting example of a set of conditions that can be caused by hypertension. Cardiac arrhythmias are a group of conditions in which the heartbeat is irregular, too slow, or too fast. Symptoms of arrhythmias include a fluttering in the chest, a racing heartbeat, a slowed heartbeat, chest pain, shortness of breath, dizziness, sweating, and fainting. Cardiac arrhythmias can be treated via implantation of a pacemaker or implantable cardioverter defibrillator, cardioversion, catheter ablation, or medication designed to decrease heart rate.

Further non-limiting examples of complications for which hypertension can be a risk factor include brain infarction, brain hemorrhage, stroke, renal injury, end-stage renal disease, and hypertensive retinopathy.

In addition to being a cause of comorbidities, persistent hypertension can exacerbate comorbidities. A non-limiting example of a condition that can be exacerbated by hypertension is DME. DME is an accumulation of fluid in the macula due to leaking blood vessels. The accumulation of fluid causes the macula to swell and thicken, and distorts vision. DME can eventually lead to blindness, the risk of which is increased by the presence of hypertension in a subject.

DME can be treated with, for example, inhibitors of vascular endothelial growth factor (VEGF). VEGF can bind to cognate VEGF receptor tyrosine kinases (VEGFRs), resulting in phosphorylation of the receptors, and of downstream signal transducers. VEGFR-mediated signaling can result in aberrant vasculogenesis, angiogenesis, and permeabilization of blood vessels, contributing to pathologic vascular instability. VEGFR-mediated signaling can also activate eNOS and prostacyclin production. Prostacyclin production and eNOS activation can both lead to relaxation of vascular smooth muscle, thereby decreasing blood pressure. Thus, inhibition of VEGF can result in decreased VEGFR-mediated signaling, leading to enhanced vascular stability, but also elevated blood pressure.

In some embodiments, activation of Tie-2 or inhibition of HPTPβ with a compound of the disclosure promotes activation of eNOS in endothelial cells, which in turn activates guanylate cyclase in smooth muscle cells, producing cGMP, which can relax smooth muscle cells, resulting in vasodilation. In some embodiments, a Tie-2 activator or a HPTPβ inhibitor increases a concentration of NO and promotes vascular density by reducing vascular leak.

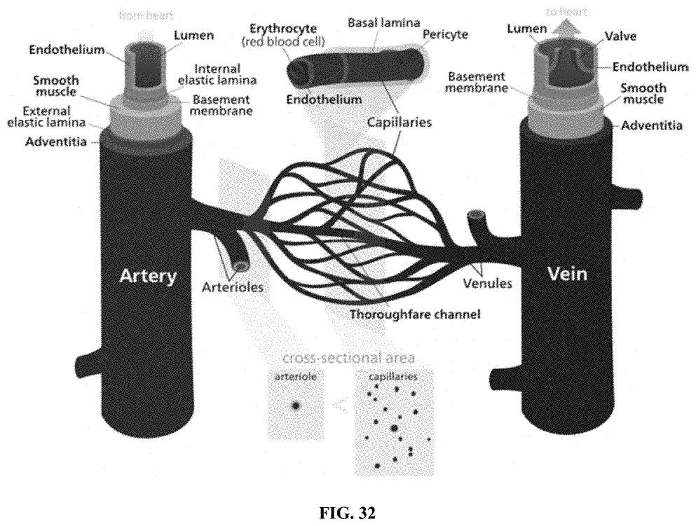

In a case of vascular leak, the endothelial cells that line blood vessels separate, allowing leakage of fluid from the circulatory system to interstitial space. Symptoms of vascular leak include hemoconcentration, hypotension, hypoalbuminemia, partial or generalized edema, monoclonal gammopathy of undetermined significance (MGUS), fatigue, and syncope. Arteries, veins, and capillaries are susceptible to the increase in vascular permeability that leads to vascular leak. FIG. 32 illustrates the nexus between the endothelial cell layer and arteries, veins, and capillaries, and supporting pericytes and smooth muscle cells.

Additional enzymes that modulate endothelial function include adenylate cyclase, guanylate cyclase, nitric oxide synthetase, and phosphodiesterases. Adenylate cyclase is an enzyme that catalyzes the conversion of ATP to 3′,5′-cyclic AMP (cAMP) and pyrophosphate. cAMP is a secondary messenger and a component of signal transduction pathways within a cell. Non-limiting examples of adenylate cyclase modulators include 9-cyclopentyladenine monomethanesulfonate, 2′,5′-dideoxyadenosine, 2′,5′-dideoxyadenosine 3′-triphosphate tetrasodium salt, (±)-2-(1H-benzimidazol-2-ylthio)propanoic acid 2-[(5-bromo-2-hydroxyphenyl)methylene]hydrazide (KH 7), and 5-(3-Bromophenyl)-5,11-dihydro-1,3-dimethyl-1H-indeno[2′,1′:5,6]pyrido[2,3-d]pyrimidine-2,4,6(3H)-trione (BPIPP).

Guanylate cyclase, also known as guanylyl cyclase or guanyl cyclase is an enzyme that catalyzes the conversion of guanosine triphosphate (GTP) to 3′,5′-cyclic guanosine monophosphate (cGMP) and pyrophosphate. cGMP is a second messenger in the signaling pathway that transmits the physiological response to peptide hormones and NO. Non-limiting examples of guanylate cyclase modulators include acenaphthenequinone, 6-anilinoquinoline-5,8-quinone, Rp-8-Bromo-β-phenyl-1,N2-ethenoguanosine 3′,5′-cyclic monophosphorothioate sodium salt, 4H-8-Bromo-1,2,4-oxadiazolo[3,4-δ]benz[o][1,4]oxazin-1-one, and 1H-[1,2,4]Oxadiazolo[4,3-a]quinoxalin-1-one.

Nitric oxide synthases are a family of enzymes catalyzing the production of NO from L-arginine. Nitric oxide synthase can exist in three different isoforms: (i) a soluble constitutively-expressed enzyme found in high concentrations in the brain (bNOS, nNOS, or NOS-1); (ii) a constitutively-expressed endothelial membrane-bound enzyme (eNOS or NOS-3); and (iii) an inducible enzyme (iNOS or NOS-2) associated with the cytotoxic function of macrophages. In mammals, the endothelial isoform of nitric oxide synthase is the primary signal generator in the control of vascular tone and insulin secretion. Non-limiting examples of eNOS modulators include aminoguanidine hemisulfate, diphenyleneiodonium chloride, 2-ethyl-2-thiopseudourea, L-N⁵-(1-Iminoethyl)ornithine dihydrochloride, S-methyl-L-thiocitruline dihydrochloride, N⁵-Nitro-L-arginine monoacetate, N⁵—Nitro-L-arginine (L-NNA), or nNOS inhibitor I.

Phosphodiesterases are a group of enzymes that break a phosphodiester bond. Cyclic nucleotide phosphodiesterases, such as phosphodiesterase 5, comprise a group of enzymes that degrade the phosphodiester bond in the second messenger molecules cAMP and cGMP.

Phosphodiesterase type 5 is most prominently expressed in the corpus cavernosum and retina. Non-limiting examples of inhibitors of phosphodiesterase 5 include sildenafil, vardenafil, tadalafil, avanafil, lodenafil, mirodenafil, udenafil, and zaprinast, and pharmaceutically-acceptable salts of the foregoing.

In some embodiments, the invention provides a method of increasing a level of a signaling molecule, the method comprising administering to a subject in need thereof a therapeutically effective amount of a Tie-2 activator.

Signaling molecules play a functional role in transmitting information in a physiological system. Non-limiting examples of classes of signaling molecules include lipids, phospholipids, amino acids, monoamines, proteins, glycoproteins, and gases. Messenger molecules can relay extracellular or intracellular signals. First messengers are often extracellular molecules, such as peptide hormones, growth factors, and neurotransmitters. Second messengers are intracellular signaling molecules that can trigger physiological changes, such as proliferation, differentiation, migration, survival, and apoptosis. Non-limiting examples of signaling or messenger molecules include nitric oxide, cyclic guanosine monophosphate, and cyclic adenosine monophosphate.

Cyclic guanosine monophosphate (cGMP) is cyclic nucleotide derived from guanosine triphosphate (GTP) and functions as a second messenger. cGMP relaxes smooth muscle tissue leading to vasodilation and increased blood flow. Cyclic adenosine monophosphate (cAMP) is a cyclic nucleotide derived from adenosine triphosphate (ATP) and functions as a second messenger. cAMP is a regulator of ion channels and hormone transport.

An increase in a local concentration of a small molecule can be about 10 nmol/L, about 50 nmol/L, about 100 nmol/L, about 150 nmol/L, about 200 nmol/L, about 250 nmol/L, about 300 nmol/L, about 350 nmol/L, about 400 nmol/L, about 450 nmol/L, about 500 nmol/L, about 550 nmol/L, about 600 nmol/L, about 650 nmol/L, about 700 nmol/L, about 750 nmol/L, about 800 nmol/L, about 850 nmol/L, about 900 nmol/L, about 950 nmol/L, about 1 μmol/L, about 2 μmol/L, about 3 μmol/L, about 4 μmol/L, about 5 μmol/L, about 6 μmol/L, about 7 μmol/L, about 8 μmol/L, about 9 μmol/L, or about 10 μmol/L.

An increase in a local concentration of a small molecule can be at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 300%, at least 400%, at least 500%, at least 600%, at least 700%, at least 800%, at least 900%, or at least 1000%. In some embodiments, the concentration of the small molecule increases by at least 1-fold, at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, or at least 15-fold.

### Pulmonary Hypertension.

A therapy of the disclosure can be used to treat pulmonary hypertension or pulmonary arterial hypertension. Pulmonary arterial hypertension is a condition in which pulmonary vascular cell proliferation and remodeling leads to elevated pulmonary arterial pressure, right ventricular hypertrophy, and ultimately, heart failure and death.

Pulmonary hypertension is elevated blood pressure in the lungs and right side of the heart. This condition results from obstruction of blood flow from the heart to the lungs through the pulmonary blood vessels. Elevated blood pressure causes pulmonary arteries to become narrowed and stiff, and in some cases, inflamed. To compensate for the reduced blood flow to the lungs, the heart must work harder to properly pump blood, making the organ become enlarged and weakened.

Unlike systemic blood pressure, which characterizes the force of blood moving through the blood vessels in your body, pulmonary blood pressure reflects the pressure that the heart exerts to pump blood from the heart through the arteries of the lungs. Normal pulmonary artery systolic pressure at rest is about 18 mmHg to 25 mmHg, with a mean pulmonary pressure ranging from 12 mmHg to 16 mmHg. Pulmonary hypertension refers to a mean pulmonary artery pressure at rest of greater than or equal to 25 mmHg, greater than 30 mmHg with exercise, or a pulmonary artery mean pressure greater than 20 mmHg. An increase in pulmonary vascular resistance or pulmonary blood flow results in pulmonary hypertension. Pulmonary hypertension can lead to a number of complications including congestive heart failure, heart enlargement, blood clots, arrhythmia, pulmonary hemorrhage, and hemoptysis.

Pulmonary hypertension is classified into five groups depending cause: 1) pulmonary arterial hypertension (PAH); 2) pulmonary hypertension due to left-sided heart disease; 3) pulmonary hypertension due to lung disease; 4) chronic thromboembolic pulmonary hypertension; and 5) pulmonary hypertension resulting from unclear mechanisms.

Group 1 PAH is the predominant form of pulmonary hypertension. PAH occurs when the arteries in the lung are narrowed, thicken, or stiff, resulting in restriction in blood flow.

Idiopathic PAH (IPAH) is PAH that occurs without a clear cause. Heritable PAH (HPAH) is linked to genetics. PAH can also develop as a result of drug or toxin exposure, HIV, portal hypertension, congenital heart disease, connective tissue disease, scleroderma, or lupus.

Eisenmenger syndrome is a type of congenital heart disease that causes pulmonary hypertension. This condition is most commonly caused by a large hole (shunt) in the heart between the ventricles, known as a ventricular septal defect. The shunt causes abnormal circulation of blood between the heart and lungs in that oxygenated blood flows back to the lungs instead to the rest of the body. As a result, the blood vessels in the lungs become stiff and narrow, increasing pressure in the pulmonary arteries.

Group 2 left-sided heart disease is often a result of coronary artery disease, high blood pressure, heart muscle damage, heart valve disease, and age. In Group 3 lung disease, pulmonary vessels are tightening in response to lung disease, such as chronic obstructive pulmonary disease (COPD), interstitial lung disease, asthma, and other lung diseases that cause low blood oxygen levels. Group 4 chronic thromboembolic pulmonary hypertension (CTEPH) occurs when the body is unable to dissolve a blood clot in the lungs. These blood clots create blockages in the pulmonary arteries and cause scar tissue to develop in the pulmonary blood vessels. In response, the heart must work harder to overcome the restriction of blood flow, resulting in elevated blood pressure. Group 5 PAH results from associated conditions, such as sarcoidosis, blood diseases, sickle cell anemia, chronic hemolytic anemia, and metabolic diseases.

Increase in venous pressure due to pulmonary hypertension can affect other organs in the body. For example, ocular complications can occur as a result of elevated venous pressure in the superior vena cava and ophthalmic veins that cause dilation of the ocular veins. Dilation of the ocular veins can result in congestion of the choroid, and leads to complications, such as ciliary detachment, central retinal vein occlusion, acute serous retinal detachment, macular edema, retinal neovascularization, choroidal effusions, chemosis, angle-closure glaucoma, transient myopia, and proptosis.

In some embodiments, a compound disclosed herein can change, modulate, increase, or decrease activity of a protein or enzyme that mediates endothelial function. The change can be at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 300%, at least 400%, at least 500%, at least 600%, at least 700%, at least 800%, at least 900%, or at least 1000% as compared to absence of administration of the compound.

In some embodiments, a compound disclosed herein can increase blood vessel dilation. The increase in blood vessel dilation can be at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 300%, at least 400%, at least 500%, at least 600%, at least 700%, at least 800%, at least 900%, or at least 1000% as compared to absence of administration of the compound. In some embodiments, the blood vessels dilate by at most 1-fold, at most 2-fold, at most 3-fold, at most 4-fold, or at most 5-fold more as compared to absence of administration of the compound.

### Assessment of Cardiovascular Health.

Hypertension can lead to problems with cardiovascular health. Cardiovascular health can be assessed by, for example, electrocardiogram (ECG) readings. ECGs record the electrical activity of the heart over time. During an ECG reading, an electrode placed on the skin can detect the electrophysiologic pattern of heart muscles as the muscles depolarize and repolarize during each heartbeat. An ECG readout is a graph of the measured voltage readings versus time.

Components of an ECG graph include the following. The P wave represents the depolarization of the atria. The QRS complex represents the depolarization of the ventricles. The T wave represents repolarization of the ventricles, P (the start of the P wave) represents the atrial systole contraction pulse. Q appears on the ECG graph as a downward deflection immediately preceding the ventricular contraction. R is the peak of the ventricular contraction. S is the downward deflection immediately after the ventricular contraction.

From the components of an ECG graph, several measurements can be made to assess potential abnormalities in the heartbeat of a subject. Measurements that can be used to identify potential abnormalities in the heartbeat include, for example, the following. The RR interval is the amount of time between the R peak of one heartbeat to R peak of the next heartbeat. The PR interval is the amount of time from the beginning of the P wave to the beginning of the QRS complex. QRS duration is the duration of the QRS complex. The QT interval is the time from the beginning of the QRS complex to the end of the T wave. The corrected QT interval corrects the QT interval based on heart rate by dividing the QT interval by the square root of the RR interval. Irregularities in an electrocardiogram can indicate an underlying cardiovascular disease.

### Animal Models.

One non-limiting example of an animal model useful for studying hypertension is the spontaneously hypertensive rat (SHR). The SHR rat strain was generated by selectively breeding Wistar Kyoto (WKY) rats with high blood pressure. SHR rats experience rising blood pressure starting at 5-6 weeks of age, and systolic pressures in SHR rats can reach values between about 180 mmHg and about 210 mmHg in adult animals. At ages of about 40 weeks to about 50 weeks, SHR rats develop characteristics of cardiac disease including, for example, vascular hypertrophy and cardiac hypertrophy.

A non-limiting example of an animal model useful for studying cardiovascular diseases frequently associated with hypertension is a canine model. Canine hearts have many similarities with the human heart on both the organ and cellular levels. Compared to other animal models frequently used for medical studies, such as mice, rats, and rabbits, the canine heart rate, body weight, and heart weight are more similar to those of humans.

Preclinical models of PAH can be used to assess the activity of a compound or combination therapy disclosed herein. An illustrative model of pulmonary hypertension is a rat model exposed to chronic hypoxia combined with vascular endothelial growth factor receptor (VEGF-R) blockage via the tyrosine kinase inhibitor, Semanixinib (SU5416), (Hy/Su). SU5416 can cause pulmonary artery endothelial cell (PAEC) apoptosis, emphysema, and an increase in pulmonary arterial pressure in rats. Exposure of SU5416-treated rats with hypoxia can trigger severe pulmonary hypertension. This model causes proliferative vascular remodeling accompanied by the formation of obstructive intimal lesions in the peripheral pulmonary arteries, which closely resemble plexiform lesions in human PAH.

As a compound or combination therapy disclosed herein can affect the VEGF/Tie-2 signaling pathway, an alternative preclinical model is the monocrotaline (MCT) lung injury rat model. Monocrotaline is a pyrrolizidine alkaloid that induces a pulmonary vascular syndrome in rats. Pulmonary vascular syndrome is characterized by proliferative pulmonary vasculitis, pulmonary hypertension, and cor pulmonale. A single, 60 mg/kg monocrotaline dose can be intraperitoneally administered in rats to induce profound PAH within 3-4 weeks. This model is characterized by pulmonary vascular endothelial injury followed by intense inflammation, leading to progressive pulmonary vascular remodeling, stiffening, increased pulmonary arterial pressure, and right ventricular hypertrophy.

Genetic analyses indicate that BMPR2 signaling in the endothelium is an initiating factor in PAH. A compound or combination therapy disclosed herein can be tested in a PAH mouse model generated by heterozygous knock-in of a human BMPR2 mutation, for example, R899X. Bmpr2-null mice, or mice homozygous for the R899X mutation, can be non-viable. However, heterozygous Bmpr2^{+/R899X} mice can develop normally and display reduced BMPR2 protein and mRNA. Bmpr2^{+/R899X} mice exhibit normal right ventricular systolic pressures (RVSP) at 3 months of age, but develop elevated RVSP by 6 months and enhanced muscularization of peripheral pulmonary arteries.

### Tie-2 Activation to Treat Ocular Conditions.

In some embodiments, a therapy of the disclosure can be used to decrease blood pressure in a subject and also treat, for example, ocular conditions. Non-limiting examples of ocular conditions that can be treated with a therapy disclosed herein include, for example, elevated intraocular pressure, ocular hypertension, glaucoma, primary open angle glaucoma, diabetic macular edema, age-related macular degeneration (wet form), choroidal neovascularization, diabetic retinopathy, ocular ischemia, retinal vein occlusion (central or branch), ocular trauma, surgery induced edema, surgery induced neovascularization, cystoid macular edema, proliferative retinopathy, ocular edema, and uveitis. Administration of a Tie-2 activator disclosed herein can treat ocular conditions via stabilization of the ocular vasculature.

### Tie-2 Activators.

Compounds disclosed herein can be effective as Tie-2 activators. The compounds can promote that activity, for example, by binding to or inhibiting HPTPβ. Such compounds can bind to HPTPβ, for example, by mimicking the binding mechanism of a native substrate, such as a phosphorylated compound. A compound can be a phosphate mimetic or bioisostere, for example, a sulfamic acid. The compound could also be derived from an amino acid building block or comprise an amino acid backbone for efficiency and economy of synthesis.

In some embodiments, a compound disclosed herein is a compound of the formula:

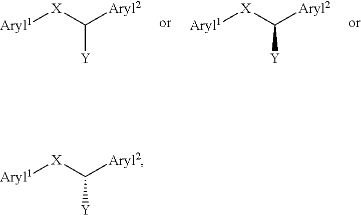

wherein: Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

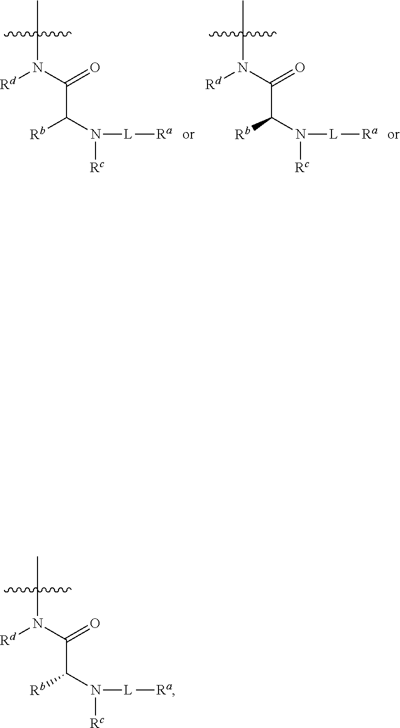

wherein:
L is alkylene, alkenylene, or alkynylene, any of which is substituted or unsubstituted, or together with the nitrogen atom to which L is bound forms an amide linkage, a carbamate linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L, R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L, R^{a}, R^{c}, and R^{d} forms a ring that is substituted or unsubstituted; R^{c} is H or alkyl which is substituted or unsubstituted, or together with any of L, R^{a}, R^{b}, and R^{d} forms a ring that is substituted or unsubstituted; R^{d} is H or alkyl which is substituted or unsubstituted, or together with any of L, R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted, or a pharmaceutically-acceptable salt, tautomer, or zwitterion thereof.

In some embodiments, Aryl¹ is substituted or unsubstituted phenyl, Aryl² is substituted or unsubstituted heteroaryl, and X is alkylene. In some embodiments, Aryl¹ is substituted phenyl, Aryl² is substituted heteroaryl, and X is methylene.

In some embodiments, a compound is of the formula:

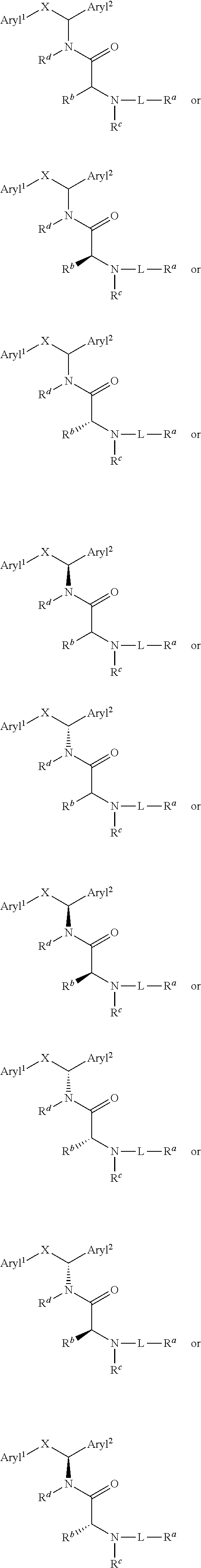

wherein Aryl¹ is para-substituted phenyl, Aryl² is substituted heteroaryl; X is methylene; L is alkylene, alkenylene, or alkynylene, any of which is substituted or unsubstituted, or together with the nitrogen atom to which L is bound forms an amide linkage, a carbamate linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl¹ is para-substituted phenyl; Aryl² is a substituted thiazole moiety; X is methylene; L together with the nitrogen atom to which L is bound forms a carbamate linkage; R^{a} is alkyl, which is substituted or unsubstituted; R^{b} is arylalkyl, which is substituted or unsubstituted; R^{c} is H; and R^{d} is H.

In some embodiments, Aryl² is:

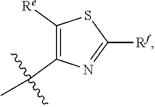

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted and R^{f} is alkyl, aryl, heterocyclyl, or heteroaryl, any of which is substituted or unsubstituted. In some embodiments, Aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl. In some embodiments, Aryl¹ is 4-phenylsulfamic acid; R^{a} is alkyl; which is substituted or unsubstituted; R^{b} is arylalkyl, which is substituted or unsubstituted; R^{e} is H; and R^{f} is alkyl.

In some embodiments, Aryl² is:

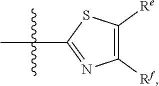

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted, R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted. In some embodiments, aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl.

In some embodiments, a substituted phenyl group is:

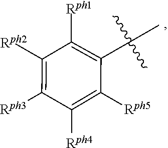

each of R^{ph1}, R^{ph2}, R^{ph3}, R^{ph4}, and R^{ph5} is independently H, OH, F, Cl, Br, I, CN, sulfamic acid, tosylate, mesylate, triflate, besylate, alkyl, alkenyl, alkynyl, an alkoxy group, a sulfhydryl group, a nitro group, an azido group, a sulfoxide group, a sulfone group, a sulfonamide group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl.

Illustrative compounds include the following:

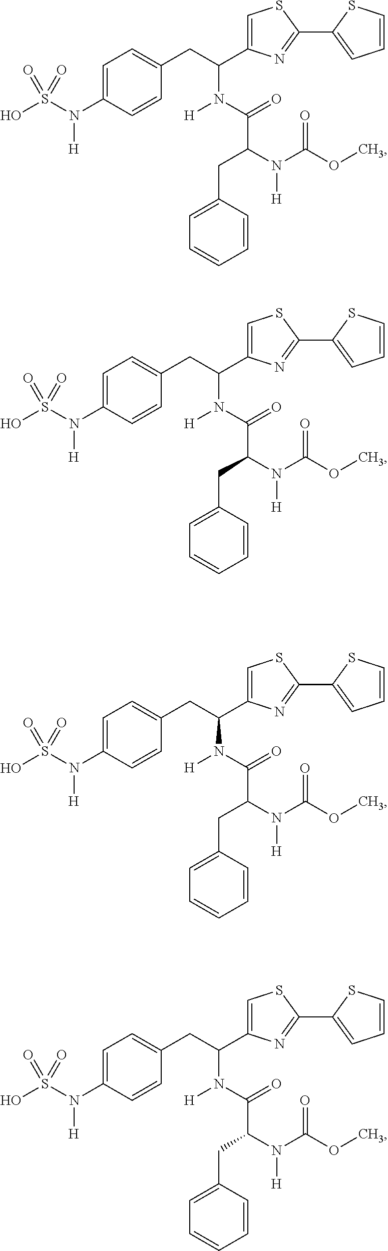 [SMILES: COC(=O)NC(Cc1ccccc1)C(=O)NC(Cc1ccc(NS(=O)(=O)O)cc1)c1csc(-c2cccs2)n1 ; COC(=O)N[C@@H](Cc1ccccc1)C(=O)NC(Cc1ccc(NS(=O)(=O)O)cc1)c1csc(-c2cccs2)n1 ; COC(=O)NC(Cc1ccccc1)C(=O)N[C@@H](Cc1ccc(NS(=O)(=O)O)cc1)c1csc(-c2cccs2)n1 ; COC(=O)N[C@H](Cc1ccccc1)C(=O)NC(Cc1ccc(NS(=O)(=O)O)cc1)c1csc(-c2cccs2)n1]

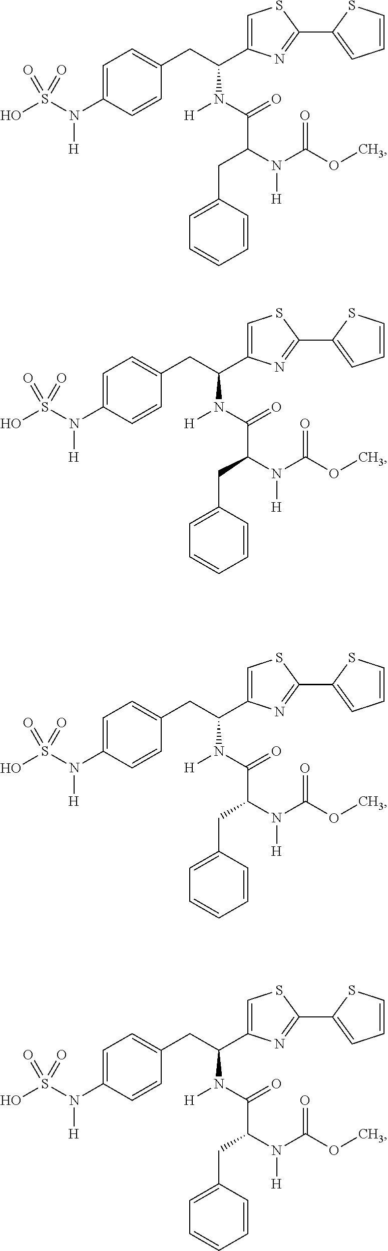 [SMILES: COC(=O)NC(Cc1ccccc1)C(=O)N[C@H](Cc1ccc(NS(=O)(=O)O)cc1)c1csc(-c2cccs2)n1 ; COC(=O)N[C@@H](Cc1ccccc1)C(=O)N[C@@H](Cc1ccc(NS(=O)(=O)O)cc1)c1csc(-c2cccs2)n1 ; COC(=O)N[C@H](Cc1ccccc1)C(=O)N[C@H](Cc1ccc(NS(=O)(=O)O)cc1)c1csc(-c2cccs2)n1 ; COC(=O)N[C@H](Cc1ccccc1)C(=O)N[C@@H](Cc1ccc(NS(=O)(=O)O)cc1)c1csc(-c2cccs2)n1]

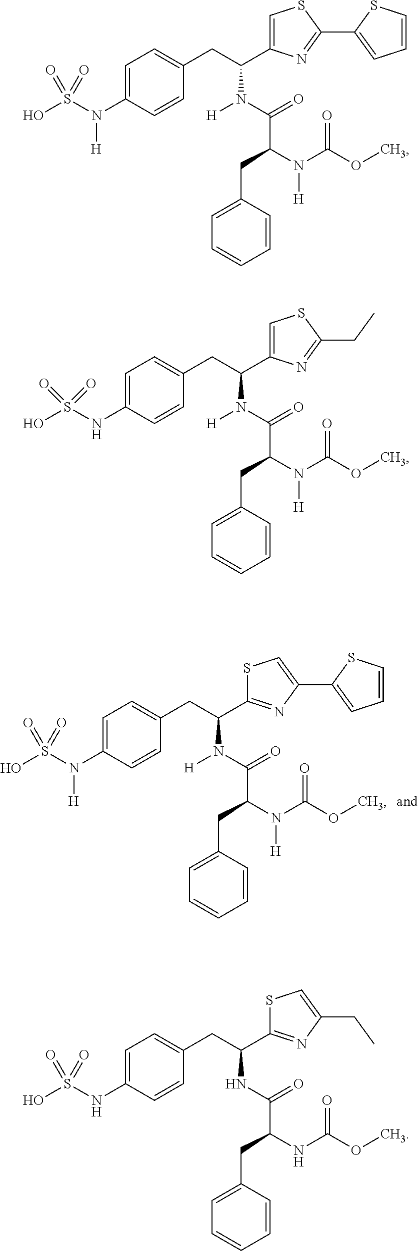 [SMILES: COC(=O)N[C@@H](Cc1ccccc1)C(=O)N[C@H](Cc1ccc(NS(=O)(=O)O)cc1)c1csc(-c2cccs2)n1 ; CCc1nc([C@H](Cc2ccc(NS(=O)(=O)O)cc2)NC(=O)[C@H](Cc2ccccc2)NC(=O)OC)cs1 ; COC(=O)N[C@@H](Cc1ccccc1)C(=O)N[C@@H](Cc1ccc(NS(=O)(=O)O)cc1)c1nc(-c2cccs2)cs1 ; "and" ; CCc1csc([C@H](Cc2ccc(NS(=O)(=O)O)cc2)NC(=O)[C@H](Cc2ccccc2)NC(=O)OC)n1]

### Optional Substituents for Chemical Groups.

Non-limiting examples of optional substituents include hydroxyl groups, sulfhydryl groups, halogens, amino groups, nitro groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, and ester groups.

Non-limiting examples of alkyl and alkylene groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

### Pharmaceutically-Acceptable Salts.

The method disclosed herein provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound disclosed herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound disclosed herein. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, piprazole, imidazole, or pyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a piprazole salt, an imidazole salt, or a pyrazine salt.

Acid addition salts can arise from the addition of an acid to a compound disclosed herein. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

A compound herein can be a salt of an acidic group, for example:

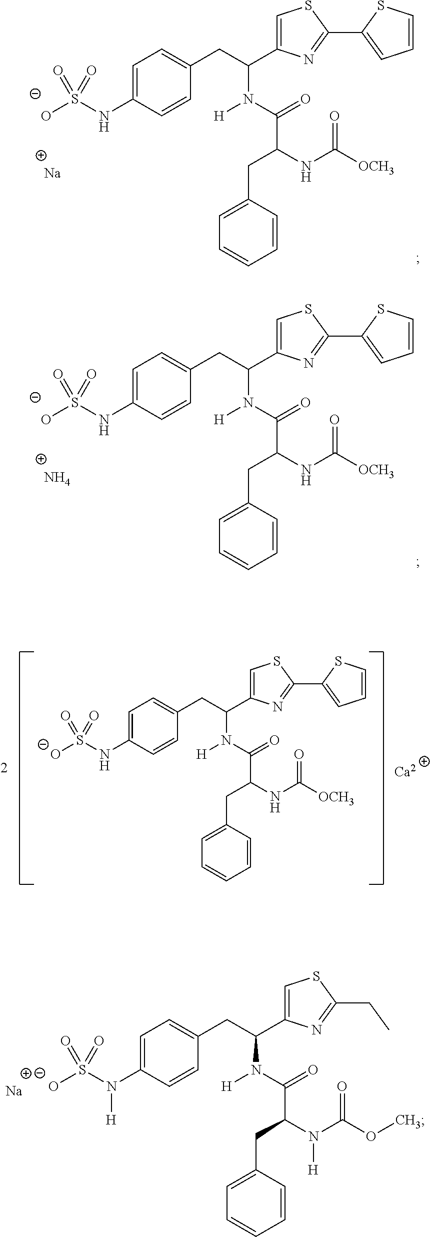

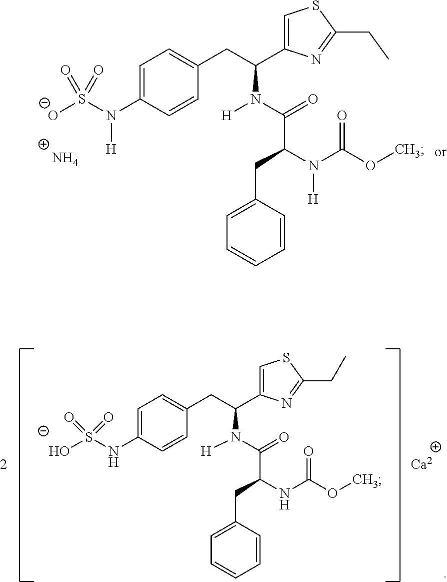

A compound herein can be a salt of a basic group formed from a strong acid, for example:

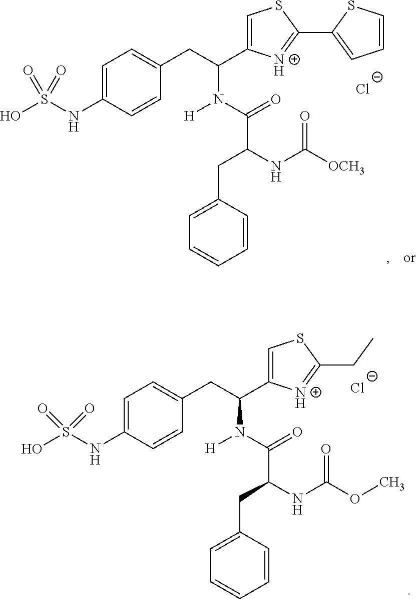 [SMILES: COC(=O)NC(Cc1ccccc1)C(=O)NC(Cc1ccc(NS(=O)(=O)O)cc1)c1csc(-c2cccs2)[nH+]1 ; [Cl-] ; CCc1[nH+]c([C@H](Cc2ccc(NS(=O)(=O)O)cc2)NC(=O)[C@H](Cc2ccccc2)NC(=O)OC)cs1 ; [Cl-]]

A compound herein can also exist in a zwitterionic form, for example:

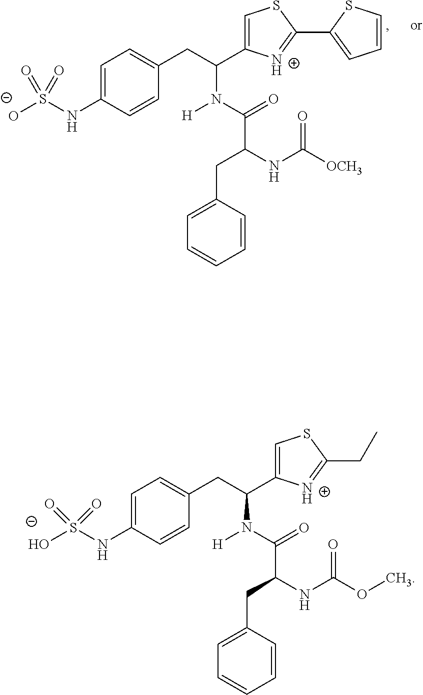 [SMILES: COC(=O)NC(Cc1ccccc1)C(=O)NC(Cc1ccc(NS(=O)(=O)[O-])cc1)c1csc(-c2cccs2)[nH+]1 ; CCc1[nH+]c([C@H](Cc2ccc(NS(=O)(=O)O)cc2)NC(=O)[C@H](Cc2ccccc2)NC(=O)OC)cs1]

### Formulations.

A pharmaceutical composition of the present disclosure can provide a therapeutically-effective amount of an activator of Tie-2.

The disclosed formulations can comprise one or more pharmaceutically-acceptable agents, which alone or in combination solubilize a compound herein or a pharmaceutically-acceptable salt thereof.

In some embodiments, a compound or pharmaceutically-acceptable salt thereof is present in a formulation in an amount of from about 0.1 mg/mL to about 100 mg/mL, from about 0.1 mg/mL to about 1 mg/mL, from about 0.1 mg/mL to about 5 mg/mL, from about 5 mg/mL to about 10 mg/mL, from about 10 mg/mL to about 15 mg/mL, from about 15 mg/mL to about 20 mg/mL, from about 20 mg/mL to about 25 mg/mL, from about 25 mg/mL to about 30 mg/mL, from about 30 mg/mL to about 35 mg/mL, from about 35 mg/mL to about 40 mg/mL, from about 40 mg/mL to about 45 mg/mL, about 45 mg/mL to about 50 mg/mL, from about 50 mg/mL to about 55 mg/mL, from about 55 mg/mL to about 60 mg/mL, from about 60 mg/mL to about 65 mg/mL, from about 65 mg/mL to about 70 mg/mL, from about 70 mg/mL to about 75 mg/mL, about 75 mg/mL to about 80 mg/mL, from about 80 mg/mL to about 85 mg/mL, from about 85 mg/mL to about 90 mg/mL, from about 90 mg/mL to about 95 mg/mL, or from about 95 mg/mL to about 100 mg/mL.

In some embodiments, a compound or pharmaceutically-acceptable salt thereof is present in a formulation in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL about 72 mg/mL, about 73 mg/mL, about 74 mg/mL, about 75 mg/mL, about 76 mg/mL, about 77 mg/mL, about 78 mg/mL, about 79 mg/mL, about 80 mg/mL, about 81 mg/mL about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/mL about 92 mg/mL, about 93 mg/mL, about 94 mg/mL, about 95 mg/mL, about 96 mg/mL, about 97 mg/mL, about 98 mg/mL, about 99 mg/mL, or about 100 mg/mL.

A formulation that is disclosed herein can be made more soluble by the addition of an additive or agent. The improvement of solubility of the formulation can increase by about 5%, about 10%, about 15%, about 20%, about 25%, about 30%, about 35%, about 40%, about 45%, about 50%, about 55%, about 60%, about 65%, about 70%, about 75% about 80%, about 85%, about 90%, about 95%, about 100%, about 110%, about 120%, about 130%, about 140%, about 150%, about 160%, about 170%, about 180%, about 190%, about 200%, about 225%, about 250%, about 275%, about 300%, about 325%, about 350%, about 375%, about 400%, about 450%, or about 500%.

A formulation disclosed herein can be stable for about 1 day, about 2 days, about 3 days, about 4 days, about 5 days, about 6 days, about 7 days, about 8 days, about 9 days, about 10 days, about 2 weeks, about 4 weeks, about 6 weeks, about 8 weeks, about 10 weeks, about 12 weeks, about 3 months, about 4 months, about 5 months, about 6 months, about 7 months, about 8 months, about 9 months, about 10 months, about 11 months, or about one year. A formulation disclosed herein can be stable, for example, at about 0° C., about 5° C., about 10° C., about 15° C., about 20° C., about 25° C., about 30° C., about 35° C., about 40° C., about 45° C., about 50° C., about 60° C., about 70° C., or about 80° C.

### Alcohols.

A non-limiting example of a solubilizing agent includes an organic solvent. Non-limiting examples of organic solvents include alcohols, for example, C₁-C₄ linear alkyl, C₃-C₄ branched alkyl, ethanol, ethylene glycol, glycerin, 2-hydroxypropanol, propylene glycol, maltitol, sorbitol, xylitol; substituted or unsubstituted aryl, and benzyl alcohol.

### Cyclodextrins.

Non-limiting examples of cyclodextrins include α-cyclodextrin, β-cyclodextrin, methyl β-cyclodextrin, 2-hydroxypropyl-β-cyclodextrin, sulfobutylether-β-cyclodextrin sodium salt, hydroxyethyl-β-cyclodextrin (HEβCD), heptakis(2,6-di-O-methyl)-β-cyclodextrin (DMβCD), 2-hydroxypropyl-β-cyclodextrin, γ-cyclodextrin, and 2-hydroxypropyl-γ-cyclodextrin (HPγCD). A cyclodextrin can possess a large cyclic structure with a channel passing through the center of the structure. The interior of the cyclodextrin can be hydrophobic, and interact favorably with hydrophobic molecules. The exterior of the cyclodextrin can be highly hydrophilic owing to the several hydroxyl groups exposed to bulk solvent. Capture of a hydrophobic molecule, such as a compound disclosed herein, in the channel of the cyclodextrin can result in the formation of a complex stabilized by non-covalent hydrophobic interactions. The complex can be soluble in water, and carry the captured hydrophobic molecule into the bulk solvent.

Formulations of the disclosure can comprise randomly methylated β-cyclodextrins (RAMEB or RMCD). The formulations of the disclosure can comprise RAMEB comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 methyl groups.

The disclosed solubilizing systems comprise 2-hydroxypropyl-β-cyclodextrin (HPβCD). 2-Hydroxypropyl-β-cyclodextrin [CAS No. 128446-35-5] is commercially available as Cavitron™. 2-Hydroxypropyl-β-cyclodextrin, also described as hydroxypropyl-β-cyclodextrin or HPβCD, can be represented by either of the following formulae:

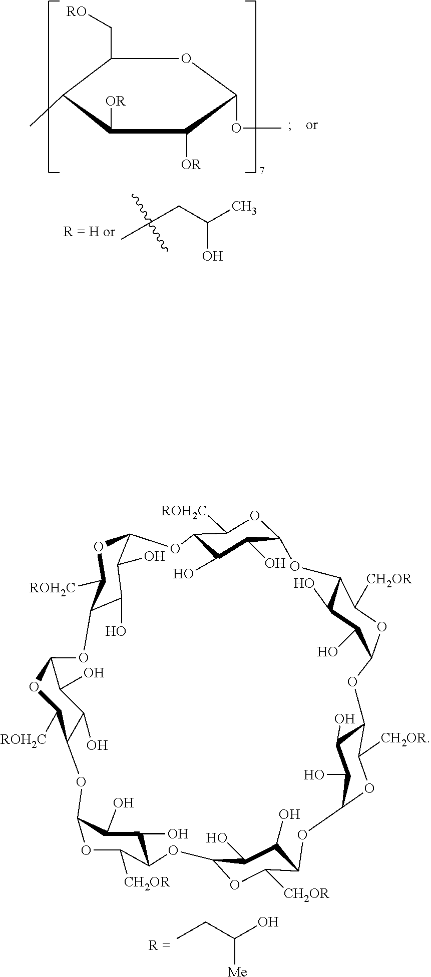

The average molecular weight of Cavitron™, is approximately 1396 Da, wherein the average degree of substitution is from about 0.5 to about 1.3 units of 2-hydroxypropyl per ring glucose unit.

The disclosed solubilizing systems comprise 2-hydroxypropyl-γ-cyclodextrin (HPγCD). 2-Hydroxypropyl-γ-cyclodextrin [CAS No. 128446-34-4], also known as hydroxypropyl-γ-cyclodextrin or HPGCD, can be represented by the following formula:

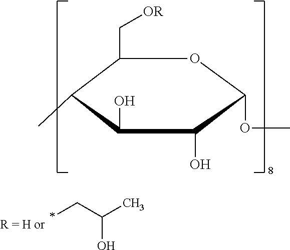

In one embodiment, a formulation disclosed herein can comprise a ratio of about 20 parts of a compound herein or a pharmaceutically-acceptable salt thereof to about 1 part solubilizing system (about 20:about 1), to about 1 part of the compound herein or a pharmaceutically-acceptable salt thereof to about 20 parts solubilizing system (about 1:about 20). For example, a formulation containing about 100 mg of a compound herein or a pharmaceutically-acceptable salt thereof can contain from about 5 mg to about 2000 mg of a solubilizing agent, such as a cyclodextrin. In another embodiment, the ratio can be based on number, or moles, or compound compared to number, or moles, of the solubilizing system.

The following are non-limiting examples of ratios of a compound herein and a solubilizing agent, such as a cyclodextrin. The following examples alternatively describe the ratio of a solubilizing agent, such as a cyclodextrin, and a compound herein. The ratio can be: about 20:about 1; about 19.9:about 1; about 19.8:about 1; about 19.7:about 1; about 19.6 about 1; about 19.5:about 1; about 19.4:about 1; about 19.3:about 1; about 19.2:about 1; about 19.1:about 1; about 19:about 1; about 18.9:about 1; about 18.8:about 1; about 18.7 about 1; about 18.6:about 1; about 18.5:about 1; about 18.4:about 1; about 18.3:about 1; about 18.2:about 1; about 18.1:about 1; about 18:about 1; about 17.9:about 1; about 17.8 about 1; about 17.7:about 1; about 17.6:about 1; about 17.5:about 1; about 17.4:about 1; about 17.3:about 1; about 17.2:about 1; about 17.1:about 1; about 17:about 1; about 16.9 about 1; about 16.8:about 1; about 16.7:about 1; about 16.6:about 1; about 16.5:about 1; about 16.4:about 1; about 16.3:about 1; about 16.2:about 1; about 16.1:about 1; about 16 about 1; about 15.9:about 1; about 15.8:about 1; about 15.7:about 1; about 15.6:about 1; about 15.5:about 1; about 15.4:about 1; about 15.3:about 1; about 15.2:about 1; about 15.1 about 1; about 15:about 1; about 14.9:about 1; about 14.8:about 1; about 14.7:about 1; about 14.6:about 1; about 14.5:about 1; about 14.4:about 1; about 14.3:about 1; about 14.2:about 1; about 14.1:about 1; about 14:about 1; about 13.9:about 1; about 13.8:about 1; about 13.7:about 1; about 13.6:about 1; about 13.5:about 1; about 13.4:about 1; about 13.3:about 1; about 13.2:about 1; about 13.1:about 1; about 13:about 1; about 12.9:about 1; about 12.8 about 1; about 12.7:about 1; about 12.6:about 1; about 12.5:about 1; about 12.4:about 1; about 12.3:about 1; about 12.2:about 1; about 12.1:about 1; about 12:about 1; about 11.9 about 1; about 11.8:about 1; about 11.7:about 1; about 11.6:about 1; about 11.5:about 1; about 11.4:about 1; about 11.3:about 1; about 11.2:about 1; about 11.1:about 1; about 11:about 1; about 10.9:about 1; about 10.8:about 1; about 10.7:about 1; about 10.6:about 1; about 10.5:about 1; about 10.4:about 1; about 10.3:about 1; about 10.2:about 1; about 10.1 about 1; about 10:about 1; about 9.9:about 1; about 9.8:about 1; about 9.7:about 1; about 9.6:about 1; about 9.5:about 1; about 9.4:about 1; about 9.3:about 1; about 9.2:about 1; about 9.1:about 1; about 9:about 1; about 8.9:about 1; about 8.8:about 1; about 8.7:about 1; about 8.6:about 1; about 8.5:about 1; about 8.4:about 1; about 8.3:about 1; about 8.2:about 1; about 8.1:about 1; about 8:about 1; about 7.9:about 1; about 7.8:about 1; about 7.7:about 1; about 7.6:about 1; about 7.5:about 1; about 7.4:about 1; about 7.3:about 1; about 7.2:about 1; about 7.1:about 1; about 7:about 1; about 6.9:about 1; about 6.8:about 1; about 6.7:about 1; about 6.6:about 1; about 6.5:about 1; about 6.4:about 1; about 6.3:about 1; about 6.2:about 1; about 6.1:about 1; about 6:about 1; about 5.9:about 1; about 5.8:about 1; about 5.7 about 1; about 5.6:about 1; about 5.5:about 1; about 5.4:about 1; about 5.3:about 1; about 5.2:about 1; about 5.1:about 1; about 5:about 1; about 4.9:about 1; about 4.8:about 1; about 4.7:about 1; about 4.6:about 1; about 4.5:about 1; about 4.4:about 1; about 4.3:about 1; about 4.2:about 1; about 4.1:about 1; about 4:about 1; about 3.9:about 1; about 3.8:about 1; about 3.7:about 1; about 3.6:about 1; about 3.5:about 1; about 3.4:about 1; about 3.3:about 1; about 3.2:about 1; about 3.1:about 1; about 3:about 1; about 2.9:about 1; about 2.8:about 1; about 2.7:about 1; about 2.6:about 1; about 2.5:about 1; about 2.4:about 1; about 2.3 about 1; about 2.2:about 1; about 2.1:about 1; about 2:about 1; about 1.9:about 1; about 1.8 about 1; about 1.7:about 1; about 1.6:about 1; about 1.5:about 1; about 1.4:about 1; about 1.3:about 1; about 1.2:about 1; about 1.1:about 1; or about 1:about 1.

### Polyvinylpyrrolidione.

Another non-limiting example of a solubilizing agent is polyvinylpyrrolidone (PVP), having the formula:

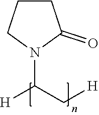

wherein the index n is from about 40 to about 200. PVP's can have an average molecular weight from about 5500 to about 28,000 g/mol. One non-limiting example is PVP-10, having an average molecular weight of approximately 10,000 g/mol.

### Polyakyleneoxides and Ethers Thereof.

Another non-limiting example of solubilizing agents includes polyalkyleneoxides, and polymers of alcohols or polyols. Polymers can be mixed, or contain a single monomeric repeat subunit. For example, polyethylene glycols (PEG) having an average molecular weight of from about 200 to about 20,000, for example, PEG 200, PEG 400, PEG 600, PEG 1000, PEG 1450, PEG 1500, PEG 4000, PEG 4600, and PEG 8000. In a same embodiment, a composition comprises one or more polyethylene glycols chosen from PEG 400, PEG 1000, PEG 1450, PEG 4600 and PEG 8000.

Other polyalkyleneoxides are polypropylene glycols having the formula:

HO[CH(CH₃)CH₂O]ₓH

wherein the index x represents the average number of propyleneoxy units in the polymer. The index x can be represented by a whole number or a fraction. For example, a polypropylene glycol having an average molecular weight of 8,000 g/mol (PEG 8000) can be represented by the formulae:

HO[CH(CH₃)CH₂O]₁₃₈H or HO[CH(CH₃)CH₂O]_{137.6}H

or the polypropylene glycol can be represented by the common, short hand notation: PEG 8000.

Another example of polypropylene glycols can have an average molecular weight from about 1,200 g/mol to about 20,000 g/mol, i.e., a polypropylene glycol having an average molecular weight of about 8,000 g/mol, for example, PEG 8000.

Another solubilizing agent is Polysorbate 80 (Tween™ 80), which is an oleate ester of sorbitol and its anhydrides copolymerized with approximately 20 moles of ethylene oxide for each mole of sorbitol and sorbitol anhydrides. Polysorbate 80 is made up of sorbitan mono-9-octadecanoate poly(oxy-1,2-ethandiyl) derivatives.

Solubilizing agents also include poloxamers having the formula:

HO(CH₂CH₂)_{y1}(CH₂CH₂CH₂O)_{y2}(CH₂CH₂O)_{y3}OH

which are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol.

### Excipients.

A pharmaceutical composition of a compound disclosed herein can be a combination of any pharmaceutical compounds described herein with other chemical components, such as carriers, stabilizers, diluents, dispersing agents, suspending agents, thickening agents, or excipients. The pharmaceutical composition facilitates administration of the compound to an organism. Pharmaceutical compositions can be administered in therapeutically-effective amounts as pharmaceutical compositions by various forms and routes including, for example, intravenous, intravitreal, intranasal, intratracheal, intrapulmonary, transmucosal, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, suspensions, and the like, for oral ingestion by a subject.

Pharmaceutical preparations for oral use can be obtained by mixing one or more solid excipient with one or more compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler such as lactose, binders such as starches, or lubricants such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

An active compound can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by means of iontophoretic patches. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a vapor, a mist, or a powder. Inhalation can occur through by nasal delivery, oral delivery, or both. Pharmaceutical compositions are conveniently delivered in the form of an aerosol spray presentation from pressurized packs, a nebulizer, or an atomizer, with the use of a suitable propellant, for example, dichlorodifluoromethane, trichlorofluoromethane, dichlorotetrafluoroethane, difluoroethane, carbon dioxide, nitrogen, oxygen, or other suitable gas. Nebulizers are available as jet nebulizers, ultrasonic nebulizers, or vibrating mesh nebulizers. Jet nebulizers operate by compressed air. Ultrasonic nebulizers use a piezoelectric transducer to create droplets from an open liquid reservoir. Vibrating mesh nebulizers use vibrating perforated membranes (mesh) actuated by an annular piezoelectric element. The holes in the membrane have a wide cross-sectional diameter on the liquid supply side and a narrow cross-section diameter on the side from where the droplets emerge.

In the case of a pressurized aerosol, the dosage unit can be determined by providing a valve to deliver a metered amount, for example, using a metered dose inhaler (MDI). Capsules and cartridges of, for example, gelatin for use in an inhaler or insufflator can be formulated to contain a powder mix of the compounds and a suitable powder base such as lactose or starch. Powder aerosols can be administered by dry powder inhalers (DPI). Aerosols can also be administered by a facemask interface, which can be a preferred delivery route for pediatric patients less than 5 years of age. Selection of a suitable inhalation device depends on favors, such as nature of the active compound and its formulation, the delivery site of interest, and pathophysiology of the lung.

Nasal or intranasal administration involves insufflation of compounds through the nose, which includes nasal drops and nasal sprays. This route of administration can result in local and/or systemic effects. Inhaler or insufflator devices can be used for nose-to-lung delivery of compounds described herein.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone and PEG. In suppository forms of the compositions, a low-melting point wax such as a mixture of fatty acid glycerides or cocoa butter, can be used.

In practicing a method of treatment or use provided herein, therapeutically-effective amounts of a compound described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, granulating, dragee-making, levigating, emulsifying, encapsulating, entrapping, or compression processes.

The pharmaceutical compositions can include at least one pharmaceutically-acceptable carrier, diluent, or excipient and compound described herein as free-base or pharmaceutically-acceptable salt form. The methods and pharmaceutical compositions described herein include the use of crystalline forms (also known as polymorphs), and active metabolites of these compounds having the same type of activity.

Methods for the preparation of compositions comprising a compound described herein include formulating a compound with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof.

A composition of a compound disclosed herein can be, for example, an immediate release form or a controlled release formulation. An immediate release formulation can be formulated to allow a compound to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

The disclosed compositions can optionally comprise from about 0.001% to about 0.005% weight by volume pharmaceutically-acceptable preservatives. One non-limiting example of a suitable preservative is benzyl alcohol.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A method disclosed herein includes, for example, administration of a Tie-2 activator, or a pharmaceutically-acceptable salt thereof, in combination with a pharmaceutically-acceptable carrier. The carrier can be selected to minimize any degradation of the active ingredient and to minimize any adverse side effects in the subject.

The Tie-2 activator or a pharmaceutically-acceptable salt thereof disclosed herein can be conveniently formulated into pharmaceutical compositions composed of one or more pharmaceutically-acceptable carriers. See e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compound described herein and which is incorporated by reference herein. Such pharmaceuticals can be standard carriers for administration of compositions to humans and non-humans, including solutions such as sterile water, saline, and buffered solutions at physiological pH. Other compositions can be administered according to standard procedures. For example, pharmaceutical compositions can also include one or more additional active ingredients such as antimicrobial agents, anti-inflammatory agents, and anesthetics.

Non-limiting examples of pharmaceutically-acceptable carriers include saline solution, Ringer's solution and dextrose solution. The pH of the solution can be from about 5 to about 8, and can be from about 7 to about 7.5. Further carriers include sustained release preparations such as semipermeable matrices of solid hydrophobic polymers containing the Tie-2 activator or a pharmaceutically-acceptable salt thereof, where the matrices are in the form of shaped articles, such as films, liposomes, microparticles, and microcapsules.

A method disclosed herein relates to administering the Tie-2 activator or a pharmaceutically-acceptable salt thereof as part of a pharmaceutical composition. In various embodiments, compositions of a compound disclosed herein can comprise a liquid comprising an active agent in solution, in suspension, or both. Liquid compositions can include gels. In one embodiment, the liquid composition is aqueous. Alternatively, the composition can take form of an ointment. In another embodiment, the composition is an in situ gellable aqueous composition. In some embodiments, the composition is an in situ gellable aqueous solution.

Pharmaceutical formulations can include additional carriers, as well as thickeners, diluents, buffers, preservatives, and surface active agents in addition to a compound disclosed herein. Pharmaceutical formulations can also include one or more additional active ingredients such as antimicrobial agents, anti-inflammatory agents, anesthetics, and the like.

An excipient can fill a role as simple and direct as being an inert filler, or an excipient as used herein can be part of a pH stabilizing system or coating to insure delivery of the ingredients safely to the stomach.

The Tie-2 activator or a pharmaceutically-acceptable salt thereof can also be present in liquids, emulsions, or suspensions for delivery of active therapeutic agents in aerosol form to cavities of the body such as the nose, throat, or bronchial passages. The ratio of Tie-2 activator or a pharmaceutically-acceptable salt thereof to the other compounding agents in these preparations can vary as the dosage form requires.

Depending on the intended mode of administration, the pharmaceutical compositions administered as part of a method disclosed herein can be in the form of solid, semi-solid or liquid dosage forms, such as, for example, tablets, suppositories, pills, capsules, powders, liquids, suspensions, lotions, creams, gels, for example, in unit dosage form suitable for single administration of a precise dosage. The compositions can contain, as noted above, an effective amount of the Tie-2 activator or a pharmaceutically-acceptable salt thereof in combination with a pharmaceutically-acceptable carrier and, in addition, can include other medicinal agents, pharmaceutical agents, carriers, adjuvants, diluents, etc.

For solid compositions, nontoxic solid carriers include, for example, pharmaceutical grades of mannitol, lactose, starch, magnesium stearate, sodium saccharin, talc, cellulose, glucose, sucrose, and magnesium carbonate. In one embodiment, a composition comprising the Tie-2 activator or a pharmaceutically-acceptable salt thereof in an amount of approximately 4 mg per 0.1 mL liquid is prepared. The liquid phase comprises sterile water and an appropriate amount of a saccharide or polysaccharide.

### Pharmaceutical Compositions.

Pharmaceutical compositions containing a compound described herein can be administered for prophylactic or therapeutic treatments. Compositions can contain any number of active agents. In therapeutic applications, the compositions can be administered to a subject already suffering from a disease or condition, in an amount sufficient to cure or at least partially arrest the symptoms of the disease or condition, or to cure, heal, improve, reduce, lessen or ameliorate the disease or condition. A compound can also be administered to lessen or reduce a likelihood of developing, contracting, or worsening a condition. Amounts effective for this use can vary based on the severity and course of the disease or condition, previous therapy, the subject's health status, weight, response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills or injections. The compounds can be packed together or separately, in a single package or in a plurality of packages. One or all of the therapeutic agents can be given in multiple doses. If not simultaneous, the timing between the multiple doses can vary.

Compounds and compositions of the present disclosure can be packaged as a kit. In some embodiments, the present disclosure provides a kit comprising a compound disclosed herein, or a pharmaceutically-acceptable salt thereof, and written instructions on use of the kit in the treatment of a condition described herein. In some embodiments, the present disclosure provides a kit comprising a compound disclosed herein, or a pharmaceutically-acceptable salt thereof, an antibody, and written instructions on use of the kit in the treatment of a condition described herein.

### Administration and Dosage.

A compound disclosed herein can be administered via subcutaneous injection. The volume of an injection can be about 0.1 mL, about 0.2 mL, about 0.3 mL, about 0.4 mL, about 0.5 mL, about 0.6 mL, about 0.7 mL, about 0.8 mL, about 0.9 mL, about 1 mL, about 1.1 mL, about 1.2 mL, about 1.3 mL, about 1.4 mL, about 1.5 mL, about 1.6 mL, about 1.7 mL, about 1.8 mL, about 1.9 mL, about 2 mL, about 2.1 mL, about 2.2 mL, about 2.3 mL, about 2.4 mL, about 2.5 mL, about 2.6 mL, about 2.7 mL, about 2.8 mL, about 2.9 mL, or about 3 mL. The individual dose administered to a subject can be about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg.

A compound disclosed herein can be administered as eye drops. The average volume of each drop administered to a subject can be about 5 μl, about 10 μl, about 15 μl, about 20 μl, about 30 μl, about 40 μl, about 50 μl, about 60 μl, about 70 μl, about 80 μl, about 90 μl, or about 100 μl. The eye drops can contain about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% of a compound described herein. The drops can contain about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 120 mg/mL, about 140 mg/mL, about 160 mg/mL, about 180 mg/mL, or about 200 mg/mL of a compound described herein. The individual dose administered to a subject can be about 0.5 μg, about 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1 mg, about 1.1 mg, about 1.2 mg, 1.3 mg, about 1.4 mg, about 1.5 mg, about 1.6 mg, about 1.7 mg, about 1.8 mg, about 1.9 mg, or about 2 mg of a compound described herein. In some embodiments, more than one drop can be administered to an eye either at one time or at multiple times throughout the day.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A Tie-2 activator described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

A Tie-2 activator described herein can be present in a composition in an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, or about 300 mg.

A Tie-2 activator described herein can be present in a composition in an amount of about 0.5 μg, about 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1 mg, about 1.1 mg, about 1.2 mg, 1.3 mg, about 1.4 mg, about 1.5 mg, about 1.6 mg, about 1.7 mg, about 1.8 mg, about 1.9 mg, or about 2 mg.

A compound described herein can be administered before, during, or after the occurrence of a disease or condition, and the timing of administering the composition containing a compound can vary. For example, a compound can be used as a prophylactic and can be administered continuously to subjects with a propensity to conditions or diseases in order to lessen or reduce a likelihood of the occurrence of the disease or condition. A compound and composition can be administered to a subject during or as soon as possible after the onset of the symptoms. The administration of a compound can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years. The length of treatment can vary for each subject.

Treatment of Subjects with a Tie-2 Activator.

Disclosed herein is a method for treating a subject afflicted with, for example, elevated blood pressure, stage 1 hypertension, stage 2 hypertension, an ongoing hypertensive crisis, or pulmonary hypertension with an activator of Tie-2. The subject can be a human. Treatment can include treating a human in a clinical trial. A treatment can comprise administering to a subject a pharmaceutical composition comprising one or more of the activators of Tie-2 described throughout the disclosure. A treatment can comprise administrating to a subject a therapy that promotes the phosphorylation of a Tie-2 molecule.

In some embodiments, the method disclosed herein provides a Tie-2 activator for use in treatment of indications disclosed herein. In some embodiments, the method disclosed herein provides a Tie-2 activator for use in the manufacture of a medicament for the treatment of indications disclosed herein. In some embodiments, the method disclosed herein provides a Tie-2 activator for use singly or in combination with one or more therapeutic agents as components of mixtures. For example, a Tie-2 activator of the disclosure can be co-formulated or co-administered with an antibody, for example, an anti-VEGF agent. An anti-VEGF agent can be a compound, an antibody, or an antibody fragment, variant, or derivative thereof. Non-limiting examples of anti-VEGF agents include bevacizumab (Avastin®), ranibizumab (Lucentis®), and aflibercept (Eylea®). In some embodiments, a Tie-2 activator of the disclosure can be co-formulated, or co-administered, with a non-inflammatory agent, for example, a VEGF modulating agent. Non-limiting examples of a VEGF-modulating agent include, for example, dexamethasone, fluocinolone, and triamcinolone. In some embodiments, a compound described herein can be used before, during, or after treatment with an anti-VEGF, or VEGF modulating, agent.

In some embodiments, subjects treated with a method disclosed herein have a cardiovascular disorder disclosed herein. In some embodiments, a method disclosed herein provides a Tie-2 activator for use alone or in combination with one or more therapeutic agents, either separately or as components of mixtures. For example, a Tie-2 activator of the disclosure can be co-formulated or co-administered with an agent used to treat a cardiovascular disorder. Non-limiting examples of agents that can be used to treat a cardiovascular disorder include, for example, statins such as atorvastatin, simvastatin, pravastatin, and lovastatin; blood thinners such as clopidogrel and aspirin; cholesterol medication such as gemfibrozil, ezetimibe, and fenofibrate; beta blockers such as atenolol and metoprolol, heart medications such as nitroglycerin and isorbide; calcium channel blockers such as amlodipine; angiotensin-converting enzyme (ACE) inhibitors; angiotensin II receptor blockers; diuretics; vasodilator agents; positive inotropes; and aldosterone antagonists. In some embodiments, a compound described herein can be used before, during, or after treatment with an agent used to treat a cardiovascular disorder.

In some embodiments, the hypertension treated with a method disclosed herein is primary hypertension. In some embodiments, the hypertension treated with a method disclosed herein is secondary hypertension. In some embodiments, a method disclosed herein can treat elevated blood pressure caused by nonspecific genetic and lifestyle factors. In some embodiments, a method disclosed herein can treat elevated blood pressure caused by identifiable causes. In some embodiments, a method disclosed herein can treat hypertensive crises caused by nonspecific genetic and lifestyle factors. In some embodiments, a method disclosed herein can treat hypertensive crises due to identifiable causes.

Non-limiting examples of possible subjects for administration include the following. Subjects can be humans, non-human primates such as chimpanzees, and other apes and monkey species; farm animals such as cattle, horses, sheep, goats, and swine; domestic animals such as rabbits, dogs, and cats; and laboratory animals including rats, mice, and guinea pigs. A subject can be of any age. Subjects can be, for example, elderly adults, adults, adolescents, pre-adolescents, children, toddlers, infants, and neonates.

Some conditions can lead to an increase in the levels of Ang-2, altering the ratio of Ang-1/Ang-2 in circulation. In some aspects, a therapy can improve the outcome of a disease state, including the indications disclosed herein, by altering the ratio of Ang-1/Ang-2 in circulation. A therapy can provide an Ang-1/Ang-2 ratio or an Ang-2/Ang-1 ratio of about 1:about 1, about 2 about 1, about 3:about 1, about 4:about 1, about 5:about 1, about 6:about 1, about 7:about 1, about 8:about 1, about 9:about 1, or about 10:about 1.

### Combination Therapies.

A Tie-2 activator described herein can be co-formulated or co-administered with one or more additional therapeutic agents for the treatment of hypertension or pulmonary hypertension. The combination can be administered consecutively, simultaneously, in a single dosage form, or in separate dosage forms. Non-limiting examples of additional therapeutic agents include vasodilators, calcium channel blockers, prostanoids, endothelin receptor antagonists (ERA), phosphodiesterase type 5 inhibitors, anticoagulants, blood thinners, angiotensin-converting enzyme (ACE) inhibitors, beta-blockers, mineralocorticoid antagonists, guanylate cyclase stimulants, diuretics, warfarin, ifedipine, diltiazem, ambrisentan, bosentan, macitentan, sitaxsentan, sildenafil, sildenafil citrate, tadalafil, vardenafil, riociguat, oxygen, digoxin, agents that interact with any of adenylate cyclase, guanylate cyclase, nitric oxide synthetase, and a phosphodiesterase, such as phosphodiesterase 5, as a modulator, an agonist, an antagonist, an activator, or an inhibitor.

Non-limiting examples of additional therapeutic agents include 9-cyclopentyladenine monomethanesulfonate, 2′,5′-dideoxyadenosine, 2′,5′-dideoxyadenosine 3′-triphosphate tetrasodium salt, (±)-2-(1H-benzimidazol-2-ylthio)propanoic acid 2-[(5-bromo-2-hydroxyphenyl)methylene]hydrazide (KH 7), 5-(3-Bromophenyl)-5,11-dihydro-1,3-dimethyl-1H-indeno[2′,1′:5,6]pyrido[2,3-d]pyrimidine-2,4,6(3H)-trione (BPIPP), acenaphthenequinone, 6-anilinoquinoline-5,8-quinone, Rp-8-Bromo-β-phenyl-1,N2-ethenoguanosine 3′,5′-cyclic monophosphorothioate sodium salt, 4H-8-Bromo-1,2,4-oxadiazolo[3,4-δ]benz[β][1,4]oxazin-1-one, 1H-[1,2,4]Oxadiazolo[4,3-α]quinoxalin-1-one, aminoguanidinehemisulfate, diphenyleneiodonium chloride, 2-ethyl-2-thiopseudourea, L-N⁵-(1-Iminoethyl)ornithine dihydrochloride, S-methyl-L-thiocitruline dihydrochloride, N^{G}—Nitro-L-arginine monoacetate, N^{G}—Nitro-L-arginine (L-NNA), or nNOS inhibitor I.

Calcium channel blockers work by relaxing the muscles of the arterial wall, thereby enlarging the arteries to reduce blood pressure. Vasodilators also function to enlarge the blood vessels, restoring circulation of blood. Non-limiting examples of vasodilators include iloprost, treprostinil, epoprostenol (prostacyclin), and selexipag.

Diuretics are therapies that remove excess fluid from the body by increasing the production and flow of urine. PAH can cause abnormal fluid retention due to heart strain, hypoxemia, and a hormonal imbalance between the heart, lungs, and kidneys. Symptoms of fluid retention include swelling (edema) in the lungs, legs, feet, abdomen, and other parts of the body. Non-limiting examples of diuretics include furosemide, bumetidine, amiloride, spironolactone, and torsamide.

Patients with PAH can have low oxygen levels in the blood. Oxygen therapy can help restore normal blood oxygen levels and relieve symptoms of PAH. Continuous oxygen administration is an illustrative therapy recommended for patients with Group 3 PH (pulmonary hypertension due to lung disease).

Severe PAH can lead to congestive heart failure, which can require medications that improve the pumping efficiency of the heart. An illustrative treatment for heart failure is a triple therapy of ACE inhibitor, beta-blocker, and mineralocorticoid antagonists. Digoxin is an alternative therapy for treatment of heart failure, which works by inhibiting sodium/potassium ATPase (Na+/K+ATPase) in the myocardium, which causes accumulation of intracellular Ca²⁺. This effect leads to increased contractility (or contracting strength) of the heart without increasing energy expenditure, which improves the efficiency of each heartbeat.

### Pharmacodynamic and Pharmacokinetic Parameters.

Pharmacokinetic and pharmacodynamic data can be obtained by various experimental techniques. Appropriate pharmacokinetic and pharmacodynamic profile components describing a particular composition can vary due to variations in the metabolism of an activator of Tie-2 in different subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 15 subjects, 20 subjects, 25 subjects, 30 subjects, 35 subjects, or more. The mean is determined by calculating the average of all subject's measurements for each parameter measured.

A therapy can be used to inhibit a specific biological or biochemical function at a lower dosage. A dose can be modulated to achieve a desired pharmacokinetic or pharmacodynamics profile, such as a desired or effective blood profile, as described herein. The half maximum inhibitory concentration (IC₅₀) is a measure of the effectiveness of a substance in inhibiting a specific biological or biochemical function. This quantitative measure indicates how much of a particular drug or compound is needed to inhibit a given biological process, such as the activity of HPTPβ by half Combination drug treatments can present lower IC₅₀ values as compared to monotherapies.

The outcome of treating a human subject with a therapy can be measured by calculating pharmacodynamic and pharmacokinetic parameters. Non-limiting examples of pharmacodynamic and pharmacokinetic parameters that can be used to determine the effect of treatment of a subject with a therapy of the disclosure include: a) the amount of drug administered, which can be represented as a dose D; b) the dosing interval, which can be represented as τ; c) the apparent volume in which a drug is distributed, which can be represented as a volume of distribution V_{d}, where V_{d}=D/C₀; d) the amount of drug in a given volume of tissue, which can be represented as concentration C₀ or Cₛₛ, where C₀ or Cₛₛ=D/Vd; e) the half-life of a drug t_{1/2}, where t_{1/2}=ln(2)/kₑ; f) the rate at which a drug is removed from the body kₑ, where kₑ=ln(2)/(t)_{1/2}=CL/V_{d}; g) the rate of infusion required to balance the equation Kᵢₙ, where Kᵢₙ=Cₛₛ,CL; h) the integral of the concentration-time curve after administration of a single dose, which can be represented as AUC_{0-∞}, wherein f₀^{∞} C dt, or in steady-state, which can be represented as AUCτ, ₛₛ, wherein ∞ₜ^{t+π} C dt; i) the volume of tissue cleared of the drug per unit time, which can be represented as CL (clearance), wherein CL=V_{d}·kₑ=D/AUC; j) the systemically available fraction of a drug, which can be represented as f, where

$$f = \frac{AUCpo \cdot Div}{AUCiv \cdot Dpo} ;$$

k) the peak tissue concentration of a drug after administration Cₘₐₓ; 1) the time taken by a drug to reach Cₘₐₓ, tₘₐₓ; m) the lowest concentration that a drug reaches before the next dose is administered Cₘᵢₙ; and n) the peak trough fluctuation within one dosing interval at steady state, which can be represented as %

$$P T F = 100 \cdot \frac{( Cmax , ss - Cmin , ss )}{Cav , ss}$$

where

$${C}_{av , ss} = \frac{A U C τ , ss}{τ} .$$

The pharmacokinetics parameters can be any parameters suitable for describing the tissue concentration profiles of a therapy of the disclosure. For example, the pharmacokinetics profile can be obtained at a time after dosing of, for example, about zero minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about zero hours, about 0.5 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, or about 24 hours.

The pharmacokinetic parameters can be any parameters suitable for describing a small molecule activator of Tie-2. The Cₘₐₓ can be, for example, not less than about 1 ng/mL; not less than about 2 ng/mL; not less than about 3 ng/mL; not less than about 4 ng/mL; not less than about 5 ng/mL; not less than about 6 ng/mL; not less than about 7 ng/mL; not less than about 8 ng/mL; not less than about 9 ng/mL; not less than about 10 ng/mL; not less than about 15 ng/mL; not less than about 20 ng/mL; not less than about 25 ng/mL; not less than about 50 ng/mL; not less than about 75 ng/mL; not less than about 100 ng/mL; not less than about 200 ng/mL; not less than about 300 ng/mL; not less than about 400 ng/mL; not less than about 500 ng/mL; not less than about 600 ng/mL; not less than about 700 ng/mL; not less than about 800 ng/mL; not less than about 900 ng/mL; not less than about 1000 ng/mL; not less than about 1250 ng/mL; not less than about 1500 ng/mL; not less than about 1750 ng/mL; not less than about 2000 ng/mL; or any other Cₘₐₓ appropriate for describing a pharmacokinetic profile of an activator of Tie-2 described herein. The Cₘₐₓ can be, for example, about 1 ng/mL to about 5,000 ng/mL; about 1 ng/mL to about 4,500 ng/mL; about 1 ng/mL to about 4,000 ng/mL; about 1 ng/mL to about 3,500 ng/mL; about 1 ng/mL to about 3,000 ng/mL; about 1 ng/mL to about 2,500 ng/mL; about 1 ng/mL to about 2,000 ng/mL; about 1 ng/mL to about 1,500 ng/mL; about 1 ng/mL to about 1,000 ng/mL; about 1 ng/mL to about 900 ng/mL; about 1 ng/mL to about 800 ng/mL; about 1 ng/mL to about 700 ng/mL; about 1 ng/mL to about 600 ng/mL; about 1 ng/mL to about 500 ng/mL; about 1 ng/mL to about 450 ng/mL; about 1 ng/mL to about 400 ng/mL; about 1 ng/mL to about 350 ng/mL; about 1 ng/mL to about 300 ng/mL; about 1 ng/mL to about 250 ng/mL; about 1 ng/mL to about 200 ng/mL; about 1 ng/mL to about 150 ng/mL; about 1 ng/mL to about 125 ng/mL; about 1 ng/mL to about 100 ng/mL; about 1 ng/mL to about 90 ng/mL; about 1 ng/mL to about 80 ng/mL; about 1 ng/mL to about 70 ng/mL; about 1 ng/mL to about 60 ng/mL; about 1 ng/mL to about 50 ng/mL; about 1 ng/mL to about 40 ng/mL; about 1 ng/mL to about 30 ng/mL; about 1 ng/mL to about 20 ng/mL; about 1 ng/mL to about 10 ng/mL; about 1 ng/mL to about 5 ng/mL; about 10 ng/mL to about 4,000 ng/mL; about 10 ng/mL to about 3,000 ng/mL; about 10 ng/mL to about 2,000 ng/mL; about 10 ng/mL to about 1,500 ng/mL; about 10 ng/mL to about 1,000 ng/mL; about 10 ng/mL to about 900 ng/mL; about 10 ng/mL to about 800 ng/mL; about 10 ng/mL to about 700 ng/mL; about 10 ng/mL to about 600 ng/mL; about 10 ng/mL to about 500 ng/mL; about 10 ng/mL to about 400 ng/mL; about 10 ng/mL to about 300 ng/mL; about 10 ng/mL to about 200 ng/mL; about 10 ng/mL to about 100 ng/mL; about 10 ng/mL to about 50 ng/mL; about 25 ng/mL to about 500 ng/mL; about 25 ng/mL to about 100 ng/mL; about 50 ng/mL to about 500 ng/mL; about 50 ng/mL to about 100 ng/mL; about 100 ng/mL to about 500 ng/mL; about 100 ng/mL to about 400 ng/mL; about 100 ng/mL to about 300 ng/mL; or about 100 ng/mL to about 200 ng/mL.

The Tₘₐₓ of an activator of Tie-2 described herein can be, for example, not greater than about 0.1 hours, about 0.2 hours, about 0.3 hours, about 0.4 hours, about 0.5 hours, not greater than about 1 hours, not greater than about 1.5 hours, not greater than about 2 hours, not greater than about 2.5 hours, not greater than about 3 hours, not greater than about 3.5 hours, not greater than about 4 hours, not greater than about 4.5 hours, not greater than about 5 hours, or any other Tₘₐₓ appropriate for describing a pharmacokinetic profile of an activator of Tie-2 described herein. The Tₘₐₓ can be, for example, about 0.1 hours to about 24 hours; about 0.1 hours to about 0.5 hours; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; or about 23.5 hours to about 24 hours.

The AUC_{(0-inf)} or AUC₍ₗₐₛₜ₎ of an activator of Tie-2 described herein can be, for example, not less than about 1 ng·hr/mL, not less than about 5 ng·hr/mL, not less than about 10 ng·hr/mL, not less than about 20 ng·hr/mL, not less than about 30 ng·hr/mL, not less than about 40 ng·hr/mL, not less than about 50 ng·hr/mL, not less than about 100 ng·hr/mL, not less than about 150 ng·hr/mL, not less than about 200 ng·hr/mL, not less than about 250 ng·hr/mL, not less than about 300 ng·hr/mL, not less than about 350 ng·hr/mL, not less than about 400 ng·hr/mL, not less than about 450 ng·hr/mL, not less than about 500 ng·hr/mL, not less than about 600 ng·hr/mL, not less than about 700 ng·hr/mL, not less than about 800 ng·hr/mL, not less than about 900 ng·hr/mL, not less than about 1000 ng·hr/mL, not less than about 1250 ng·hr/mL, not less than about 1500 ng·hr/mL, not less than about 1750 ng·hr/mL, not less than about 2000 ng·hr/mL, not less than about 2500 ng·hr/mL, not less than about 3000 ng·hr/mL, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, not less than about 10,000 ng·hr/mL, or any other AUC_{(0-inf)} appropriate for describing a pharmacokinetic profile of a compound described herein. The AUC_{(0-inf)} of an activator of Tie-2 can be, for example, about 1 ng·hr/mL to about 10,000 ng·hr/mL; about 1 ng·hr/mL to about 10 ng·hr/mL; about 10 ng·hr/mL to about 25 ng·hr/mL; about 25 ng·hr/mL to about 50 ng·hr/mL; about 50 ng·hr/mL to about 100 ng·hr/mL; about 100 ng·hr/mL to about 200 ng·hr/mL; about 200 ng·hr/mL to about 300 ng·hr/mL; about 300 ng·hr/mL to about 400 ng·hr/mL; about 400 ng·hr/mL to about 500 ng·hr/mL; about 500 ng·hr/mL to about 600 ng·hr/mL; about 600 ng·hr/mL to about 700 ng·hr/mL; about 700 ng·hr/mL to about 800 ng·hr/mL; about 800 ng·hr/mL to about 900 ng·hr/mL; about 900 ng·hr/mL to about 1,000 ng·hr/mL; about 1,000 ng·hr/mL to about 1,250 ng·hr/mL; about 1,250 ng·hr/mL to about 1,500 ng·hr/mL; about 1,500 ng·hr/mL to about 1,750 ng·hr/mL; about 1,750 ng·hr/mL to about 2,000 ng·hr/mL; about 2,000 ng·hr/mL to about 2,500 ng·hr/mL; about 2,500 ng·hr/mL to about 3,000 ng·hr/mL; about 3,000 ng·hr/mL to about 3,500 ng·hr/mL; about 3,500 ng·hr/mL to about 4,000 ng·hr/mL; about 4,000 ng·hr/mL to about 4,500 ng·hr/mL; about 4,500 ng·hr/mL to about 5,000 ng·hr/mL; about 5,000 ng·hr/mL to about 5,500 ng·hr/mL; about 5,500 ng·hr/mL to about 6,000 ng·hr/mL; about 6,000 ng·hr/mL to about 6,500 ng·hr/mL; about 6,500 ng·hr/mL to about 7,000 ng·hr/mL; about 7,000 ng·hr/mL to about 7,500 ng·hr/mL; about 7,500 ng·hr/mL to about 8,000 ng·hr/mL; about 8,000 ng·hr/mL to about 8,500 ng·hr/mL; about 8,500 ng·hr/mL to about 9,000 ng·hr/mL; about 9,000 ng·hr/mL to about 9,500 ng·hr/mL; or about 9,500 ng·hr/mL to about 10,000 ng·hr/mL.

Administration of a Tie-2 activator subcutaneously can reduce the systolic blood pressure of a subject, for example by about 1 mmHg, about 1.1 mmHg, about 1.2 mmHg, about 1.3 mmHg, about 1.4 mmHg, about 1.5 mmHg, about 1.6 mmHg, about 1.7 mmHg, about 1.8 mmHg, about 1.9 mmHg, about 2 mmHg, about 2.1 mmHg, about 2.2 mmHg, about 2.3 mmHg, about 2.4 mmHg, about 2.5 mmHg, about 2.6 mmHg, about 2.7 mmHg, about 2.8 mmHg, about 2.9 mmHg, about 3 mmHg, about 3.1 mmHg, about 3.2 mmHg, about 3.3 mmHg, about 3.4 mmHg, about 3.5 mmHg, about 3.6 mmHg, about 3.7 mmHg, about 3.8 mmHg, about 3.9 mmHg, about 4 mmHg, about 4.1 mmHg, about 4.2 mmHg, about 4.3 mmHg, about 4.4 mmHg, about 4.5 mmHg, about 4.6 mmHg, about 4.7 mmHg, about 4.8 mmHg, about 4.9 mmHg, about 5 mmHg, about 5.1 mmHg, about 5.2 mmHg, about 5.3 mmHg, about 5.4 mmHg, about 5.5 mmHg, about 5.6 mmHg, about 5.7 mmHg, about 5.8 mmHg, about 5.9 mmHg, about 6 mmHg, about 6.1 mmHg, about 6.2 mmHg, about 6.3 mmHg, about 6.4 mmHg, about 6.5 mmHg, about 6.6 mmHg, about 6.7 mmHg, about 6.8 mmHg, about 6.9 mmHg, about 7 mmHg, about 7.1 mmHg, about 7.2 mmHg, about 7.3 mmHg, about 7.4 mmHg, about 7.5 mmHg, about 7.6 mmHg, about 7.7 mmHg, about 7.8 mmHg, about 7.9 mmHg, about 8 mmHg, about 8.1 mmHg, about 8.2 mmHg, about 8.3 mmHg, about 8.4 mmHg, about 8.5 mmHg, about 8.6 mmHg, about 8.7 mmHg, about 8.8 mmHg, about 8.9 mmHg, about 9 mmHg, about 9.1 mmHg, about 9.2 mmHg, about 9.3 mmHg, about 9.4 mmHg, about 9.5 mmHg, about 9.6 mmHg, about 9.7 mmHg, about 9.8 mmHg, about 9.9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 21 mmHg, about 22 mmHg, about 23 mmHg, about 24 mmHg, about 25 mmHg, about 26 mmHg, about 27 mmHg, about 28 mmHg, about 29 mmHg, about 30 mmHg, about 31 mmHg, about 32 mmHg, about 33 mmHg, about 34 mmHg, about 35 mmHg, about 36 mmHg, about 37 mmHg, about 38 mmHg, about 39 mmHg, about 40 mmHg about 41 mmHg, about 42 mmHg, about 43 mmHg, about 44 mmHg, about 45 mmHg, about 46 mmHg, about 47 mmHg, about 48 mmHg, about 49 mmHg, about 50 mmHg, about 51 mmHg, about 52 mmHg, about 53 mmHg, about 54 mmHg, about 55 mmHg, about 56 mmHg, about 57 mmHg, about 58 mmHg, about 59 mmHg, about 60 mmHg, about 61 mmHg, about 62 mmHg, about 63 mmHg, about 64 mmHg, about 65 mmHg, about 66 mmHg, about 67 mmHg, about 68 mmHg, about 69 mmHg, about 70 mmHg, about 71 mmHg, about 72 mmHg, about 73 mmHg, about 74 mmHg, about 75 mmHg, about 76 mmHg, about 77 mmHg, about 78 mmHg, about 79 mmHg, about 80 mmHg, about 81 mmHg, about 82 mmHg, about 83 mmHg, about 84 mmHg, about 85 mmHg, about 86 mmHg, about 87 mmHg, about 88 mmHg, about 89 mmHg, about 90 mmHg, about 91 mmHg, about 92 mmHg, about 93 mmHg, about 94 mmHg, about 95 mmHg, about 96 mmHg, about 97 mmHg, about 98 mmHg, about 99 mmHg, or about 100 mmHg.

Administration of a Tie-2 activator subcutaneously can reduce the systolic blood pressure of a subject, for example, by at least 100 mmHg, by about 1 mmHg to about 100 mmHg, by about 1 mmHg to about 95 mmHg, by about 1 mmHg to about 90 mmHg, by about 1 mmHg to about 85 mmHg, by about 1 mmHg to about 80 mmHg, by about 1 mmHg to about 75 mmHg, by about 1 mmHg to about 70 mmHg, by about 1 mmHg to about 65 mmHg, by about 1 mmHg to about 60 mmHg, by about 1 mmHg to about 55 mmHg, by about 1 mmHg to about 50 mmHg, by about 1 mmHg to about 45 mmHg, by about 1 mmHg to about 40 mmHg, by about 1 mmHg to about 35 mmHg, by about 1 mmHg to about 30 mmHg, by about 1 mmHg to about 25 mmHg, by about 1 mmHg to about 20 mmHg, by about 1 mmHg to about 15 mmHg, by about 1 mmHg to about 10 mmHg, by about 1 mmHg to about 9 mmHg, by about 1 mmHg to about 8 mmHg, by about 1 mmHg to about 7 mmHg, by about 1 mmHg to about 6 mmHg, by about 1 mmHg to about 5 mmHg, by about 1 mmHg to about 4 mmHg, by about 1 mmHg to about 3 mmHg, by about 1 mmHg to about 2 mmHg, by about 5 mmHg to about 100 mmHg, by about 5 mmHg to about 95 mmHg, by about 5 mmHg to about 90 mmHg, by about 5 mmHg to about 85 mmHg, by about 5 mmHg to about 80 mmHg, by about 5 mmHg to about 75 mmHg, by about 5 mmHg to about 70 mmHg, by about 5 mmHg to about 65 mmHg, by about 5 mmHg to about 60 mmHg, by about 5 mmHg to about 55 mmHg, by about 5 mmHg to about 50 mmHg, by about 5 mmHg to about 45 mmHg, by about 5 mmHg to about 40 mmHg, by about 1 mmHg to about 35 mmHg, by about 5 mmHg to about 30 mmHg, by about 5 mmHg to about 25 mmHg, by about 5 mmHg to about 20 mmHg, by about 5 mmHg to about 15 mmHg, by about 5 mmHg to about 10 mmHg, by about 5 mmHg to about 9 mmHg, by about 5 mmHg to about 8 mmHg, by about 5 mmHg to about 7 mmHg, by about 5 mmHg to about 6 mmHg, by about 10 mmHg to about 100 mmHg, by about 10 mmHg to about 95 mmHg, by about 10 mmHg to about 90 mmHg, by about 10 mmHg to about 85 mmHg, by about 10 mmHg to about 80 mmHg, by about 10 mmHg to about 75 mmHg, by about 10 mmHg to about 70 mmHg, by about 10 mmHg to about 65 mmHg, by about 10 mmHg to about 60 mmHg, by about 10 mmHg to about 55 mmHg, by about 10 mmHg to about 50 mmHg, by about 10 mmHg to about 45 mmHg, by about 10 mmHg to about 40 mmHg, by about 1 mmHg to about 35 mmHg, by about 10 mmHg to about 30 mmHg, by about 10 mmHg to about 25 mmHg, by about 10 mmHg to about 20 mmHg, by about 10 mmHg to about 15 mmHg, by about 10 mmHg to about 14 mmHg, by about 10 mmHg to about 13 mmHg, by about 10 mmHg, to about 12 mmHg, or by about 10 mmHg, to about 11 mmHg.

Administration of a Tie-2 activator subcutaneously can reduce the diastolic blood pressure of a subject, for example by about 1 mmHg, about 1.1 mmHg, about 1.2 mmHg, about 1.3 mmHg, about 1.4 mmHg, about 1.5 mmHg, about 1.6 mmHg, about 1.7 mmHg, about 1.8 mmHg, about 1.9 mmHg, about 2 mmHg, about 2.1 mmHg, about 2.2 mmHg, about 2.3 mmHg, about 2.4 mmHg, about 2.5 mmHg, about 2.6 mmHg, about 2.7 mmHg, about 2.8 mmHg, about 2.9 mmHg, about 3 mmHg, about 3.1 mmHg, about 3.2 mmHg, about 3.3 mmHg, about 3.4 mmHg, about 3.5 mmHg, about 3.6 mmHg, about 3.7 mmHg, about 3.8 mmHg, about 3.9 mmHg, about 4 mmHg, about 4.1 mmHg, about 4.2 mmHg, about 4.3 mmHg, about 4.4 mmHg, about 4.5 mmHg, about 4.6 mmHg, about 4.7 mmHg, about 4.8 mmHg, about 4.9 mmHg, about 5 mmHg, about 5.1 mmHg, about 5.2 mmHg, about 5.3 mmHg, about 5.4 mmHg, about 5.5 mmHg, about 5.6 mmHg, about 5.7 mmHg, about 5.8 mmHg, about 5.9 mmHg, about 6 mmHg, about 6.1 mmHg, about 6.2 mmHg, about 6.3 mmHg, about 6.4 mmHg, about 6.5 mmHg, about 6.6 mmHg, about 6.7 mmHg, about 6.8 mmHg, about 6.9 mmHg, about 7 mmHg, about 7.1 mmHg, about 7.2 mmHg, about 7.3 mmHg, about 7.4 mmHg, about 7.5 mmHg, about 7.6 mmHg, about 7.7 mmHg, about 7.8 mmHg, about 7.9 mmHg, about 8 mmHg, about 8.1 mmHg, about 8.2 mmHg, about 8.3 mmHg, about 8.4 mmHg, about 8.5 mmHg, about 8.6 mmHg, about 8.7 mmHg, about 8.8 mmHg, about 8.9 mmHg, about 9 mmHg, about 9.1 mmHg, about 9.2 mmHg, about 9.3 mmHg, about 9.4 mmHg, about 9.5 mmHg, about 9.6 mmHg, about 9.7 mmHg, about 9.8 mmHg, about 9.9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 21 mmHg, about 22 mmHg, about 23 mmHg, about 24 mmHg, about 25 mmHg, about 26 mmHg, about 27 mmHg, about 28 mmHg, about 29 mmHg, about 30 mmHg, about 31 mmHg, about 32 mmHg, about 33 mmHg, about 34 mmHg, about 35 mmHg, about 36 mmHg, about 37 mmHg, about 38 mmHg, about 39 mmHg, about 40 mmHg about 41 mmHg, about 42 mmHg, about 43 mmHg, about 44 mmHg, about 45 mmHg, about 46 mmHg, about 47 mmHg, about 48 mmHg, about 49 mmHg, or about 50 mmHg.

Administration of a Tie-2 activator subcutaneously can reduce the diastolic blood pressure of a subject, for example, by at least 50 mmHg, by about 1 mmHg to about 50 mmHg, by about 1 mmHg to about 45 mmHg, by about 1 mmHg to about 40 mmHg, by about 1 mmHg to about 35 mmHg, by about 1 mmHg to about 30 mmHg, by about 1 mmHg to about 25 mmHg, by about 1 mmHg to about 20 mmHg, by about 1 mmHg to about 15 mmHg, by about 1 mmHg to about 10 mmHg, by about 1 mmHg to about 9 mmHg, by about 1 mmHg to about 8 mmHg, by about 1 mmHg to about 7 mmHg, by about 1 mmHg to about 6 mmHg, by about 1 mmHg to about 5 mmHg, by about 1 mmHg to about 4 mmHg, by about 1 mmHg to about 3 mmHg, by about 1 mmHg to about 2 mmHg, by about 5 mmHg to about 50 mmHg, by about 5 mmHg to about 45 mmHg, by about 5 mmHg to about 40 mmHg, by about 1 mmHg to about 35 mmHg, by about 5 mmHg to about 30 mmHg, by about 5 mmHg to about 25 mmHg, by about 5 mmHg to about 20 mmHg, by about 5 mmHg to about 15 mmHg, by about 5 mmHg to about 10 mmHg, by about 5 mmHg to about 9 mmHg, by about 5 mmHg to about 8 mmHg, by about 5 mmHg to about 7 mmHg, by about 5 mmHg to about 6 mmHg, by about 10 mmHg to about 50 mmHg, by about 10 mmHg to about 45 mmHg, by about 10 mmHg to about 40 mmHg, by about 1 mmHg to about 35 mmHg, by about 10 mmHg to about 30 mmHg, by about 10 mmHg to about 25 mmHg, by about 10 mmHg to about 20 mmHg, by about 10 mmHg to about 15 mmHg, by about 10 mmHg to about 14 mmHg, by about 10 mmHg to about 13 mmHg, or by about 10 mmHg to about 12 mmHg, or by about 10 mmHg to about 11 mmHg.

Administration of a Tie-2 activator subcutaneously can reduce the mean arterial pressure of a subject, for example by about 0.1 mmHg, about 0.2 mmHg, about 0.3 mmHg, about 0.4 mmHg, about 0.5 mmHg, about 0.6 mmHg, about 0.7 mmHg, about 0.8 mmHg, about 0.9 mmHg, about 1 mmHg, about 1.1 mmHg, about 1.2 mmHg, about 1.3 mmHg, about 1.4 mmHg, about 1.5 mmHg, about 1.6 mmHg, about 1.7 mmHg, about 1.8 mmHg, about 1.9 mmHg, about 2 mmHg, about 2.1 mmHg, about 2.2 mmHg, about 2.3 mmHg, about 2.4 mmHg, about 2.5 mmHg, about 2.6 mmHg, about 2.7 mmHg, about 2.8 mmHg, about 2.9 mmHg, about 3 mmHg, about 3.1 mmHg, about 3.2 mmHg, about 3.3 mmHg, about 3.4 mmHg, about 3.5 mmHg, about 3.6 mmHg, about 3.7 mmHg, about 3.8 mmHg, about 3.9 mmHg, about 4 mmHg, about 4.1 mmHg, about 4.2 mmHg, about 4.3 mmHg, about 4.4 mmHg, about 4.5 mmHg, about 4.6 mmHg, about 4.7 mmHg, about 4.8 mmHg, about 4.9 mmHg, about 5 mmHg, about 5.1 mmHg, about 5.2 mmHg, about 5.3 mmHg, about 5.4 mmHg, about 5.5 mmHg, about 5.6 mmHg, about 5.7 mmHg, about 5.8 mmHg, about 5.9 mmHg, about 6 mmHg, about 6.1 mmHg, about 6.2 mmHg, about 6.3 mmHg, about 6.4 mmHg, about 6.5 mmHg, about 6.6 mmHg, about 6.7 mmHg, about 6.8 mmHg, about 6.9 mmHg, about 7 mmHg, about 7.1 mmHg, about 7.2 mmHg, about 7.3 mmHg, about 7.4 mmHg, about 7.5 mmHg, about 7.6 mmHg, about 7.7 mmHg, about 7.8 mmHg, about 7.9 mmHg, about 8 mmHg, about 8.1 mmHg, about 8.2 mmHg, about 8.3 mmHg, about 8.4 mmHg, about 8.5 mmHg, about 8.6 mmHg, about 8.7 mmHg, about 8.8 mmHg, about 8.9 mmHg, about 9 mmHg, about 9.1 mmHg, about 9.2 mmHg, about 9.3 mmHg, about 9.4 mmHg, about 9.5 mmHg, about 9.6 mmHg, about 9.7 mmHg, about 9.8 mmHg, about 9.9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 21 mmHg, about 22 mmHg, about 23 mmHg, about 24 mmHg, about 25 mmHg, about 26 mmHg, about 27 mmHg, about 28 mmHg, about 29 mmHg, about 30 mmHg, about 31 mmHg, about 32 mmHg, about 33 mmHg, about 34 mmHg, about 35 mmHg, about 36 mmHg, about 37 mmHg, about 38 mmHg, about 39 mmHg, about 40 mmHg about 41 mmHg, about 42 mmHg, about 43 mmHg, about 44 mmHg, about 45 mmHg, about 46 mmHg, about 47 mmHg, about 48 mmHg, about 49 mmHg, about 50 mmHg, about 51 mmHg, about 52 mmHg, about 53 mmHg, about 54 mmHg, about 55 mmHg, about 56 mmHg, about 57 mmHg, about 58 mmHg, about 59 mmHg, or about 60 mmHg.

Administration of a Tie-2 activator subcutaneously can reduce the mean arterial pressure of a subject, for example, by at least 60 mmHg, by about 0.1 mmHg to about 60 mmHg, by about 0.1 mmHg to about 55 mmHg, by about 0.1 mmHg to about 50 mmHg, by about 0.1 mmHg to about 45 mmHg, by about 0.1 mmHg to about 40 mmHg, by about 1 mmHg to about 35 mmHg, by about 0.1 mmHg to about 30 mmHg, by about 0.1 mmHg to about 25 mmHg, by about 0.1 mmHg to about 20 mmHg, by about 0.1 mmHg to about 15 mmHg, by about 0.1 mmHg to about 10 mmHg, by about 0.1 mmHg to about 9 mmHg, by about 0.1 mmHg to about 8 mmHg, by about 0.1 mmHg to about 7 mmHg, by about 0.1 mmHg to about 6 mmHg, by about 0.1 mmHg to about 5 mmHg, by about 0.1 mmHg to about 4 mmHg, by about 0.1 mmHg to about 3 mmHg, by about 0.1 mmHg to about 2 mmHg, by about 0.1 mmHg to about 1 mmHg, by about 0.5 mmHg to about 60 mmHg, by about 0.5 mmHg to about 55 mmHg, by about 0.5 mmHg to about 50 mmHg, by about 0.5 mmHg to about 45 mmHg, by about 0.5 mmHg to about 40 mmHg, by about 1 mmHg to about 35 mmHg, by about 0.5 mmHg to about 30 mmHg, by about 0.5 mmHg to about 25 mmHg, by about 0.5 mmHg to about 20 mmHg, by about 0.5 mmHg to about 15 mmHg, by about 0.5 mmHg to about 10 mmHg, by about 0.5 mmHg to about 9 mmHg, by about 0.5 mmHg to about 8 mmHg, by about 0.5 mmHg to about 7 mmHg, by about 0.5 mmHg to about 6 mmHg, by about 0.5 mmHg to about 5 mmHg, by about 0.5 mmHg to about 4 mmHg, by about 0.5 mmHg to about 3 mmHg, by about 0.5 mmHg to about 2 mmHg, by about 0.5 mmHg to about 1 mmHg, by about 1 mmHg to about 60 mmHg, by about 1 mmHg to about 55 mmHg, by about 1 mmHg to about 50 mmHg, by about 1 mmHg to about 45 mmHg, by about 1 mmHg to about 40 mmHg, by about 1 mmHg to about 35 mmHg, by about 1 mmHg to about 30 mmHg, by about 1 mmHg to about 25 mmHg, by about 1 mmHg to about 20 mmHg, by about 1 mmHg to about 15 mmHg, by about 1 mmHg to about 10 mmHg, by about 1 mmHg to about 9 mmHg, by about 1 mmHg to about 8 mmHg, by about 1 mmHg to about 7 mmHg, by about 1 mmHg to about 6 mmHg, by about 1 mmHg to about 5 mmHg, by about 1 mmHg to about 4 mmHg, by about 1 mmHg to about 3 mmHg, or by about 1 mmHg to about 2 mmHg.

Administration of a Tie-2 activator subcutaneously can reduce the pulse pressure of a subject, for example by about 1 mmHg, about 2 mmHg, about 3 mmHg, about 4 mmHg, about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, about 21 mmHg, about 22 mmHg, about 23 mmHg, about 24 mmHg, about 25 mmHg, about 26 mmHg, about 27 mmHg, about 28 mmHg, about 29 mmHg, about 30 mmHg, about 31 mmHg, about 32 mmHg, about 33 mmHg, about 34 mmHg, about 35 mmHg, about 36 mmHg, about 37 mmHg, about 38 mmHg, about 39 mmHg, about 40 mmHg about 41 mmHg, about 42 mmHg, about 43 mmHg, about 44 mmHg, about 45 mmHg, about 46 mmHg, about 47 mmHg, about 48 mmHg, about 49 mmHg, or about 50 mmHg.

Administration of a Tie-2 activator subcutaneously can reduce the pulse pressure of a subject, for example, by at least 50 mmHg, by about 1 mmHg to about 50 mmHg, by about 1 mmHg to about 45 mmHg, by about 1 mmHg to about 40 mmHg, by about 1 mmHg to about 35 mmHg, by about 1 mmHg to about 30 mmHg, by about 1 mmHg to about 25 mmHg, by about 1 mmHg to about 20 mmHg, by about 1 mmHg to about 15 mmHg, by about 1 mmHg to about 10 mmHg, by about 1 mmHg to about 9 mmHg, by about 1 mmHg to about 8 mmHg, by about 1 mmHg to about 7 mmHg, by about 1 mmHg to about 6 mmHg, by about 1 mmHg to about 5 mmHg, by about 1 mmHg to about 4 mmHg, by about 1 mmHg to about 3 mmHg, or by about 1 mmHg to about 2 mmHg.

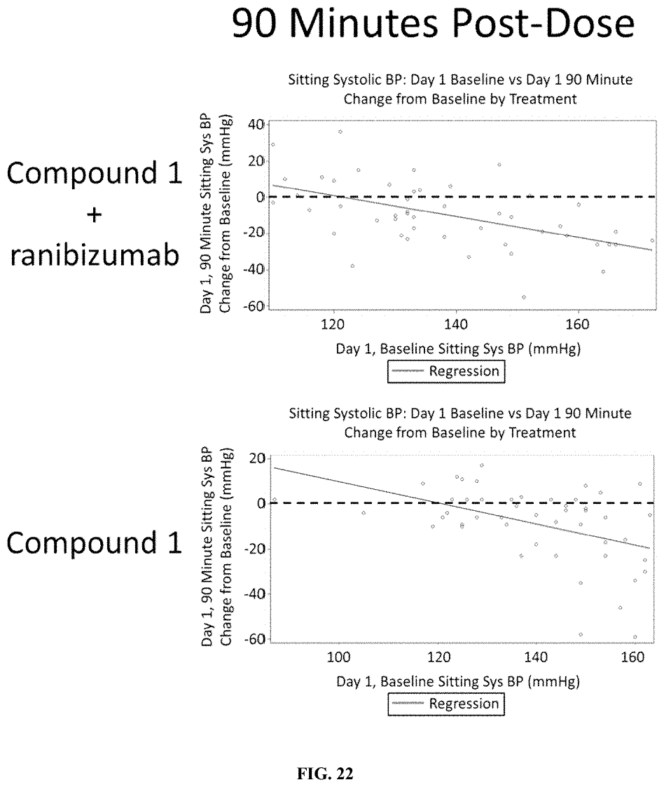

In some instances, in a study of a human with hypertension, subcutaneous administration of a Tie-2 activator can modulate blood pressure in the human 90 minutes after administration of the Tie-2 activator. In some embodiments, the modulation of blood pressure in the human can correlate to, for example, a baseline sitting blood pressure of the human as illustrated in the bottom panel of FIG. 22, with at most a 5%, 10%, 15%, 20%, 25%, 30%, 35%, 40%, 45%, or 50% deviation from the regression line shown in the bottom panel of FIG. 22.

### Examples

### Example 1. Compounds with Inhibitory Activity to HPTPβ

Non-limiting examples of the HPTPβ IC₅₀ (μM) activity for illustrative compounds are listed in TABLE 1.

TABLE 1

| | | HPTPβ |
|---|---|---|
| No. | Compound | IC₅₀ μM |
| AA1 | 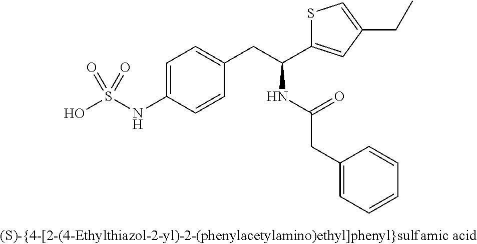 [SMILES: CCc1csc([C@H](Cc2ccc(NS(=O)(=O)O)cc2)NC(=O)Cc2ccccc2)c1] | 0.000157 |
| AA2 | 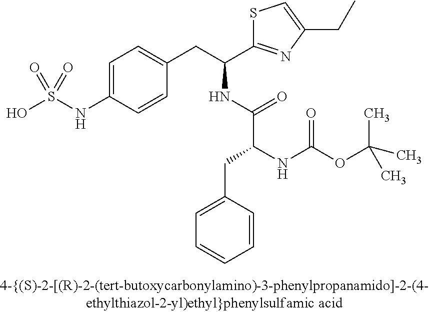 [SMILES: CCc1csc([C@H](Cc2ccc(NS(=O)(=O)O)cc2)NC(=O)[C@@H](Cc2ccccc2)NC(=O)OC(C)(C)C)n1] | 0.004 |
| AA3 | 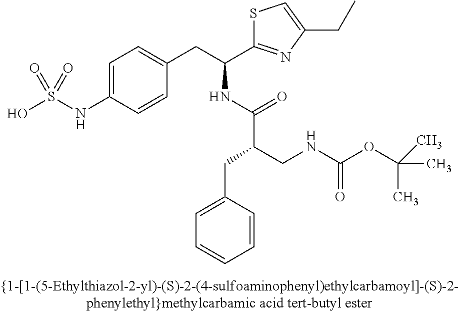 [SMILES: CCc1csc([C@H](Cc2ccc(NS(=O)(=O)O)cc2)NC(=O)[C@H](CNC(=O)OC(C)(C)C)Cc2ccccc2)n1] | 0.031 |
| AA4 | 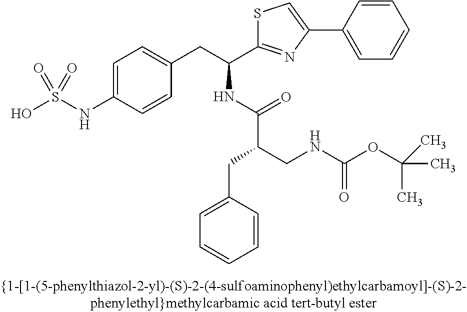 [SMILES: CC(C)(C)OC(=O)NC[C@H](Cc1ccccc1)C(=O)N[C@@H](Cc1ccc(NS(=O)(=O)O)cc1)c1nc(-c2ccccc2)cs1] | <5 × 10⁻⁸ |
| AA5 | 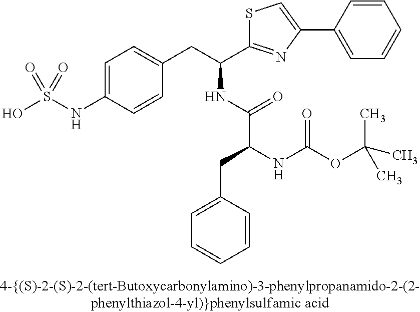 [SMILES: CC(C)(C)OC(=O)N[C@@H](Cc1ccccc1)C(=O)N[C@@H](Cc1ccc(NS(=O)(=O)O)cc1)c1nc(-c2ccccc2)cs1] | <5 × 10⁻⁸ |
| AA6 | 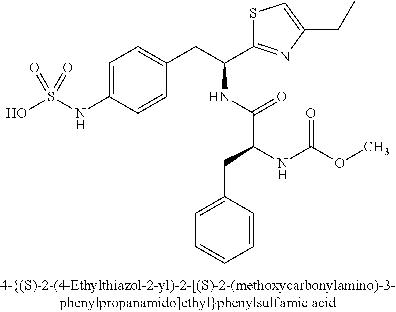 [SMILES: CCc1csc([C@H](Cc2ccc(NS(=O)(=O)O)cc2)NC(=O)[C@H](Cc2ccccc2)NC(=O)OC)n1] | 0.000162 |
| AA7 | 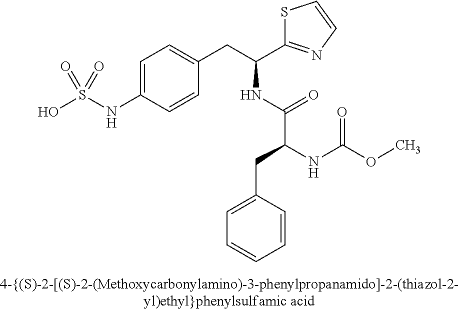 [SMILES: COC(=O)N[C@@H](Cc1ccccc1)C(=O)N[C@@H](Cc1ccc(NS(=O)(=O)O)cc1)c1nccs1] | 0.006 |
| AA8 | 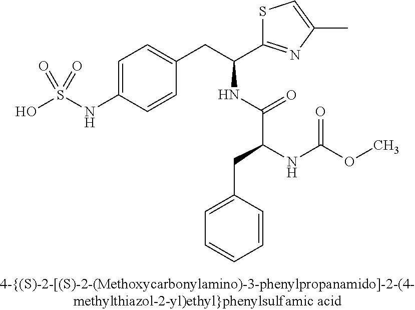 [SMILES: COC(=O)N[C@@H](Cc1ccccc1)C(=O)N[C@@H](Cc1ccc(NS(=O)(=O)O)cc1)c1nc(C)cs1] | 0.001 |
| AA9 | 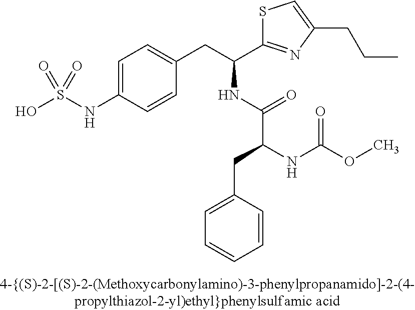 [SMILES: CCCc1csc([C@H](Cc2ccc(NS(=O)(=O)O)cc2)NC(=O)[C@H](Cc2ccccc2)NC(=O)OC)n1] | 0.0001 |
| AA10 | 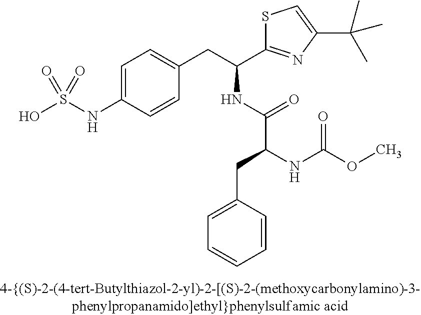 [SMILES: COC(=O)N[C@@H](Cc1ccccc1)C(=O)N[C@@H](Cc1ccc(NS(=O)(=O)O)cc1)c1nc(C(C)(C)C)cs1] | 0.0002 |
| AA11 | 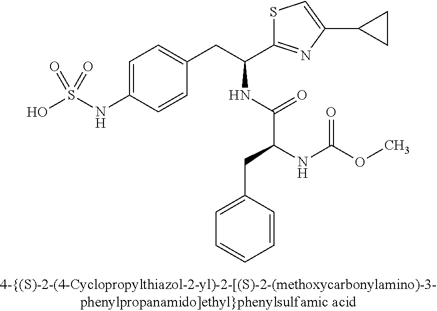 [SMILES: COC(=O)N[C@@H](Cc1ccccc1)C(=O)N[C@@H](Cc1ccc(NS(=O)(=O)O)cc1)c1nc(C2CC2)cs1] | 0.00001 |
| AA12 | 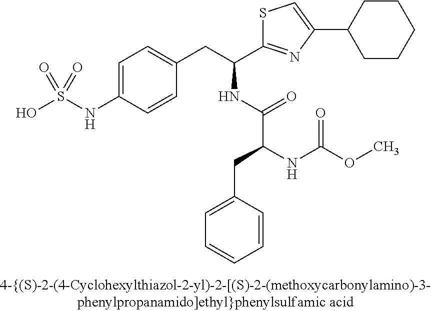 [SMILES: COC(=O)N[C@@H](Cc1ccccc1)C(=O)N[C@@H](Cc1ccc(NS(=O)(=O)O)cc1)c1nc(C2CCCCC2)cs1] | <5 × 10⁻⁸ |
| AA13 | 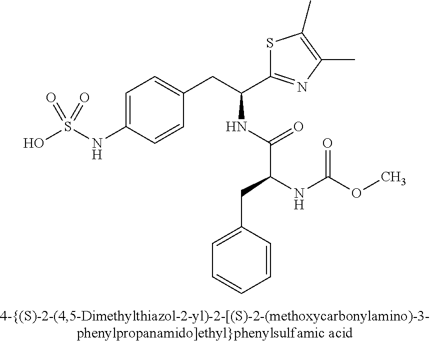 [SMILES: COC(=O)N[C@@H](Cc1ccccc1)C(=O)N[C@@H](Cc1ccc(NS(=O)(=O)O)cc1)c1nc(C)c(C)s1] | 0.001 |
| AA14 | 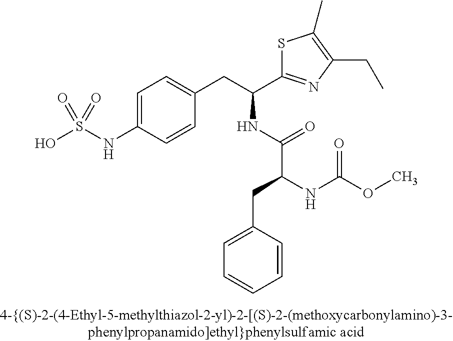 [SMILES: CCc1nc([C@H](Cc2ccc(NS(=O)(=O)O)cc2)NC(=O)[C@H](Cc2ccccc2)NC(=O)OC)sc1C] | 0.0001 |
| AA15 | 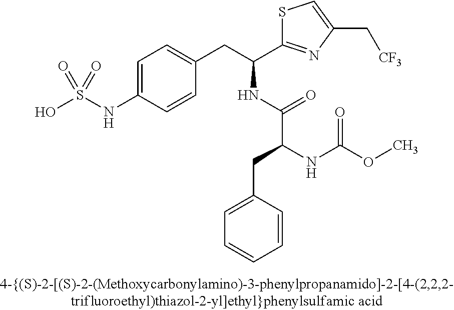 [SMILES: COC(=O)N[C@@H](Cc1ccccc1)C(=O)N[C@@H](Cc1ccc(NS(=O)(=O)O)cc1)c1nc(CC(F)(F)F)cs1] | 0.0003 |
| AA16 | 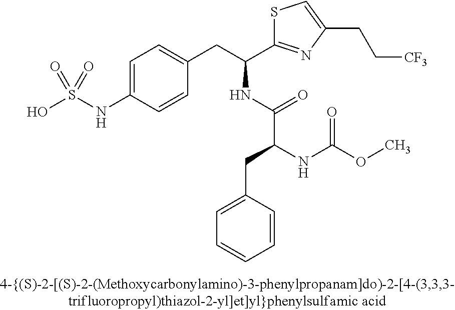 [SMILES: COC(=O)N[C@@H](Cc1ccccc1)C(=O)N[C@@H](Cc1ccc(NS(=O)(=O)O)cc1)c1nc(CCC(F)(F)F)cs1] | 0.00008 |
| AA17 | 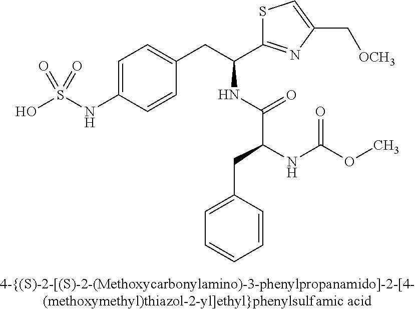 [SMILES: COCc1csc([C@H](Cc2ccc(NS(=O)(=O)O)cc2)NC(=O)[C@H](Cc2ccccc2)NC(=O)OC)n1] | 0.001 |
| AA18 | 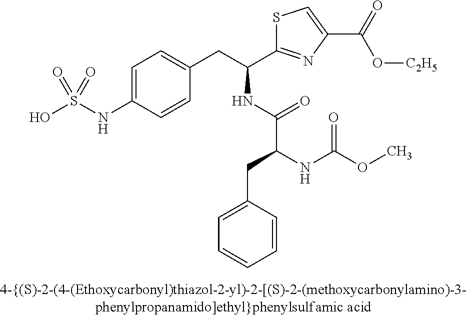 [SMILES: CCOC(=O)c1csc([C@H](Cc2ccc(NS(=O)(=O)O)cc2)NC(=O)[C@H](Cc2ccccc2)NC(=O)OC)n1] | 0.0002 |
| AA19 | 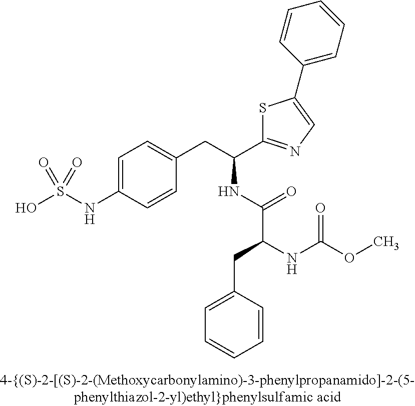 [SMILES: COC(=O)N[C@@H](Cc1ccccc1)C(=O)N[C@@H](Cc1ccc(NS(=O)(=O)O)cc1)c1ncc(-c2ccccc2)s1] | 0.0003 |
| AA20 | 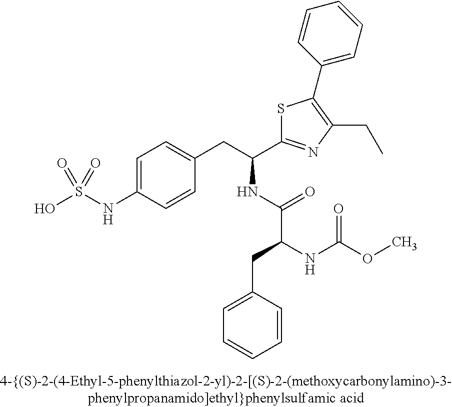 [SMILES: CCc1nc([C@H](Cc2ccc(NS(=O)(=O)O)cc2)NC(=O)[C@H](Cc2ccccc2)NC(=O)OC)sc1-c1ccccc1] | <5 × 10⁻⁸ |
| AA21 | 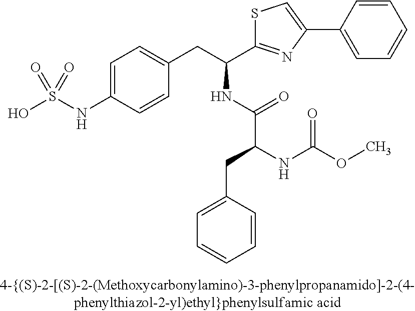 [SMILES: COC(=O)N[C@@H](Cc1ccccc1)C(=O)N[C@@H](Cc1ccc(NS(=O)(=O)O)cc1)c1nc(-c2ccccc2)cs1] | <2 × 10⁻⁶ |
| AA22 | 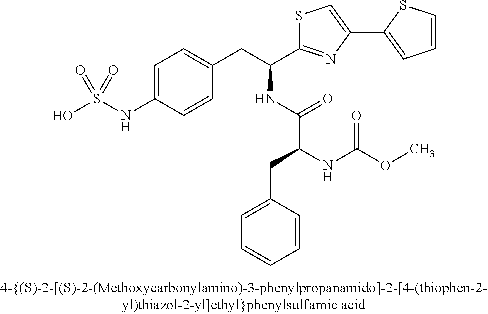 [SMILES: COC(=O)N[C@@H](Cc1ccccc1)C(=O)N[C@@H](Cc1ccc(NS(=O)(=O)O)cc1)c1nc(-c2cccs2)cs1] | <5 × 10⁻⁸ |
| AA23 | 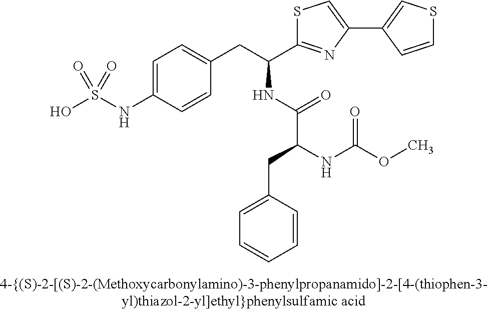 [SMILES: COC(=O)N[C@@H](Cc1ccccc1)C(=O)N[C@@H](Cc1ccc(NS(=O)(=O)O)cc1)c1nc(-c2ccsc2)cs1] | 0.00009 |
| AA24 | 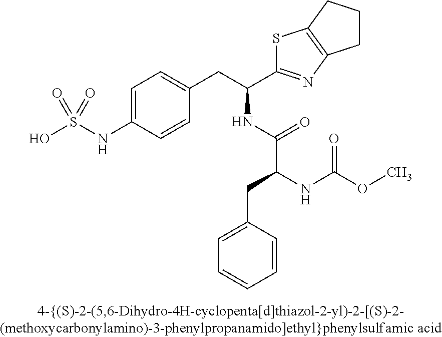 [SMILES: COC(=O)N[C@@H](Cc1ccccc1)C(=O)N[C@@H](Cc1ccc(NS(=O)(=O)O)cc1)c1nc2c(s1)CCC2] | 0.001 |
| AA25 | 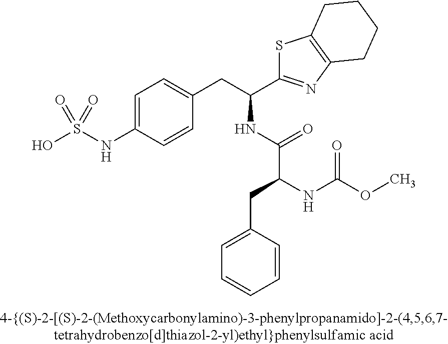 [SMILES: COC(=O)N[C@@H](Cc1ccccc1)C(=O)N[C@@H](Cc1ccc(NS(=O)(=O)O)cc1)c1nc2c(s1)CCCC2] | 0.0004 |
| AA26 | 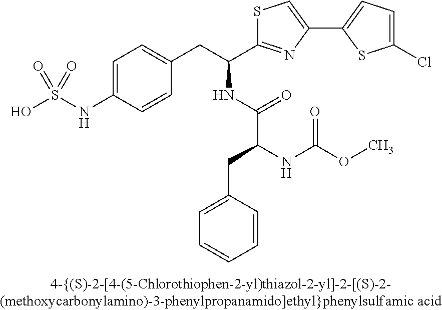 [SMILES: COC(=O)N[C@@H](Cc1ccccc1)C(=O)N[C@@H](Cc1ccc(NS(=O)(=O)O)cc1)c1nc(-c2ccc(Cl)s2)cs1] | <5 × 10⁻⁸ |
| AA27 | 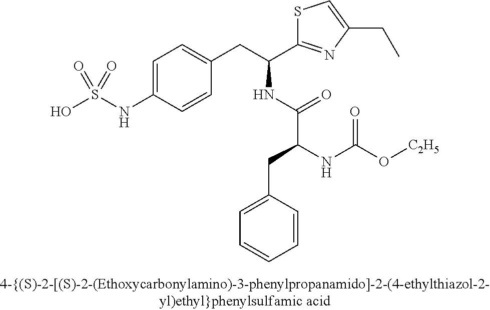 [SMILES: CCOC(=O)N[C@@H](Cc1ccccc1)C(=O)N[C@@H](Cc1ccc(NS(=O)(=O)O)cc1)c1nc(CC)cs1] | 0.00014 |
| AA28 | 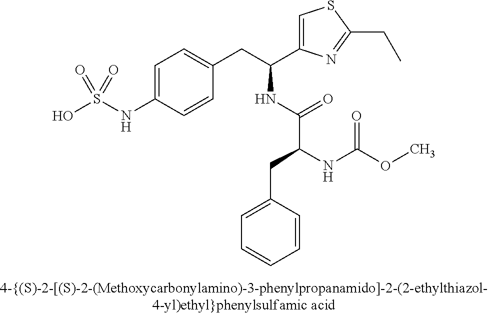 [SMILES: CCc1nc([C@H](Cc2ccc(NS(=O)(=O)O)cc2)NC(=O)[C@H](Cc2ccccc2)NC(=O)OC)cs1] | 0.0001 |
| AA29 | 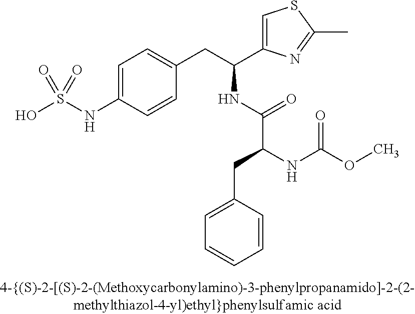 [SMILES: COC(=O)N[C@@H](Cc1ccccc1)C(=O)N[C@@H](Cc1ccc(NS(=O)(=O)O)cc1)c1csc(C)n1] | 0.001 |
| AA30 | 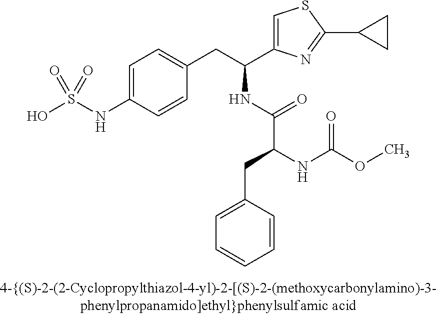 [SMILES: COC(=O)N[C@@H](Cc1ccccc1)C(=O)N[C@@H](Cc1ccc(NS(=O)(=O)O)cc1)c1csc(C2CC2)n1] | 0.0002 |
| AA31 | 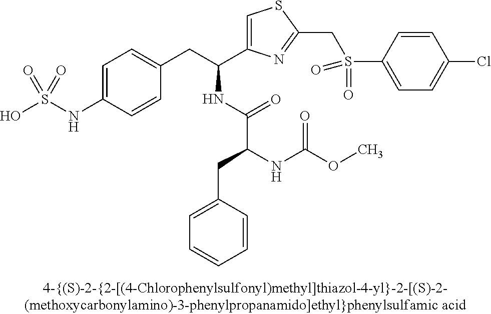 [SMILES: COC(=O)N[C@@H](Cc1ccccc1)C(=O)N[C@@H](Cc1ccc(NS(=O)(=O)O)cc1)c1csc(CS(=O)(=O)c2ccc(Cl)cc2)n1] | 0.00008 |
| AA32 | 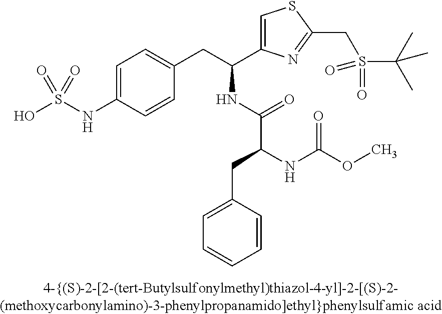 [SMILES: COC(=O)N[C@@H](Cc1ccccc1)C(=O)N[C@@H](Cc1ccc(NS(=O)(=O)O)cc1)c1csc(CS(=O)(=O)C(C)(C)C)n1] | 0.002 |
| AA33 | 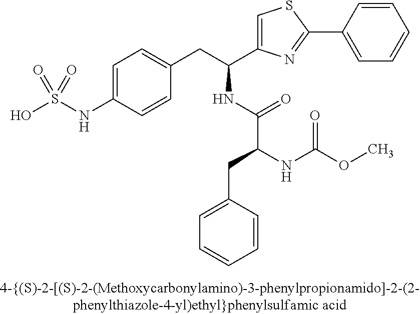 [SMILES: COC(=O)N[C@@H](Cc1ccccc1)C(=O)N[C@@H](Cc1ccc(NS(=O)(=O)O)cc1)c1csc(-c2ccccc2)n1] | 7 × 10⁻⁷ |
| AA34 | 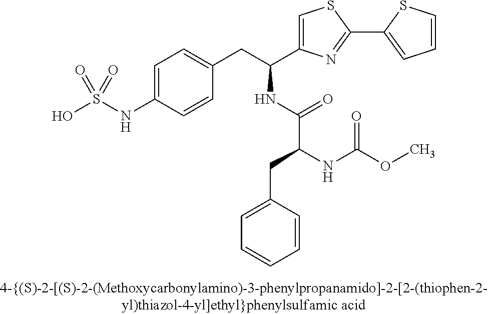 [SMILES: COC(=O)N[C@@H](Cc1ccccc1)C(=O)N[C@@H](Cc1ccc(NS(=O)(=O)O)cc1)c1csc(-c2cccs2)n1] | 5 × 10⁻⁸ |
| AA35 | 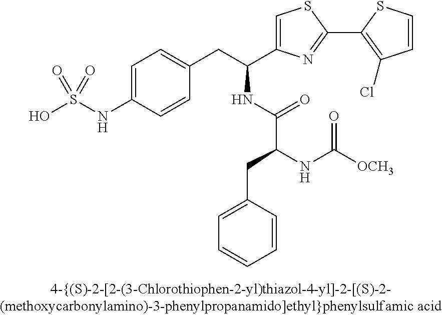 [SMILES: COC(=O)N[C@@H](Cc1ccccc1)C(=O)N[C@@H](Cc1ccc(NS(=O)(=O)O)cc1)c1csc(-c2sccc2Cl)n1] | <5 × 10⁻⁸ |
| AA36 | 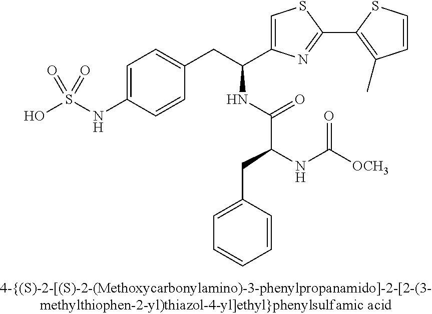 [SMILES: COC(=O)N[C@@H](Cc1ccccc1)C(=O)N[C@@H](Cc1ccc(NS(=O)(=O)O)cc1)c1csc(-c2sccc2C)n1] | <5 × 10⁻⁸ |
| AA37 | 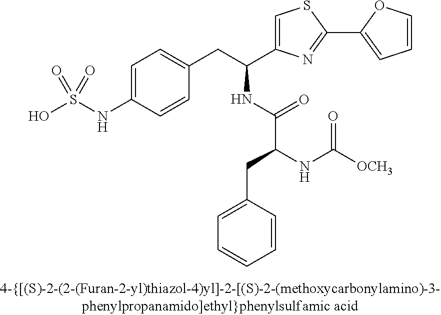 [SMILES: COC(=O)N[C@@H](Cc1ccccc1)C(=O)N[C@@H](Cc1ccc(NS(=O)(=O)O)cc1)c1csc(-c2ccco2)n1] | 0.0004 |
| AA38 | 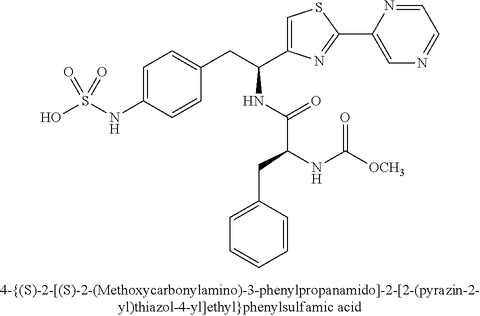 [SMILES: COC(=O)N[C@@H](Cc1ccccc1)C(=O)N[C@@H](Cc1ccc(NS(=O)(=O)O)cc1)c1csc(-c2cnccn2)n1] | 0.003 |
| AA39 | 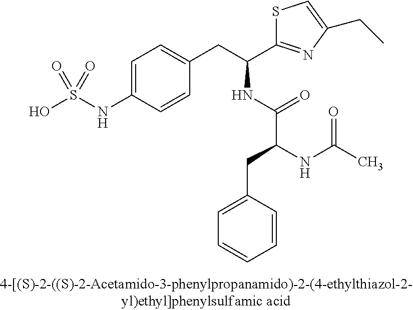 [SMILES: CCc1csc([C@H](Cc2ccc(NS(=O)(=O)O)cc2)NC(=O)[C@H](Cc2ccccc2)NC(C)=O)n1] | 0.001 |
| AA40 | 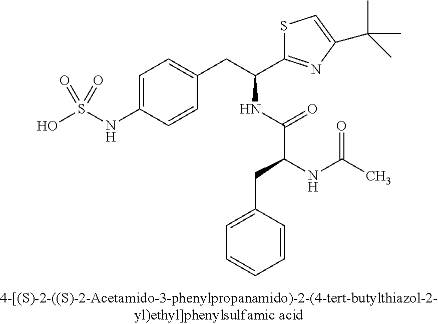 [SMILES: CC(=O)N[C@@H](Cc1ccccc1)C(=O)N[C@@H](Cc1ccc(NS(=O)(=O)O)cc1)c1nc(C(C)(C)C)cs1] | 0.0003 |
| AA41 | 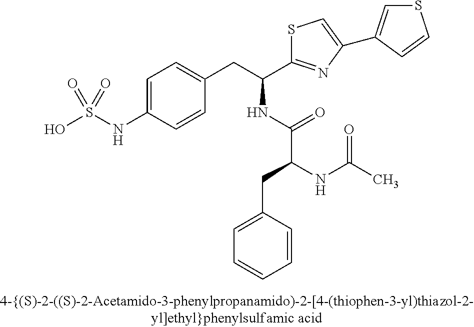 [SMILES: CC(=O)N[C@@H](Cc1ccccc1)C(=O)N[C@@H](Cc1ccc(NS(=O)(=O)O)cc1)c1nc(-c2ccsc2)cs1] | 0.00024 |
| AA42 | 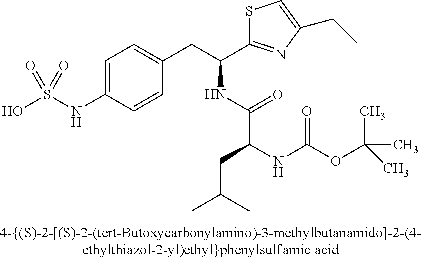 [SMILES: CCc1csc([C@H](Cc2ccc(NS(=O)(=O)O)cc2)NC(=O)[C@H](CC(C)C)NC(=O)OC(C)(C)C)n1] | 0.006 |
| AA43 | 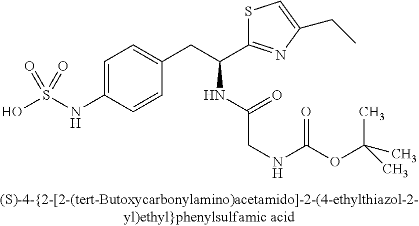 [SMILES: CCc1csc([C@H](Cc2ccc(NS(=O)(=O)O)cc2)NC(=O)CNC(=O)OC(C)(C)C)n1] | 0.028 |
| AA44 | 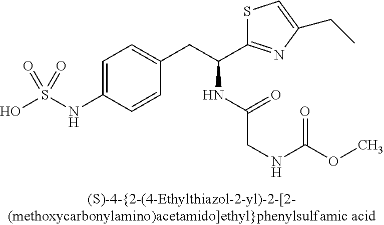 [SMILES: CCc1csc([C@H](Cc2ccc(NS(=O)(=O)O)cc2)NC(=O)CNC(=O)OC)n1] | 0.020 |
| AA45 | 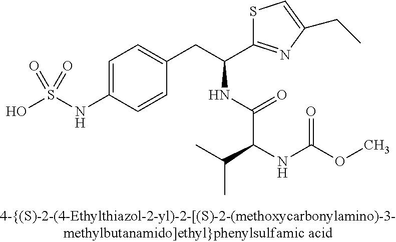 [SMILES: CCc1csc([C@H](Cc2ccc(NS(=O)(=O)O)cc2)NC(=O)[C@@H](NC(=O)OC)C(C)C)n1] | 0.003 |
| AA46 | 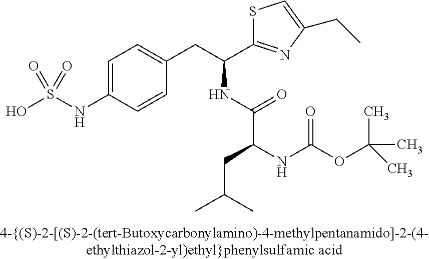 [SMILES: CCc1csc([C@H](Cc2ccc(NS(=O)(=O)O)cc2)NC(=O)[C@H](CC(C)C)NC(=O)OC(C)(C)C)n1] | 0.001 |
| AA47 | 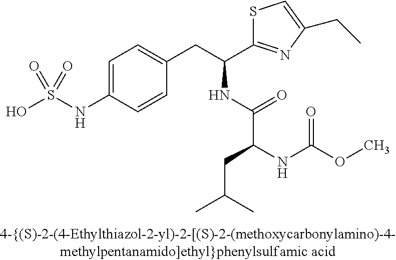 [SMILES: CCc1csc([C@H](Cc2ccc(NS(=O)(=O)O)cc2)NC(=O)[C@H](CC(C)C)NC(=O)OC)n1] | 0.0003 |
| AA48 | 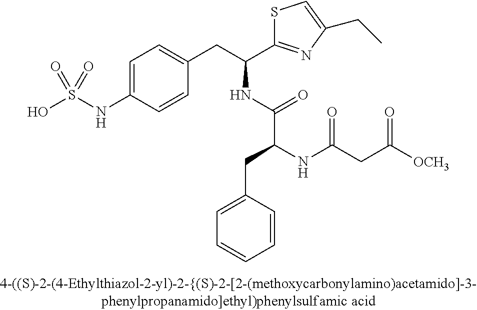 [SMILES: CCc1csc([C@H](Cc2ccc(NS(=O)(=O)O)cc2)NC(=O)[C@H](Cc2ccccc2)NC(=O)CC(=O)OC)n1] | 0.0003 |
| AA49 | 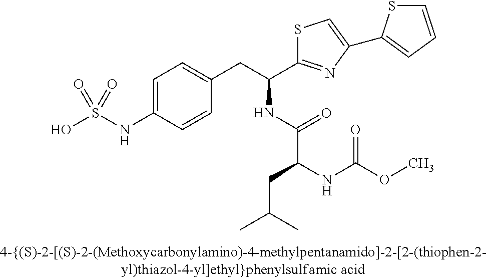 [SMILES: COC(=O)N[C@@H](CC(C)C)C(=O)N[C@@H](Cc1ccc(NS(=O)(=O)O)cc1)c1nc(-c2cccs2)cs1] | <5 × 10⁻⁸ |
| AA50 | 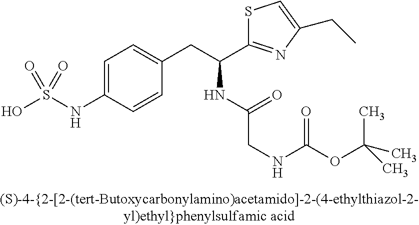 [SMILES: CCc1csc([C@H](Cc2ccc(NS(=O)(=O)O)cc2)NC(=O)CNC(=O)OC(C)(C)C)n1] | 0.028 |
| AA51 | 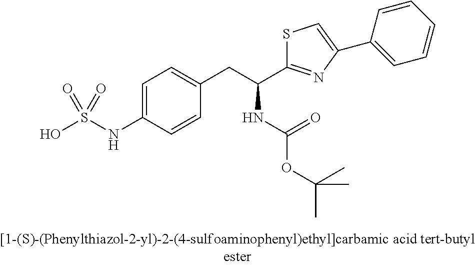 [SMILES: CC(C)(C)OC(=O)N[C@@H](Cc1ccc(NS(=O)(=O)O)cc1)c1nc(-c2ccccc2)cs1] | 0.049 |
| AA52 | 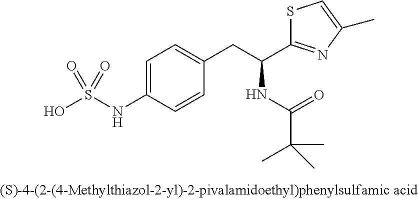 [SMILES: Cc1csc([C@H](Cc2ccc(NS(=O)(=O)O)cc2)NC(=O)C(C)(C)C)n1] | 0.112 |
| AA53 | 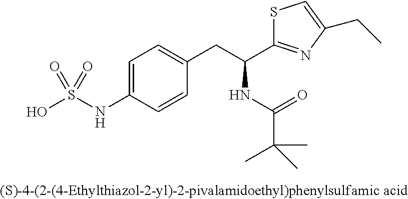 [SMILES: CCc1csc([C@H](Cc2ccc(NS(=O)(=O)O)cc2)NC(=O)C(C)(C)C)n1] | 0.085 |
| AA54 | 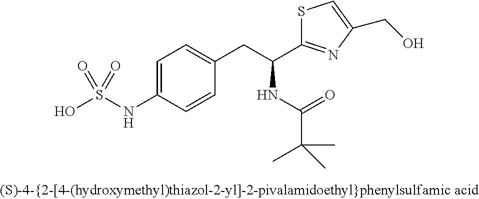 [SMILES: CC(C)(C)C(=O)N[C@@H](Cc1ccc(NS(=O)(=O)O)cc1)c1nc(CO)cs1] | 0.266 |
| AA55 | 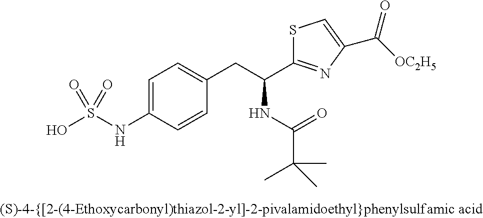 [SMILES: CCOC(=O)c1csc([C@H](Cc2ccc(NS(=O)(=O)O)cc2)NC(=O)C(C)(C)C)n1] | 0.584 |
| AA56 | 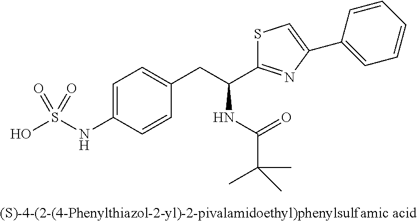 [SMILES: CC(C)(C)C(=O)N[C@@H](Cc1ccc(NS(=O)(=O)O)cc1)c1nc(-c2ccccc2)cs1] | 0.042 |
| AA57 | 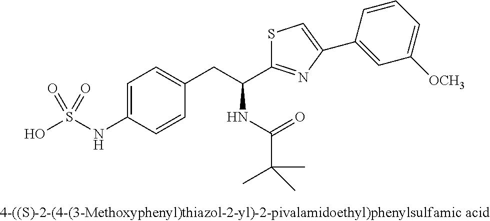 [SMILES: COc1cccc(-c2csc([C@H](Cc3ccc(NS(=O)(=O)O)cc3)NC(=O)C(C)(C)C)n2)c1] | 0.110 |
| AA58 | 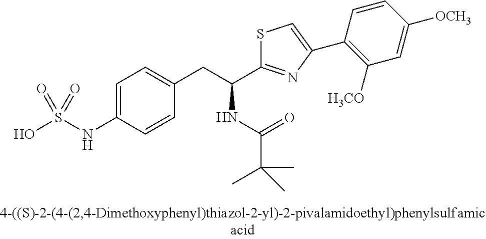 [SMILES: COc1ccc(-c2csc([C@H](Cc3ccc(NS(=O)(=O)O)cc3)NC(=O)C(C)(C)C)n2)c(OC)c1] | 0.086 |
| AA59 | 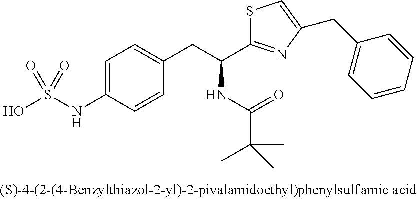 [SMILES: CC(C)(C)C(=O)N[C@@H](Cc1ccc(NS(=O)(=O)O)cc1)c1nc(Cc2ccccc2)cs1] | 0.113 |
| AA60 | 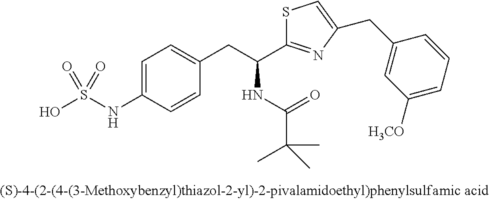 [SMILES: COc1cccc(Cc2csc([C@H](Cc3ccc(NS(=O)(=O)O)cc3)NC(=O)C(C)(C)C)n2)c1] | 0.132 |
| AA61 | 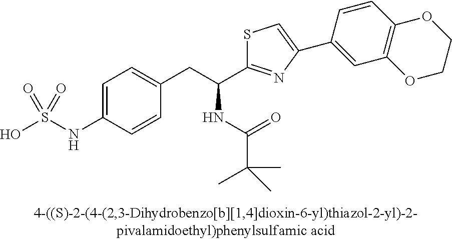 [SMILES: CC(C)(C)C(=O)N[C@@H](Cc1ccc(NS(=O)(=O)O)cc1)c1nc(-c2ccc3c(c2)OCCO3)cs1] | 0.138 |
| AA62 | 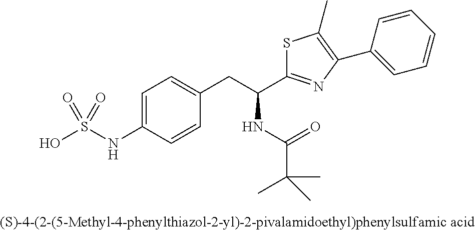 [SMILES: Cc1sc([C@H](Cc2ccc(NS(=O)(=O)O)cc2)NC(=O)C(C)(C)C)nc1-c1ccccc1] | 0.098 |
| AA63 | 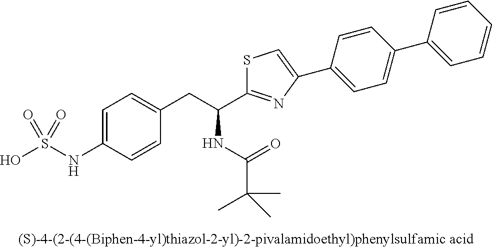 [SMILES: CC(C)(C)C(=O)N[C@@H](Cc1ccc(NS(=O)(=O)O)cc1)c1nc(-c2ccc(-c3ccccc3)cc2)cs1] | 0.381 |
| AA64 | 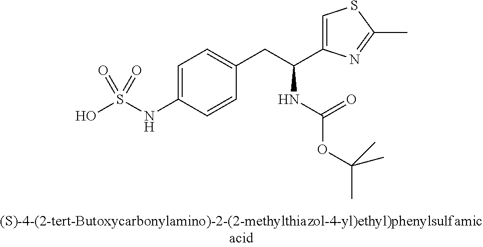 [SMILES: Cc1nc([C@H](Cc2ccc(NS(=O)(=O)O)cc2)NC(=O)OC(C)(C)C)cs1] | 0.033 |
| AA65 | 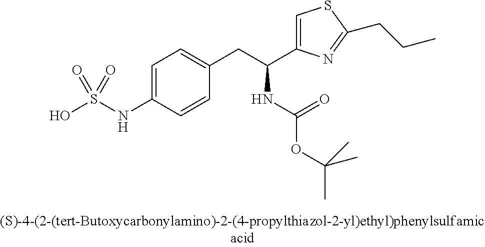 [SMILES: CCCc1nc([C@H](Cc2ccc(NS(=O)(=O)O)cc2)NC(=O)OC(C)(C)C)cs1] | 0.04 |
| AA66 | 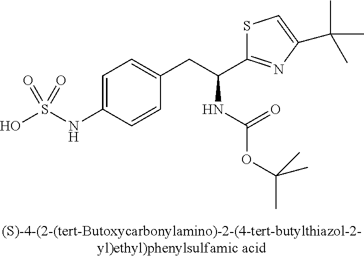 [SMILES: CC(C)(C)OC(=O)N[C@@H](Cc1ccc(NS(=O)(=O)O)cc1)c1nc(C(C)(C)C)cs1] | 0.027 |
| AA67 | 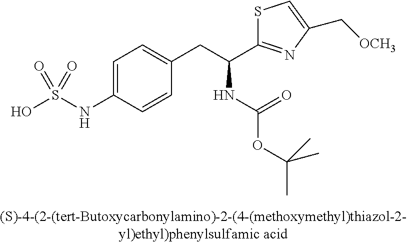 [SMILES: COCc1csc([C@H](Cc2ccc(NS(=O)(=O)O)cc2)NC(=O)OC(C)(C)C)n1] | 0.18 |
| AA68 | 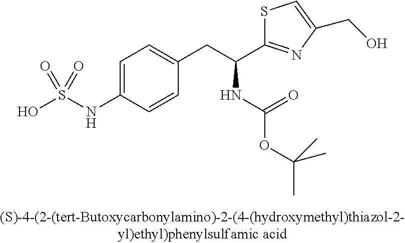 [SMILES: CC(C)(C)OC(=O)N[C@@H](Cc1ccc(NS(=O)(=O)O)cc1)c1nc(CO)cs1] | 0.644 |
| AA69 | 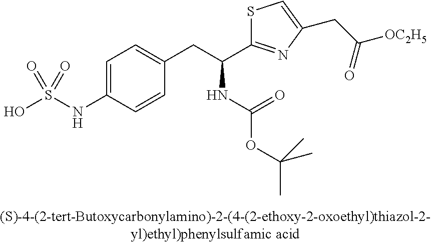 [SMILES: CCOC(=O)Cc1csc([C@H](Cc2ccc(NS(=O)(=O)O)cc2)NC(=O)OC(C)(C)C)n1] | 0.167 |
| AA70 | 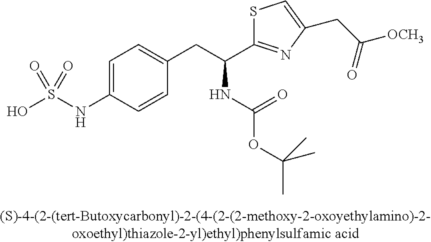 [SMILES: COC(=O)Cc1csc([C@H](Cc2ccc(NS(=O)(=O)O)cc2)NC(=O)OC(C)(C)C)n1] | 0.132 |
| AA71 | 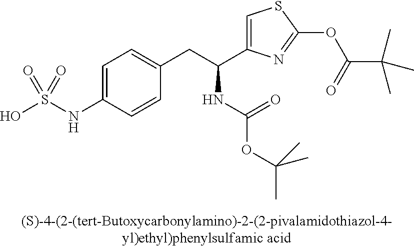 [SMILES: CC(C)(C)OC(=O)N[C@@H](Cc1ccc(NS(=O)(=O)O)cc1)c1csc(OC(=O)C(C)(C)C)n1] | 0.555 |
| AA72 | 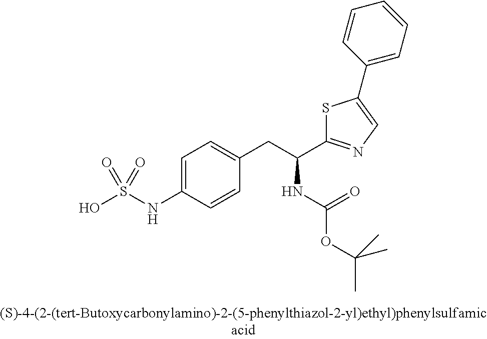 [SMILES: CC(C)(C)OC(=O)N[C@@H](Cc1ccc(NS(=O)(=O)O)cc1)c1ncc(-c2ccccc2)s1] | 0.308 |
| AA73 | 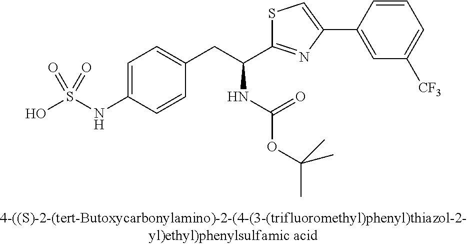 [SMILES: CC(C)(C)OC(=O)N[C@@H](Cc1ccc(NS(=O)(=O)O)cc1)c1nc(-c2cccc(C(F)(F)F)c2)cs1] | 0.253 |
| AA74 | 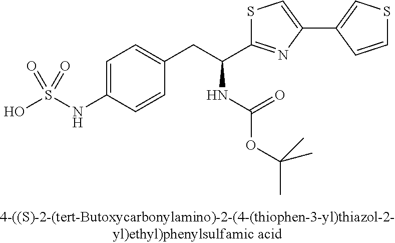 [SMILES: CC(C)(C)OC(=O)N[C@@H](Cc1ccc(NS(=O)(=O)O)cc1)c1nc(-c2ccsc2)cs1] | 0.045 |
| AA75 | 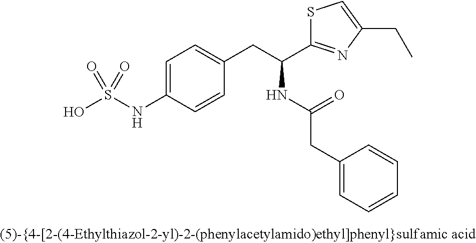 [SMILES: CCc1csc([C@H](Cc2ccc(NS(=O)(=O)O)cc2)NC(=O)Cc2ccccc2)n1] | 0.05 |
| AA76 | 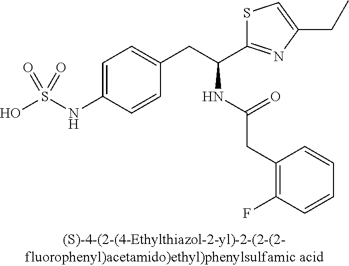 [SMILES: CCc1csc([C@H](Cc2ccc(NS(=O)(=O)O)cc2)NC(=O)Cc2ccccc2F)n1] | 0.012 |
| AA77 | 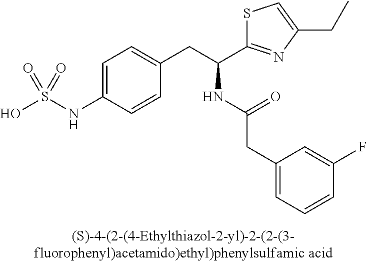 [SMILES: CCc1csc([C@H](Cc2ccc(NS(=O)(=O)O)cc2)NC(=O)Cc2cccc(F)c2)n1] | 0.0003 |
| AA78 | 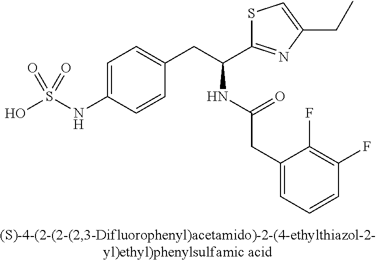 [SMILES: CCc1csc([C@H](Cc2ccc(NS(=O)(=O)O)cc2)NC(=O)Cc2cccc(F)c2F)n1] | 0.028 |
| AA79 | 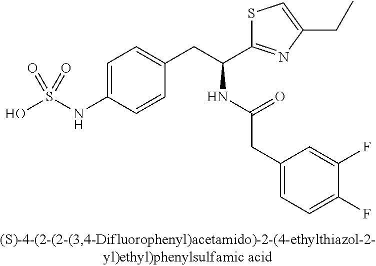 [SMILES: CCc1csc([C@H](Cc2ccc(NS(=O)(=O)O)cc2)NC(=O)Cc2ccc(F)c(F)c2)n1] | 0.075 |
| AA80 | 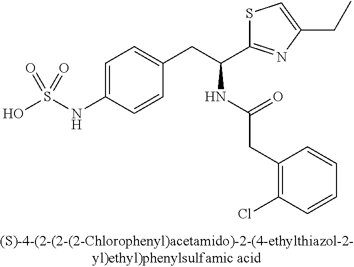 [SMILES: CCc1csc([C@H](Cc2ccc(NS(=O)(=O)O)cc2)NC(=O)Cc2ccccc2Cl)n1] | 0.056 |
| AA81 | 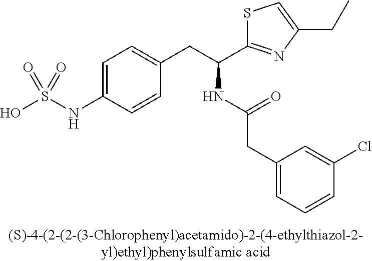 [SMILES: CCc1csc([C@H](Cc2ccc(NS(=O)(=O)O)cc2)NC(=O)Cc2cccc(Cl)c2)n1] | 0.033 |
| AA82 | 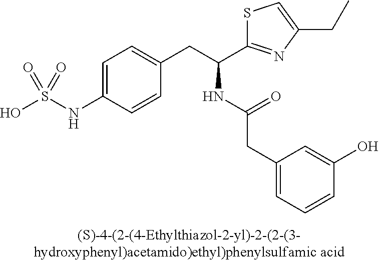 [SMILES: CCc1csc([C@H](Cc2ccc(NS(=O)(=O)O)cc2)NC(=O)Cc2cccc(O)c2)n1] | 0.04 |
| AA83 | 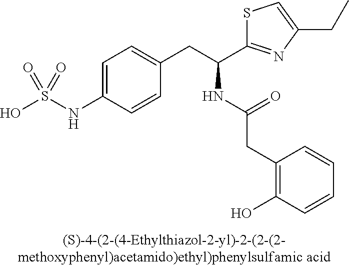 [SMILES: CCc1csc([C@H](Cc2ccc(NS(=O)(=O)O)cc2)NC(=O)Cc2ccccc2O)n1] | 0.014 |
| AA84 | 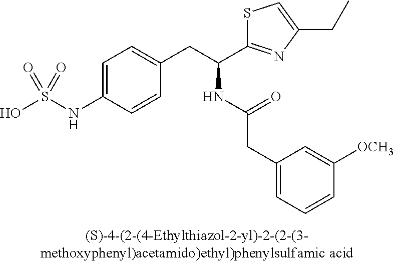 [SMILES: CCc1csc([C@H](Cc2ccc(NS(=O)(=O)O)cc2)NC(=O)Cc2cccc(OC)c2)n1] | 0.008 |
| AA85 | 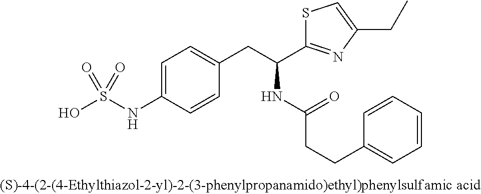 [SMILES: CCc1csc([C@H](Cc2ccc(NS(=O)(=O)O)cc2)NC(=O)CCc2ccccc2)n1] | 0.002 |
| AA86 | 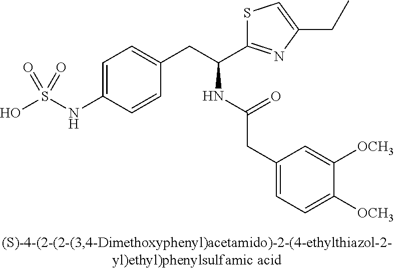 [SMILES: CCc1csc([C@H](Cc2ccc(NS(=O)(=O)O)cc2)NC(=O)Cc2ccc(OC)c(OC)c2)n1] | 0.028 |
| AA87 | 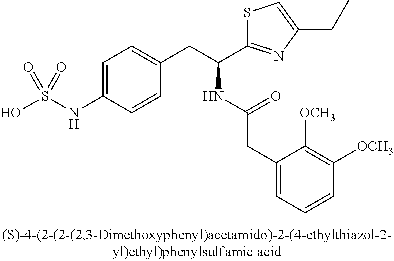 [SMILES: CCc1csc([C@H](Cc2ccc(NS(=O)(=O)O)cc2)NC(=O)Cc2cccc(OC)c2OC)n1] | 0.037 |
| AA88 | 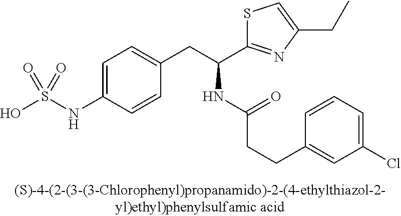 [SMILES: CCc1csc([C@H](Cc2ccc(NS(=O)(=O)O)cc2)NC(=O)CCc2cccc(Cl)c2)n1] | 0.0002 |
| AA89 | 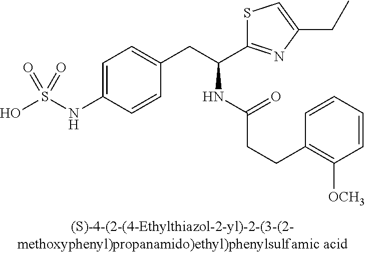 [SMILES: CCc1csc([C@H](Cc2ccc(NS(=O)(=O)O)cc2)NC(=O)CCc2ccccc2OC)n1] | 0.003 |
| AA90 | 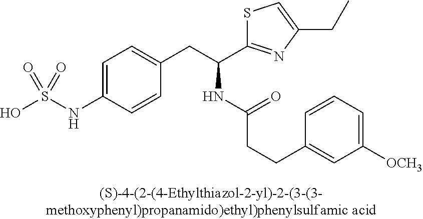 [SMILES: CCc1csc([C@H](Cc2ccc(NS(=O)(=O)O)cc2)NC(=O)CCc2cccc(OC)c2)n1] | 0.01 |
| AA91 | 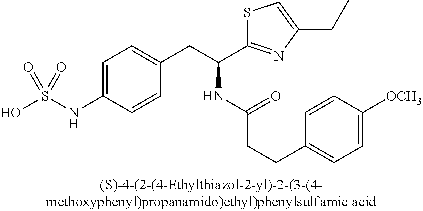 [SMILES: CCc1csc([C@H](Cc2ccc(NS(=O)(=O)O)cc2)NC(=O)CCc2ccc(OC)cc2)n1] | 0.006 |
| AA92 | 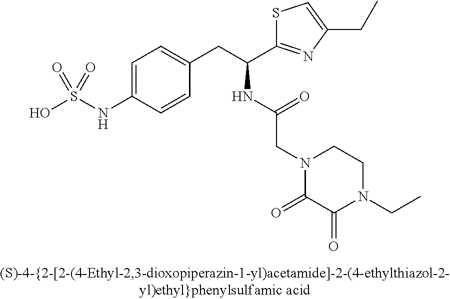 [SMILES: CCc1csc([C@H](Cc2ccc(NS(=O)(=O)O)cc2)NC(=O)CN2CCN(CC)C(=O)C2=O)n1] | 0.002 |
| AA93 | 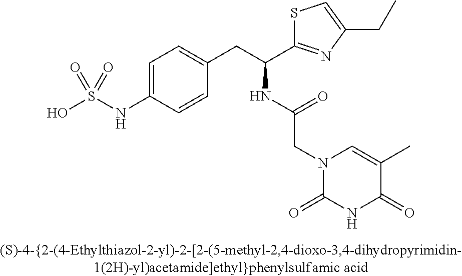 [SMILES: CCc1csc([C@H](Cc2ccc(NS(=O)(=O)O)cc2)NC(=O)Cn2cc(C)c(=O)[nH]c2=O)n1] | 0.002 |
| AA94 | 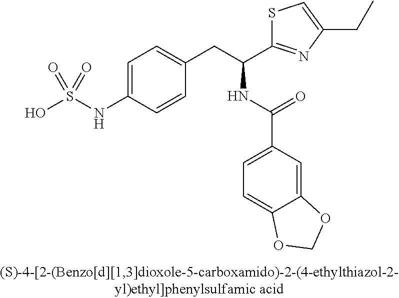 [SMILES: CCc1csc([C@H](Cc2ccc(NS(=O)(=O)O)cc2)NC(=O)c2ccc3c(c2)OCO3)n1] | 0.042 |
| AA95 | 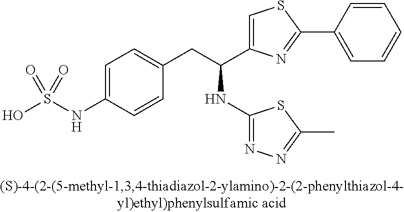 [SMILES: Cc1nnc(N[C@@H](Cc2ccc(NS(=O)(=O)O)cc2)c2csc(-c3ccccc3)n2)s1] | 0.003 |
| AA96 | 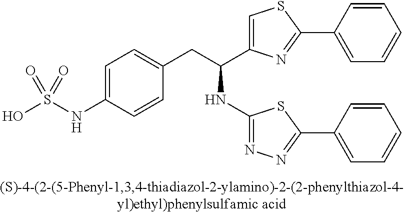 [SMILES: O=S(=O)(O)Nc1ccc(C[C@H](Nc2nnc(-c3ccccc3)s2)c2csc(-c3ccccc3)n2)cc1] | 0.046 |
| AA97 | 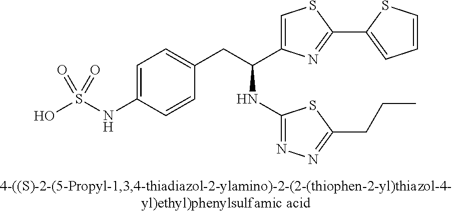 [SMILES: CCCc1nnc(N[C@@H](Cc2ccc(NS(=O)(=O)O)cc2)c2csc(-c3cccs3)n2)s1] | 0.0002 |
| AA98 | 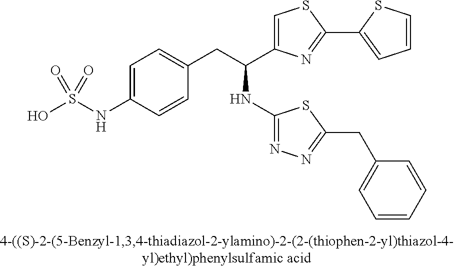 [SMILES: O=S(=O)(O)Nc1ccc(C[C@H](Nc2nnc(Cc3ccccc3)s2)c2csc(-c3cccs3)n2)cc1] | 0.0006 |
| AA99 | 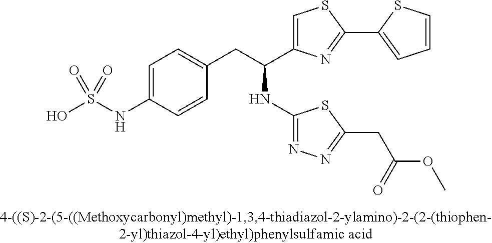 [SMILES: COC(=O)Cc1nnc(N[C@@H](Cc2ccc(NS(=O)(=O)O)cc2)c2csc(-c3cccs3)n2)s1] | 0.002 |
| AA100 | 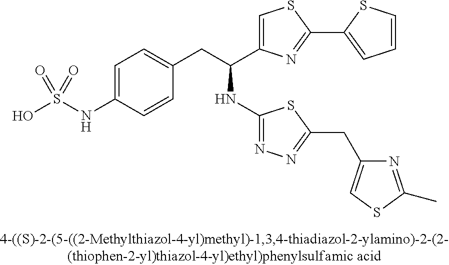 [SMILES: Cc1nc(Cc2nnc(N[C@@H](Cc3ccc(NS(=O)(=O)O)cc3)c3csc(-c4cccs4)n3)s2)cs1] | 9 × 10⁻⁶ |

### Example 2. Effect of Hypoxia on VE-PTP Expression in Human Umbilical Vein Endothelial Cells (HUVECs)

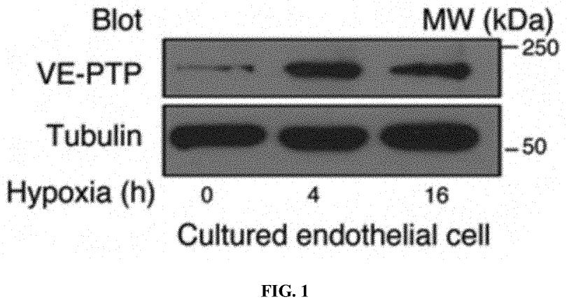

HUVECs were cultured under normoxic (21% oxygen), or hypoxic (5% oxygen) conditions for 4 or 16 hours. Western blot analysis was then performed using a rabbit polyclonal antibody against the C terminus of human VE-PTP. Blotting of tubulin protein served as a loading control. As seen in FIG. 1, VE-PTP was upregulated in cells exposed to hypoxic conditions.

### Example 3. Effect of Compound on Tie-2 Phosphorylation in Hypoxic HUVECs

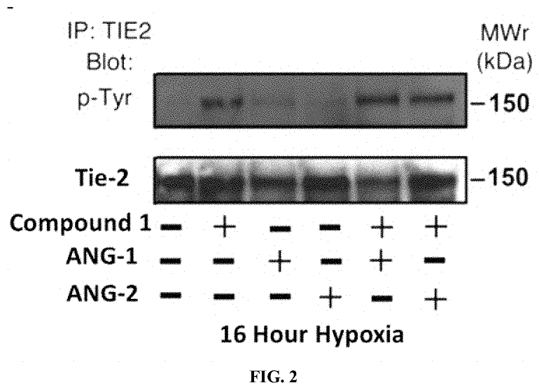

HUVECs cultured under hypoxic conditions for 16 hours were treated with 5 μM Compound 1 for 10 minutes in the presence or absence of ANG-1 or ANG-2 (500 ng/mL). Untreated cells served as controls. The cells were then lysed and Tie-2 was immunoprecipitated and probed with anti-phosphotyrosine (p-Tyr) to indicate Tie-2 activation, or anti-Tie-2 as a loading control. As shown in FIG. 2, treatment with Compound 1 increased Tie-2 phosphorylation, alone or in the presence of ANG-1 or ANG-2. ANG-1 treatment alone did not increase Tie-2 phosphorylation under these conditions.

### Example 4. Effect of Compound on Tie-2 Downstream Signaling in Hypoxic HUVECs

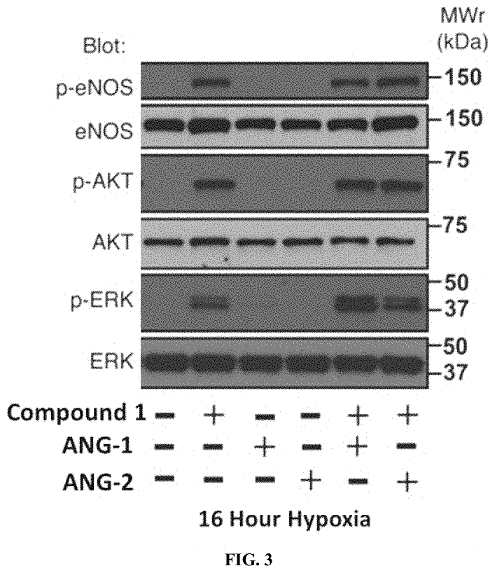

HUVECs cultured under hypoxic conditions for 16 hours were treated with 5 μM Compound 1 for 10 minutes in the presence or absence of ANG-1 or ANG-2 (500 ng/mL). Untreated cells served as controls. Following the treatment period, the cells were lysed and the lysates were probed with antibodies against total (as a loading control) and phosphorylated-AKT, ERK, and eNOS. FIG. 3 shows that treatment with Compound 1, but not ANG-1, led to an increase in phosphorylation of AKT, ERK, and eNOS, even in the presence of ANG-2.

### Example 5. Effects of Compound 1 on Cardiac Physiology in a Canine Model

A study was conducted to evaluate the potential pharmacological effects of Compound 1 in 10% HPßCD with 0.1% or 0.3% NaCl in Sterile Water for Injection, USP, on the cardiovascular system (arterial blood pressures, heart rate, electrocardiogram, and pulse pressure) in conscious, freely moving male dogs. Non-naïve dogs, previously instrumented with radio telemetry transmitter Implants® (DSI PhysioTel® D70-PCT or D70-PCTP, Data Science International, St. Paul, Minn.) were used in this study. The same four male beagle dogs were administered the vehicle, 10% HPßCD with 0.3% NaCl in Sterile Water for Injection, USP (0 mg/kg), and Compound 1, at dose levels of 10, 45, and 120 mg/kg according to a Latin square design, where one animal/treatment was dosed once followed by a 7 day washout period between administrations, until each animal received all treatments. The vehicle and Compound 1 were administered to all animals via subcutaneous injection at dose volumes of 1.71 mL/kg (×2 injection sites) for and 120 mg/kg, 1.25 mL/kg (×1 injection site) for 10 mg/kg, and 1.29 mL/kg (×1 injection site) for 45 mg/kg. Details of the treatment procedure and dosing schedule are shown in TABLE 2 and TABLE 3.

TABLE 2
Experimental Design

| | | Active | Dose Volume | |
|---|---|---|---|---|
| Treatment | Dose level | Concentration | (mL/kg)/Number of | Number of |
| number | (mg/kg)^{a} | (mg/mL) | Injection Sites | Animals^{b} |
| 1 | 0 | 0 | 1.71/2 | 4 |
| 2 | 10 | 8 | 1.25/1 | 4 |
| 3 | 45 | 35 | 1.29/1 | 4 |
| 4 | 120 | 35 | 1.71/2 | 4 |

^{a}The vehicle for dosing Treatment 1 was 10% HPβCD + 0.3% NaCl and was also used for the preparation of the test article for Treatment 2; the vehicle used for the preparation for the test article for Treatments 3 and 4 was 10% HPβCD + 0.1% NaCl.
^{b}Each treatment was administered to the same four animals according to a Latin square design with a 7-day washout between each treatment.

TABLE 3
Dosing Schedule

Dose Level (mg/kg)

| Animal Number | 0 | 10 | 45 | 120 |
|---|---|---|---|---|
| 3001 | Day 1 | Day 15 | Day 22 | Day 8 |
| 3002 | Day 8 | Day 1 | Day 15 | Day 22 |
| 3003 | Day 22 | Day 8 | Day 1 | Day 15 |
| 3004 | Day 15 | Day 22 | Day 8 | Day 1 |

Following treatment, mean plasma concentrations of Compound 1 (evaluated at 4 hours post-dose) were found to be dose proportional. The plasma concentration of Compound 1 for animal number 3001, which had been administered vehicle on Day 1, was below the limit of detection. Compound 1 was detected in the plasma of other animals following vehicle control administration, but at levels below those of animals dosed with Compound 1 four hours prior. Although these three animals had previously received Compound 1 treatments, the detectable levels of Compound 1 in plasma were not proportional to the last dose level administered and were not expected due to the 7-day washout period and short half-life of Compound 1. No test article was detected in the vehicle control formulations, and no source of cross contamination was identified. The mean concentration of Compound 1 detected for each dose is shown below in TABLE 4.

TABLE 4
Compound 1 Plasma Exposure Analysis

| | | Mean Plasma | |
|---|---|---|---|
| Dose Level | Time | Concentration | |
| (mg/kg) | (hour) | (ng/mL) | SD |
| 0.0 | 4 | 194 | 188 |
| 10 | 4 | 989 | 244 |
| 45 | 4 | 3962 | 1685 |
| 120 | 4 | 11436 | 2938 |

Systolic, diastolic, and derived mean arterial blood pressures and pulse pressures, heart rate, and ECG parameters (QRS duration and the RR, PR, and QT intervals) were monitored continuously in dogs from at least 2 hours pre-dose until at least 22 hours post-dose. ECG tracings were printed at designated time points from the cardiovascular monitoring data, and were qualitatively evaluated by a board-certified veterinary cardiologist. Ten days prior to the first administration, untreated animals were continuously monitored for cardiovascular endpoints for at least 24 hours. These data were used in the calculation of the heart rate corrected QT interval (QTc) throughout the study.

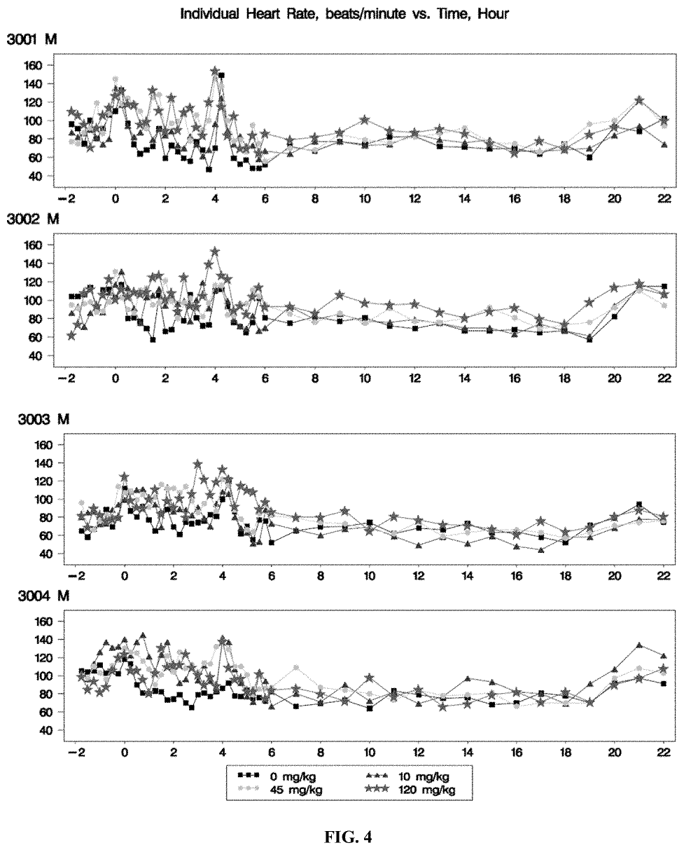

Least squares mean (LSMean) and mean heart rate values following treatment with Compound 1 are summarized in TABLE 5-6. Individual heart rate values are illustrated in FIG. 4. Beginning at approximately 30 minutes post-dose, heart rate was increased (about 20-50 bpm) at all dose levels of Compound 1 and generally returned to or near control and/or baseline values by the end of the 22-hour post-dose monitoring session. These changes are not considered to be adverse in magnitude or duration. Mean changes in heart rate reached statistical significance between 30 minutes to 6 hours post-dose at 10 and 45 mg/kg. At 10 mg/kg, statistical significance was reached for 10 out of 24 data points. At 45 mg/kg, statistical significance was reached for 18 out of 24 data points. Statistical significance was reached on 3 occasions between 6 and 22 hours post-dose at 45 mg/kg. The high dose of 120 mg/kg produced statistically significant increases in heart rate between 30 minutes to 15 hours for 28 out of 32 occasions and also at 19 hours post-dose. Changes in heart rate seen at approximately 4 hours post-dose were considered to be induced by the general restraint and handling of animals during blood collection.

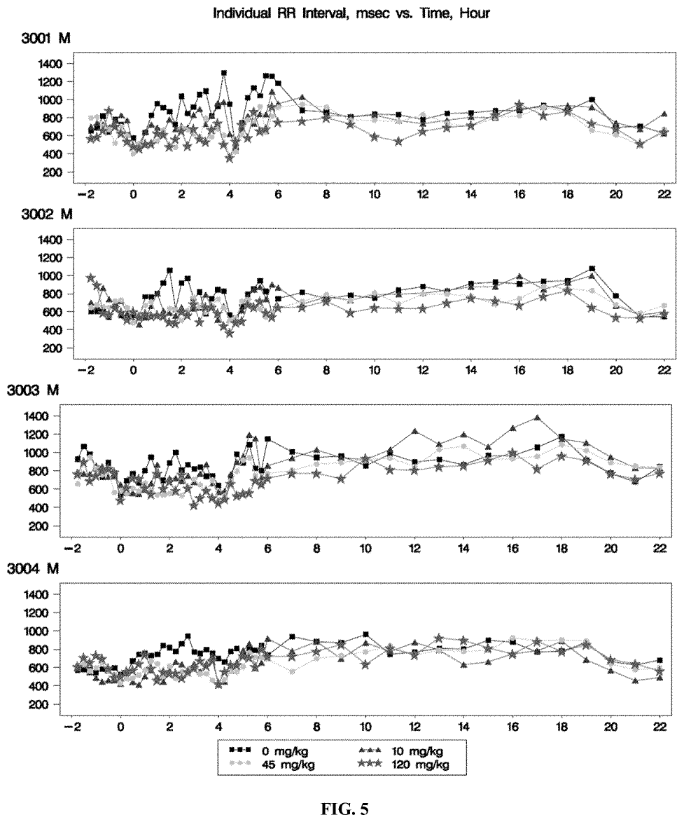

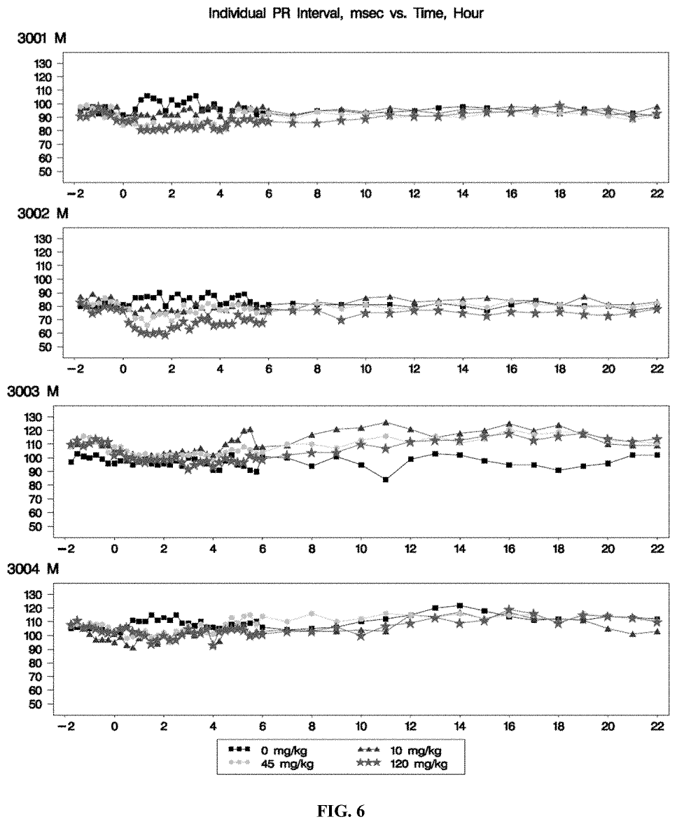

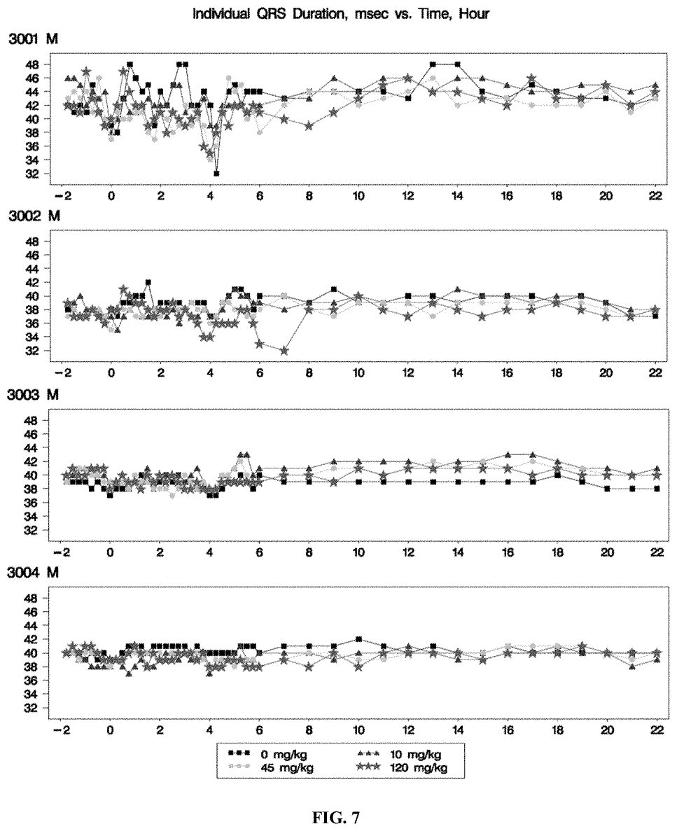

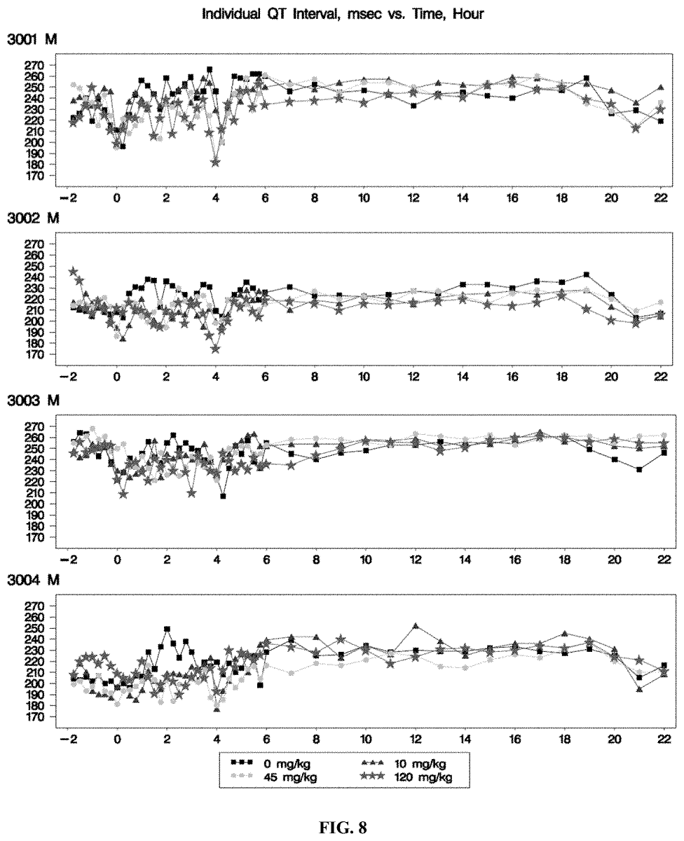

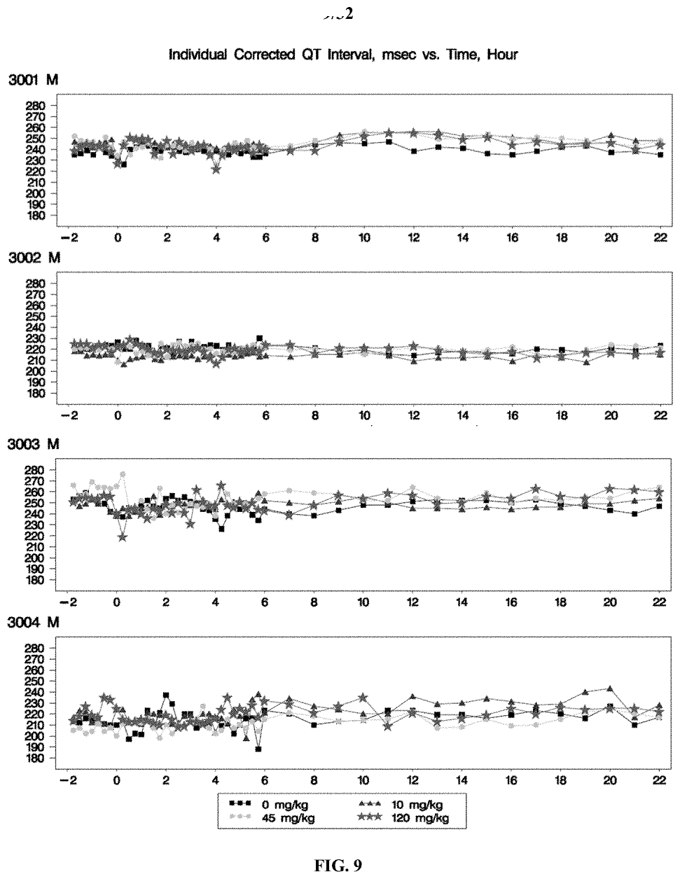

LS mean and mean values for RR interval, PR interval, QRS duration, QT interval, and QTc interval are summarized in TABLE 7-16. Data for each individual dog tested are shown in FIG. 5-9. Consistent with observed increases in heart rate, the RR, PR, and (uncorrected) QT interval durations were slightly decreased following Compound 1 administration at all dose levels, and were inversely related to the effect on heart rate described above. Mean changes in PR and QT intervals frequently reached statistical significance between 30 minutes to 4 hours post-dose at 10 and 45 mg/kg, and between 30 minutes to 6 hours post-dose at 120 mg/kg.

Compound 1 treatment had no observed effect on QRS duration, QTc, and qualitative aspects of the ECG in male beagle dogs. Any changes that were seen were not physiologically relevant, not dose dependent, and not considered to be outside the normal range of variability.

Consistent with the observed increases in heart rate, the RR, PR, QRS, and QT intervals were briefly decreased for most animals immediately following each dose, including the vehicle control treatment, and at approximately 4 hours post-dose. These changes were considered to be induced by the general restraint and handling of the animals, or the presence of technical staff in the study room.

### Example 6. Effect of Compound 1 on Blood Pressure in a Canine Study

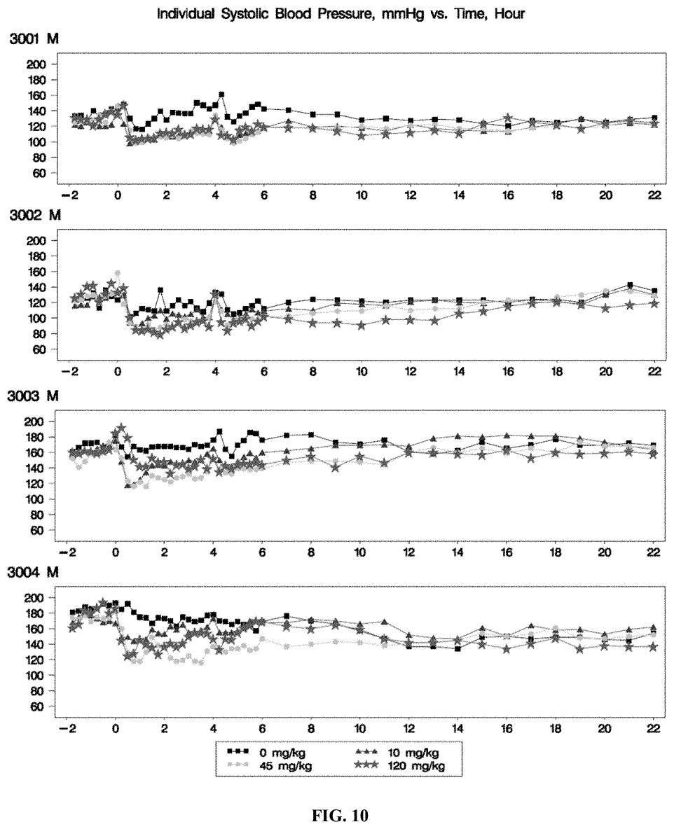

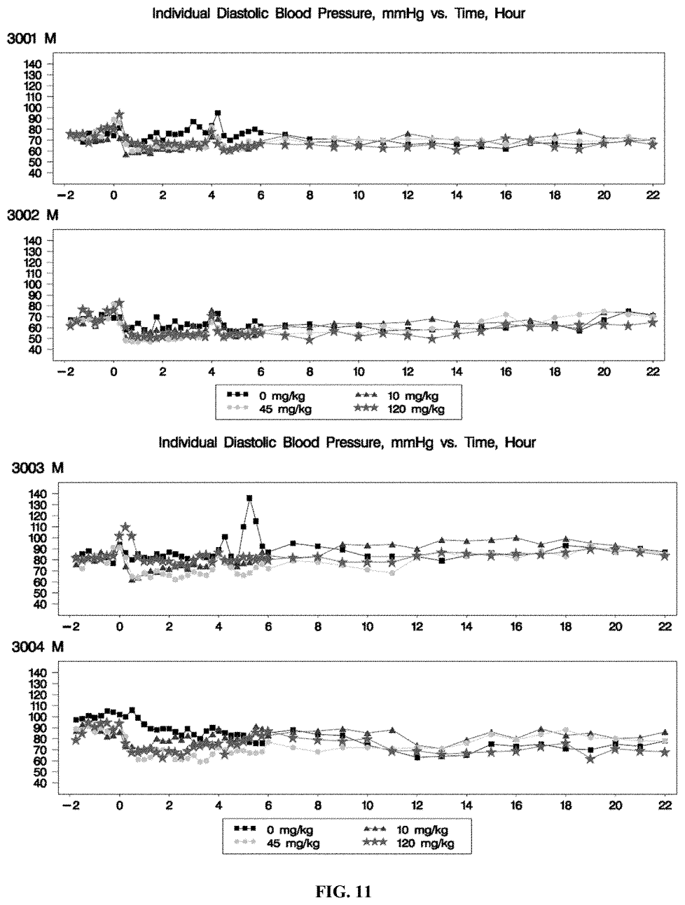

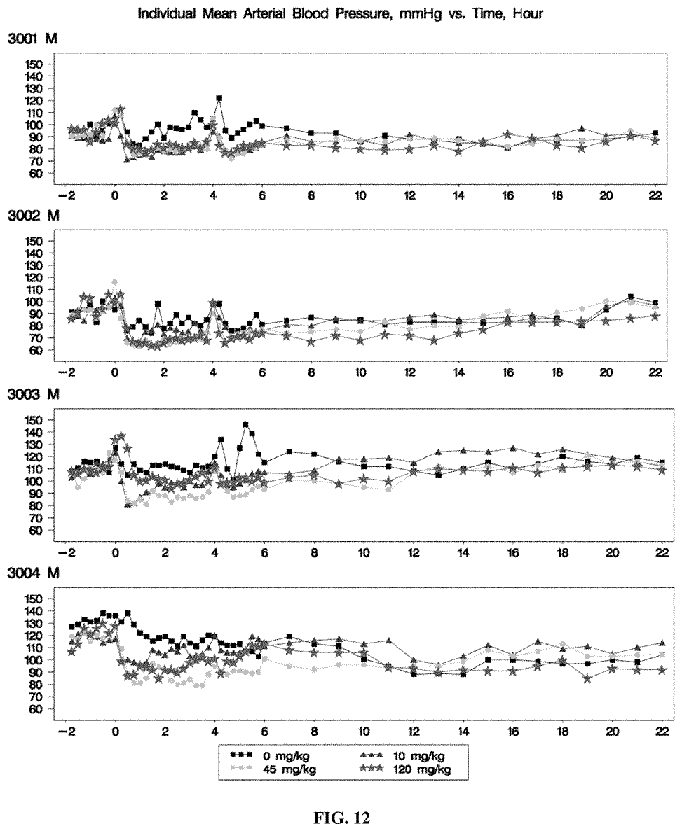

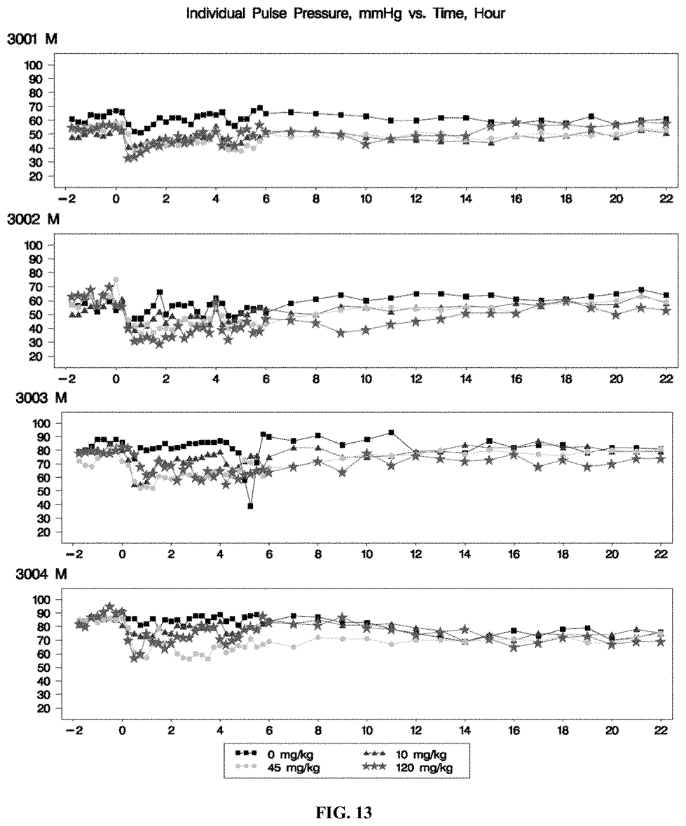

Following treatment with Compound 1 as described above in TABLE 2 and TABLE 3, blood pressure in dogs was monitored over a 22-hour period. LSMean and mean systolic, diastolic, mean arterial, and pulse pressure values are summarized in TABLE 17-24. Systolic blood pressure, diastolic blood pressure, mean arterial pressure, and pulse pressure values measured for each of the 4 dogs studied is summarized in FIG. 10-13.

Beginning at approximately 30 minutes post-dose, decreases in systolic blood pressure, diastolic blood pressure, mean arterial pressure, and pulse pressure were seen at all doses of Compound 1 treatment. For systolic blood pressure, a decrease of about 20-40 mmHg (about 15-25%) was observed. For diastolic blood pressure, a decrease of about 5-20 mmHg (about 10-25%) was observed. For mean arterial pressure, a decrease of about 10-30 mmHg (about 10-25%) was observed. For pulse pressure a decrease of about 10-20 mmHg was observed. Observed decreases generally returned to or near control and/or baseline values by 11 hours post-dose for 10 and 45 mg/kg doses, and at the end of the 22-hour post-dose monitoring session for the 120 mg/kg dose.

Changes were found to be dependent on the dose of Compound 1 administered. For the 10 mg/kg dose, the decrease in systolic blood pressure reached statistical significance on only two occasions over the first 5 hours post-dose. The decrease in mean arterial pressure reached statistical significance at only 7 hours post-dose. Mean decreases in blood pressure frequently reached statistical significance for systolic blood pressure at 120 mg/kg between 45 minutes to 11 hours post-dose and 21 to 22 hours post-dose, and for diastolic blood pressure, between 7 to 9 hours post-dose at 45 mg/kg, and between 7 to 11 and at 22 hours post-dose at 120 mg/kg. Statistically significant decreases in mean arterial blood pressures were similarly observed between 7 to 10 hours post-dose at 45 mg/kg, and between 7 to 11 hours and 21 to 22 hours post-dose at 120 mg/kg. Pulse pressure was statistically significantly reduced between 7 to 11 hours post-dose at 45 mg/kg and for 10 out of 13 observations between 7 to 19 hours post-dose at 120 mg/kg.

Systolic blood pressure, diastolic blood pressure, and mean arterial pressure were briefly increased for most animals immediately following each dose and at approximately 4 hours post-dose. These increases were similar in magnitude in all groups and are considered to be induced by the general restraint and handling of the animas for the dose administration and/or the presence of technical staff in the study room during dosing or at approximately 4 hours post-dose for blood collection, and are not considered to be related to Compound 1 administration.

TABLE 5A shown below presents a summary of heart rate values (bpm) measured in EXAMPLE 5 described above. Statistical analysis was based on a mixed model analysis of 0.25 through 6 Hour Time Interval Values (Segment 1). The covariate mean is the average of 2-hour pre-dose data with an autoregressive(1) [AR(1)] covariance structure.

TABLE 5A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 95.78 | Mean | 78.38 | 112.50 | 86.75 | 82.00 | 74.50 | 71.25 | 73.75 | 89.50 | 67.00 | 70.25 |
| mg/kg | | N | 4 | 4 | 4 | 4 | 4 | 4 | 4 | 4 | 4 | 4 |
| | | LSMean | 78.78 | 112.91 | 87.16 | 82.41 | 74.91 | 71.66 | 74.16 | 89.91 | 67.41 | 70.66 |
| | | LSM s.e. | 1.86 | 5.10 | 5.10 | 5.10 | 5.10 | 5.10 | 5.10 | 5.10 | 5.10 | 5.10 |
| 10 | 97.19 | Mean | 92.79 | 119.50 | 113.50 | 107.25 | 96.25 | 99.50 | 94.00 | 113.25 | 92.75 | 93.50 |
| mg/kg | | N | 4 | 4 | 4 | 4 | 4 | 4 | 4 | 4 | 4 | 4 |
| | | LSMean | 92.62 | 119.32 | 113.32 | 107.07 | 96.07 | 99.32 | 93.82 | 113.07 | 92.57 | 93.32 |
| | | LSM s.e. | 1.85 | 5.10 | 5.10 | 5.10 | 5.10 | 5.10 | 5.10 | 5.10 | 5.10 | 5.10 |
| | | Trend | 0.003* | NT | 0.000* | 0.001* | 0.004* | 0.000* | 0.008* | 0.002* | 0.001* | 0.002* |
| | | p-value | | | | | | | | | | |
| 45 | 97.75 | Mean | 101.35 | 114.00 | 109.50 | 106.25 | 104.75 | 99.00 | 109.25 | 114.75 | 106.50 | 107.25 |
| mg/kg | | N | 4 | 4 | 4 | 4 | 4 | 4 | 4 | 4 | 4 | 4 |
| | | LSMean | 100.95 | 113.59 | 109.09 | 105.84 | 104.34 | 98.59 | 108.84 | 114.34 | 106.09 | 106.84 |
| | | LSM s.e. | 1.86 | 5.10 | 5.10 | 5.10 | 5.10 | 5.10 | 5.10 | 5.10 | 5.10 | 5.10 |
| | | Trend | 0.000* | NT | 0.003* | 0.002* | 0.000* | 0.000* | 0.000* | 0.001* | 0.000* | 0.000* |
| | | p-value | | | | | | | | | | |
| 120 | 96.34 | Mean | 104.32 | 112.25 | 104.75 | 103.00 | 96.00 | 105.50 | 118.25 | 111.50 | 98.25 | 111.50 |
| mg/kg | | N | 4 | 4 | 4 | 4 | 4 | 4 | 4 | 4 | 4 | 4 |
| | | LSMean | 104.50 | 112.43 | 104.93 | 103.18 | 96.18 | 105.68 | 118.43 | 111.68 | 98.43 | 111.68 |
| | | LSM s.e. | 1.85 | 5.10 | 5.10 | 5.10 | 5.10 | 5.10 | 5.10 | 5.10 | 5.10 | 5.10 |
| | | Trend | 0.000* | 0.753 | 0.034* | 0.009* | 0.002* | 0.000* | 0.000* | 0.004* | 0.000* | 0.000* |
| | | p-value | | | | | | | | | | |

TABLE 5B shown below presents a summary of heart rate values (bpm) measured in EXAMPLE 5 described above. Statistical analysis was based on a mixed model analysis of 0.25 through 6 Hour Time Interval Values (Segment 1). The covariate mean is the average of 2-hour pre-dose data with an AR(1) covariance structure.

TABLE 5B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 95.78 | Mean | 74.50 | 68.75 | 78.75 | 78.75 | 75.00 | 70.75 | 91.50 | 118.50 | 85.50 | 68.50 |
| mg/kg | | N | 4 | 4 | 4 | 4 | 4 | 4 | 4 | 4 | 4 | 4 |
| | | LSMean | 74.91 | 69.16 | 79.16 | 79.16 | 75.41 | 71.16 | 91.91 | 118.91 | 85.91 | 68.91 |
| | | LSM s.e. | 5.10 | 5.10 | 5.10 | 5.10 | 5.10 | 5.10 | 5.10 | 5.10 | 5.10 | 5.10 |
| 10 | 97.19 | Mean | 88.25 | 90.75 | 86.50 | 91.00 | 86.00 | 84.50 | 114.25 | 120.75 | 93.75 | 78.25 |
| mg/kg | | N | 4 | 4 | 4 | 4 | 4 | 4 | 4 | 4 | 4 | 4 |
| | | LSMean | 88.07 | 90.57 | 86.32 | 90.82 | 85.82 | 84.32 | 114.07 | 120.57 | 93.57 | 78.07 |
| | | LSM s.e. | 5.10 | 5.10 | 5.10 | 5.10 | 5.10 | 5.10 | 5.10 | 5.10 | 5.10 | 5.10 |
| | | Trend | 0.071 | 0.004* | NT | 0.109 | 0.152 | 0.071 | 0.003* | NT | NT | 0.207 |
| | | p-value | | | | | | | | | | |
| 45 | 97.75 | Mean | 100.25 | 103.50 | 92.25 | 101.50 | 97.25 | 104.00 | 130.25 | 124.25 | 95.50 | 87.25 |
| mg/kg | | N | 4 | 4 | 4 | 4 | 4 | 4 | 4 | 4 | 4 | 4 |
| | | LSMean | 99.84 | 103.09 | 91.84 | 101.09 | 96.84 | 103.59 | 129.84 | 123.84 | 95.09 | 86.84 |
| | | LSM s.e. | 5.10 | 5.10 | 5.10 | 5.10 | 5.10 | 5.10 | 5.10 | 5.10 | 5.10 | 5.10 |
| | | Trend | 0.001* | 0.000* | 0.082 | 0.003* | 0.004* | 0.000* | 0.000* | NT | NT | 0.015* |
| | | p-value | | | | | | | | | | |
| 120 | 96.34 | Mean | 95.50 | 112.25 | 112.00 | 99.50 | 98.25 | 116.75 | 144.50 | 118.25 | 98.75 | 101.50 |
| mg/kg | | N | 4 | 4 | 4 | 4 | 4 | 4 | 4 | 4 | 4 | 4 |
| | | LSMean | 95.68 | 112.43 | 112.18 | 99.68 | 98.43 | 116.93 | 144.68 | 118.43 | 98.93 | 101.68 |
| | | LSM s.e. | 5.10 | 5.10 | 5.10 | 5.10 | 5.10 | 5.10 | 5.10 | 5.10 | 5.10 | 5.10 |
| | | Trend | 0.002* | 0.000* | 0.000* | 0.002* | 0.001* | 0.000* | 0.000* | 0.937 | 0.078 | 0.000* |
| | | p-value | | | | | | | | | | |

TABLE 5C shown below presents a summary of heart rate values (bpm) and statistical analysis measured in EXAMPLE 5 described above. Statistical analysis was based on a mixed model analysis of 0.25 through Hour Time Interval Values (Segment 1). The covariate mean is the average of 2-hour pre-dose data with an AR(1)covariance structure.

For TABLE 5A-C,N=numbers of measures t calculate mean; LSMean=Least squares mean; LSM s.e.=Least squares standard error; *=p<0.05; NT=not tested.

TABLE 5C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 95.78 | Mean | 69.50 | 63.00 | 69.25 | 74.50 | 66.75 |
| mg/kg | | N | 4 | 4 | 4 | 4 | 4 |
| | | LSMean | 69.91 | 63.41 | 69.66 | 74.91 | 67.16 |
| | | LSM s.e. | 5.10 | 5.10 | 5.10 | 5.10 | 5.10 |
| 10 | 97.19 | Mean | 73.75 | 65.75 | 78.25 | 76.75 | 69.00 |
| mg/kg | | N | 4 | 4 | 4 | 4 | 4 |
| | | LSMean | 73.57 | 65.57 | 78.07 | 76.57 | 68.82 |
| | | LSM s.e. | 5.10 | 5.10 | 5.10 | 5.10 | 5.10 |
| | | Trend p-value | 0.612 | NT | 0.246 | 0.818 | NT |
| 45 | 97.75 | Mean | 85.50 | 76.25 | 93.75 | 89.75 | 80.00 |
| mg/kg | | N | 4 | 4 | 4 | 4 | 4 |
| | | LSMean | 85.09 | 75.84 | 93.34 | 89.34 | 79.59 |
| | | LSM s.e. | 5.10 | 5.10 | 5.10 | 5.10 | 5.10 |
| | | Trend p-value | 0.038* | 0.088 | 0.001* | 0.048* | 0.088 |
| 120 | 96.34 | Mean | 89.25 | 86.50 | 94.75 | 87.75 | 87.25 |
| mg/kg | | N | 4 | 4 | 4 | 4 | 4 |
| | | LSMean | 89.43 | 86.68 | 94.93 | 87.93 | 87.43 |
| | | LSM s.e. | 5.10 | 5.10 | 5.10 | 5.10 | 5.10 |
| | | Trend p-value | 0.003* | 0.001* | 0.000* | 0.025* | 0.002* |

| Effects | Statistics | Value |
|---|---|---|
| MAIN EFFECT | Group F-test p-value | 0.001* |
| INTERACTION | Group*Time p-value | 0.000* |
| | Group Linear Trend*Linear Time p-value | 0.668 |
| | Group Linear Trend*Quadratic Time p-value | 0.000* |

TABLE 6A shown below presents a summary of heart rate values (bpm) measured in EXAMPLE 5 described above. Statistical analysis was based on a mixed model analysis of 7 through 22 Hour Time Interval Values (Segment 2). The covariate mean is the average of 2-hour pre-dose data with an AR(1) covariance structure.

TABLE 6A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 95.78 | Mean | 74.80 | 69.50 | 71.75 | 74.25 | 73.25 | 74.75 | 74.75 | 72.00 | 71.75 | 66.75 |
| mg/kg | | N | 4 | 4 | 4 | 4 | 4 | 4 | 4 | 4 | 4 | 4 |
| | | LSMean | 75.03 | 69.73 | 71.98 | 74.48 | 73.48 | 74.98 | 74.98 | 72.23 | 71.98 | 66.98 |
| | | LSM s.e. | 1.56 | 3.61 | 3.61 | 3.61 | 3.61 | 3.61 | 3.61 | 3.61 | 3.61 | 3.61 |
| 10 | 97.19 | Mean | 76.17 | 75.75 | 71.75 | 79.75 | 72.75 | 72.75 | 70.50 | 72.25 | 73.50 | 75.25 |
| mg/kg | | N | 4 | 4 | 4 | 4 | 4 | 4 | 4 | 4 | 4 | 4 |
| | | LSMean | 76.07 | 75.65 | 71.65 | 79.65 | 72.65 | 72.65 | 70.40 | 72.15 | 73.40 | 75.15 |
| | | LSM s.e. | 1.55 | 3.61 | 3.61 | 3.61 | 3.61 | 3.61 | 3.61 | 3.61 | 3.61 | 3.61 |
| | | Trend p-value | NT | 0.251 | NT | NT | NT | NT | NT | NT | NT | 0.114 |
| 45 | 97.75 | Mean | 79.72 | 85.75 | 76.25 | 82.00 | 75.00 | 75.50 | 79.50 | 74.75 | 78.50 | 78.75 |
| mg/kg | | N | 4 | 4 | 4 | 4 | 4 | 4 | 4 | 4 | 4 | 4 |
| | | LSMean | 79.49 | 85.52 | 76.02 | 81.77 | 74.77 | 75.27 | 79.27 | 74.52 | 78.27 | 78.52 |
| | | LSM s.e. | 1.56 | 3.61 | 3.61 | 3.61 | 3.61 | 3.61 | 3.61 | 3.61 | 3.61 | 3.61 |
| | | Trend p-value | 0.100 | 0.003* | 0.432 | 0.159 | 0.802 | 0.955 | 0.405 | NT | NT | 0.027* |
| 120 | 96.34 | Mean | 84.59 | 84.50 | 82.00 | 88.00 | 90.25 | 85.50 | 86.25 | 79.00 | 76.75 | 77.25 |
| mg/kg | | N | 4 | 4 | 4 | 4 | 4 | 4 | 4 | 4 | 4 | 4 |
| | | LSMean | 84.69 | 84.60 | 82.10 | 88.10 | 90.35 | 85.60 | 86.35 | 79.10 | 76.85 | 77.35 |
| | | LSM s.e. | 1.55 | 3.61 | 3.61 | 3.61 | 3.61 | 3.61 | 3.61 | 3.61 | 3.61 | 3.61 |
| | | Trend p-value | 0.005* | 0.001* | 0.035* | 0.010* | 0.002* | 0.036* | 0.010* | 0.159 | 0.232 | 0.036* |

TABLE 6B shown below presents a summary of heart rate values (bpm) measured in EXAMPLE 5 described above. Statistical analysis was based on a mixed model analysis of 7 through 22 Hour Time Interval Values (Segment 2). The covariate mean is the average of 2-hour pre-dose data with an AR(1) covariance structure.

TABLE 6B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 95.78 | Mean | 68.00 | 67.00 | 67.75 | 64.50 | 86.50 | 98.50 | 95.75 |
| mg/kg | | N | 4 | 4 | 4 | 4 | 4 | 4 | 4 |
| | | LSMean | 68.23 | 67.23 | 67.98 | 64.73 | 86.73 | 98.73 | 95.98 |
| | | LSM s.e. | 3.61 | 3.61 | 3.61 | 3.61 | 3.61 | 3.61 | 3.61 |
| 10 | 97.19 | Mean | 65.00 | 66.25 | 65.50 | 70.00 | 88.25 | 104.75 | 94.75 |
| mg/kg | | N | 4 | 4 | 4 | 4 | 4 | 4 | 4 |
| | | LSMean | 64.90 | 66.15 | 65.40 | 69.90 | 88.15 | 104.65 | 94.65 |
| | | LSM s.e. | 3.61 | 3.61 | 3.61 | 3.61 | 3.61 | 3.61 | 3.61 |
| | | Trend p-value | NT | NT | NT | 0.315 | NT | NT | NT |
| 45 | 97.75 | Mean | 72.00 | 67.00 | 68.50 | 76.25 | 90.00 | 104.00 | 91.75 |
| mg/kg | | N | 4 | 4 | 4 | 4 | 4 | 4 | 4 |
| | | LSMean | 71.77 | 66.77 | 68.27 | 76.02 | 89.77 | 103.77 | 91.52 |
| | | LSM s.e. | 3.61 | 3.61 | 3.61 | 3.61 | 3.61 | 3.61 | 3.61 |
| | | Trend p-value | NT | NT | NT | 0.031* | NT | NT | NT |
| 120 | 96.34 | Mean | 75.00 | 76.25 | 72.25 | 80.75 | 94.50 | 106.25 | 99.00 |
| mg/kg | | N | 4 | 4 | 4 | 4 | 4 | 4 | 4 |
| | | LSMean | 75.10 | 76.35 | 72.35 | 80.85 | 94.60 | 106.35 | 99.10 |
| | | LSM s.e. | 3.61 | 3.61 | 3.61 | 3.61 | 3.61 | 3.61 | 3.61 |
| | | Trend p-value | 0.093 | 0.088 | 0.326 | 0.001* | 0.123 | 0.178 | 0.701 |

TABLE 6C shown below presents statistical analysis of data presented in TABLE 6A-B. For TABLE 6A-B, N=numbers of measures to calculate mean; LSMean=Least squares mean; LSM s.e.=Least squares standard error; *=p<0.05; NT=not tested.

TABLE 6C

| Effects | Statistics | Value |
|---|---|---|
| MAIN EFFECT | Group F-test p-value | 0.024* |
| INTERACTION | Group*Time p-value | 0.794 |
| | Group Linear Trend*Linear Time p-value | 0.037* |
| | Group Linear Trend*Quadratic Time p-value | 0.856 |

TABLE 7A shown below presents a summary of RR interval values (msec) measured in EXAMPLE 5 described above. Statistical analysis was based on a mixed Model analysis of 0.25 through 6 Hour Time Interval Values (Segment 1). The covariate mean is the average of 2-hour pre-dose data with an AR(1) covariance structure.

TABLE 7A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 669.53 | Mean | 819.98 | 554.50 | 709.50 | 749.50 | 828.00 | 879.00 | 854.00 | 721.50 | 915.00 | 898.75 |
| mg/kg | | N | 4 | 4 | 4 | 4 | 4 | 4 | 4 | 4 | 4 | 4 |
| | | LSMean | 817.74 | 552.26 | 707.26 | 747.26 | 825.76 | 876.76 | 851.76 | 719.26 | 912.76 | 896.51 |
| | | LSM s.e. | 12.12 | 15.70 | 22.14 | 19.67 | 14.04 | 18.36 | 66.72 | 21.79 | 42.68 | 45.35 |
| 10 | 667.44 | Mean | 691.85 | 506.00 | 544.25 | 587.00 | 646.50 | 618.25 | 682.75 | 557.50 | 660.00 | 662.75 |
| mg/kg | | N | 4 | 4 | 4 | 4 | 4 | 4 | 4 | 4 | 4 | 4 |
| | | LSMean | 690.79 | 504.93 | 543.18 | 585.93 | 645.43 | 617.18 | 681.68 | 556.43 | 658.93 | 661.68 |
| | | LSM s.e. | 12.11 | 15.69 | 22.14 | 19.66 | 14.03 | 18.35 | 66.72 | 21.78 | 42.68 | 45.35 |
| 45 | 655.94 | Mean | 617.80 | 530.50 | 564.50 | 579.25 | 581.25 | 618.25 | 576.00 | 534.25 | 590.75 | 580.00 |
| mg/kg | | N | 4 | 4 | 4 | 4 | 4 | 4 | 4 | 4 | 4 | 4 |
| | | LSMean | 623.18 | 535.88 | 569.88 | 584.63 | 586.63 | 623.63 | 581.38 | 539.63 | 596.13 | 585.38 |
| | | LSM s.e. | 12.19 | 15.76 | 22.18 | 19.71 | 14.10 | 18.41 | 66.74 | 21.83 | 42.70 | 45.37 |
| 120 | 669.22 | Mean | 597.56 | 543.00 | 593.75 | 604.00 | 636.50 | 575.50 | 548.50 | 548.00 | 618.75 | 543.00 |
| mg/kg | | N | 4 | 4 | 4 | 4 | 4 | 4 | 4 | 4 | 4 | 4 |
| | | LSMean | 595.50 | 540.93 | 591.68 | 601.93 | 634.43 | 573.43 | 546.43 | 545.93 | 616.68 | 540.93 |
| | | LSM s.e. | 12.12 | 15.70 | 22.14 | 19.67 | 14.04 | 18.36 | 66.72 | 21.79 | 42.68 | 45.35 |

TABLE 7B shown below presents a summary of RR interval values (msec) measured in EXAMPLE 5 described above. Statistical analysis was based on a mixed Model analysis of 0.25 through 6 Hour Time Interval Values (Segment 1). The covariate mean is the average of 2-hour pre-dose data with an AR(1) covariance structure.

TABLE 7B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 669.53 | Mean | 833.25 | 920.00 | 814.75 | 789.25 | 827.00 | 907.50 | 714.25 | 528.50 | 719.00 | 899.75 |
| mg/kg | | N | 4 | 4 | 4 | 4 | 4 | 4 | 4 | 4 | 4 | 4 |
| | | LSMean | 831.01 | 917.76 | 812.51 | 787.01 | 824.76 | 905.26 | 712.01 | 526.26 | 716.76 | 897.51 |
| | | LSM s.e. | 36.06 | 19.78 | 40.95 | 40.47 | 47.81 | 60.36 | 47.65 | 36.54 | 33.94 | 36.08 |
| 10 | 667.44 | Mean | 697.00 | 713.50 | 709.00 | 698.00 | 746.25 | 757.50 | 526.50 | 510.00 | 655.75 | 784.25 |
| mg/kg | | N | 4 | 4 | 4 | 4 | 4 | 4 | 4 | 4 | 4 | 4 |
| | | LSMean | 695.93 | 712.43 | 707.93 | 696.93 | 745.18 | 756.43 | 525.43 | 508.93 | 654.68 | 783.18 |
| | | LSM s.e. | 36.05 | 19.77 | 40.95 | 40.47 | 47.81 | 60.36 | 47.65 | 36.54 | 33.93 | 36.07 |
| 45 | 655.94 | Mean | 615.50 | 601.00 | 669.50 | 607.50 | 629.75 | 603.50 | 446.25 | 487.25 | 642.75 | 713.75 |
| mg/kg | | N | 4 | 4 | 4 | 4 | 4 | 4 | 4 | 4 | 4 | 4 |
| | | LSMean | 620.88 | 606.38 | 674.88 | 612.88 | 635.13 | 608.88 | 451.63 | 492.63 | 648.13 | 719.13 |
| | | LSM s.e. | 36.08 | 19.83 | 40.97 | 40.49 | 47.83 | 60.38 | 47.67 | 36.56 | 33.96 | 36.10 |
| 120 | 669.22 | Mean | 652.75 | 554.50 | 552.25 | 624.50 | 631.50 | 532.50 | 398.00 | 515.50 | 627.00 | 603.50 |
| mg/kg | | N | 4 | 4 | 4 | 4 | 4 | 4 | 4 | 4 | 4 | 4 |
| | | LSMean | 650.68 | 552.43 | 550.18 | 622.43 | 629.43 | 530.43 | 395.93 | 513.43 | 624.93 | 601.43 |
| | | LSM s.e. | 36.06 | 19.78 | 40.95 | 40.47 | 47.81 | 60.36 | 47.65 | 36.54 | 33.94 | 36.07 |

TABLE 7C shown below presents a summary of RR interval values (msec) measured in EXAMPLE 5 described above. Statistical analysis was based on a mixed Model analysis of 0.25 through 6 Hour Time Interval Values (Segment 1). The covariate mean is the average of 2-hour pre-dose data with an AR(1) covariance structure.

For TABLE 7A-C, N=numbers of measures to calculate mean; LSMean=Least squares mean; LSM s.e.=Least squares standard error; *=p<0.05; NT=not tested.

TABLE 7C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 669.53 | Mean | 898.75 | 967.00 | 927.25 | 870.75 | 952.75 |
| mg/kg | | N | 4 | 4 | 4 | 4 | 4 |
| | | LSMean | 896.51 | 964.76 | 925.01 | 868.51 | 950.51 |
| | | LSM s.e. | 46.62 | 38.70 | 72.30 | 34.91 | 35.01 |
| 10 | 667.44 | Mean | 830.75 | 941.75 | 830.50 | 841.25 | 897.50 |
| mg/kg | | N | 4 | 4 | 4 | 4 | 4 |
| | | LSMean | 829.68 | 940.68 | 829.43 | 840.18 | 896.43 |
| | | LSM s.e. | 46.62 | 38.69 | 72.30 | 34.91 | 35.01 |
| 45 | 655.94 | Mean | 733.50 | 813.50 | 660.00 | 694.75 | 754.00 |
| mg/kg | | N | 4 | 4 | 4 | 4 | 4 |
| | | LSMean | 738.88 | 818.88 | 665.38 | 700.13 | 759.38 |
| | | LSM s.e. | 46.64 | 38.72 | 72.31 | 34.94 | 35.03 |
| 120 | 669.22 | Mean | 698.25 | 659.25 | 641.50 | 730.50 | 709.00 |
| mg/kg | | N | 4 | 4 | 4 | 4 | 4 |
| | | LSMean | 696.18 | 657.18 | 639.43 | 728.43 | 706.93 |
| | | LSM s.e. | 46.62 | 38.70 | 72.30 | 34.91 | 35.01 |

TABLE 8A shown below presents a summary of RR interval values (msec) measured in EXAMPLE 5 described above. Statistical analysis was based on a mixed model analysis of 7 through 22 Hour Time Interval Values (Segment 2). The covariate mean is the average of 2-hour pre-dose data with an AR(1) covariance structure.

TABLE 8A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 669.53 | Mean | 847.86 | 910.00 | 859.00 | 855.75 | 851.75 | 850.00 | 831.00 | 849.75 | 859.25 | 916.25 |
| mg/kg | | N | 4 | 4 | 4 | 4 | 4 | 4 | 4 | 4 | 4 | 4 |
| | | LSMean | 846.52 | 908.66 | 857.66 | 854.41 | 850.41 | 848.66 | 829.66 | 848.41 | 857.91 | 914.91 |
| | | LSM s.e. | 22.54 | 38.76 | 38.76 | 38.76 | 38.76 | 38.76 | 38.76 | 38.76 | 38.76 | 38.76 |
| 10 | 667.44 | Mean | 853.28 | 858.25 | 875.75 | 793.75 | 853.25 | 842.00 | 913.25 | 872.50 | 878.25 | 850.00 |
| mg/kg | | N | 4 | 4 | 4 | 4 | 4 | 4 | 4 | 4 | 4 | 4 |
| | | LSMean | 852.64 | 857.61 | 875.11 | 793.11 | 852.61 | 841.36 | 912.61 | 871.86 | 877.61 | 849.36 |
| | | LSM s.e. | 22.51 | 38.74 | 38.74 | 38.74 | 38.74 | 38.74 | 38.74 | 38.74 | 38.74 | 38.74 |
| 45 | 655.94 | Mean | 801.13 | 756.25 | 819.75 | 779.75 | 818.50 | 804.00 | 804.25 | 838.25 | 829.00 | 799.00 |
| mg/kg | | N | 4 | 4 | 4 | 4 | 4 | 4 | 4 | 4 | 4 | 4 |
| | | LSMean | 804.33 | 759.46 | 822.96 | 782.96 | 821.71 | 807.21 | 807.46 | 841.46 | 832.21 | 802.21 |
| | | LSM s.e. | 22.74 | 38.87 | 38.87 | 38.87 | 38.87 | 38.87 | 38.87 | 38.87 | 38.87 | 38.87 |
| 120 | 669.22 | Mean | 747.47 | 727.00 | 765.75 | 722.00 | 702.00 | 700.00 | 708.00 | 788.75 | 805.75 | 820.25 |
| mg/kg | | N | 4 | 4 | 4 | 4 | 4 | 4 | 4 | 4 | 4 | 4 |
| | | LSMean | 746.24 | 725.77 | 764.52 | 720.77 | 700.77 | 698.77 | 706.77 | 787.52 | 804.52 | 819.02 |
| | | LSM s.e. | 22.53 | 38.75 | 38.75 | 38.75 | 38.75 | 38.75 | 38.75 | 38.75 | 38.75 | 38.75 |

TABLE 8B shown below presents a summary of RR interval values (msec) measured in EXAMPLE 5 described above. Statistical analysis was based on a mixed model analysis of 7 through 22 Hour Time Interval Values (Segment 2). The covariate mean is the average of 2-hour pre-dose data with an AR(1) covariance structure.

For TABLE 8A-B, N=numbers of measures to calculate mean; LSMean=Least squares mean; LSM s.e.=Least squares standard error; *=p<0.05; NT=not tested.

TABLE 8B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 669.53 | Mean | 908.25 | 923.50 | 944.50 | 968.00 | 726.50 | 639.75 | 672.50 |
| mg/kg | | N | 4 | 4 | 4 | 4 | 4 | 4 | 4 |
| | | LSMean | 906.91 | 922.16 | 943.16 | 966.66 | 725.16 | 638.41 | 671.16 |
| | | LSM s.e. | 38.76 | 38.76 | 38.76 | 38.76 | 38.76 | 38.76 | 38.76 |
| 10 | 667.44 | Mean | 979.25 | 983.25 | 973.75 | 925.75 | 731.25 | 631.00 | 691.25 |
| mg/kg | | N | 4 | 4 | 4 | 4 | 4 | 4 | 4 |
| | | LSMean | 978.61 | 982.61 | 973.11 | 925.11 | 730.61 | 630.36 | 690.61 |
| | | LSM s.e. | 38.74 | 38.74 | 38.74 | 38.74 | 38.74 | 38.74 | 38.74 |
| 45 | 655.94 | Mean | 855.75 | 912.75 | 926.00 | 850.50 | 703.75 | 626.50 | 694.00 |
| mg/kg | | N | 4 | 4 | 4 | 4 | 4 | 4 | 4 |
| | | LSMean | 858.96 | 915.96 | 929.21 | 853.71 | 706.96 | 629.71 | 697.21 |
| | | LSM s.e. | 38.87 | 38.87 | 38.87 | 38.87 | 38.87 | 38.87 | 38.87 |
| 120 | 669.22 | Mean | 842.25 | 825.50 | 860.75 | 786.25 | 668.25 | 597.50 | 639.50 |
| mg/kg | | N | 4 | 4 | 4 | 4 | 4 | 4 | 4 |
| | | LSMean | 841.02 | 824.27 | 859.52 | 785.02 | 667.02 | 596.27 | 638.27 |
| | | LSM s.e. | 38.75 | 38.75 | 38.75 | 38.75 | 38.75 | 38.75 | 38.75 |

TABLE 9A shown below presents a summary of PR interval values (msec) measured in EXAMPLE 5 described above. Statistical analysis was based on mixed model analysis of 0.25 through 6 Hour Time Interval Values (Segment 1). The covariate mean is the average of 2-hour pre-dose data with an AR(1) covariance structure.

TABLE 9A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 94.75 | Mean | 96.84 | 92.50 | 97.00 | 98.75 | 100.00 | 99.75 | 100.75 | 95.25 | 99.50 | 98.50 |
| mg/kg | | N | 4 | 4 | 4 | 4 | 4 | 4 | 4 | 4 | 4 | 4 |
| | | LSMean | 98.65 | 94.31 | 98.81 | 100.56 | 101.81 | 101.56 | 102.56 | 97.06 | 101.31 | 100.31 |
| | | LSM s.e. | 1.24 | 1.81 | 1.81 | 1.81 | 1.81 | 1.81 | 1.81 | 1.81 | 1.81 | 1.81 |
| 10 | 97.88 | Mean | 94.54 | 92.75 | 89.75 | 91.00 | 92.50 | 92.00 | 92.50 | 90.50 | 93.50 | 92.50 |
| mg/kg | | N | 4 | 4 | 4 | 4 | 4 | 4 | 4 | 4 | 4 | 4 |
| | | LSMean | 93.79 | 91.99 | 88.99 | 90.24 | 91.74 | 91.24 | 91.74 | 89.74 | 92.74 | 91.74 |
| | | LSM s.e. | 1.03 | 1.67 | 1.67 | 1.67 | 1.67 | 1.67 | 1.67 | 1.67 | 1.67 | 1.67 |
| | | Trend | 0.039* | NT | 0.001* | 0.001* | 0.001* | 0.001* | 0.000* | 0.009* | 0.003* | 0.003* |
| | | p-value | | | | | | | | | | |
| 45 | 98.59 | Mean | 92.52 | 95.25 | 89.75 | 88.75 | 88.00 | 91.00 | 88.75 | 89.25 | 88.75 | 89.00 |
| mg/kg | | N | 4 | 4 | 4 | 4 | 4 | 4 | 4 | 4 | 4 | 4 |
| | | LSMean | 91.18 | 93.91 | 88.41 | 87.41 | 86.66 | 89.66 | 87.41 | 87.91 | 87.41 | 87.66 |
| | | LSM s.e. | 1.13 | 1.73 | 1.73 | 1.73 | 1.73 | 1.73 | 1.73 | 1.73 | 1.73 | 1.73 |
| | | Trend | 0.012* | NT | 0.001* | 0.000* | 0.000* | 0.000* | 0.000* | 0.003* | 0.000* | 0.000* |
| | | p-value | | | | | | | | | | |
| 120 | 96.59 | Mean | 87.71 | 91.25 | 90.00 | 86.25 | 85.25 | 84.50 | 84.00 | 84.00 | 87.00 | 85.75 |
| mg/kg | | N | 4 | 4 | 4 | 4 | 4 | 4 | 4 | 4 | 4 | 4 |
| | | LSMean | 88.00 | 91.54 | 90.29 | 86.54 | 85.54 | 84.79 | 84.29 | 84.29 | 87.29 | 86.04 |
| | | LSM s.e. | 0.98 | 1.64 | 1.64 | 1.64 | 1.64 | 1.64 | 1.64 | 1.64 | 1.64 | 1.64 |
| | | Trend | 0.001* | 0.411 | 0.002* | 0.000* | 0.000* | 0.000* | 0.000* | 0.000* | 0.000* | 0.000* |
| | | p-value | | | | | | | | | | |

TABLE 9B shown below presents a summary of PR interval values (msec) measured in EXAMPLE 5 above. Statistical analysis was based on a mixed model analysis of 0.25 through 6 Hour Time Interval Values (Segment 1). The covariate mean is the average of 2-hour pre-dose data with an AR(1) covariance structure.

TABLE 9B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 94.75 | Mean | 99.50 | 98.25 | 98.75 | 97.00 | 98.25 | 97.50 | 93.50 | 90.00 | 96.50 | 98.75 |
| mg/kg | | N | 4 | 4 | 4 | 4 | 4 | 4 | 4 | 4 | 4 | 4 |
| | | LSMean | 101.31 | 100.06 | 100.56 | 98.81 | 100.06 | 99.31 | 95.31 | 91.81 | 98.31 | 100.56 |
| | | LSM s.e. | 1.81 | 1.81 | 1.81 | 1.81 | 1.81 | 1.81 | 1.81 | 1.81 | 1.81 | 1.81 |
| 10 | 97.88 | Mean | 94.25 | 95.00 | 95.00 | 94.75 | 95.50 | 96.50 | 91.00 | 90.75 | 97.25 | 100.25 |
| mg/kg | | N | 4 | 4 | 4 | 4 | 4 | 4 | 4 | 4 | 4 | 4 |
| | | LSMean | 93.49 | 94.24 | 94.24 | 93.99 | 94.74 | 95.74 | 90.24 | 89.99 | 96.49 | 99.49 |
| | | LSM s.e. | 1.67 | 1.67 | 1.67 | 1.67 | 1.67 | 1.67 | 1.67 | 1.67 | 1.67 | 1.67 |
| | | Trend | 0.006* | 0.034* | 0.022* | 0.074 | 0.050 | 0.178 | 0.061 | NT | NT | NT |
| | | p-value | | | | | | | | | | |
| 45 | 98.59 | Mean | 92.00 | 91.25 | 92.75 | 92.25 | 95.00 | 92.25 | 89.00 | 90.75 | 95.25 | 99.00 |
| mg/kg | | N | 4 | 4 | 4 | 4 | 4 | 4 | 4 | 4 | 4 | 4 |
| | | LSMean | 90.66 | 89.91 | 91.41 | 90.91 | 93.66 | 90.91 | 87.66 | 89.41 | 93.91 | 97.66 |
| | | LSM s.e. | 1.73 | 1.73 | 1.73 | 1.73 | 1.73 | 1.73 | 1.73 | 1.73 | 1.73 | 1.73 |
| | | Trend | 0.001* | 0.001* | 0.003* | 0.008* | 0.028* | 0.006* | 0.010* | NT | 0.117 | 0.292 |
| | | p-value | | | | | | | | | | |
| 120 | 96.59 | Mean | 87.50 | 86.25 | 86.75 | 88.00 | 89.00 | 87.75 | 83.75 | 87.50 | 90.00 | 90.75 |
| mg/kg | | N | 4 | 4 | 4 | 4 | 4 | 4 | 4 | 4 | 4 | 4 |
| | | LSMean | 87.79 | 86.54 | 87.04 | 88.29 | 89.29 | 88.04 | 84.04 | 87.79 | 90.29 | 91.04 |
| | | LSM s.e. | 1.64 | 1.64 | 1.64 | 1.64 | 1.64 | 1.64 | 1.64 | 1.64 | 1.64 | 1.64 |
| | | Trend | 0.000* | 0.000* | 0.000* | 0.000* | 0.000* | 0.000* | 0.000* | 0.110 | 0.002* | 0.000* |
| | | p-value | | | | | | | | | | |

TABLE 9C shown below presents a summary of PR interval values (msec) and statistical analysis measured in EXAMPLE above. Statistical analysis was based on a mixed model analysis of 0.25 through 6 Hour Time Interval Values (Segment 1). The covariate mean is the average of 2-hour pre-dose data with an AR(1) covariance structure.

For TABLE 9A-C, N=numbers of measures t calculate mean; LSMean=Least squares mean; LSM s.e. Least squares standard error;*=p<0.05; NT=not tested.

TABLE 9C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 94.75 | Mean | 97.50 | 95.25 | 93.25 | 93.00 | 95.25 |
| mg/kg | | N | 4 | 4 | 4 | 4 | 4 |
| | | LSMean | 99.31 | 97.06 | 95.06 | 94.81 | 97.06 |
| | | LSM s.e. | 1.81 | 1.81 | 1.81 | 1.81 | 1.81 |
| 10 | 97.88 | Mean | 99.25 | 101.25 | 99.00 | 96.25 | 96.00 |
| mg/kg | | N | 4 | 4 | 4 | 4 | 4 |
| | | LSMean | 98.49 | 100.49 | 98.24 | 95.49 | 95.24 |
| | | LSM s.e. | 1.67 | 1.67 | 1.67 | 1.67 | 1.67 |
| | | Trend p-value | NT | NT | NT | NT | NT |
| 45 | 98.59 | Mean | 96.50 | 99.00 | 95.75 | 94.75 | 96.50 |
| mg/kg | | N | 4 | 4 | 4 | 4 | 4 |
| | | LSMean | 95.16 | 97.66 | 94.41 | 93.41 | 95.16 |
| | | LSM s.e. | 1.73 | 1.73 | 1.73 | 1.73 | 1.73 |
| | | Trend p-value | 0.138 | 0.825 | 0.810 | 0.607 | 0.486 |
| 120 | 96.59 | Mean | 90.25 | 90.25 | 89.00 | 89.25 | 91.00 |
| mg/kg | | N | 4 | 4 | 4 | 4 | 4 |
| | | LSMean | 90.54 | 90.54 | 89.29 | 89.54 | 91.29 |
| | | LSM s.e. | 1.64 | 1.64 | 1.64 | 1.64 | 1.64 |
| | | Trend p-value | 0.001* | 0.007* | 0.010* | 0.027* | 0.031* |

| Effects | Statistics | Value |
|---|---|---|
| MAIN EFFECT | Group F-test p-value | 0.004* |
| INTERACTION | Group*Time p-value | 0.000* |
| | Group Linear Trend*Linear Time p-value | 0.001* |
| | Group Linear Trend*Quadratic Time p-value | 0.000* |

TABLE 10A shown below presents a summary of PR interval values (msec) measured in EXAMPLE 5 above. Statistical analysis was based on a mixed model analysis of 7 through 22 Hour Time Interval Values (Segment 2). The covariate mean is the average of 2-hour pre-dose data with an AR(1) covariance structure.

TABLE 10A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 94.75 | Mean | 96.05 | 94.25 | 93.75 | 95.75 | 94.75 | 92.75 | 97.00 | 100.50 | 100.50 | 97.50 |
| mg/kg | | N | 4 | 4 | 4 | 4 | 4 | 4 | 4 | 4 | 4 | 4 |
| | | LSMean | 97.52 | 95.72 | 95.22 | 97.22 | 96.22 | 94.22 | 98.47 | 101.97 | 101.97 | 98.97 |
| | | LSM s.e. | 1.20 | 1.76 | 2.54 | 2.29 | 2.33 | 2.81 | 1.59 | 1.38 | 1.65 | 1.89 |
| 10 | 97.88 | Mean | 101.27 | 95.50 | 99.50 | 100.25 | 101.50 | 103.25 | 103.50 | 101.50 | 104.00 | 103.50 |
| mg/kg | | N | 4 | 4 | 4 | 4 | 4 | 4 | 4 | 4 | 4 | 4 |
| | | LSMean | 100.65 | 94.88 | 98.88 | 99.63 | 100.88 | 102.63 | 102.88 | 100.88 | 103.38 | 102.88 |
| | | LSM s.e. | 1.04 | 1.65 | 2.47 | 2.21 | 2.25 | 2.74 | 1.47 | 1.24 | 1.54 | 1.79 |
| | | Trend p-value | NT | | | | | | | | | |
| 45 | 98.59 | Mean | 99.73 | 97.00 | 100.50 | 96.75 | 99.75 | 100.00 | 98.75 | 100.50 | 99.75 | 99.50 |
| mg/kg | | N | 4 | 4 | 4 | 4 | 4 | 4 | 4 | 4 | 4 | 4 |
| | | LSMean | 98.64 | 95.90 | 99.40 | 95.65 | 98.65 | 98.90 | 97.65 | 99.40 | 98.65 | 98.40 |
| | | LSM s.e. | 1.12 | 1.70 | 2.50 | 2.24 | 2.29 | 2.77 | 1.53 | 1.31 | 1.59 | 1.84 |
| | | Trend p-value | NT | | | | | | | | | |
| 120 | 96.59 | Mean | 97.16 | 92.00 | 92.50 | 92.00 | 93.50 | 95.25 | 97.25 | 98.50 | 97.50 | 98.50 |
| mg/kg | | N | 4 | 4 | 4 | 4 | 4 | 4 | 4 | 4 | 4 | 4 |
| | | LSMean | 97.40 | 92.24 | 92.74 | 92.24 | 93.74 | 95.49 | 97.49 | 98.74 | 97.74 | 98.74 |
| | | LSM s.e. | 1.01 | 1.63 | 2.45 | 2.19 | 2.24 | 2.73 | 1.45 | 1.22 | 1.52 | 1.77 |
| | | Trend p-value | 0.636 | | | | | | | | | |

TABLE 10B shown below presents a summary of PR interval values (msec) measured in EXAMPLE 5 above. Statistical analysis was based on a mixed model analysis of 7 through 22 Hour Time Interval Values (Segment 2). The covariate mean is the average of 2-hour pre-dose data with an AR(1) covariance structure.

TABLE 10B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 94.75 | Mean | 96.25 | 96.50 | 94.25 | 95.25 | 95.50 | 96.25 | 96.00 |
| mg/kg | | N | 4 | 4 | 4 | 4 | 4 | 4 | 4 |
| | | LSMean | 97.72 | 97.97 | 95.72 | 96.72 | 96.97 | 97.72 | 97.47 |
| | | LSM s.e. | 2.19 | 1.45 | 2.80 | 2.03 | 1.46 | 1.22 | 1.08 |
| 10 | 97.88 | Mean | 106.00 | 103.25 | 103.50 | 102.50 | 98.25 | 96.00 | 98.25 |
| mg/kg | | N | 4 | 4 | 4 | 4 | 4 | 4 | 4 |
| | | LSMean | 105.38 | 102.63 | 102.88 | 101.88 | 97.63 | 95.38 | 97.63 |
| | | LSM s.e. | 2.11 | 1.32 | 2.73 | 1.94 | 1.33 | 1.06 | 0.90 |
| | | Trend p-value | | | | | | | |
| 45 | 98.59 | Mean | 103.75 | 101.25 | 100.50 | 101.75 | 99.50 | 97.75 | 98.75 |
| mg/kg | | N | 4 | 4 | 4 | 4 | 4 | 4 | 4 |
| | | LSMean | 102.65 | 100.15 | 99.40 | 100.65 | 98.40 | 96.65 | 97.65 |
| | | LSM s.e. | 2.15 | 1.38 | 2.76 | 1.98 | 1.39 | 1.14 | 0.99 |
| | | Trend p-value | | | | | | | |
| 120 | 96.59 | Mean | 101.75 | 100.00 | 100.00 | 100.50 | 99.00 | 97.50 | 98.75 |
| mg/kg | | N | 4 | 4 | 4 | 4 | 4 | 4 | 4 |
| | | LSMean | 101.99 | 100.24 | 100.24 | 100.74 | 99.24 | 97.74 | 98.99 |
| | | LSM s.e. | 2.09 | 1.30 | 2.72 | 1.92 | 1.31 | 1.03 | 0.86 |
| | | Trend p-value | | | | | | | |

TABLE 10C shown below presents a summary of statistical analysis of PR interval values (msec) measured in EXAMPLE 5 above. Statistical analysis was based on a mixed model analysis of 7 through 22 Hour Time Interval Values (Segment 2).

For TABLE 10A-B, N=numbers of measures to calculate mean; LSMean=Least squares mean; LSM s.e.=Least squares standard error; *=p<0.05; NT=not tested.

TABLE 10C

| Effects | Statistics | Value |
|---|---|---|
| MAIN EFFECT | Group F-test p-value | 0.207 |
| INTERACTION | Group*Time p-value | 0.336 |
| | Group Linear Trend*Linear Time p-value | 0.077 |
| | Group Linear Trend*Quadratic Time p-value | 0.928 |

TABLE 1A shown below presents a summary of QRS duration values (msec) measured in EXAMPLE 5 above. Statistical analysis was based on mixed model analysis of 0.25 through 6 Hour Time Interval Values (Segment 1). The covariate mean is the average of 2-hour pre-dose data with an AR(1) covariance structure.

TABLE 11A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 39.22 | Mean | 40.38 | 38.00 | 40.00 | 41.50 | 41.50 | 41.25 | 41.50 | 39.00 | 40.75 | 40.50 |
| mg/kg | | N | 4 | 4 | 4 | 4 | 4 | 4 | 4 | 4 | 4 | 4 |
| | | LSMean | 40.33 | 37.95 | 39.95 | 41.45 | 41.45 | 41.20 | 41.45 | 38.95 | 40.70 | 40.45 |
| | | LSM s.e. | 0.32 | 0.69 | 0.69 | 0.69 | 0.69 | 0.69 | 0.69 | 0.69 | 0.69 | 0.69 |
| 10 | 40.19 | Mean | 39.65 | 38.25 | 39.25 | 38.50 | 39.25 | 39.25 | 39.75 | 39.00 | 39.75 | 39.75 |
| mg/kg | | N | 4 | 4 | 4 | 4 | 4 | 4 | 4 | 4 | 4 | 4 |
| | | LSMean | 39.68 | 38.29 | 39.29 | 38.54 | 39.29 | 39.29 | 39.79 | 39.04 | 39.79 | 39.79 |
| | | LSM s.e. | 0.30 | 0.68 | 0.68 | 0.68 | 0.68 | 0.68 | 0.68 | 0.68 | 0.68 | 0.68 |
| | | Trend | 0.246 | NT | NT | NT | NT | NT | 0.099 | NT | NT | NT |
| | | p-value | | | | | | | | | | |
| 45 | 39.72 | Mean | 39.05 | 39.25 | 39.00 | 39.00 | 39.50 | 39.25 | 38.75 | 38.25 | 39.00 | 38.75 |
| mg/kg | | N | 4 | 4 | 4 | 4 | 4 | 4 | 4 | 4 | 4 | 4 |
| | | LSMean | 39.05 | 39.25 | 39.00 | 39.00 | 39.50 | 39.25 | 38.75 | 38.25 | 39.00 | 38.75 |
| | | LSM s.e. | 0.27 | 0.67 | 0.67 | 0.67 | 0.67 | 0.67 | 0.67 | 0.67 | 0.67 | 0.67 |
| | | Trend | 0.026* | NT | NT | NT | NT | NT | 0.006* | NT | NT | 0.078 |
| | | p-value | | | | | | | | | | |
| 120 | 39.91 | Mean | 38.93 | 39.50 | 41.75 | 40.75 | 40.25 | 39.75 | 38.75 | 39.00 | 39.50 | 38.50 |
| mg/kg | | N | 4 | 4 | 4 | 4 | 4 | 4 | 4 | 4 | 4 | 4 |
| | | LSMean | 38.94 | 39.51 | 41.76 | 40.76 | 40.26 | 39.76 | 38.76 | 39.01 | 39.51 | 38.51 |
| | | LSM s.e. | 0.27 | 0.67 | 0.67 | 0.67 | 0.67 | 0.67 | 0.67 | 0.67 | 0.67 | 0.67 |
| | | Trend | 0.014* | 0.066 | 0.094 | 0.595 | 0.270 | 0.153 | 0.004* | 0.839 | 0.153 | 0.026* |
| | | p-value | | | | | | | | | | |

TABLE 11B shown below presents a summary of QRS duration values (msec) measured in EXAMPLE 5 above. Statistical analysis was based on mixed model analysis of 0.25 through 6 Hour Time Interval Values (Segment 1). The covariate mean is the average of 2-hour pre-dose data with an AR(1) covariance structure.

TABLE 11B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 39.22 | Mean | 41.00 | 42.00 | 41.50 | 40.00 | 40.00 | 40.25 | 39.00 | 36.50 | 39.50 | 40.75 |
| mg/kg | | N | 4 | 4 | 4 | 4 | 4 | 4 | 4 | 4 | 4 | 4 |
| | | LSMean | 40.95 | 41.95 | 41.45 | 39.95 | 39.95 | 40.20 | 38.95 | 36.45 | 39.45 | 40.70 |
| | | LSM s.e. | 0.69 | 0.69 | 0.69 | 0.69 | 0.69 | 0.69 | 0.69 | 0.69 | 0.69 | 0.69 |
| 10 | 40.19 | Mean | 40.75 | 40.00 | 39.50 | 39.50 | 39.50 | 40.00 | 37.75 | 38.00 | 39.75 | 40.25 |
| mg/kg | | N | 4 | 4 | 4 | 4 | 4 | 4 | 4 | 4 | 4 | 4 |
| | | LSMean | 40.79 | 40.04 | 39.54 | 39.54 | 39.54 | 40.04 | 37.79 | 38.04 | 39.79 | 40.29 |
| | | LSM s.e. | 0.68 | 0.68 | 0.68 | 0.68 | 0.68 | 0.68 | 0.68 | 0.68 | 0.68 | 0.68 |
| | | Trend | NT | 0.058 | NT | NT | NT | NT | 0.245 | NT | NT | NT |
| | | p-value | | | | | | | | | | |
| 45 | 39.72 | Mean | 38.50 | 38.75 | 40.00 | 39.25 | 39.00 | 38.75 | 36.50 | 37.50 | 39.75 | 41.00 |
| mg/kg | | N | 4 | 4 | 4 | 4 | 4 | 4 | 4 | 4 | 4 | 4 |
| | | LSMean | 38.50 | 38.75 | 40.00 | 39.25 | 39.00 | 38.75 | 36.50 | 37.50 | 39.75 | 41.00 |
| | | LSM s.e. | 0.67 | 0.67 | 0.67 | 0.67 | 0.67 | 0.67 | 0.67 | 0.67 | 0.67 | 0.67 |
| | | Trend | NT | 0.001* | 0.132 | NT | NT | 0.132 | 0.012* | NT | NT | 0.761 |
| | | p-value | | | | | | | | | | |
| 120 | 39.91 | Mean | 39.75 | 39.00 | 38.75 | 38.75 | 38.75 | 37.00 | 36.25 | 37.50 | 38.50 | 38.25 |
| mg/kg | | N | 4 | 4 | 4 | 4 | 4 | 4 | 4 | 4 | 4 | 4 |
| | | LSMean | 39.76 | 39.01 | 38.76 | 38.76 | 38.76 | 37.01 | 36.26 | 37.51 | 38.51 | 38.26 |
| | | LSM s.e. | 0.67 | 0.67 | 0.67 | 0.67 | 0.67 | 0.67 | 0.67 | 0.67 | 0.67 | 0.67 |
| | | Trend | 0.056 | 0.001* | 0.014* | 0.205 | 0.178 | 0.001* | 0.003* | 0.387 | 0.347 | 0.032* |
| | | p-value | | | | | | | | | | |

TABLE 11C shown below presents a summary of QRS duration values (msec) and statistical analysis measured in EXAMPLE 5 above. Statistical analysis was based on mixed model analysis of 0.25 through Hour Time Interval Values (Segment 1). The covariate mean is the average of 2-hour pre-dose data with an AR(1)covariance structure.

For TABLE 1A-C, N=numbers of measures t calculate mean; LSMean=Least squares mean; LSM s.e. Least squares standard error;*=p<0.05; NT=not tested.

TABLE 11C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 39.22 | Mean | 41.25 | 41.00 | 41.00 | 40.25 | 41.00 |
| mg/kg | | N | 4 | 4 | 4 | 4 | 4 |
| | | LSMean | 41.20 | 40.95 | 40.95 | 40.20 | 40.95 |
| | | LSM s.e. | 0.69 | 0.69 | 0.69 | 0.69 | 0.69 |
| 10 | 40.19 | Mean | 41.00 | 41.50 | 40.75 | 40.00 | 40.50 |
| mg/kg | | N | 4 | 4 | 4 | 4 | 4 |
| | | LSMean | 41.04 | 41.54 | 40.79 | 40.04 | 40.54 |
| | | LSM s.e. | 0.68 | 0.68 | 0.68 | 0.68 | 0.68 |
| | | Trend p-value | NT | NT | 0.866 | NT | 0.676 |
| 45 | 39.72 | Mean | 40.25 | 41.00 | 39.00 | 39.00 | 38.25 |
| mg/kg | | N | 4 | 4 | 4 | 4 | 4 |
| | | LSMean | 40.25 | 41.00 | 39.00 | 39.00 | 38.25 |
| | | LSM s.e. | 0.67 | 0.67 | 0.67 | 0.67 | 0.67 |
| | | Trend p-value | 0.320 | NT | 0.044* | NT | 0.006* |
| 120 | 39.91 | Mean | 39.00 | 39.50 | 39.00 | 38.75 | 37.75 |
| mg/kg | | N | 4 | 4 | 4 | 4 | 4 |
| | | LSMean | 39.01 | 39.51 | 39.01 | 38.76 | 37.76 |
| | | LSM s.e. | 0.67 | 0.67 | 0.67 | 0.67 | 0.67 |
| | | Trend p-value | 0.017* | 0.112 | 0.014* | 0.080 | 0.000* |

| Effects | Statistics | Value |
|---|---|---|
| MAIN EFFECT | Group F-test p-value | 0.057 |
| INTERACTION | Group*Time p-value | 0.016* |
| | Group Linear Trend*Linear Time p-value | 0.012* |
| | Group Linear Trend*Quadratic Time p-value | 0.089 |

TABLE 12A shown below presents a summary of QRS duration values (msec) measured in EXAMPLE 5 above. Statistical analysis was based on mixed model analysis of 7 through 22 Hour Time Interval Values (Segment 2). The covariate mean is the average of 2-hour pre-dose data with an AR(1) covariance structure.

TABLE 12A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 39.22 | Mean | 40.69 | 40.75 | 40.75 | 41.25 | 41.00 | 40.75 | 40.50 | 42.00 | 41.50 | 40.75 |
| mg/kg | | N | 4 | 4 | 4 | 4 | 4 | 4 | 4 | 4 | 4 | 4 |
| | | LSMean | 40.96 | 41.02 | 41.02 | 41.52 | 41.27 | 41.02 | 40.77 | 42.27 | 41.77 | 41.02 |
| | | LSM s.e. | 0.21 | 0.87 | 0.65 | 0.70 | 0.66 | 0.36 | 0.51 | 0.70 | 0.58 | 0.26 |
| 10 | 40.19 | Mean | 41.34 | 40.50 | 40.75 | 41.50 | 41.50 | 41.75 | 42.00 | 41.00 | 42.00 | 41.75 |
| mg/kg | | N | 4 | 4 | 4 | 4 | 4 | 4 | 4 | 4 | 4 | 4 |
| | | LSMean | 41.13 | 40.28 | 40.53 | 41.28 | 41.28 | 41.53 | 41.78 | 40.78 | 41.78 | 41.53 |
| | | LSM s.e. | 0.20 | 0.87 | 0.65 | 0.70 | 0.65 | 0.36 | 0.50 | 0.69 | 0.58 | 0.25 |
| | | Trend p-value | NT | NT | NT | NT | NT | NT | NT | NT | NT | NT |
| 45 | 39.72 | Mean | 40.56 | 40.25 | 40.50 | 40.50 | 40.25 | 40.25 | 41.00 | 41.25 | 40.50 | 41.00 |
| mg/kg | | N | 4 | 4 | 4 | 4 | 4 | 4 | 4 | 4 | 4 | 4 |
| | | LSMean | 40.58 | 40.27 | 40.52 | 40.52 | 40.27 | 40.27 | 41.02 | 41.27 | 40.52 | 41.02 |
| | | LSM s.e. | 0.18 | 0.87 | 0.64 | 0.69 | 0.65 | 0.35 | 0.50 | 0.69 | 0.57 | 0.24 |
| | | Trend p-value | 0.202 | 0.563 | 0.601 | NT | NT | NT | NT | NT | NT | 0.993 |
| 120 | 39.91 | Mean | 40.20 | 37.75 | 38.75 | 39.50 | 40.50 | 40.75 | 41.00 | 41.00 | 40.75 | 40.00 |
| mg/kg | | N | 4 | 4 | 4 | 4 | 4 | 4 | 4 | 4 | 4 | 4 |
| | | LSMean | 40.13 | 37.67 | 38.67 | 39.42 | 40.42 | 40.67 | 40.92 | 40.92 | 40.67 | 39.92 |
| | | LSM s.e. | 0.18 | 0.87 | 0.64 | 0.69 | 0.65 | 0.35 | 0.50 | 0.69 | 0.58 | 0.24 |
| | | Trend p-value | 0.007* | 0.041* | 0.048* | 0.062 | 0.266 | 0.186 | 0.895 | 0.291 | 0.125 | 0.011* |

TABLE 12B shown below presents a summary of QRS duration values (msec) measured in EXAMPLE 5 above. Statistical analysis was based on mixed model analysis of 7 through 22 Hour Time Interval Values (Segment 2). The covariate mean is the average of 2-hour pre-dose data with an AR(1) covariance structure.

TABLE 12B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 39.22 | Mean | 40.75 | 41.00 | 40.75 | 40.50 | 40.00 | 39.25 | 39.50 |
| mg/kg | | N | 4 | 4 | 4 | 4 | 4 | 4 | 4 |
| | | LSMean | 41.02 | 41.27 | 41.02 | 40.77 | 40.27 | 39.52 | 39.77 |
| | | LSM s.e. | 0.32 | 0.53 | 0.40 | 0.31 | 0.35 | 0.24 | 0.28 |
| 10 | 40.19 | Mean | 42.00 | 41.50 | 41.75 | 41.50 | 41.25 | 40.00 | 40.75 |
| mg/kg | | N | 4 | 4 | 4 | 4 | 4 | 4 | 4 |
| | | LSMean | 41.78 | 41.28 | 41.53 | 41.28 | 41.03 | 39.78 | 40.53 |
| | | LSM s.e. | 0.32 | 0.53 | 0.40 | 0.30 | 0.34 | 0.24 | 0.27 |
| | | Trend p-value | NT | NT | NT | NT | NT | NT | NT |
| 45 | 39.72 | Mean | 41.00 | 41.00 | 40.75 | 40.75 | 40.50 | 39.25 | 40.25 |
| mg/kg | | N | 4 | 4 | 4 | 4 | 4 | 4 | 4 |
| | | LSMean | 41.02 | 41.02 | 40.77 | 40.77 | 40.52 | 39.27 | 40.27 |
| | | LSM s.e. | 0.30 | 0.52 | 0.39 | 0.29 | 0.33 | 0.22 | 0.26 |
| | | Trend p-value | NT | NT | NT | NT | NT | NT | NT |
| 120 | 39.91 | Mean | 40.25 | 41.00 | 40.75 | 40.50 | 40.50 | 39.75 | 40.50 |
| mg/kg | | N | 4 | 4 | 4 | 4 | 4 | 4 | 4 |
| | | LSMean | 40.17 | 40.92 | 40.67 | 40.42 | 40.42 | 39.67 | 40.42 |
| | | LSM s.e. | 0.30 | 0.52 | 0.39 | 0.29 | 0.33 | 0.22 | 0.26 |
| | | Trend p-value | 0.050 | 0.598 | 0.337 | 0.285 | 0.971 | 0.957 | 0.194 |

TABLE 12C shown below presents a summary of statistical analysis on data from TABLE 12A-B. Statistical analysis was based on a mixed model analysis of 7 through 22 Hour Time Interval Values (Segment 2).

TABLE 12C

| Effects | Statistics | Value |
|---|---|---|
| MAIN EFFECT | Group F-test p-value | 0.016* |
| INTERACTION | Group*Time p-value | 0.592 |
| | Group Linear Trend*Linear Time p-value | 0.001* |
| | Group Linear Trend*Quadratic Time p-value | 0.503 |

TABLE 13A shown below presents a summary of QT interval values (msec) measured in EXAMPLE 5 above. Statistical analysis was based on mixed model analysis of 0.25 through 6 Hour Time Interval Values (Segment 1). The covariate mean is the average of 2-hour pre-dose data with an AR(1) covariance structure.

TABLE 13A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 221.38 | Mean | 232.86 | 206.75 | 221.75 | 228.50 | 234.25 | 243.25 | 234.00 | 228.25 | 249.50 | 243.50 |
| mg/kg | | N | 4 | 4 | 4 | 4 | 4 | 4 | 4 | 4 | 4 | 4 |
| | | LSMean | 234.17 | 208.06 | 223.06 | 229.81 | 235.56 | 244.56 | 235.31 | 229.56 | 250.81 | 244.81 |
| | | LSM s.e. | 2.21 | 4.30 | 4.30 | 4.30 | 4.30 | 4.30 | 4.30 | 4.30 | 4.30 | 4.30 |
| 10 | 222.84 | Mean | 225.06 | 208.75 | 211.50 | 216.50 | 221.25 | 221.00 | 226.00 | 212.00 | 224.25 | 221.75 |
| mg/kg | | N | 4 | 4 | 4 | 4 | 4 | 4 | 4 | 4 | 4 | 4 |
| | | LSMean | 225.56 | 209.25 | 212.00 | 217.00 | 221.75 | 221.50 | 226.50 | 212.50 | 224.75 | 222.25 |
| | | LSM s.e. | 2.13 | 4.26 | 4.26 | 4.26 | 4.26 | 4.26 | 4.26 | 4.26 | 4.26 | 4.26 |
| | | Trend | 0.036* | NT | NT | NT | 0.026* | 0.000* | 0.149 | 0.006* | 0.000* | 0.000* |
| | | p-value | | | | | | | | | | |
| 45 | 224.09 | Mean | 219.57 | 220.25 | 213.25 | 215.25 | 217.25 | 220.50 | 209.50 | 212.25 | 214.50 | 216.25 |
| mg/kg | | N | 4 | 4 | 4 | 4 | 4 | 4 | 4 | 4 | 4 | 4 |
| | | LSMean | 219.39 | 220.07 | 213.07 | 215.07 | 217.07 | 220.32 | 209.32 | 212.07 | 214.32 | 216.07 |
| | | LSM s.e. | 2.12 | 4.25 | 4.25 | 4.25 | 4.25 | 4.25 | 4.25 | 4.25 | 4.25 | 4.25 |
| | | Trend | 0.005* | NT | NT | NT | 0.003* | 0.000* | 0.000* | 0.005* | 0.000* | 0.000* |
| | | p-value | | | | | | | | | | |
| 120 | 226.72 | Mean | 218.86 | 208.50 | 219.75 | 220.00 | 223.50 | 216.25 | 208.75 | 212.25 | 223.75 | 211.50 |
| mg/kg | | N | 4 | 4 | 4 | 4 | 4 | 4 | 4 | 4 | 4 | 4 |
| | | LSMean | 217.24 | 206.87 | 218.12 | 218.37 | 221.87 | 214.62 | 207.12 | 210.62 | 222.12 | 209.87 |
| | | LSM s.e. | 2.26 | 4.32 | 4.32 | 4.32 | 4.32 | 4.32 | 4.32 | 4.32 | 4.32 | 4.32 |
| | | Trend | 0.003* | 0.712 | 0.485 | 0.070 | 0.023* | 0.000* | 0.000* | 0.005* | 0.000* | 0.000* |
| | | p-value | | | | | | | | | | |

TABLE 13B shown below presents a summary of QT interval values (msec) measured in EXAMPLE 5 above. Statistical analysis was based on mixed model analysis of 0.25 through 6 Hour Time Interval Values (Segment 1). The covariate mean is the average of 2-hour pre-dose data with an AR(1) covariance structure.

TABLE 13B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 221.38 | Mean | 236.75 | 242.50 | 237.75 | 231.25 | 234.25 | 237.75 | 224.50 | 204.75 | 224.50 | 236.50 |
| mg/kg | | N | 4 | 4 | 4 | 4 | 4 | 4 | 4 | 4 | 4 | 4 |
| | | LSMean | 238.06 | 243.81 | 239.06 | 232.56 | 235.56 | 239.06 | 225.81 | 206.06 | 225.81 | 237.81 |
| | | LSM s.e. | 4.30 | 4.30 | 4.30 | 4.30 | 4.30 | 4.30 | 4.30 | 4.30 | 4.30 | 4.30 |
| 10 | 222.84 | Mean | 226.75 | 227.00 | 227.75 | 225.00 | 231.00 | 231.50 | 208.00 | 210.25 | 221.00 | 234.25 |
| mg/kg | | N | 4 | 4 | 4 | 4 | 4 | 4 | 4 | 4 | 4 | 4 |
| | | LSMean | 227.25 | 227.50 | 228.25 | 225.50 | 231.50 | 232.00 | 208.50 | 210.75 | 221.50 | 234.75 |
| | | LSM s.e. | 4.26 | 4.26 | 4.26 | 4.26 | 4.26 | 4.26 | 4.26 | 4.26 | 4.26 | 4.26 |
| | | Trend | 0.078 | 0.009* | 0.078 | NT | NT | 0.246 | 0.006* | NT | NT | NT |
| | | p-value | | | | | | | | | | |
| 45 | 224.09 | Mean | 220.00 | 217.00 | 226.50 | 220.75 | 225.00 | 216.75 | 196.25 | 208.25 | 224.75 | 226.25 |
| mg/kg | | N | 4 | 4 | 4 | 4 | 4 | 4 | 4 | 4 | 4 | 4 |
| | | LSMean | 219.82 | 216.82 | 226.32 | 220.57 | 224.82 | 216.57 | 196.07 | 208.07 | 224.57 | 226.07 |
| | | LSM s.e. | 4.25 | 4.25 | 4.25 | 4.25 | 4.25 | 4.25 | 4.25 | 4.25 | 4.25 | 4.25 |
| | | Trend | 0.004* | 0.000* | 0.040* | NT | 0.081 | 0.000* | 0.000* | NT | NT | 0.057 |
| | | p-value | | | | | | | | | | |
| 120 | 226.72 | Mean | 222.25 | 212.00 | 211.50 | 225.50 | 222.00 | 210.00 | 194.50 | 215.50 | 226.00 | 220.75 |
| mg/kg | | N | 4 | 4 | 4 | 4 | 4 | 4 | 4 | 4 | 4 | 4 |
| | | LSMean | 220.62 | 210.37 | 209.87 | 223.87 | 220.37 | 208.37 | 192.87 | 213.87 | 224.37 | 219.12 |
| | | LSM s.e. | 4.32 | 4.32 | 4.32 | 4.32 | 4.32 | 4.32 | 4.32 | 4.32 | 4.32 | 4.32 |
| | | Trend | 0.004* | 0.000* | 0.000* | 0.119 | 0.010* | 0.000* | 0.000* | 0.294 | 0.949 | 0.002* |
| | | p-value | | | | | | | | | | |

TABLE 13C shown below presents a summary of QT interval values (msec) and statistical analysis measured in EXAMPLE 5 above. Statistical analysis was based on mixed model analysis of 0.25 through Hour Time Interval Values (Segment 1). The covariate mean is the average of 2-hour pre-dose data with an AR(1) covariance structure.

For TABLE 13A-C, N=numbers of measures to calculate mean; LSMean=Least squares mean; LSM s.e.=Least squares standard error; *=p<0.05; NT=not tested.

TABLE 13C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 221.38 | Mean | 236.25 | 243.50 | 238.75 | 227.75 | 242.25 |
| mg/kg | | N | 4 | 4 | 4 | 4 | 4 |
| | | LSMean | 237.56 | 244.81 | 240.06 | 229.06 | 243.56 |
| | | LSM s.e. | 4.30 | 4.30 | 4.30 | 4.30 | 4.30 |
| 10 | 222.84 | Mean | 236.00 | 237.00 | 238.50 | 243.00 | 241.50 |
| mg/kg | | N | 4 | 4 | 4 | 4 | 4 |
| | | LSMean | 236.50 | 237.50 | 239.00 | 243.50 | 242.00 |
| | | LSM s.e. | 4.26 | 4.26 | 4.26 | 4.26 | 4.26 |
| | | Trend p-value | NT | NT | 0.861 | NT | NT |
| 45 | 224.09 | Mean | 228.25 | 234.50 | 224.50 | 225.75 | 236.25 |
| mg/kg | | N | 4 | 4 | 4 | 4 | 4 |
| | | LSMean | 228.07 | 234.32 | 224.32 | 225.57 | 236.07 |
| | | LSM s.e. | 4.25 | 4.25 | 4.25 | 4.25 | 4.25 |
| | | Trend p-value | NT | 0.089 | 0.012* | NT | 0.221 |
| 120 | 226.72 | Mean | 231.00 | 231.00 | 226.00 | 229.25 | 231.25 |
| mg/kg | | N | 4 | 4 | 4 | 4 | 4 |
| | | LSMean | 229.37 | 229.37 | 224.37 | 227.62 | 229.62 |
| | | LSM s.e. | 4.32 | 4.32 | 4.32 | 4.32 | 4.32 |
| | | Trend o-value | 0.098 | 0.015* | 0.003* | 0.261 | 0.018* |

| Effects | Statistics | Value |
|---|---|---|
| MAIN EFFECT | Group F-test p-value | 0.013* |
| INTERACTION | Group*Time p-value | 0.000* |
| | Group Linear Trend*Linear Time p-value | 0.434 |
| | Group Linear Trend*Quadratic Time p-value | 0.000* |

TABLE 14A shown below presents a summary of QT interval values (msec) measured in EXAMPLE 5 above. Statistical analysis was based on mixed model analysis of 7 through 22 Hour Time Interval Values (Segment 2). The covariate mean is the average of 2-hour pre-dose data with an AR(1) covariance structure.

TABLE 14A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 221.38 | Mean | 236.09 | 240.25 | 235.00 | 235.00 | 237.75 | 237.00 | 235.75 | 238.50 | 239.75 | 240.75 |
| mg/kg | | N | 4 | 4 | 4 | 4 | 4 | 4 | 4 | 4 | 4 | 4 |
| | | LSMean | 237.39 | 241.55 | 236.30 | 236.30 | 239.05 | 238.30 | 237.05 | 239.80 | 241.05 | 242.05 |
| | | LSM s.e. | 2.56 | 3.73 | 3.73 | 3.73 | 3.73 | 3.73 | 3.73 | 3.73 | 3.73 | 3.73 |
| 10 | 222.84 | Mean | 239.47 | 240.00 | 240.75 | 236.75 | 243.00 | 240.00 | 243.75 | 241.25 | 239.25 | 240.50 |
| mg/kg | | N | 4 | 4 | 4 | 4 | 4 | 4 | 4 | 4 | 4 | 4 |
| | | LSMean | 239.97 | 240.50 | 241.25 | 237.25 | 243.50 | 240.50 | 244.25 | 241.75 | 239.75 | 241.00 |
| | | LSM s.e. | 2.47 | 3.67 | 3.67 | 3.67 | 3.67 | 3.67 | 3.67 | 3.67 | 3.67 | 3.67 |
| | | Trend p-value | NT | | | | | | | | | |
| 45 | 224.09 | Mean | 236.47 | 234.50 | 240.25 | 234.75 | 238.00 | 237.50 | 241.25 | 236.25 | 233.75 | 238.25 |
| mg/kg | | N | 4 | 4 | 4 | 4 | 4 | 4 | 4 | 4 | 4 | 4 |
| | | LSMean | 236.29 | 234.32 | 240.07 | 234.57 | 237.82 | 237.32 | 241.07 | 236.07 | 233.57 | 238.07 |
| | | LSM s.e. | 2.46 | 3.66 | 3.66 | 3.66 | 3.66 | 3.66 | 3.66 | 3.66 | 3.66 | 3.66 |
| | | Trend p-value | NT | | | | | | | | | |
| 120 | 226.72 | Mean | 233.95 | 230.75 | 231.50 | 235.00 | 234.75 | 233.25 | 235.25 | 235.00 | 236.00 | 238.25 |
| mg/kg | | N | 4 | 4 | 4 | 4 | 4 | 4 | 4 | 4 | 4 | 4 |
| | | LSMean | 232.34 | 229.14 | 229.89 | 233.39 | 233.14 | 231.64 | 233.64 | 233.39 | 234.39 | 236.64 |
| | | LSM s.e. | 2.61 | 3.77 | 3.77 | 3.77 | 3.77 | 3.77 | 3.77 | 3.77 | 3.77 | 3.77 |
| | | Trend p-value | 0.183 | | | | | | | | | |

TABLE 14B shown below presents a summary of QT interval values (msec) measured in EXAMPLE 5 above. Statistical analysis was based on mixed model analysis of 7 through 22 Hour Time Interval Values (Segment 2). The covariate mean is the average of 2-hour pre-dose data with an AR(1) covariance structure.

TABLE 14B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 221.38 | Mean | 239.25 | 243.25 | 242.50 | 245.00 | 228.75 | 217.00 | 222.00 |
| mg/kg | | N | 4 | 4 | 4 | 4 | 4 | 4 | 4 |
| | | LSMean | 240.55 | 244.55 | 243.80 | 246.30 | 230.05 | 218.30 | 223.30 |
| | | LSM s.e. | 3.73 | 3.73 | 3.73 | 3.73 | 3.73 | 3.73 | 3.73 |
| 10 | 222.84 | Mean | 245.25 | 245.75 | 245.50 | 244.75 | 235.75 | 220.75 | 228.50 |
| mg/kg | | N | 4 | 4 | 4 | 4 | 4 | 4 | 4 |
| | | LSMean | 245.75 | 246.25 | 246.00 | 245.25 | 236.25 | 221.25 | 229.00 |
| | | LSM s.e. | 3.67 | 3.67 | 3.67 | 3.67 | 3.67 | 3.67 | 3.67 |
| | | Trend p-value | | | | | | | |
| 45 | 224.09 | Mean | 238.75 | 242.25 | 242.25 | 240.25 | 230.75 | 223.50 | 231.25 |
| mg/kg | | N | 4 | 4 | 4 | 4 | 4 | 4 | 4 |
| | | LSMean | 238.57 | 242.07 | 242.07 | 240.07 | 230.57 | 223.32 | 231.07 |
| | | LSM s.e. | 3.66 | 3.66 | 3.66 | 3.66 | 3.66 | 3.66 | 3.66 |
| | | Trend p-value | | | | | | | |
| 120 | 226.72 | Mean | 239.50 | 240.00 | 241.25 | 235.75 | 229.75 | 221.75 | 225.50 |
| mg/kg | | N | 4 | 4 | 4 | 4 | 4 | 4 | 4 |
| | | LSMean | 237.89 | 238.39 | 239.64 | 234.14 | 228.14 | 220.14 | 223.89 |
| | | LSM s.e. | 3.77 | 3.77 | 3.77 | 3.77 | 3.77 | 3.77 | 3.77 |
| | | Trend p-value | | | | | | | |

TABLE 14C shown below presents a summary of statistical analysis of data shown in TABLE 14A-B. Statistical analysis was based on a mixed model analysis of 7 through 22 Hour Time Interval Values (Segment 2).

For TABLE 14A-B, N=numbers of measures to calculate mean; LSMean=Least squares mean; LSM s.e.=Least squares standard error; *=p<0.05; NT=not tested.

TABLE 14C

| Effects | Statistics | Value |
|---|---|---|
| MAIN EFFECT | Group F-test p-value | 0.333 |
| INTERACTION | Group*Time p-value | 0.855 |
| | Group Linear Trend*Linear Time p-value | 0.143 |
| | Group Linear Trend*Quadratic Time p-value | 0.504 |

TABLE 15A shown below presents a summary of Corrected QT interval values (msec) measured in EXAMPLE 5 above. Statistical analysis was based on mixed model analysis of 0.25 through 6 Hour Time Interval Values (Segment 1). The covariate mean is the average of 2-hour pre-dose data with an AR(1) covariance structure.

TABLE 15A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 230.38 | Mean | 229.28 | 224.00 | 225.50 | 228.50 | 229.25 | 235.25 | 227.25 | 231.75 | 238.50 | 235.75 |
| mg/kg | | N | 4 | 4 | 4 | 4 | 4 | 4 | 4 | 4 | 4 | 4 |
| | | LSMean | 230.21 | 224.93 | 226.43 | 229.43 | 230.18 | 236.18 | 228.18 | 232.68 | 239.43 | 236.68 |
| | | LSM s.e. | 1.23 | 7.20 | 3.71 | 2.25 | 2.23 | 2.47 | 3.03 | 5.36 | 3.18 | 3.07 |
| 10 | 230.91 | Mean | 230.76 | 229.50 | 227.75 | 229.00 | 229.00 | 231.75 | 233.25 | 228.00 | 231.50 | 229.00 |
| mg/kg | | N | 4 | 4 | 4 | 4 | 4 | 4 | 4 | 4 | 4 | 4 |
| | | LSMean | 231.43 | 230.17 | 228.42 | 229.67 | 229.67 | 232.42 | 233.92 | 228.67 | 232.17 | 229.67 |
| | | LSM s.e. | 1.22 | 7.20 | 3.71 | 2.25 | 2.22 | 2.46 | 3.02 | 5.36 | 3.17 | 3.07 |
| | | Trend | NT | | | | | | | | | |
| | | p-value | | | | | | | | | | |
| 45 | 233.06 | Mean | 230.33 | 239.50 | 228.75 | 228.75 | 231.00 | 230.00 | 222.50 | 229.50 | 226.75 | 229.25 |
| mg/kg | | N | 4 | 4 | 4 | 4 | 4 | 4 | 4 | 4 | 4 | 4 |
| | | LSMean | 229.94 | 239.11 | 228.36 | 228.36 | 230.61 | 229.61 | 222.11 | 229.11 | 226.36 | 228.86 |
| | | LSM s.e. | 1.21 | 7.20 | 3.70 | 2.24 | 2.22 | 2.46 | 3.02 | 5.36 | 3.17 | 3.06 |
| | | Trend | NT | | | | | | | | | |
| | | p-value | | | | | | | | | | |
| 120 | 234.72 | Mean | 230.79 | 225.50 | 234.25 | 233.00 | 232.00 | 229.75 | 227.50 | 228.00 | 233.75 | 227.75 |
| mg/kg | | N | 4 | 4 | 4 | 4 | 4 | 4 | 4 | 4 | 4 | 4 |
| | | LSMean | 229.59 | 224.30 | 233.05 | 231.80 | 230.80 | 228.55 | 226.30 | 226.80 | 232.55 | 226.55 |
| | | LSM s.e. | 1.25 | 7.21 | 3.72 | 2.26 | 2.24 | 2.48 | 3.03 | 5.37 | 3.18 | 3.08 |
| | | Trend | 0.588 | | | | | | | | | |
| | | p-value | | | | | | | | | | |

TABLE 15B shown below presents a summary of Corrected QT interval values (msec) measured in EXAMPLE 5 above. Statistical analysis was based on mixed model analysis of 0.25 through 6 Hour Time Interval Values (Segment 1). The covariate mean is the average of 2-hour pre-dose data with an AR(1) covariance structure.

TABLE 15B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 230.38 | Mean | 231.75 | 232.25 | 234.50 | 229.50 | 228.75 | 227.75 | 228.00 | 222.50 | 227.00 | 227.75 |
| mg/kg | | N | 4 | 4 | 4 | 4 | 4 | 4 | 4 | 4 | 4 | 4 |
| | | LSMean | 232.68 | 233.18 | 235.43 | 230.43 | 229.68 | 228.68 | 228.93 | 223.43 | 227.93 | 228.68 |
| | | LSM s.e. | 1.89 | 2.54 | 2.17 | 2.92 | 2.57 | 2.01 | 2.55 | 4.44 | 2.45 | 2.26 |
| 10 | 230.91 | Mean | 230.50 | 231.25 | 230.75 | 230.25 | 232.50 | 231.25 | 226.00 | 230.25 | 229.25 | 232.75 |
| mg/kg | | N | 4 | 4 | 4 | 4 | 4 | 4 | 4 | 4 | 4 | 4 |
| | | LSMean | 231.17 | 231.92 | 231.42 | 230.92 | 233.17 | 231.92 | 226.67 | 230.92 | 229.92 | 233.42 |
| | | LSM s.e. | 1.88 | 2.54 | 2.16 | 2.91 | 2.56 | 2.00 | 2.54 | 4.43 | 2.45 | 2.25 |
| | | Trend | | | | | | | | | | |
| | | p-value | | | | | | | | | | |
| 45 | 233.06 | Mean | 229.75 | 229.00 | 233.00 | 232.00 | 234.00 | 229.00 | 220.50 | 231.75 | 233.50 | 230.00 |
| mg/kg | | N | 4 | 4 | 4 | 4 | 4 | 4 | 4 | 4 | 4 | 4 |
| | | LSMean | 229.36 | 228.61 | 232.61 | 231.61 | 233.61 | 228.61 | 220.11 | 231.36 | 233.11 | 229.61 |
| | | LSM s.e. | 1.87 | 2.53 | 2.16 | 2.91 | 2.56 | 1.99 | 2.54 | 4.43 | 2.44 | 2.24 |
| | | Trend | | | | | | | | | | |
| | | p-value | | | | | | | | | | |
| 120 | 234.72 | Mean | 231.00 | 227.50 | 225.75 | 234.50 | 231.00 | 227.00 | 223.25 | 234.50 | 236.25 | 231.50 |
| mg/kg | | N | 4 | 4 | 4 | 4 | 4 | 4 | 4 | 4 | 4 | 4 |
| | | LSMean | 229.80 | 226.30 | 224.55 | 233.30 | 229.80 | 225.80 | 222.05 | 233.30 | 235.05 | 230.30 |
| | | LSM s.e. | 1.90 | 2.55 | 2.18 | 2.93 | 2.58 | 2.02 | 2.56 | 4.44 | 2.46 | 2.27 |
| | | Trend | | | | | | | | | | |
| | | p-value | | | | | | | | | | |

TABLE 15C shown below presents a summary of Corrected QT interval values (msec) and statistical analysis measured in EXAMPLE 5 above. Statistical analysis was based on mixed model analysis of 0.25 through Hour Time Interval Values (Segment 1). The covariate mean is the average of 2-hour pre-dose data with an AR(1) covariance structure.

For TABLE 15A-C, N=numbers of measures to calculate mean; LSMean=Least squares mean; LSM s.e.=Least squares standard error;*=p<0.05; NT=not tested.

TABLE 15C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 230.38 | Mean | 226.75 | 230.00 | 227.75 | 221.25 | 231.50 |
| mg/kg | | N | 4 | 4 | 4 | 4 | 4 |
| | | LSMean | 227.68 | 230.93 | 228.68 | 222.18 | 232.43 |
| | | LSM s.e. | 1.53 | 3.93 | 2.35 | 5.29 | 2.63 |
| 10 | 230.91 | Mean | 231.75 | 227.25 | 236.75 | 237.25 | 231.75 |
| mg/kg | | N | 4 | 4 | 4 | 4 | 4 |
| | | LSMean | 232.42 | 227.92 | 237.42 | 237.92 | 232.42 |
| | | LSM s.e. | 1.52 | 3.92 | 2.34 | 5.29 | 2.63 |
| | | Trend p-value | | | | | |
| 45 | 233.06 | Mean | 231.25 | 231.50 | 232.25 | 230.50 | 234.00 |
| mg/kg | | N | 4 | 4 | 4 | 4 | 4 |
| | | LSMean | 230.86 | 231.11 | 231.86 | 230.11 | 233.61 |
| | | LSM s.e. | 1.51 | 3.92 | 2.34 | 5.28 | 2.62 |
| | | Trend p-value | | | | | |
| 120 | 234.72 | Mean | 234.00 | 232.25 | 233.50 | 230.75 | 234.75 |
| mg/kg | | N | 4 | 4 | 4 | 4 | 4 |
| | | LSMean | 232.80 | 231.05 | 232.30 | 229.55 | 233.55 |
| | | LSM s.e. | 1.55 | 3.93 | 2.36 | 5.29 | 2.64 |
| | | Trend o-value | | | | | |

| Effects | Statistics | Value |
|---|---|---|
| MAIN EFFECT | Group F-test p-value | 0.753 |
| INTERACTION | Group*Time p-value | 0.464 |
| | Group Linear Trend*Linear Time p-value | 0.217 |
| | Group Linear Trend*Quadratic Time p-value | 0.060 |

TABLE 16A shown below presents a summary of Corrected QT interval values (msec) measured in EXAMPLE 5 above. Statistical analysis was based on mixed model analysis of 7 through 22 Hour Time Interval Values (Segment 2). The covariate mean is the average of 2-hour pre-dose data with an AR(1) covariance structure.

TABLE 16A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 230.38 | Mean | 231.05 | 230.75 | 228.25 | 230.00 | 231.50 | 233.25 | 231.50 | 232.75 | 232.50 | 230.25 |
| mg/kg | | N | 4 | 4 | 4 | 4 | 4 | 4 | 4 | 4 | 4 | 4 |
| | | LSMean | 232.27 | 231.97 | 229.47 | 231.22 | 232.72 | 234.47 | 232.72 | 233.97 | 233.72 | 231.47 |
| | | LSM s.e. | 1.60 | 2.53 | 2.53 | 2.53 | 2.53 | 2.53 | 2.53 | 2.53 | 2.53 | 2.53 |
| 10 | 230.91 | Mean | 235.34 | 234.25 | 234.00 | 235.75 | 236.75 | 234.75 | 236.50 | 235.50 | 234.50 | 236.50 |
| mg/kg | | N | 4 | 4 | 4 | 4 | 4 | 4 | 4 | 4 | 4 | 4 |
| | | LSMean | 236.22 | 235.13 | 234.88 | 236.63 | 237.63 | 235.63 | 237.38 | 236.38 | 235.38 | 237.38 |
| | | LSM s.e. | 1.58 | 2.52 | 2.52 | 2.52 | 2.52 | 2.52 | 2.52 | 2.52 | 2.52 | 2.52 |
| | | Trend p-value | NT | | | | | | | | | |
| 45 | 233.06 | Mean | 235.17 | 236.00 | 236.25 | 234.75 | 234.25 | 235.25 | 240.25 | 232.75 | 231.75 | 236.75 |
| mg/kg | | N | 4 | 4 | 4 | 4 | 4 | 4 | 4 | 4 | 4 | 4 |
| | | LSMean | 234.66 | 235.49 | 235.74 | 234.24 | 233.74 | 234.74 | 239.74 | 232.24 | 231.24 | 236.24 |
| | | LSM s.e. | 1.57 | 2.51 | 2.51 | 2.51 | 2.51 | 2.51 | 2.51 | 2.51 | 2.51 | 2.51 |
| | | Trend p-value | NT | | | | | | | | | |
| 120 | 234.72 | Mean | 235.55 | 232.75 | 231.00 | 238.00 | 240.50 | 236.00 | 239.00 | 233.50 | 233.00 | 235.25 |
| mg/kg | | N | 4 | 4 | 4 | 4 | 4 | 4 | 4 | 4 | 4 | 4 |
| | | LSMean | 233.97 | 231.17 | 229.42 | 236.42 | 238.92 | 234.42 | 237.42 | 231.92 | 231.42 | 233.67 |
| | | LSM s.e. | 1.63 | 2.55 | 2.55 | 2.55 | 2.55 | 2.55 | 2.55 | 2.55 | 2.55 | 2.55 |
| | | Trend p-value | 0.659 | | | | | | | | | |

TABLE 16B shown below presents a summary of Corrected QT interval values (msec) measured in EXAMPLE 5 above. Statistical analysis was based on mixed model analysis of 7 through 22 Hour Time Interval Values (Segment 2). The covariate mean is the average of 2-hour pre-dose data with an AR(1) covariance structure.

TABLE 16B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 230.38 | Mean | 230.00 | 233.50 | 232.50 | 230.75 | 232.00 | 226.75 | 230.50 |
| mg/kg | | N | 4 | 4 | 4 | 4 | 4 | 4 | 4 |
| | | LSMean | 231.22 | 234.72 | 233.72 | 231.97 | 233.22 | 227.97 | 231.72 |
| | | LSM s.e. | 2.53 | 2.53 | 2.53 | 2.53 | 2.53 | 2.53 | 2.53 |
| 10 | 230.91 | Mean | 233.75 | 234.50 | 233.00 | 235.75 | 240.50 | 233.25 | 236.25 |
| mg/kg | | N | 4 | 4 | 4 | 4 | 4 | 4 | 4 |
| | | LSMean | 234.63 | 235.38 | 233.88 | 236.63 | 241.38 | 234.13 | 237.13 |
| | | LSM s.e. | 2.52 | 2.52 | 2.52 | 2.52 | 2.52 | 2.52 | 2.52 |
| | | Trend p-value | | | | | | | |
| 45 | 233.06 | Mean | 232.50 | 232.50 | 232.75 | 236.00 | 236.75 | 236.75 | 237.50 |
| mg/kg | | N | 4 | 4 | 4 | 4 | 4 | 4 | 4 |
| | | LSMean | 231.99 | 231.99 | 232.24 | 235.49 | 236.24 | 236.24 | 236.99 |
| | | LSM s.e. | 2.51 | 2.51 | 2.51 | 2.51 | 2.51 | 2.51 | 2.51 |
| | | Trend p-value | | | | | | | |
| 120 | 234.72 | Mean | 235.25 | 235.50 | 235.00 | 235.00 | 237.75 | 235.50 | 235.75 |
| mg/kg | | N | 4 | 4 | 4 | 4 | 4 | 4 | 4 |
| | | LSMean | 233.67 | 233.92 | 233.42 | 233.42 | 236.17 | 233.92 | 234.17 |
| | | LSM s.e. | 2.55 | 2.55 | 2.55 | 2.55 | 2.55 | 2.55 | 2.55 |
| | | Trend p-value | | | | | | | |

TABLE 16C shown below presents statistical analysis of data presented in TABLE 16A-B. Statistical analysis was based on a mixed model analysis of 7 through 22 Hour Time Interval Values (Segment 2).

For TABLE 16A-B, N=numbers of measures to calculate mean; LSMean=Least squares mean; LSM s.e.=Least squares standard error; *=p<0.05; NT=not tested.

TABLE 16C

| Effects | Statistics | Value |
|---|---|---|
| MAIN EFFECT | Group F-test p-value | 0.430 |
| INTERACTION | Group*Time p-value | 0.559 |
| | Group Linear Trend*Linear Time p-value | 0.806 |
| | Group Linear Trend*Quadratic Time p-value | 0.370 |

TABLE 17A shown below presents a summary of systolic blood pressure values (msec) measured in EXAMPLE 6 above. Statistical analysis was based on mixed model analysis of 0.25 through 6 Hour Time Interval Values (Segment 1). The covariate mean is the average of 2-hour pre-dose data with an AR(1) covariance structure.

TABLE 17A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 154.56 | Mean | 148.46 | 157.25 | 144.50 | 143.00 | 141.50 | 142.50 | 143.25 | 154.25 | 144.50 | 148.00 |
| mg/kg | | N | 4 | 4 | 4 | 4 | 4 | 4 | 4 | 4 | 4 | 4 |
| | | LSMean | 144.10 | 152.89 | 140.14 | 138.64 | 137.14 | 138.14 | 138.89 | 149.89 | 140.14 | 143.64 |
| | | LSM s.e. | 4.78 | 9.41 | 10.09 | 6.74 | 6.54 | 5.56 | 7.01 | 7.17 | 5.45 | 5.15 |
| 10 | 146.59 | Mean | 129.67 | 137.50 | 114.75 | 113.00 | 116.25 | 121.25 | 127.00 | 129.25 | 126.00 | 131.00 |
| mg/kg | | N | 4 | 4 | 4 | 4 | 4 | 4 | 4 | 4 | 4 | 4 |
| | | LSMean | 133.59 | 141.42 | 118.67 | 116.92 | 120.17 | 125.17 | 130.92 | 133.17 | 129.92 | 134.92 |
| | | LSM s.e. | 4.67 | 9.36 | 10.03 | 6.66 | 6.46 | 5.47 | 6.94 | 7.10 | 5.35 | 5.05 |
| | | Trend | 0.193 | NT | NT | 0.047* | 0.102 | 0.152 | NT | 0.134 | 0.244 | 0.295 |
| | | p-value | | | | | | | | | | |
| 45 | 148.19 | Mean | 117.31 | 143.75 | 115.25 | 105.25 | 105.50 | 111.00 | 117.50 | 114.75 | 114.75 | 110.00 |
| mg/kg | | N | 4 | 4 | 4 | 4 | 4 | 4 | 4 | 4 | 4 | 4 |
| | | LSMean | 119.58 | 146.01 | 117.51 | 107.51 | 107.76 | 113.26 | 119.76 | 117.01 | 117.01 | 112.26 |
| | | LSM s.e. | 4.35 | 9.20 | 9.89 | 6.44 | 6.23 | 5.19 | 6.72 | 6.89 | 5.07 | 4.75 |
| | | Trend | 0.007* | NT | NT | 0.006* | 0.007* | 0.009* | 0.075 | 0.006* | 0.013* | 0.002* |
| | | p-value | | | | | | | | | | |
| 120 | 152.13 | Mean | 125.50 | 155.75 | 127.75 | 116.50 | 119.00 | 118.00 | 119.00 | 116.00 | 120.75 | 118.50 |
| mg/kg | | N | 4 | 4 | 4 | 4 | 4 | 4 | 4 | 4 | 4 | 4 |
| | | LSMean | 123.67 | 153.92 | 125.92 | 114.67 | 117.17 | 116.17 | 117.17 | 114.17 | 118.92 | 116.67 |
| | | LSM s.e. | 4.29 | 9.17 | 9.86 | 6.40 | 6.19 | 5.14 | 6.68 | 6.85 | 5.02 | 4.69 |
| | | Trend | 0.003* | 0.854 | 0.337 | 0.012* | 0.019* | 0.005* | 0.022* | 0.001* | 0.005* | 0.000* |
| | | p-value | | | | | | | | | | |

TABLE 17B shown below presents a summary of systolic blood pressure values (msec) measured in EXAMPLE 6 above. Statistical analysis was based on mixed model analysis of 0.25 through 6 Hour Time Interval Values (Segment 1). The covariate mean is the average of 2-hour pre-dose data with an AR(1) covariance structure.

TABLE 17B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 154.56 | Mean | 147.25 | 148.25 | 148.25 | 150.50 | 148.50 | 151.75 | 158.50 | 162.25 | 144.00 | 137.75 |
| mg/kg | | N | 4 | 4 | 4 | 4 | 4 | 4 | 4 | 4 | 4 | 4 |
| | | LSMean | 142.89 | 143.89 | 143.89 | 146.14 | 144.14 | 147.39 | 154.14 | 157.89 | 139.64 | 133.39 |
| | | LSM s.e. | 5.68 | 5.97 | 5.54 | 4.72 | 5.22 | 5.46 | 8.05 | 5.76 | 6.64 | 5.47 |
| 10 | 146.59 | Mean | 129.25 | 130.75 | 130.75 | 132.25 | 130.25 | 133.75 | 149.25 | 133.75 | 127.00 | 124.50 |
| mg/kg | | N | 4 | 4 | 4 | 4 | 4 | 4 | 4 | 4 | 4 | 4 |
| | | LSMean | 133.17 | 134.67 | 134.67 | 136.17 | 134.17 | 137.67 | 153.17 | 137.67 | 130.92 | 128.42 |
| | | LSM s.e. | 5.59 | 5.88 | 5.44 | 4.61 | 5.12 | 5.36 | 7.99 | 5.67 | 6.56 | 5.38 |
| | | Trend | 0.279 | 0.322 | 0.295 | 0.212 | 0.239 | 0.265 | NT | 0.040* | 0.393 | NT |
| | | p-value | | | | | | | | | | |
| 45 | 148.19 | Mean | 110.25 | 113.25 | 116.75 | 112.50 | 112.25 | 119.00 | 135.75 | 123.50 | 114.75 | 114.75 |
| mg/kg | | N | 4 | 4 | 4 | 4 | 4 | 4 | 4 | 4 | 4 | 4 |
| | | LSMean | 112.51 | 115.51 | 119.01 | 114.76 | 114.51 | 121.26 | 138.01 | 125.76 | 117.01 | 117.01 |
| | | LSM s.e. | 5.32 | 5.63 | 5.16 | 4.28 | 4.83 | 5.08 | 7.80 | 5.40 | 6.33 | 5.10 |
| | | Trend | 0.003* | 0.005* | 0.009* | 0.002* | 0.002* | 0.006* | NT | 0.002* | 0.035* | 0.063 |
| | | p-value | | | | | | | | | | |
| 120 | 152.13 | Mean | 123.00 | 120.50 | 122.50 | 127.25 | 129.75 | 124.50 | 139.75 | 118.00 | 120.50 | 120.50 |
| mg/kg | | N | 4 | 4 | 4 | 4 | 4 | 4 | 4 | 4 | 4 | 4 |
| | | LSMean | 121.17 | 118.67 | 120.67 | 125.42 | 127.92 | 122.67 | 137.92 | 116.17 | 118.67 | 118.67 |
| | | LSM s.e. | 5.27 | 5.58 | 5.11 | 4.22 | 4.77 | 5.03 | 7.77 | 5.36 | 6.29 | 5.05 |
| | | Trend | 0.002* | 0.002* | 0.002* | 0.001* | 0.007* | 0.001* | 0.097 | 0.000* | 0.019* | 0.030* |
| | | p-value | | | | | | | | | | |

TABLE 17C shown below presents a summary of systolic blood pressure values (msec) and statistical analysis measured in EXAMPLE 6 above. Statistical analysis was based on mixed model analysis of 0.25 through Hour Time Interval Values (Segment 1). The covariate mean is the average of 2-hour pre-dose data with an AR(1) covariance structure.

For TABLE 17A-C, N=numbers of measures to calculate mean; LSMean=Least squares mean; LSM s.e.=Least squares standard error;*=p<0.05; NT=not tested.

TABLE 17C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 154.56 | Mean | 144.50 | 147.25 | 153.00 | 152.75 | 149.75 |
| mg/kg | | N | 4 | 4 | 4 | 4 | 4 |
| | | LSMean | 140.14 | 142.89 | 148.64 | 148.39 | 145.39 |
| | | LSM s.e. | 4.91 | 4.58 | 5.21 | 5.77 | 3.99 |
| 10 | 146.59 | Mean | 129.00 | 132.75 | 138.00 | 136.00 | 138.75 |
| mg/kg | | N | 4 | 4 | 4 | 4 | 4 |
| | | LSMean | 132.92 | 136.67 | 141.92 | 139.92 | 142.67 |
| | | LSM s.e. | 4.81 | 4.46 | 5.10 | 5.67 | 3.85 |
| | | Trend p-value | 0.368 | 0.412 | 0.417 | 0.348 | 0.689 |
| 45 | 148.19 | Mean | 118.25 | 120.25 | 119.50 | 120.50 | 126.50 |
| mg/kg | | N | 4 | 4 | 4 | 4 | 4 |
| | | LSMean | 120.51 | 122.51 | 121.76 | 122.76 | 128.76 |
| | | LSM s.e. | 4.49 | 4.12 | 4.81 | 5.41 | 3.45 |
| | | Trend p-value | 0.023* | 0.017* | 0.004* | 0.009* | 0.039* |
| 120 | 152.13 | Mean | 127.50 | 131.25 | 128.25 | 134.00 | 133.50 |
| mg/kg | | N | 4 | 4 | 4 | 4 | 4 |
| | | LSMean | 125.67 | 129.42 | 126.42 | 132.17 | 131.67 |
| | | LSM s.e. | 4.43 | 4.06 | 4.75 | 5.36 | 3.38 |
| | | Trend p-value | 0.018* | 0.014* | 0.001* | 0.016* | 0.015* |

| Effects | Statistics | Value |
|---|---|---|
| MAIN EFFECT | Group F-test p-value | 0.014* |
| INTERACTION | Group*Time p-value | 0.008* |
| | Group Linear Trend*Linear Time p-value | 0.816 |
| | Group Linear Trend*Quadratic Time p-value | 0.040* |

TABLE 18A shown below presents a summary of systolic blood pressure values (msec) measured in EXAMPLE 6 above. Statistical analysis was based on mixed model analysis of 7 through 22 Hour Time Interval Values (Segment 2). The covariate mean is the average of 2-hour pre-dose data with an AR(1) covariance structure.

TABLE 18A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 154.56 | Mean | 143.83 | 154.75 | 153.00 | 149.25 | 144.50 | 143.25 | 137.00 | 136.75 | 136.75 | 142.00 |
| mg/kg | | N | 4 | 4 | 4 | 4 | 4 | 4 | 4 | 4 | 4 | 4 |
| | | LSMean | 143.44 | 154.36 | 152.61 | 148.86 | 144.11 | 142.86 | 136.61 | 136.36 | 136.36 | 141.61 |
| | | LSM s.e. | 3.00 | 4.05 | 4.05 | 4.05 | 4.05 | 4.05 | 4.05 | 4.05 | 4.05 | 4.05 |
| 10 | 146.59 | Mean | 143.53 | 142.25 | 141.50 | 144.50 | 142.75 | 142.25 | 140.75 | 141.50 | 140.75 | 143.50 |
| mg/kg | | N | 4 | 4 | 4 | 4 | 4 | 4 | 4 | 4 | 4 | 4 |
| | | LSMean | 143.88 | 142.60 | 141.85 | 144.85 | 143.10 | 142.60 | 141.10 | 141.85 | 141.10 | 143.85 |
| | | LSM s.e. | 2.90 | 3.98 | 3.98 | 3.98 | 3.98 | 3.98 | 3.98 | 3.98 | 3.98 | 3.98 |
| | | Trend p-value | NT | 0.076 | 0.102 | 0.524 | 0.872 | 0.967 | NT | NT | NT | NT |
| 45 | 148.19 | Mean | 135.77 | 126.75 | 127.75 | 129.75 | 129.50 | 128.75 | 133.00 | 135.50 | 133.50 | 139.00 |
| mg/kg | | N | 4 | 4 | 4 | 4 | 4 | 4 | 4 | 4 | 4 | 4 |
| | | LSMean | 135.97 | 126.95 | 127.95 | 129.95 | 129.70 | 128.95 | 133.20 | 135.70 | 133.70 | 139.20 |
| | | LSM s.e. | 2.62 | 3.78 | 3.78 | 3.78 | 3.78 | 3.78 | 3.78 | 3.78 | 3.78 | 3.78 |
| | | Trend p-value | 0.145 | 0.000* | 0.001* | 0.005* | 0.025* | 0.030* | NT | NT | NT | NT |
| 120 | 152.13 | Mean | 131.78 | 132.50 | 131.75 | 128.50 | 128.25 | 125.50 | 128.00 | 128.50 | 130.00 | 132.25 |
| mg/kg | | N | 4 | 4 | 4 | 4 | 4 | 4 | 4 | 4 | 4 | 4 |
| | | LSMean | 131.62 | 132.34 | 131.59 | 128.34 | 128.09 | 125.34 | 127.84 | 128.34 | 129.84 | 132.09 |
| | | LSM s.e. | 2.57 | 3.74 | 3.74 | 3.74 | 3.74 | 3.74 | 3.74 | 3.74 | 3.74 | 3.74 |
| | | Trend p-value | 0.011* | 0.000* | 0.000* | 0.000* | 0.001* | 0.001* | 0.051 | 0.083 | 0.119 | 0.058 |

TABLE 18B shown below presents a summary of systolic blood pressure values (msec) measured in EXAMPLE 6 above. Statistical analysis was based on mixed model analysis of 7 through 22 Hour Time Interval Values (Segment 2). The covariate mean is the average of 2-hour pre-dose data with an AR(1) covariance structure.

TABLE 18B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 154.56 | Mean | 138.75 | 142.00 | 143.75 | 141.75 | 143.25 | 147.25 | 147.25 |
| mg/kg | | N | 4 | 4 | 4 | 4 | 4 | 4 | 4 |
| | | LSMean | 138.36 | 141.61 | 143.36 | 141.36 | 142.86 | 146.86 | 146.86 |
| | | LSM s.e. | 4.05 | 4.05 | 4.05 | 4.05 | 4.05 | 4.05 | 4.05 |
| 10 | 146.59 | Mean | 142.00 | 146.75 | 146.00 | 146.00 | 144.25 | 147.25 | 144.50 |
| mg/kg | | N | 4 | 4 | 4 | 4 | 4 | 4 | 4 |
| | | LSMean | 142.35 | 147.10 | 146.35 | 146.35 | 144.60 | 147.60 | 144.85 |
| | | LSM s.e. | 3.98 | 3.98 | 3.98 | 3.98 | 3.98 | 3.98 | 3.98 |
| | | Trend p-value | NT | NT | NT | NT | NT | NT | NT |
| 45 | 148.19 | Mean | 136.50 | 138.50 | 142.00 | 142.25 | 142.50 | 144.75 | 142.25 |
| mg/kg | | N | 4 | 4 | 4 | 4 | 4 | 4 | 4 |
| | | LSMean | 136.70 | 138.70 | 142.20 | 142.45 | 142.70 | 144.95 | 142.45 |
| | | LSM s.e. | 3.78 | 3.78 | 3.78 | 3.78 | 3.78 | 3.78 | 3.78 |
| | | Trend p-value | NT | NT | NT | NT | NT | 0.747 | 0.459 |
| 120 | 152.13 | Mean | 135.75 | 134.50 | 137.75 | 131.75 | 133.50 | 135.50 | 134.50 |
| mg/kg | | N | 4 | 4 | 4 | 4 | 4 | 4 | 4 |
| | | LSMean | 135.59 | 134.34 | 137.59 | 131.59 | 133.34 | 135.34 | 134.34 |
| | | LSM s.e. | 3.74 | 3.74 | 3.74 | 3.74 | 3.74 | 3.74 | 3.74 |
| | | Trend p-value | 0.409 | 0.083 | 0.209 | 0.058 | 0.080 | 0.036* | 0.025* |

TABLE 18C shown below presents statistical analysis of the data presented in TABLE 18A-B. Statistical analysis was based on a mixed model analysis of 7 through 22 Hour Time Interval Values (Segment 2).

For TABLE 18A-B, N=numbers of measures to calculate mean; LSMean=Least squares mean; LSM s.e.=Least squares standard error; *=p<0.05; NT=not tested.

TABLE 18C

| Effects | Statistics | Value |
|---|---|---|
| MAIN EFFECT | Group F-test p-value | 0.042* |
| INTERACTION | Group*Time p-value | 0.310 |
| | Group Linear Trend*Linear Time p-value | 0.014* |
| | Group Linear Trend*Quadratic Time p-value | 0.019* |

TABLE 19A shown below presents a summary of diastolic blood pressure values (msec) measured in EXAMPLE 6 above. Statistical analysis was based on mixed model analysis of 0.25 through 6 Hour Time Interval Values (Segment 1). The covariate mean is the average of 2-hour pre-dose data with an AR(1) covariance structure.

TABLE 19A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 81.47 | Mean | 78.60 | 84.25 | 79.25 | 77.50 | 76.00 | 74.25 | 74.75 | 79.75 | 76.25 | 76.75 |
| mg/kg | | N | 4 | 4 | 4 | 4 | 4 | 4 | 4 | 4 | 4 | 4 |
| | | LSMean | 78.13 | 83.77 | 78.77 | 77.02 | 75.52 | 73.77 | 74.27 | 79.27 | 75.77 | 76.27 |
| | | LSM s.e. | 1.87 | 3.45 | 3.45 | 3.45 | 3.45 | 3.45 | 3.45 | 3.45 | 3.45 | 3.45 |
| 10 | 78.59 | Mean | 68.98 | 72.00 | 60.75 | 60.75 | 62.00 | 65.00 | 65.75 | 67.75 | 66.75 | 68.50 |
| mg/kg | | N | 4 | 4 | 4 | 4 | 4 | 4 | 4 | 4 | 4 | 4 |
| | | LSMean | 69.36 | 72.38 | 61.13 | 61.13 | 62.38 | 65.38 | 66.13 | 68.13 | 67.13 | 68.88 |
| | | LSM s.e. | 1.84 | 3.43 | 3.43 | 3.43 | 3.43 | 3.43 | 3.43 | 3.43 | 3.43 | 3.43 |
| | | Trend | 0.020* | | | | | | | | | |
| | | p-value | | | | | | | | | | |
| 45 | 79.00 | Mean | 63.83 | 78.50 | 61.75 | 58.00 | 59.25 | 59.50 | 62.75 | 62.50 | 62.00 | 59.00 |
| mg/kg | | N | 4 | 4 | 4 | 4 | 4 | 4 | 4 | 4 | 4 | 4 |
| | | LSMean | 64.09 | 78.76 | 62.01 | 58.26 | 59.51 | 59.76 | 63.01 | 62.76 | 62.26 | 59.26 |
| | | LSM s.e. | 1.80 | 3.41 | 3.41 | 3.41 | 3.41 | 3.41 | 3.41 | 3.41 | 3.41 | 3.41 |
| | | Trend | 0.002* | | | | | | | | | |
| | | p-value | | | | | | | | | | |
| 120 | 80.38 | Mean | 69.76 | 90.75 | 75.75 | 68.25 | 67.00 | 66.50 | 65.25 | 65.50 | 66.25 | 66.25 |
| mg/kg | | N | 4 | 4 | 4 | 4 | 4 | 4 | 4 | 4 | 4 | 4 |
| | | LSMean | 69.61 | 90.60 | 75.60 | 68.10 | 66.85 | 66.35 | 65.10 | 65.35 | 66.10 | 66.10 |
| | | LSM s.e. | 1.78 | 3.40 | 3.40 | 3.40 | 3.40 | 3.40 | 3.40 | 3.40 | 3.40 | 3.40 |
| | | Trend | 0.009* | | | | | | | | | |
| | | p-value | | | | | | | | | | |

TABLE 19B shown below presents a summary of diastolic blood pressure values (msec) measured in EXAMPLE 6 above. Statistical analysis was based on mixed model analysis of 0.25 through 6 Hour Time Interval Values (Segment 1). The covariate mean is the average of 2-hour pre-dose data with an AR(1) covariance structure.

TABLE 19B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 81.47 | Mean | 76.75 | 76.50 | 76.25 | 78.00 | 78.00 | 78.25 | 82.50 | 88.50 | 75.50 | 72.00 |
| mg/kg | | N | 4 | 4 | 4 | 4 | 4 | 4 | 4 | 4 | 4 | 4 |
| | | LSMean | 76.27 | 76.02 | 75.77 | 77.52 | 77.52 | 77.77 | 82.02 | 88.02 | 75.02 | 71.52 |
| | | LSM s.e. | 3.45 | 3.45 | 3.45 | 3.45 | 3.45 | 3.45 | 3.45 | 3.45 | 3.45 | 3.45 |
| 10 | 78.59 | Mean | 68.50 | 67.50 | 68.50 | 69.50 | 68.25 | 72.00 | 81.00 | 75.75 | 70.00 | 67.25 |
| mg/kg | | N | 4 | 4 | 4 | 4 | 4 | 4 | 4 | 4 | 4 | 4 |
| | | LSMean | 68.88 | 67.88 | 68.88 | 69.88 | 68.63 | 72.38 | 81.38 | 76.13 | 70.38 | 67.63 |
| | | LSM s.e. | 3.43 | 3.43 | 3.43 | 3.43 | 3.43 | 3.43 | 3.43 | 3.43 | 3.43 | 3.43 |
| | | Trend | | | | | | | | | | |
| | | p-value | | | | | | | | | | |
| 45 | 79.00 | Mean | 59.25 | 60.25 | 63.75 | 61.50 | 61.00 | 63.50 | 75.75 | 69.75 | 63.75 | 62.00 |
| mg/kg | | N | 4 | 4 | 4 | 4 | 4 | 4 | 4 | 4 | 4 | 4 |
| | | LSMean | 59.51 | 60.51 | 64.01 | 61.76 | 61.26 | 63.76 | 76.01 | 70.01 | 64.01 | 62.26 |
| | | LSM s.e. | 3.41 | 3.41 | 3.41 | 3.41 | 3.41 | 3.41 | 3.41 | 3.41 | 3.41 | 3.41 |
| | | Trend | | | | | | | | | | |
| | | p-value | | | | | | | | | | |
| 120 | 80.38 | Mean | 65.50 | 65.75 | 68.00 | 69.75 | 70.00 | 68.00 | 78.00 | 67.50 | 67.00 | 67.50 |
| mg/kg | | N | 4 | 4 | 4 | 4 | 4 | 4 | 4 | 4 | 4 | 4 |
| | | LSMean | 65.35 | 65.60 | 67.85 | 69.60 | 69.85 | 67.85 | 77.85 | 67.35 | 66.85 | 67.35 |
| | | LSM s.e. | 3.40 | 3.40 | 3.40 | 3.40 | 3.40 | 3.40 | 3.40 | 3.40 | 3.40 | 3.40 |
| | | Trend | | | | | | | | | | |
| | | p-value | | | | | | | | | | |

TABLE 19C shown below presents a summary of diastolic blood pressure values (msec) and statistical analysis measured in EXAMPLE 6 above. Statistical analysis was based on mixed model analysis of 0.25 through Hour Time Interval Values (Segment 1). The covariate mean is the average of 2-hour pre-dose data with an AR(1) covariance structure.

For TABLE 19A-C, N=numbers of measures to calculate mean; LSMean=Least squares mean; LSM s.e.=Least squares standard error;*=p<0.05; NT=not tested.

TABLE 19C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 81.47 | Mean | 80.50 | 86.50 | 82.50 | 78.50 | 77.50 |
| mg/kg | | N | 4 | 4 | 4 | 4 | 4 |
| | | LSMean | 80.02 | 86.02 | 82.02 | 78.02 | 77.02 |
| | | LSM s.e. | 3.45 | 3.45 | 3.45 | 3.45 | 3.45 |
| 10 | 78.59 | Mean | 68.50 | 69.50 | 73.50 | 73.50 | 73.00 |
| mg/kg | | N | 4 | 4 | 4 | 4 | 4 |
| | | LSMean | 68.88 | 69.88 | 73.88 | 73.88 | 73.38 |
| | | LSM s.e. | 3.43 | 3.43 | 3.43 | 3.43 | 3.43 |
| | | Trend p-value | | | | | |
| 45 | 79.00 | Mean | 63.00 | 63.25 | 66.25 | 67.00 | 68.75 |
| mg/kg | | N | 4 | 4 | 4 | 4 | 4 |
| | | LSMean | 63.26 | 63.51 | 66.51 | 67.26 | 69.01 |
| | | LSM s.e. | 3.41 | 3.41 | 3.41 | 3.41 | 3.41 |
| | | Trend p-value | | | | | |
| 120 | 80.38 | Mean | 69.75 | 70.75 | 71.00 | 72.00 | 72.25 |
| mg/kg | | N | 4 | 4 | 4 | 4 | 4 |
| | | LSMean | 69.60 | 70.60 | 70.85 | 71.85 | 72.10 |
| | | LSM s.e. | 3.40 | 3.40 | 3.40 | 3.40 | 3.40 |
| | | Trend p-value | | | | | |

| Effects | Statistics | Value |
|---|---|---|
| MAIN EFFECT | Group F-test p-value | 0.013* |
| INTERACTION | Group*Time p-value | 0.015* |
| | Group Linear Trend*Linear Time p-value | 0.388 |
| | Group Linear Trend*Quadratic Time p-value | 0.115 |

TABLE 20A shown below presents a summary of diastolic blood pressure values (msec) measured in EXAMPLE 6 above. Statistical analysis was based on mixed model analysis of 7 through 22 Hour Time Interval Values (Segment 2). The covariate mean is the average of 2-hour pre-dose data with an AR(1) covariance structure.

TABLE 20A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 81.47 | Mean | 72.64 | 80.00 | 77.50 | 75.75 | 71.00 | 69.75 | 67.50 | 67.00 | 68.50 | 71.00 |
| mg/kg | | N | 4 | 4 | 4 | 4 | 4 | 4 | 4 | 4 | 4 | 4 |
| | | LSMean | 72.95 | 80.31 | 77.81 | 76.06 | 71.31 | 70.06 | 67.81 | 67.31 | 68.81 | 71.31 |
| | | LSM s.e. | 1.08 | 2.07 | 2.07 | 2.07 | 2.07 | 2.07 | 2.07 | 2.07 | 2.07 | 2.07 |
| 10 | 78.59 | Mean | 78.09 | 75.50 | 74.50 | 78.75 | 78.00 | 78.50 | 76.25 | 77.25 | 77.50 | 79.50 |
| mg/kg | | N | 4 | 4 | 4 | 4 | 4 | 4 | 4 | 4 | 4 | 4 |
| | | LSMean | 77.85 | 75.25 | 74.25 | 78.50 | 77.75 | 78.25 | 76.00 | 77.00 | 77.25 | 79.25 |
| | | LSM s.e. | 1.06 | 2.06 | 2.06 | 2.06 | 2.06 | 2.06 | 2.06 | 2.06 | 2.06 | 2.06 |
| | | Trend p-value | NT | 0.094 | 0.235 | 0.414 | NT | NT | NT | NT | NT | NT |
| 45 | 79.00 | Mean | 72.95 | 69.00 | 67.00 | 68.75 | 66.75 | 67.50 | 70.25 | 72.00 | 72.00 | 76.50 |
| mg/kg | | N | 4 | 4 | 4 | 4 | 4 | 4 | 4 | 4 | 4 | 4 |
| | | LSMean | 72.79 | 68.83 | 66.83 | 68.58 | 66.58 | 67.33 | 70.08 | 71.83 | 71.83 | 76.33 |
| | | LSM s.e. | 1.04 | 2.05 | 2.05 | 2.05 | 2.05 | 2.05 | 2.05 | 2.05 | 2.05 | 2.05 |
| | | Trend p-value | 0.917 | 0.000* | 0.001* | 0.014* | 0.114 | 0.358 | NT | NT | NT | NT |
| 120 | 80.38 | Mean | 69.75 | 70.50 | 69.25 | 69.25 | 68.75 | 66.25 | 67.50 | 67.25 | 67.00 | 69.00 |
| mg/kg | | N | 4 | 4 | 4 | 4 | 4 | 4 | 4 | 4 | 4 | 4 |
| | | LSMean | 69.85 | 70.60 | 69.35 | 69.35 | 68.85 | 66.35 | 67.60 | 67.35 | 67.10 | 69.10 |
| | | LSM s.e. | 1.03 | 2.05 | 2.05 | 2.05 | 2.05 | 2.05 | 2.05 | 2.05 | 2.05 | 2.05 |
| | | Trend p-value | 0.026* | 0.000* | 0.001* | 0.002* | 0.048* | 0.020* | 0.477 | 0.583 | 0.254 | 0.302 |

TABLE 20B shown below presents a summary of diastolic blood pressure values (msec) measured in EXAMPLE 6 above. Statistical analysis was based on mixed model analysis of 7 through 22 Hour Time Interval Values (Segment 2). The covariate mean is the average of 2-hour pre-dose data with an AR(1) covariance structure.

TABLE 20B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 81.47 | Mean | 69.75 | 72.75 | 73.50 | 71.00 | 74.00 | 76.75 | 76.50 |
| mg/kg | | N | 4 | 4 | 4 | 4 | 4 | 4 | 4 |
| | | LSMean | 70.06 | 73.06 | 73.81 | 71.31 | 74.31 | 77.06 | 76.81 |
| | | LSM s.e. | 2.07 | 2.07 | 2.07 | 2.07 | 2.07 | 2.07 | 2.07 |
| 10 | 78.59 | Mean | 77.50 | 80.50 | 79.50 | 79.25 | 79.75 | 79.00 | 78.25 |
| mg/kg | | N | 4 | 4 | 4 | 4 | 4 | 4 | 4 |
| | | LSMean | 77.25 | 80.25 | 79.25 | 79.00 | 79.50 | 78.75 | 78.00 |
| | | LSM s.e. | 2.06 | 2.06 | 2.06 | 2.06 | 2.06 | 2.06 | 2.06 |
| | | Trend p-value | NT | NT | NT | NT | NT | NT | NT |
| 45 | 79.00 | Mean | 74.50 | 75.25 | 77.75 | 78.75 | 77.75 | 78.00 | 75.50 |
| mg/kg | | N | 4 | 4 | 4 | 4 | 4 | 4 | 4 |
| | | LSMean | 74.33 | 75.08 | 77.58 | 78.58 | 77.58 | 77.83 | 75.33 |
| | | LSM s.e. | 2.05 | 2.05 | 2.05 | 2.05 | 2.05 | 2.05 | 2.05 |
| | | Trend p-value | NT | NT | NT | NT | NT | NT | 0.617 |
| 120 | 80.38 | Mean | 72.50 | 72.25 | 72.00 | 69.25 | 72.75 | 71.75 | 70.75 |
| mg/kg | | N | 4 | 4 | 4 | 4 | 4 | 4 | 4 |
| | | LSMean | 72.60 | 72.35 | 72.10 | 69.35 | 72.85 | 71.85 | 70.85 |
| | | LSM s.e. | 2.05 | 2.05 | 2.05 | 2.05 | 2.05 | 2.05 | 2.05 |
| | | Trend p-value | 0.611 | 0.429 | 0.461 | 0.494 | 0.494 | 0.077 | 0.029* |

TABLE 20C shown below presents statistical analysis of the data shown in TABLE 20A-B. Statistical analysis was based on a mixed model analysis of 7 through 22 Hour Time Interval Values (Segment 2).

For TABLE 20A-B, N=numbers of measures to calculate mean; LSMean=Least squares mean; LSM s.e.=Least squares standard error; *=p<0.05; NT=not tested.

TABLE 20C

| Effects | Statistics | Value |
|---|---|---|
| MAIN EFFECT | Group F-test p-value | 0.015* |
| INTERACTION | Group*Time p-value | 0.048* |
| | Group Linear Trend*Linear Time p-value | 0.026* |
| | Group Linear Trend*Quadratic Time p-value | 0.004* |

TABLE 21A shown below shows a summary of mean arterial pressure values (msec) measured in EXAMPLE 6 above. Statistical analysis is based on a mixed model analysis of 0.25 through 6 Hour Time Interval Values (Segment 1). The covariate mean is the average of 2-hour pre-dose data with an AR(1) covariance structure.

TABLE 21A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 108.81 | Mean | 103.44 | 112.25 | 103.25 | 101.50 | 99.50 | 98.25 | 99.00 | 107.25 | 100.00 | 101.75 |
| mg/kg | | N | 4 | 4 | 4 | 4 | 4 | 4 | 4 | 4 | 4 | 4 |
| | | LSMean | 102.14 | 110.95 | 101.95 | 100.20 | 98.20 | 96.95 | 97.70 | 105.95 | 98.70 | 100.45 |
| | | LSM s.e. | 2.63 | 4.12 | 4.12 | 4.12 | 4.12 | 4.12 | 4.12 | 4.12 | 4.12 | 4.12 |
| 10 | 103.66 | Mean | 90.36 | 96.75 | 80.50 | 79.50 | 81.50 | 85.00 | 87.25 | 91.00 | 87.50 | 90.75 |
| mg/kg | | N | 4 | 4 | 4 | 4 | 4 | 4 | 4 | 4 | 4 | 4 |
| | | LSMean | 91.45 | 97.84 | 81.59 | 80.59 | 82.59 | 86.09 | 88.34 | 92.09 | 88.59 | 91.84 |
| | | LSM s.e. | 2.52 | 4.05 | 4.05 | 4.05 | 4.05 | 4.05 | 4.05 | 4.05 | 4.05 | 4.05 |
| | | Trend | 0.042* | | | | | | | | | |
| | | p-value | | | | | | | | | | |
| 45 | 104.81 | Mean | 82.99 | 102.50 | 81.00 | 75.50 | 76.25 | 77.75 | 83.00 | 82.25 | 81.50 | 77.75 |
| mg/kg | | N | 4 | 4 | 4 | 4 | 4 | 4 | 4 | 4 | 4 | 4 |
| | | LSMean | 83.54 | 103.05 | 81.55 | 76.05 | 76.80 | 78.30 | 83.55 | 82.80 | 82.05 | 78.30 |
| | | LSM s.e. | 2.33 | 3.94 | 3.94 | 3.94 | 3.94 | 3.94 | 3.94 | 3.94 | 3.94 | 3.94 |
| | | Trend | 0.003* | | | | | | | | | |
| | | p-value | | | | | | | | | | |
| 120 | 106.75 | Mean | 89.33 | 113.75 | 94.00 | 85.00 | 85.25 | 84.75 | 84.75 | 83.50 | 85.00 | 84.75 |
| mg/kg | | N | 4 | 4 | 4 | 4 | 4 | 4 | 4 | 4 | 4 | 4 |
| | | LSMean | 88.99 | 113.41 | 93.66 | 84.66 | 84.91 | 84.41 | 84.41 | 83.16 | 84.66 | 84.41 |
| | | LSM s.e. | 2.29 | 3.91 | 3.91 | 3.91 | 3.91 | 3.91 | 3.91 | 3.91 | 3.91 | 3.91 |
| | | Trend | 0.005* | | | | | | | | | |
| | | p-value | | | | | | | | | | |

TABLE 21B shown below shows a summary of mean arterial pressure values (msec) measured in EXAMPLE 6 above. Statistical analysis is based on a mixed model analysis of 0.25 through 6 Hour Time Interval Values (Segment 1). The covariate mean is the average of 2-hour pre-dose data with an AR(1) covariance structure.

TABLE 21B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 108.81 | Mean | 102.00 | 101.50 | 101.50 | 104.00 | 102.75 | 103.75 | 110.50 | 117.00 | 99.75 | 94.50 |
| mg/kg | | N | 4 | 4 | 4 | 4 | 4 | 4 | 4 | 4 | 4 | 4 |
| | | LSMean | 100.70 | 100.20 | 100.20 | 102.70 | 101.45 | 102.45 | 109.20 | 115.70 | 98.45 | 93.20 |
| | | LSM s.e. | 4.12 | 4.12 | 4.12 | 4.12 | 4.12 | 4.12 | 4.12 | 4.12 | 4.12 | 4.12 |
| 10 | 103.66 | Mean | 89.75 | 89.50 | 89.75 | 91.25 | 89.25 | 93.50 | 107.00 | 97.75 | 90.25 | 86.50 |
| mg/kg | | N | 4 | 4 | 4 | 4 | 4 | 4 | 4 | 4 | 4 | 4 |
| | | LSMean | 90.84 | 90.59 | 90.84 | 92.34 | 90.34 | 94.59 | 108.09 | 98.84 | 91.34 | 87.59 |
| | | LSM s.e. | 4.05 | 4.05 | 4.05 | 4.05 | 4.05 | 4.05 | 4.05 | 4.05 | 4.05 | 4.05 |
| | | Trend | | | | | | | | | | |
| | | p-value | | | | | | | | | | |
| 45 | 104.81 | Mean | 77.75 | 79.25 | 81.50 | 79.75 | 79.25 | 83.25 | 99.50 | 89.50 | 81.50 | 80.00 |
| mg/kg | | N | 4 | 4 | 4 | 4 | 4 | 4 | 4 | 4 | 4 | 4 |
| | | LSMean | 78.30 | 79.80 | 82.05 | 80.30 | 79.80 | 83.80 | 100.05 | 90.05 | 82.05 | 80.55 |
| | | LSM s.e. | 3.94 | 3.94 | 3.94 | 3.94 | 3.94 | 3.94 | 3.94 | 3.94 | 3.94 | 3.94 |
| | | Trend | | | | | | | | | | |
| | | p-value | | | | | | | | | | |
| 120 | 106.75 | Mean | 85.25 | 85.25 | 87.25 | 89.75 | 90.50 | 88.25 | 102.75 | 86.00 | 85.25 | 86.00 |
| mg/kg | | N | 4 | 4 | 4 | 4 | 4 | 4 | 4 | 4 | 4 | 4 |
| | | LSMean | 84.91 | 84.91 | 86.91 | 89.41 | 90.16 | 87.91 | 102.41 | 85.66 | 84.91 | 85.66 |
| | | LSM s.e. | 3.91 | 3.91 | 3.91 | 3.91 | 3.91 | 3.91 | 3.91 | 3.91 | 3.91 | 3.91 |
| | | Trend | | | | | | | | | | |
| | | p-value | | | | | | | | | | |

TABLE 21C shown below shows a summary of mean arterial pressure values (msec) and statistical analysis measured in EXAMPLE 6 above. Statistical analysis is based on a mixed model analysis of 0.25 through Hour Time Interval Values (Segment 1). The covariate mean is the average of 2-hour pre-dose data with an AR(1) covariance structure.

For TABLE 21A-C, N=numbers of measures to calculate mean; LSMean=Least squares mean; LSM s.e.=Least squares standard error; *=p<0.05; NT=not tested.

TABLE 21C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 108.81 | Mean | 102.25 | 106.75 | 107.00 | 104.25 | 102.25 |
| mg/kg | | N | 4 | 4 | 4 | 4 | 4 |
| | | LSMean | 100.95 | 105.45 | 105.70 | 102.95 | 100.95 |
| | | LSM s.e. | 4.12 | 4.12 | 4.12 | 4.12 | 4.12 |
| 10 | 103.66 | Mean | 88.75 | 90.75 | 95.50 | 94.75 | 94.75 |
| mg/kg | | N | 4 | 4 | 4 | 4 | 4 |
| | | LSMean | 89.84 | 91.84 | 96.59 | 95.84 | 95.84 |
| | | LSM s.e. | 4.05 | 4.05 | 4.05 | 4.05 | 4.05 |
| | | Trend p-value | | | | | |
| 45 | 104.81 | Mean | 82.00 | 82.25 | 85.00 | 85.25 | 88.50 |
| mg/kg | | N | 4 | 4 | 4 | 4 | 4 |
| | | LSMean | 82.55 | 82.80 | 85.55 | 85.80 | 89.05 |
| | | LSM s.e. | 3.94 | 3.94 | 3.94 | 3.94 | 3.94 |
| | | Trend p-value | | | | | |
| 120 | 106.75 | Mean | 89.25 | 91.25 | 90.75 | 93.00 | 92.75 |
| mg/kg | | N | 4 | 4 | 4 | 4 | 4 |
| | | LSMean | 88.91 | 90.91 | 90.41 | 92.66 | 92.41 |
| | | LSM s.e. | 3.91 | 3.91 | 3.91 | 3.91 | 3.91 |
| | | Trend p-value | | | | | |

| Effects | Statistics | Value |
|---|---|---|
| MAIN EFFECT | Group F-test p-value | 0.013* |
| INTERACTION | Group*Time p-value | 0.001* |
| | Group Linear Trend*Linear Time p-value | 0.620 |
| | Group Linear Trend*Quadratic Time p-value | 0.094 |

TABLE 22A shown below shows a summary of mean arterial pressure values (msec) measured in EXAMPLE 6 above. Statistical analysis is based on a mixed model analysis of 7 through 22 Hour Time Interval Values (Segment 2). The covariate mean is the average of 2-hour pre-dose data with an AR(1) covariance structure.

TABLE 22A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 108.81 | Mean | 97.42 | 106.00 | 103.75 | 101.00 | 96.00 | 94.75 | 91.75 | 91.25 | 92.25 | 95.25 |
| mg/kg | | N | 4 | 4 | 4 | 4 | 4 | 4 | 4 | 4 | 4 | 4 |
| | | LSMean | 97.77 | 106.34 | 104.09 | 101.34 | 96.34 | 95.09 | 92.09 | 91.59 | 92.59 | 95.59 |
| | | LSM s.e. | 1.73 | 2.68 | 2.68 | 2.68 | 2.68 | 2.68 | 2.68 | 2.68 | 2.68 | 2.68 |
| 10 | 103.66 | Mean | 101.03 | 98.00 | 97.75 | 101.75 | 100.50 | 100.50 | 98.50 | 99.00 | 99.50 | 101.75 |
| mg/kg | | N | 4 | 4 | 4 | 4 | 4 | 4 | 4 | 4 | 4 | 4 |
| | | LSMean | 100.74 | 97.71 | 97.46 | 101.46 | 100.21 | 100.21 | 98.21 | 98.71 | 99.21 | 101.46 |
| | | LSM s.e. | 1.66 | 2.64 | 2.64 | 2.64 | 2.64 | 2.64 | 2.64 | 2.64 | 2.64 | 2.64 |
| | | Trend p-value | NT | 0.039* | 0.106 | 0.977 | 0.337 | NT | NT | NT | NT | NT |
| 45 | 104.81 | Mean | 95.02 | 89.25 | 87.50 | 89.75 | 88.25 | 88.75 | 91.75 | 94.00 | 93.25 | 98.50 |
| mg/kg | | N | 4 | 4 | 4 | 4 | 4 | 4 | 4 | 4 | 4 | 4 |
| | | LSMean | 94.87 | 89.10 | 87.35 | 89.60 | 88.10 | 88.60 | 91.60 | 93.85 | 93.10 | 98.35 |
| | | LSM s.e. | 1.53 | 2.56 | 2.56 | 2.56 | 2.56 | 2.56 | 2.56 | 2.56 | 2.56 | 2.56 |
| | | Trend p-value | 0.291 | 0.000* | 0.000* | 0.005* | 0.039* | 0.099 | NT | NT | NT | NT |
| 120 | 106.75 | Mean | 91.06 | 91.50 | 90.25 | 89.25 | 89.00 | 86.50 | 88.25 | 87.75 | 88.25 | 90.50 |
| mg/kg | | N | 4 | 4 | 4 | 4 | 4 | 4 | 4 | 4 | 4 | 4 |
| | | LSMean | 91.15 | 91.59 | 90.34 | 89.34 | 89.09 | 86.59 | 88.34 | 87.84 | 88.34 | 90.59 |
| | | LSM s.e. | 1.50 | 2.54 | 2.54 | 2.54 | 2.54 | 2.54 | 2.54 | 2.54 | 2.54 | 2.54 |
| | | Trend p-value | 0.013* | 0.000* | 0.000* | 0.000* | 0.006* | 0.003* | 0.127 | 0.167 | 0.108 | 0.122 |

TABLE 22B shown below shows a summary of mean arterial pressure values (msec) measured in EXAMPLE 6 above. Statistical analysis is based on a mixed model analysis of 7 through 22 Hour Time Interval Values (Segment 2). The covariate mean is the average of 2-hour pre-dose data with an AR(1) covariance structure.

TABLE 22B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 108.81 | Mean | 93.50 | 96.50 | 97.50 | 95.00 | 98.75 | 102.75 | 102.75 |
| mg/kg | | N | 4 | 4 | 4 | 4 | 4 | 4 | 4 |
| | | LSMean | 93.84 | 96.84 | 97.84 | 95.34 | 99.09 | 103.09 | 103.09 |
| | | LSM s.e. | 2.68 | 2.68 | 2.68 | 2.68 | 2.68 | 2.68 | 2.68 |
| 10 | 103.66 | Mean | 99.75 | 103.50 | 102.75 | 102.75 | 102.75 | 104.75 | 103.00 |
| mg/kg | | N | 4 | 4 | 4 | 4 | 4 | 4 | 4 |
| | | LSMean | 99.46 | 103.21 | 102.46 | 102.46 | 102.46 | 104.46 | 102.71 |
| | | LSM s.e. | 2.64 | 2.64 | 2.64 | 2.64 | 2.64 | 2.64 | 2.64 |
| | | Trend p-value | NT | NT | NT | NT | NT | NT | NT |
| 45 | 104.81 | Mean | 96.00 | 96.75 | 100.25 | 101.25 | 101.25 | 103.50 | 100.25 |
| mg/kg | | N | 4 | 4 | 4 | 4 | 4 | 4 | 4 |
| | | LSMean | 95.85 | 96.60 | 100.10 | 101.10 | 101.10 | 103.35 | 100.10 |
| | | LSM s.e. | 2.56 | 2.56 | 2.56 | 2.56 | 2.56 | 2.56 | 2.56 |
| | | Trend p-value | NT | NT | NT | NT | NT | 0.946 | 0.438 |
| 120 | 106.75 | Mean | 94.25 | 93.50 | 94.25 | 90.50 | 94.00 | 95.25 | 94.00 |
| mg/kg | | N | 4 | 4 | 4 | 4 | 4 | 4 | 4 |
| | | LSMean | 94.34 | 93.59 | 94.34 | 90.59 | 94.09 | 95.34 | 94.09 |
| | | LSM s.e. | 2.54 | 2.54 | 2.54 | 2.54 | 2.54 | 2.54 | 2.54 |
| | | Trend p-value | 0.854 | 0.161 | 0.268 | 0.181 | 0.161 | 0.040* | 0.014* |

TABLE 22C shown below presents statistical analysis of the data of TABLE 22A-B. Statistical analysis was based on a mixed model analysis of 7 through 22 Hour Time Interval Values (Segment 2).

For TABLE 22A-B, N=numbers of measures to calculate mean; LSMean=Least squares mean; LSM s.e.=Least squares standard error; *=p<0.05; NT=not tested.

TABLE 22C

| Effects | Statistics | Value |
|---|---|---|
| MAIN EFFECT | Group F-test p-value | 0.028* |
| INTERACTION | Group*Time p-value | 0.086 |
| | Group Linear Trend*Linear Time p-value | 0.011* |
| | Group Linear Trend*Quadratic Time p-value | 0.006* |

TABLE 23A shown below shows a summary of pulse pressure values (msec) measured in EXAMPLE 6 above. Statistical analysis was based on a mixed model analysis of 0.25 through 6 Hour Time Interval Values (Segment 1). The covariate mean is the average of 2-hour pre-dose data with an AR(1) covariance structure.

TABLE 23A

| | Covariate | | | 0.25 | 0.5 | 0.75 | 1 | 1.25 | 1.5 | 1.75 | 2 | 2.25 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 72.81 | Mean | 69.82 | 73.00 | 65.25 | 65.50 | 65.00 | 68.25 | 68.50 | 74.50 | 68.50 | 71.25 |
| mg/kg | | N | 4 | 4 | 4 | 4 | 4 | 4 | 4 | 4 | 4 | 4 |
| | | LSMean | 68.33 | 71.50 | 63.75 | 64.00 | 63.50 | 66.75 | 67.00 | 73.00 | 67.00 | 69.75 |
| | | LSM s.e. | 2.12 | 3.68 | 4.25 | 3.18 | 2.78 | 2.31 | 3.44 | 3.32 | 2.66 | 2.93 |
| 10 | 67.91 | Mean | 60.74 | 65.50 | 53.50 | 52.25 | 54.00 | 56.25 | 61.25 | 61.50 | 59.00 | 62.50 |
| mg/kg | | N | 4 | 4 | 4 | 4 | 4 | 4 | 4 | 4 | 4 | 4 |
| | | LSMean | 62.14 | 66.90 | 54.90 | 53.65 | 55.40 | 57.65 | 62.65 | 62.90 | 60.40 | 63.90 |
| | | LSM s.e. | 2.09 | 3.66 | 4.24 | 3.16 | 2.76 | 2.29 | 3.42 | 3.31 | 2.64 | 2.91 |
| | | Trend | 0.115 | | | | | | | | | |
| | | p-value | | | | | | | | | | |
| 45 | 69.06 | Mean | 53.44 | 65.00 | 53.50 | 47.50 | 46.00 | 51.25 | 54.75 | 52.25 | 52.75 | 51.00 |
| mg/kg | | N | 4 | 4 | 4 | 4 | 4 | 4 | 4 | 4 | 4 | 4 |
| | | LSMean | 54.16 | 65.72 | 54.22 | 48.22 | 46.72 | 51.97 | 55.47 | 52.97 | 53.47 | 51.72 |
| | | LSM s.e. | 1.96 | 3.59 | 4.18 | 3.08 | 2.66 | 2.16 | 3.34 | 3.22 | 2.53 | 2.82 |
| | | Trend | 0.005* | | | | | | | | | |
| | | p-value | | | | | | | | | | |
| 120 | 71.34 | Mean | 55.76 | 65.25 | 51.75 | 48.25 | 51.50 | 51.50 | 53.75 | 50.75 | 54.50 | 52.25 |
| mg/kg | | N | 4 | 4 | 4 | 4 | 4 | 4 | 4 | 4 | 4 | 4 |
| | | LSMean | 55.13 | 64.62 | 51.12 | 47.62 | 50.87 | 50.87 | 53.12 | 50.12 | 53.87 | 51.62 |
| | | LSM s.e. | 1.95 | 3.58 | 4.17 | 3.07 | 2.65 | 2.15 | 3.34 | 3.22 | 2.52 | 2.81 |
| | | Trend | 0.002* | | | | | | | | | |
| | | p-value | | | | | | | | | | |

TABLE 23B shown below shows a summary of pulse pressure values (msec) measured in EXAMPLE 6 above. Statistical analysis was based on a mixed model analysis of 0.25 through 6 Hour Time Interval Values (Segment 1). The covariate mean is the average of 2-hour pre-dose data with an AR(1) covariance structure.

TABLE 23B

| | Covariate | | 2.5 | 2.75 | 3 | 3.25 | 3.5 | 3.75 | 4 | 4.25 | 4.5 | 4.75 |
|---|---|---|---|---|---|---|---|---|---|---|---|---|
| Group | Mean | Statistics | Hour | Hour | Hour | Hour | Hour | Hour | Hour | Hour | Hour | Hour |
| 0 | 72.81 | Mean | 70.50 | 71.75 | 72.00 | 72.25 | 70.25 | 73.75 | 75.50 | 73.50 | 68.50 | 65.75 |
| mg/kg | | N | 4 | 4 | 4 | 4 | 4 | 4 | 4 | 4 | 4 | 4 |
| | | LSMean | 69.00 | 70.25 | 70.50 | 70.75 | 68.75 | 72.25 | 74.00 | 72.00 | 67.00 | 64.25 |
| | | LSM s.e. | 2.81 | 3.15 | 2.89 | 2.56 | 2.73 | 3.07 | 2.87 | 3.05 | 3.19 | 1.96 |
| 10 | 67.91 | Mean | 60.50 | 62.50 | 62.50 | 63.00 | 62.25 | 62.00 | 68.25 | 58.75 | 57.00 | 57.25 |
| mg/kg | | N | 4 | 4 | 4 | 4 | 4 | 4 | 4 | 4 | 4 | 4 |
| | | LSMean | 61.90 | 63.90 | 63.90 | 64.40 | 63.65 | 63.40 | 69.65 | 60.15 | 58.40 | 58.65 |
| | | LSM s.e. | 2.79 | 3.13 | 2.88 | 2.54 | 2.71 | 3.05 | 2.85 | 3.04 | 3.17 | 1.94 |
| | | Trend | | | | | | | | | | |
| | | p-value | | | | | | | | | | |
| 45 | 69.06 | Mean | 51.50 | 52.25 | 52.75 | 51.25 | 51.50 | 55.50 | 60.00 | 53.75 | 50.75 | 53.00 |
| mg/kg | | N | 4 | 4 | 4 | 4 | 4 | 4 | 4 | 4 | 4 | 4 |
| | | LSMean | 52.22 | 52.97 | 53.47 | 51.97 | 52.22 | 56.22 | 60.72 | 54.47 | 51.47 | 53.72 |
| | | LSM s.e. | 2.69 | 3.05 | 2.78 | 2.43 | 2.61 | 2.96 | 2.75 | 2.95 | 3.09 | 1.79 |
| | | Trend | | | | | | | | | | |
| | | p-value | | | | | | | | | | |
| 120 | 71.34 | Mean | 57.25 | 54.75 | 54.75 | 57.50 | 59.25 | 56.50 | 61.75 | 50.75 | 53.25 | 53.25 |
| mg/kg | | N | 4 | 4 | 4 | 4 | 4 | 4 | 4 | 4 | 4 | 4 |
| | | LSMean | 56.62 | 54.12 | 54.12 | 56.87 | 58.62 | 55.87 | 61.12 | 50.12 | 52.62 | 52.62 |
| | | LSM s.e. | 2.68 | 3.04 | 2.77 | 2.42 | 2.60 | 2.95 | 2.75 | 2.94 | 3.08 | 1.78 |
| | | Trend | | | | | | | | | | |
| | | p-value | | | | | | | | | | |

TABLE 23C shown below shows a summary of pulse pressure values (msec) and statistical analysis measured in EXAMPLE 6 above. Statistical analysis was based on a mixed model analysis of 0.25 through Hour Time Interval Values (Segment 1). The covariate mean is the average of 2-hour pre-dose data with an AR(1)covariance structure.

For TABLE 23A-C, N=numbers of measures t calculate mean; LSMean=Least squares mean; LSM s.e.=Least squares standard error;*=p<0.05; NT=not tested.

TABLE 23C

| | Covariate | | 5 | 5.25 | 5.5 | 5.75 | 6 |
|---|---|---|---|---|---|---|---|
| Group | Mean | Statistics | Hour | Hour | Hour | Hour | Hour |
| 0 | 72.81 | Mean | 64.25 | 60.75 | 70.25 | 74.50 | 72.50 |
| mg/kg | | N | 4 | 4 | 4 | 4 | 4 |
| | | LSMean | 62.75 | 59.25 | 68.75 | 73.00 | 71.00 |
| | | LSM s.e. | 3.70 | 5.49 | 2.98 | 4.59 | 1.92 |
| 10 | 67.91 | Mean | 61.25 | 63.50 | 64.25 | 63.00 | 66.00 |
| mg/kg | | N | 4 | 4 | 4 | 4 | 4 |
| | | LSMean | 62.65 | 64.90 | 65.65 | 64.40 | 67.40 |
| | | LSM s.e. | 3.69 | 5.48 | 2.96 | 4.58 | 1.89 |
| | | Trend p-value | | | | | |
| 45 | 69.06 | Mean | 55.25 | 57.00 | 53.25 | 53.50 | 57.25 |
| mg/kg | | N | 4 | 4 | 4 | 4 | 4 |
| | | LSMean | 55.97 | 57.72 | 53.97 | 54.22 | 57.97 |
| | | LSM s.e. | 3.62 | 5.44 | 2.87 | 4.52 | 1.74 |
| | | Trend p-value | | | | | |
| 120 | 71.34 | Mean | 58.25 | 60.50 | 57.25 | 62.25 | 61.50 |
| mg/kg | | N | 4 | 4 | 4 | 4 | 4 |
| | | LSMean | 57.62 | 59.87 | 56.62 | 61.62 | 60.87 |
| | | LSM s.e. | 3.61 | 5.43 | 2.86 | 4.51 | 1.73 |
| | | Trend p-value | | | | | |

| Effects | Statistics | Value |
|---|---|---|
| MAIN EFFECT | Group F-test p-value | 0.009* |
| INTERACTION | Group*Time p-value | 0.356 |
| | Group Linear Trend*Linear Time p-value | 0.516 |
| | Group Linear Trend*Quadratic Time p-value | 0.068 |

TABLE 24A shown below shows a summary of pulse pressure values (msec) measured in EXAMPLE 6 above. Statistical analysis was based on a mixed model analysis of 7 through 22 Hour Time Interval Values (Segment 2). The covariate mean is the average of 2-hour pre-dose data with an AR(1) covariance structure.

TABLE 24A

| | Covariate | | | 7 | 8 | 9 | 10 | 11 | 12 | 13 | 14 | 15 |
|---|---|---|---|---|---|---|---|---|---|---|---|---|
| Group | Mean | Statistics | Overall | Hour | Hour | Hour | Hour | Hour | Hour | Hour | Hour | Hour |
| 0 | 72.81 | Mean | 71.14 | 74.75 | 76.00 | 73.75 | 73.50 | 73.25 | 69.50 | 69.50 | 68.00 | 70.75 |
| mg/kg | | N | 4 | 4 | 4 | 4 | 4 | 4 | 4 | 4 | 4 | 4 |
| | | LSMean | 70.43 | 74.04 | 75.29 | 73.04 | 72.79 | 72.54 | 68.79 | 68.79 | 67.29 | 70.04 |
| | | LSM s.e. | 1.84 | 2.47 | 2.47 | 2.47 | 2.47 | 2.47 | 2.47 | 2.47 | 2.47 | 2.47 |
| 10 | 67.91 | Mean | 65.45 | 67.00 | 67.00 | 65.75 | 64.75 | 64.00 | 64.75 | 64.25 | 63.50 | 63.75 |
| mg/kg | | N | 4 | 4 | 4 | 4 | 4 | 4 | 4 | 4 | 4 | 4 |
| | | LSMean | 66.12 | 67.66 | 67.66 | 66.41 | 65.41 | 64.66 | 65.41 | 64.91 | 64.16 | 64.41 |
| | | LSM s.e. | 1.81 | 2.45 | 2.45 | 2.45 | 2.45 | 2.45 | 2.45 | 2.45 | 2.45 | 2.45 |
| | | Trend p-value | 0.187 | 0.104 | 0.056 | 0.092 | 0.064 | 0.050* | NT | NT | NT | NT |
| 45 | 69.06 | Mean | 62.89 | 57.25 | 60.50 | 61.25 | 63.25 | 61.25 | 63.00 | 63.50 | 61.75 | 63.00 |
| mg/kg | | N | 4 | 4 | 4 | 4 | 4 | 4 | 4 | 4 | 4 | 4 |
| | | LSMean | 63.23 | 57.59 | 60.84 | 61.59 | 63.59 | 61.59 | 63.34 | 63.84 | 62.09 | 63.34 |
| | | LSM s.e. | 1.68 | 2.35 | 2.35 | 2.35 | 2.35 | 2.35 | 2.35 | 2.35 | 2.35 | 2.35 |
| | | Trend p-value | 0.040* | 0.000* | 0.001* | 0.005* | 0.019* | 0.007* | 0.140 | 0.178 | NT | 0.075 |
| 120 | 71.34 | Mean | 62.05 | 62.00 | 62.25 | 59.50 | 59.75 | 59.25 | 61.00 | 61.50 | 62.50 | 62.75 |
| mg/kg | | N | 4 | 4 | 4 | 4 | 4 | 4 | 4 | 4 | 4 | 4 |
| | | LSMean | 61.75 | 61.70 | 61.95 | 59.20 | 59.45 | 58.95 | 60.70 | 61.20 | 62.20 | 62.45 |
| | | LSM s.e. | 1.67 | 2.34 | 2.34 | 2.34 | 2.34 | 2.34 | 2.34 | 2.34 | 2.34 | 2.34 |
| | | Trend p-value | 0.011* | 0.000* | 0.000* | 0.000* | 0.001* | 0.000* | 0.021* | 0.034* | 0.113 | 0.034* |

TABLE 24B shown below shows a summary of pulse pressure values (msec) measured in EXAMPLE 6 above. Statistical analysis was based on a mixed model analysis of 7 through 22 Hour Time Interval Values (Segment 2). The covariate mean is the average of 2-hour pre-dose data with an AR(1) covariance structure.

TABLE 24B

| | Covariate | | 16 | 17 | 18 | 19 | 20 | 21 | 22 |
|---|---|---|---|---|---|---|---|---|---|
| Group | Mean | Statistics | Hour | Hour | Hour | Hour | Hour | Hour | Hour |
| 0 | 72.81 | Mean | 69.50 | 69.25 | 70.25 | 70.75 | 68.50 | 70.50 | 70.50 |
| mg/kg | | N | 4 | 4 | 4 | 4 | 4 | 4 | 4 |
| | | LSMean | 68.79 | 68.54 | 69.54 | 70.04 | 67.79 | 69.79 | 69.79 |
| | | LSM s.e. | 2.47 | 2.47 | 2.47 | 2.47 | 2.47 | 2.47 | 2.47 |
| 10 | 67.91 | Mean | 64.75 | 66.25 | 66.25 | 66.50 | 64.50 | 68.50 | 65.75 |
| mg/kg | | N | 4 | 4 | 4 | 4 | 4 | 4 | 4 |
| | | LSMean | 65.41 | 66.91 | 66.91 | 67.16 | 65.16 | 69.16 | 66.41 |
| | | LSM s.e. | 2.45 | 2.45 | 2.45 | 2.45 | 2.45 | 2.45 | 2.45 |
| | | Trend p-value | NT | NT | NT | NT | NT | NT | NT |
| 45 | 69.06 | Mean | 62.00 | 63.50 | 64.00 | 63.50 | 64.50 | 67.00 | 67.00 |
| mg/kg | | N | 4 | 4 | 4 | 4 | 4 | 4 | 4 |
| | | LSMean | 62.34 | 63.84 | 64.34 | 63.84 | 64.84 | 67.34 | 67.34 |
| | | LSM s.e. | 2.35 | 2.35 | 2.35 | 2.35 | 2.35 | 2.35 | 2.35 |
| | | Trend p-value | NT | 0.199 | NT | 0.097 | NT | NT | NT |
| 120 | 71.34 | Mean | 63.00 | 62.25 | 65.50 | 62.75 | 61.00 | 64.25 | 63.50 |
| mg/kg | | N | 4 | 4 | 4 | 4 | 4 | 4 | 4 |
| | | LSMean | 62.70 | 61.95 | 65.20 | 62.45 | 60.70 | 63.95 | 63.20 |
| | | LSM s.e. | 2.34 | 2.34 | 2.34 | 2.34 | 2.34 | 2.34 | 2.34 |
| | | Trend p-value | 0.055 | 0.041* | 0.151 | 0.022* | 0.052 | 0.079 | 0.087 |

TABLE 24C shows statistical analysis of the data of TABLE 24A-B. Statistical analysis was based on a mixed model analysis of 7-22 Time Interval Values (Segment 2).

For TABLE 24A-B, N=numbers of measures to calculate mean; LSMean=Least squares mean; LSM s.e.=Least squares standard error; *=p<0.05; NT=not tested.

TABLE 24C

| Effects | Statistics | Value |
|---|---|---|
| MAIN EFFECT | Group F-test p-value | 0.053 |
| INTERACTION | Group*Time p-value | 0.604 |
| | Group Linear Trend*Linear Time p-value | 0.005* |
| | Group Linear Trend*Quadratic Time p-value | 0.141 |

### Example 7. Effect of Compound 1 on Blood Pressure in a Rodent Study

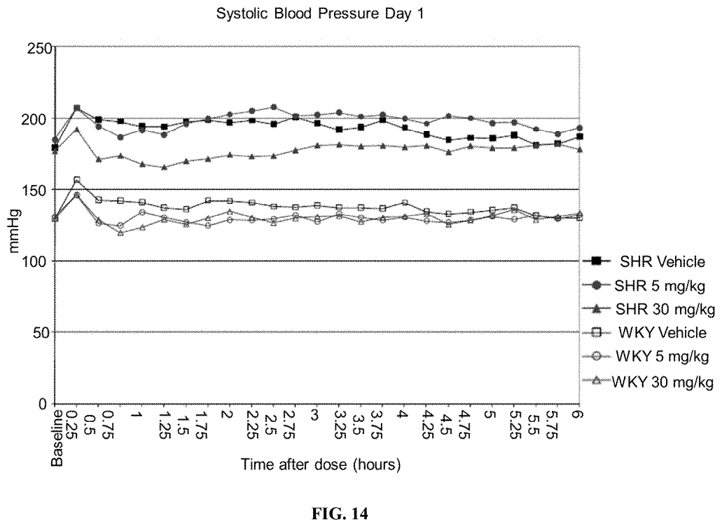

Normotensive Wistar Kyoto (WKY), or spontaneously hypertensive (SHR), rats were treated with a single subcutaneous dose of Compound 1 at 0 (vehicle), 5, or 30 mg/kg. This treatment resulted in a Tₘₐₓ of about 0.75 hours, and a Cₘₐₓ of about 12 μg/mL at the 30 mg/kg dose. Following treatment, blood pressure was monitored over the course of 6 hours. As shown in FIG. 14, an about 10-20 mmHg decrease in systolic blood pressure was seen 0.75 hours post-dose. Compound 1-induced changes in systolic blood pressure were resolved at about 4.25 hours post-dose in WKY rats, and at about 5.75 hours in SHR rats.

### Example 8. Administration of Compound 1 Subcutaneously to Subjects with DME

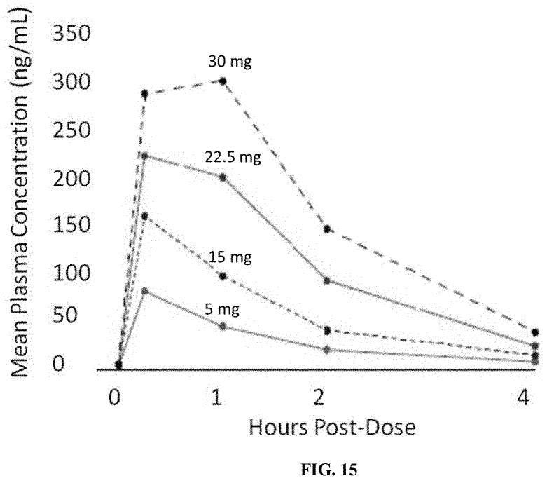

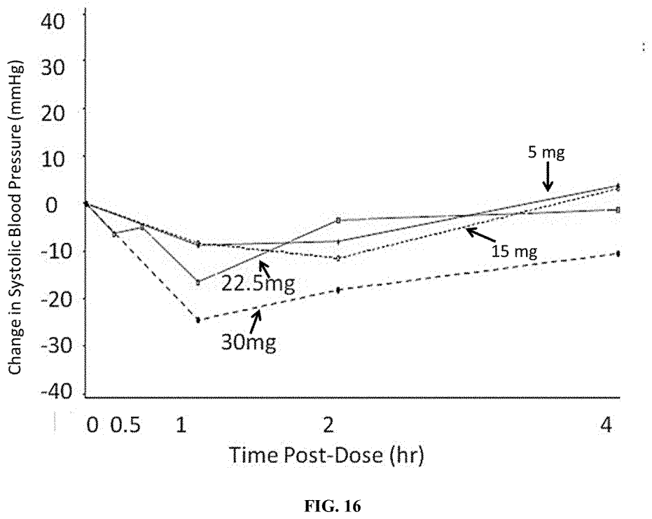

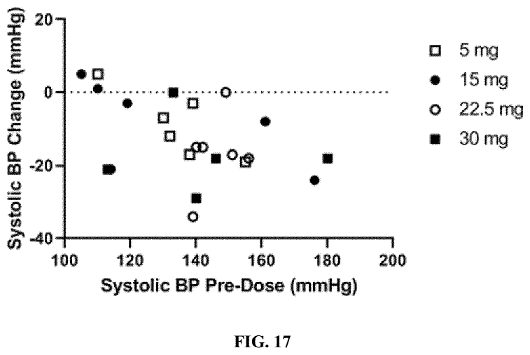

A total of 24 subjects with DME were divided into 4 equally sized cohorts and treated with 5, 15, 22.5, or 30 mg of Compound 1 via subcutaneous injection. As can be seen in FIG. 15, measurements of Compound 1 concentration in plasma indicated a dose-proportional increase in Cₘₐₓ following injection with rapid clearance. For subjects receiving 5, 15, or 30 mg doses of Compound 1 blood pressure was measured at 1, 2, and 4 hours after treatment. Subjects receiving a 30 mg dose of Compound 1 had their blood pressure measured 0.25, 0.5, 1, 2, and 4 hours after treatment. Results showed a transient and dose-dependent decrease in the systolic blood pressure of subjects, as can be seen in FIG. 16. The magnitude of this decrease in blood pressure correlated with the pre-dose blood pressure of subjects as shown in FIG. 17. Detailed results from blood pressure measurements on all groups at 1 hour post-dose can be seen in TABLE 25.

TABLE 25

| | Compound 1 | Compound 1 | Compound 1 | Compound 1 |
|---|---|---|---|---|
| | 5 mg BID | 15 mg BID | 22.5 mg BID | 30 mg BID |
| | (N = 6) | (N = 6) | (N = 6) | (N = 6) |
| Mean BP | 134 (14.7) | 131 (29.9) | 146 (6.4) | 143 (20.2) |
| Pre-dose | | | | |
| (SD) | | | | |
| Min-Max | 110-155 | 105-176 | 139-156 | 113-180 |
| Mean BP | 125 (8.8) | 122 (24.5) | 130 (14.8) | 119 (27.6) |
| 1 hr | | | | |
| Post-dose | | | | |
| Min-Max | 115-136 | 93-153 | 105-149 | 89-162 |
| Mean | −8.8 (9.04) | −8.3 (11.83) | −16.5 (10.82) | −24.5 (20.25) |
| Change | | | | |
| (SD) | | | | |
| Min-Max | −19-5 | −24-5 | −34-0 | −61-0 |

BP = blood pressure
Min = minimum value in cohort
Max = maximum value in cohort
SD = standard deviation

### Example 9. Administration of Ranibizumab and Compound 1 Combination Treatment in Subjects with DME

A total of 144 subjects with DME were split into 3 treatment groups. Group 1 received a subcutaneous injection of Compound 1 (15 mg) and a sham intravitreal injection. Group 2 received a subcutaneous injection of Compound 1 (15 mg) and an intravitreal injection of ranibizumab (0.3 mg). Group 3 received a subcutaneous injection of a placebo, and an intravitreal injection of ranibizumab (0.3 mg). Detailed demographic information on the subjects of this study is shown in TABLE 26.

TABLE 26

| | Compound 1 + | Compound 1 + | Placebo + |
|---|---|---|---|
| | Sham | Ranibizumab | Ranibizumab |
| | (Group 1, | (Group 2, | (Group 3, |
| | N = 48) | N = 49) | N = 47) |
| Age | | | |
| Mean (SD) | 61.3 (7.14) | 61.1 (9.34) | 61.2 (8.99) |
| Median | 62.0 | 61.0 | 62.0 |
| Min, Max | 47, 78 | 34, 81 | 41, 79 |
| <65 years, n (%) | 30 (62.5%) | 32 (65.3%) | 27 (57.4%) |
| ≥65 years, n (%) | 18 (37.5%) | 17 (34.7%) | 20 (42.6%) |
| Gender | | | |
| Male, n (%) | 26 (54.2%) | 26 (53.1%) | 33 (70.2%) |
| Female, n (%) | 22 (45.8%) | 23 (46.9%) | 14 (29.8%) |

Min = minimum value in treatment group
Max = maximum value in treatment group
SD = standard deviation

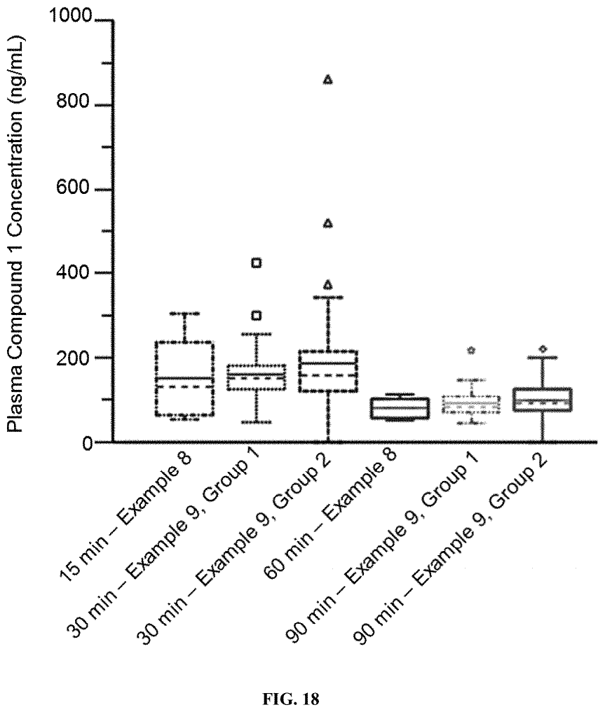

FIG. 18 shows mean plasma concentrations of Compound 1 at 30 and 90 minutes post-dose for subjects of this EXAMPLE (EXAMPLE 9), and at 15 and 60 minutes post-dose for subjects of EXAMPLE 8 who received a dose of 15 mg. Similar mean plasma Compound 1 concentrations were seen between subjects given Compound 1 alone (Group 1) or in combination with ranibizumab (Group 2) at both 30 and 90 minutes. The mean plasma concentration of Compound 1 in subjects of EXAMPLE 8 at 15 minutes was similar to mean Compound 1 concentrations seen in the plasma of subjects of EXAMPLE 9 at 30 minutes. The mean plasma concentration of Compound 1 in subjects of EXAMPLE 8 at 60 minutes was similar to mean Compound 1 concentrations seen in the plasma of subjects of EXAMPLE 9 at 90 minutes.

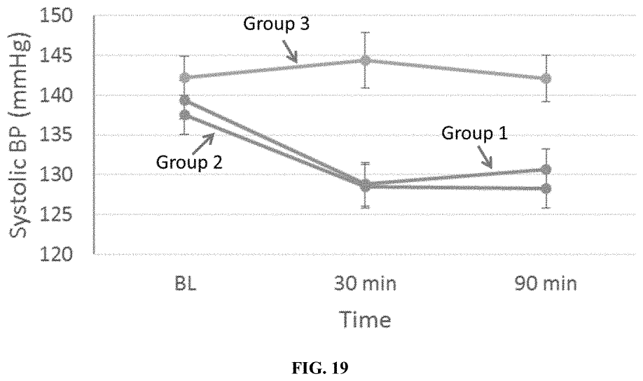

FIG. 19 and TABLE 27 show the effect of Compound 1 treatment, both alone and in combination with ranibizumab, on the sitting systolic blood pressure of subjects. Blood pressure measurements taken at 30 and 90 minutes post-dose show that subjects given Compound 1 alone (Group 1), and Compound 1 in combination with ranibizumab (Group 2), displayed a decrease in sitting systolic blood pressure compared to baseline levels. This decrease in sitting systolic blood pressure was similar between subjects of Groups 1 and 2, and was not seen in subjects who received ranibizumab alone (Group 3).

TABLE 27

| | Compound 1 + | Compound 1 + | Placebo + |
|---|---|---|---|
| | Sham | Ranibizumab | Ranibizumab |
| Baseline | | | |
| (Day 1 pre-dose) | | | |
| n | 48 | 49 | 47 |
| Mean (SD) | 139.4 (16.37) | 137.5 (16.78) | 142.2 (19.04) |
| Median | 141.5 | 133.0 | 141.0 |
| Min, Max | 87, 163 | 110, 172 | 109, 193 |
| Change from pre- | | | |
| dose to 30 | | | |
| minutes post-dose | | | |
| n | 48 | 48 | 47 |
| Mean (SD) | −10.6 (17.63) | −9.4 (20.68) | 2.2 (14.32) |
| Median | −10.5 | −5.5 | 1.0 |
| Min, Max | −65, 24 | −65, 27 | −37, 31 |
| Change from pre- | | | |
| dose to 90 | | | |
| minutes post-dose | | | |
| n | 48 | 48 | 47 |
| Mean (SD) | −8.7 (16.90) | −9.7 (17.97) | −0.1 (14.46) |
| Median | −5.0 | −10.5 | 2.0 |
| Min, Max | −59, 17 | −55, 36 | −30, 35 |

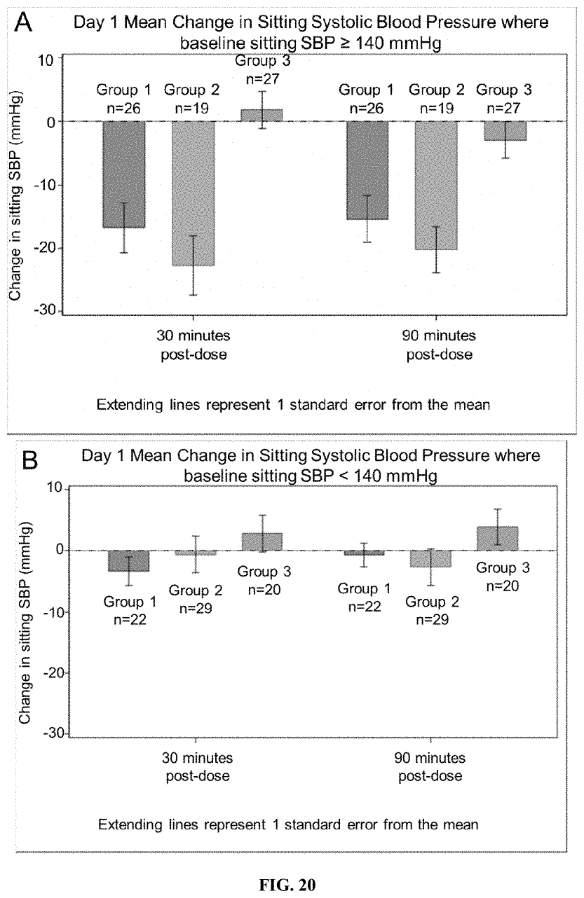

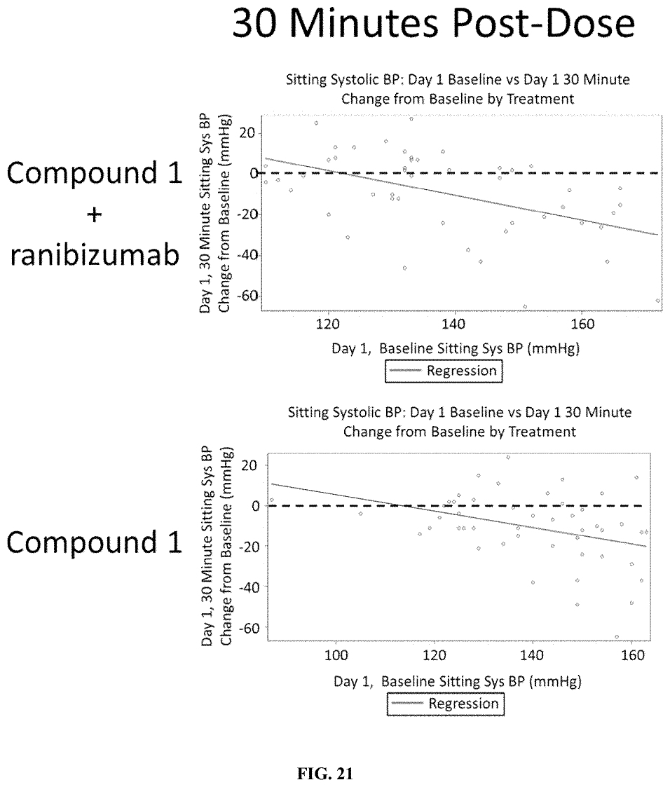

FIG. 20 compares the effects of Compound 1 treatment, alone (Group 1), or in combination with ranibizumab (Group 2), on subjects with a baseline sitting systolic blood pressure of 140 mmHg or greater, versus those with a baseline sitting systolic blood pressure of less than 140 mmHg. A substantial decrease in blood pressure was seen upon treatment with Compound 1, both alone and in combination with ranibizumab, in subjects with a sitting systolic blood pressure of 140 mmHg or greater. Minimal change in blood pressure was seen in such subjects treated with ranibizumab alone (Group 3). In subjects with sitting baseline systolic blood pressures of less than 140 mmHg, treatment with Compound 1, alone or in combination with ranibizumab, had minimal effect on blood pressure. The correlation between baseline sitting systolic blood pressure and the change in blood pressure from baseline seen 30 and 90 minutes following specified treatments is shown in FIG. 21 and FIG. 22, respectively.

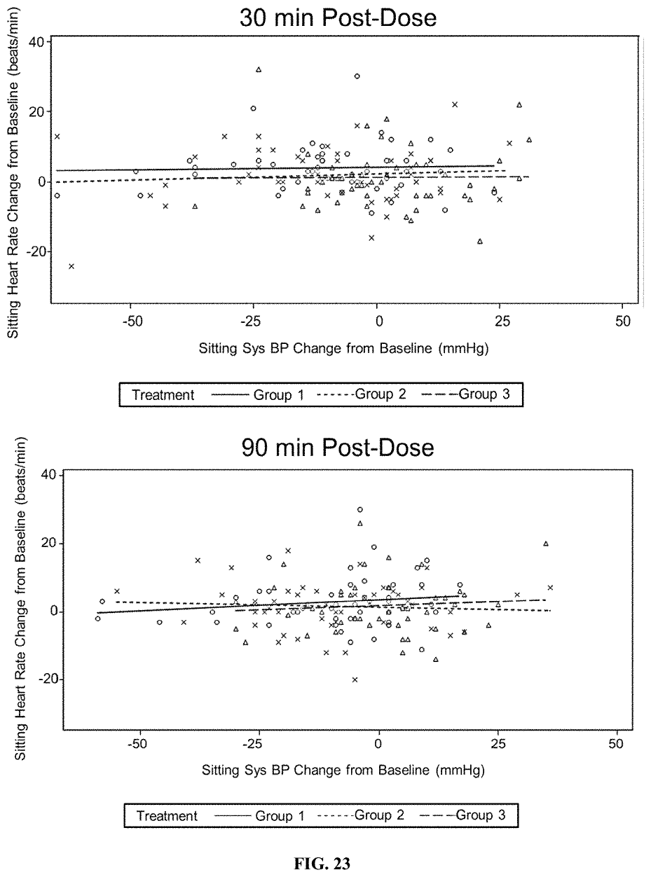

Comparison of pulse rate and blood pressure changes in all three groups (Compound 1 alone, Group 1; Compound 1 in combination with ranibizumab, Group 2; and ranibizumab alone, Group 3) did not show a significant correlation between pulse rate and blood pressure change at either 30 or 90 minutes post-dose, as seen in FIG. 23.

### Example 10. Effect of Compound 2 on eNOS Activity

Regulation of eNOS activity can affect endothelial cellular function, for example, in diabetic vessels. Thus, regulation of eNOS activity can be a treatment mechanism for diabetes. The role for VE-PTP in regulating eNOS was assessed using a VE-PTP inhibitor, Compound 2.

### Animals

C57/BL6 mice were purchased from Charles River Laboratories. Ins2^{Akita}(C57BL/6-Ins2^{Akita}/J) mice carrying a mutation in the insulin 2 gene were obtained from The Jackson Laboratory. The colony was generated by breeding a C57BL/6J inbred female with a heterozygous male. Animals were housed in compliance to the Guide for the Care and Use of Laboratory Animals published by the US National Institutes of Health under a 12-hour light-dark cycle with free access to water and a normal chow diet. Studies were performed using male age- and strain-matched animals throughout. For the isolation of aortae, mice were killed by terminal inhalation anesthesia.

### Vascular Reactivity Experiments

Aortic rings from eight to ten-week old male C57/BL6 mice were used to investigate the effects of Compound 2 on endothelial function. Twelve to fourteen-week old male Ins2^{Akita} mice and non-diabetic littermate controls were used to study the effects of Compound 2 on diabetes-associated endothelial dysfunction. Myograph experiments were performed in modified Krebs-Henseleit solution. Aortae were dissected free of adhering tissue, cut into 2-mm segments, and mounted in 5-mL myograph chambers under a basal tension of 1 g. Relaxation to cumulatively increasing concentrations (0.001-100 μmol/L) of Compound 2 or solvent (DMSO) was assessed in endothelium-intact aortic segments pre-contracted with phenylephrine (1 μmol/L) in the absence or presence of N^{ω}-nitro-L-arginine methyl ester (L-NAME; 300 μmol/L). Responses to cumulatively increasing concentrations of phenylephrine, acetylcholine, or sodium nitroprusside were assessed in endothelium-intact and endothelium-denuded aortic segments after incubation with solvent (DMSO) or Compound 2 (1, 3, or 10 μmol/L) for 30 minutes. pEC50 (−log mol/L) and Emax values were calculated from the linear regression of the data.

Cell Culture

Human umbilical vein endothelial cells (HUVECs) were isolated and cultured. HEK293 cells were obtained from the American Type Culture Collection and were cultured in minimal essential medium containing 8% heat inactivated fetal calf serum (FCS), gentamycin (25 μg/mL), non-essential amino acids, and sodium pyruvate (1 mmol/L). All cells were negative for mycoplasma contamination. Cultured cells were kept in a humidified incubator at 37° C. containing 5% CO₂.

Adenoviral Transduction of Endothelial Cells

Human endothelial cells (90% confluent) were starved of serum in MCDB131 containing 0.1% bovine serum albumin (BSA) and infected with adenoviruses (100 MOI) carrying FLAG-tagged wild-type eNOS overnight. The culture medium was then replaced with MCDB131 containing 8% heat inactivated FCS, ECGS/heparin, basic fibroblast growth factor (1 ng/mL), epidermal growth factor (0.1 ng/mL). Cells were allowed to grow for 24 hours before use.

Transfection of Cells

Myc-tagged human wild-type ABL1 in pcDNA3 was used as backbone for site-directed mutagenesis to generate ABL1 dominant negative (DN) kinase-dead mutant (K290M) using the following primers:

forward—
(SEQ ID NO: 1)
5′ GCCTCACTGTGGCCGTGATGACCTTGAAGGAGGACAC 3′
reverse—
(SEQ ID NO: 2)
5′GTGTCCTCCTTCAAGGTCATCACGGCCACAGTGAGGC 3′

HEK293 cells were co-transfected with plasmids expressing Myc-tagged human wild-type eNOS in pcDNA3.1myc/His21 and human wild-type or DN ABL1 using Lipofectamin2000 according to the manufacturer's instructions.

ABL1 Knockdown

Human endothelial cells were cultured in 6-well plates until 80% confluent and transfected with 15 μmol of functionally verified siRNA directed against human ABL1 or a control siRNA using Lipofectamine RNAiMAX Transfection Reagent in serum-free OptiMEM media. After 5 hours, cells were washed with PBS and the culture medium was replaced with MCDB131 containing 8% heat inactivated FCS, ECGS/heparin, basic fibroblast growth factor (1 ng/mL), epidermal growth factor (0.1 ng/mL). Cells were allowed to grow for 48 hours before use.

Treatment of Cells and NO Assay

Confluent human endothelial cell monolayers were cultured overnight in MCDB131 containing 0.1% BSA and sepiapterin (10 μmol/L). The monolayers were then incubated with increasing concentrations of Compound 2 (0.3-30 μmol/L) or solvent (DMSO) for 30 minutes. In separate experiments, cells treated with a control siRNA or siRNA directed against ABL1 were cultured in MCDB131 containing 0.1% BSA and sepiapterin (10 μmol/L), and then treated with Compound 2 (30 μmol/L) or solvent (DMSO) for 30 minutes before the addition of Yoda1 (1 μmol/L) for an additional 30 minutes. HEK293 cells co-transfected with wild-type eNOS and wild-type ABL1, DN ABL1, or GFP (as a control) were cultured overnight in minimal essential medium containing 0.5% heat-inactivated FCS and sepiapterin (10 μmol/L). Aliquots of the culture media were collected before and after each treatment. Potential cell debris was removed by centrifugation of the media at 1000 rpm for 5 minutes. NO release was assessed by determining the amount of nitrite in the cell supernatants using a Nitric Oxide Analyzer after reaction with iodide and acetic acid under nitrogen at room temperature.

Cell Lysis and Immunoprecipitation

Confluent monolayers of human endothelial cells expressing FLAG-tagged eNOS cultured overnight in MCDB131 containing 0.1% BSA and then incubated with increasing concentrations of Compound 2 (0.3-30 μmol/L) or solvent (DMSO) for 30 minutes. In separate experiments, human endothelial cells were cultured overnight in MCDB131 containing 0.1% BSA and then incubated treated with Compound 2 (30 μmol/L) or solvent for 30 minutes and then stimulated with Yoda1 (1 μmol/L) or solvent for an additional 30 minutes. To inhibit Src, cells were pre-incubated with PP2 (1 μmol/L) for 30 min before incubation with Compound 2 and stimulation with Yoda1. After two washes with cold PBS, cells were collected with the aid of cell scrapers in a lysis buffer containing Tris/HCl pH 7.5 (50 mmol/L), NaCl (150 mmol/L), Triton X-100 (1%), NaPPi (10 mmol/L), NaF (20 mmol/L), orthovanadate (2 mmol/L), okadaic acid (10 nmol/L), β-glycerophosphate (50 mmol/L), phenylmethylsulfonyl fluoride (230 μmol/L), and EDTA-free protease inhibitors mix. The cell mixture was incubated for 30 minutes at 4° C. with vortexing, followed by centrifugation at 13000 rpm for 10 minutes at 4° C. To assess the eNOS-VE-PTP interaction, the following modifications were applied: 1) cells were incubated with the lysis buffer for one hour on an end-over-end rocker at 4° C. and gently vortexed until no cell clumps were observed; 2) lysates were not centrifuged after lysis. FLAG-tagged eNOS was immunoprecipitated using 30 μL of packed Anti-FLAG M2 Affinity Gel per mg protein lysate overnight. The recovered immunoprecipitates were washed three times with the lysis buffer, eluted by boiling samples for 10 minutes in SDS sample buffer, and then analyzed by SDS-PAGE and immunoblotting.

Immunoblotting

Protein samples were separated by SDS-PAGE and then transferred to 0.45 mm nitrocellulose membranes. Membranes were incubated overnight with primary antibodies against phospho-Tyr81 eNOS, phospho-Ser1177 eNOS, phospho-Ser633 eNOS, eNOS, phospho-Ser473 Akt, Akt, ABL1, VE-PTP, GAPDH, β-actin, followed by species-specific secondary antibodies anti-IgG conjugated with HRP. Proteins were visualized by enhanced chemiluminescence.

Human VE-PTP (HPTPβ) Phosphatase Assay

Confluent monolayers of human endothelial cells expressing FLAG-tagged eNOS were cultured overnight in MCDB131 containing 0.1% BSA and then stimulated with Yoda1 (1 μmol/L) for 30 minutes to elicit phosphorylation of eNOS on Tyr81. eNOS-FLAG immunoprecipitates were used as substrates to test the ability of recombinant human VE-PTP to dephosphorylate eNOS Tyr81 in a cell-free in vitro reaction. Briefly after washing the FLAG-eNOS, immunoprecipitates were suspended in phosphatase assay buffer containing Tris/HCl pH 7.5 (50 mmol/L), NaCl (150 mmol/L), EDTA (1 mmol/L), and EDTA-free protease inhibitors mix at room temperature. An aliquot of the eNOS-FLAG immunoprecipitate was immediately processed for immunoblotting (input) and the remainder was divided into 6 identical aliquots and incubated for up to 10 minutes at room temperature in phosphatase assay buffer containing DTT (3 mmol/L) and recombinant human VE-PTP (100 U/50 μl reaction). Experiments were performed in the presence of solvent (1% DMSO) or Compound 2 (10 μmol/L) and reactions were stopped by boiling samples for 10 minutes in SDS sample buffer. eNOS phosphorylation was then analyzed by SDS-PAGE and immunoblotting.

### Results and Conclusions

VE-PTP Inhibition Enhances Endothelial Function.

To assess the role for VE-PTP in the regulation of vascular reactivity, endothelium-intact rings of murine aortae were incubated with increasing concentrations of Compound 2.

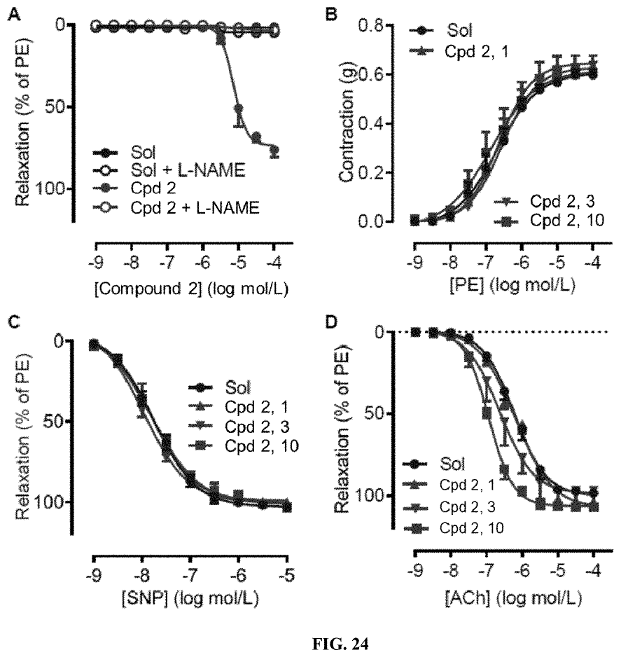

FIG. 24 panels A-D illustrate the effect of Compound 2 on endothelial function. FIG. 24 panel A illustrates concentration dependent effect of Compound 2 on the tone of endothelium-intact aortic rings. Experiments were performed in the absence and presence of an eNOS inhibitor, L-NAME (300 μmol/L). FIG. 24 panels B-D illustrate the effect of solvent (Sol, DMSO) and Compound 2 (1, 3, or 10 μmol/L) on the contractile response to phenylephrine (PE; FIG. 24 panel B), and relaxation induced by either sodium nitroprusside (SNP; FIG. 24 panel C) or acetylcholine (ACh; FIG. 24 panel D). The dotted line in FIG. 24 panel D represents responses in endothelium-denuded aortic rings; n=5 mice/group.

In arteries precontracted with phenylephrine, Compound 2 consistently induced concentration-dependent relaxation (pEC50: 5.14±0.05 log mol/L, Emax: 73.4±2.9%, n=5 mice/group, P<0.001). This response was abolished in the presence of the eNOS inhibitor, L-NAME (FIG. 24). Neither phenylephrine-induced contractions nor sodium nitroprusside-induced relaxations were affected by Compound 2. Acetylcholine-induced relaxations of endothelium-intact vessels were also significantly enhanced by Compound 2 in a concentration-dependent manner (FIG. 24 panels B-D and TABLE 28). TABLE 28 summarizes the effect of solvent (DMSO) and Compound 2 (1, 3, or 10 μmol/L) on the acetylcholine-induced relaxation of endothelium-intact aortic rings (related to FIG. 24 panel D). **P<0.01, ***P<0.001 versus solvent (1-way ANOVA and Bonferroni). These studies demonstrate that diabetes-induced endothelial dysfunction was abrogated by VE-PTP inhibition.

TABLE 28

| Treatment | pEC50 | Emax (%) |
|---|---|---|
| Solvent | 6.22 ± 0.05 | 100.1 ± 2.6 |
| Compound 2 (1 μmol/L) | 6.19 ± 0.05 | 107.5 ± 2.6 |
| Compound 2 (3 μmol/L) | 6.63 ± 0.13** | 98.68 ± 5.4 |
| Compound 2 (10 μmol/L) | 6.93 ± 0.04*** | 106.3 ± 1.4 |

VE-PTP Inhibition Increases eNOS Activity Through Enhanced Phosphorylation on Tyr81 and Ser1177.

To investigate the role of VE-PTP on the regulation of eNOS, human endothelial cells were treated with increasing concentrations of Compound 2 (for 30 minutes) and the generation of NO was assessed using a NO analyzer.

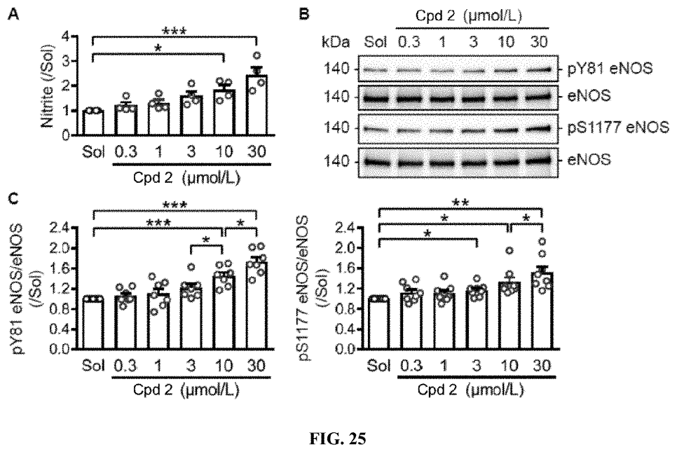

FIG. 25 panels A-D illustrate the effect of Compound 2 on eNOS activity in human endothelial cells. FIG. 25 panel A illustrates nitrite levels in the supernatant of human endothelial cells treated with Compound 2 (0.3, 1, 3, 10, and 30 μmol/L) or solvent (Sol) for 30 minutes. FIG. 25 panel B illustrates Compound 2-dependent changes in eNOS phosphorylation on Tyr81 (Y81; assessed in eNOS immunoprecipitates) and Ser1177 (S1177; assessed in whole cell lysates); n=4-8 independent cell batches. *P<0.05, **P<0.01, ***P<0.001 (1-way ANOVA and Bonferroni). FIG. 25 panel C illustrates the quantitative effect of Compound 2 on eNOS phosphorylation on Tyr81. FIG. 25 panel D illustrates the quantitative effect of Compound 2 on eNOS phosphorylation on Ser1177. In line with the vascular reactivity data, Compound 2 enhanced basal NO production (FIG. 25 panel A) and led to phosphorylation of eNOS on Tyr81 and Ser1177 (FIG. 25 panels B-C).

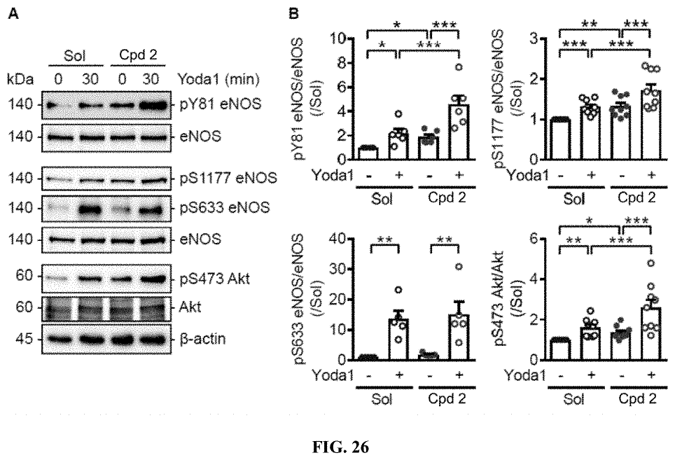

Flow-induced activation of eNOS relies on the activation of PIEZO1. Thus, responses to a PIEZO1 agonist, Yoda1, were also studied. Human endothelial cells were treated with Compound 2 (30 μmol/L) or solvent (Sol) for 30 minutes and then stimulated with Yoda1 (1 μmol/L) for an additional 30 min. FIG. 26 panels A-B illustrate the effect of Compound 2 on eNOS activity in the presence of a PIEZO1 agonist, Yoda1, in human endothelial cells. FIG. 26 panel A illustrates the effect of Compound 2 on eNOS phosphorylation on Tyr81 (Y81; assessed in eNOS immunoprecipitates), Ser1177 (S1177; assessed in whole cell lysates), and Ser633 (S633), as well as Akt phosphorylation on Ser473 (S473; assessed in whole cell lysates). FIG. 26 panel B illustrates the quantification of the changes in eNOS and Akt phosphorylation; n=5-9 independent cell batches. *P<0.05, **P<0.01, ***P<0.001 (2-way ANOVA and Holm-Sidak).

Yoda1 elicited the phosphorylation of Akt on Ser473, and phosphorylation of eNOS on Ser1177 and Ser633. Yoda1 also increased the phosphorylation of eNOS on Tyr81, an effect that was markedly enhanced following VE-PTP inhibition by Compound 2 (FIG. 26 panels A-B). The Yoda1-induced phosphorylation of Akt on Ser473 and eNOS on Ser1177 also increased. These effects on Akt and eNOS were more significantly enhanced by VE-PTP inhibition. The Yoda1-induced phosphorylation of eNOS on Ser633 was not affected by VE-PTP inhibition (FIG. 26 panels A-B).

The Tyrosine Kinases, Src and ABL1, Mediate the Phosphorylation of eNOS on Tyr81.

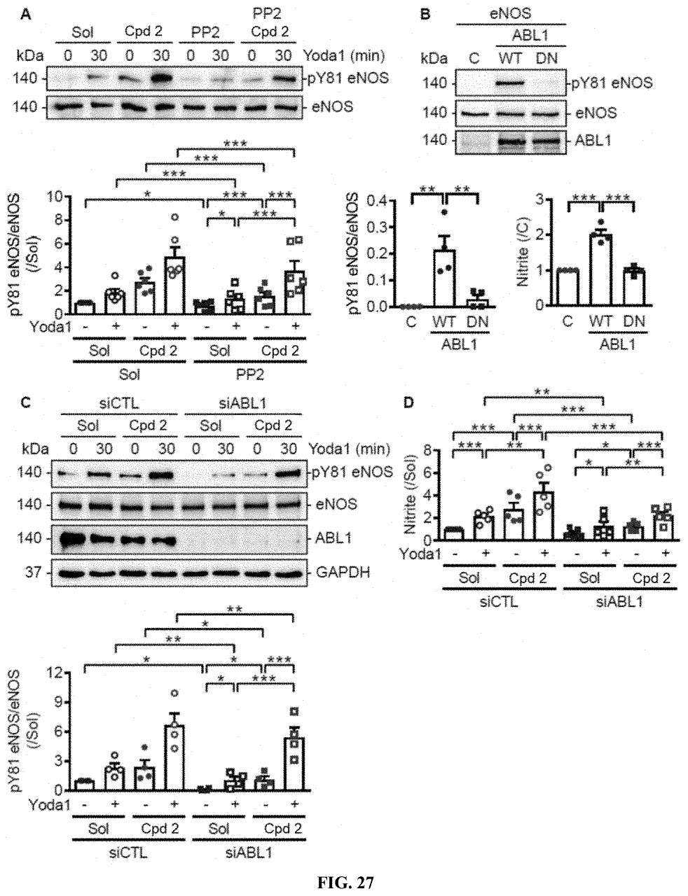

FIG. 27 panels A-D illustrate the effect of Compound 2 on eNOS activity in the presence of Yoda1 in human endothelial cells treated with a Src inhibitor, PP2, or ABL1. Human endothelial cells were treated with the Src inhibitor, PP2 (1 μmol/L), prior to the addition of Compound 2 (30 μmol/L, 30 minutes) and Yoda1 (1 μmol/L, 30 minutes). eNOS phosphorylation on Tyr81 (Y81) was assessed in eNOS immunoprecipitates, as shown in FIG. 27 panel A. Src inhibition significantly reduced basal phosphorylation of eNOS, as well as Yoda1-induced tyrosine phosphorylation of eNOS (FIG. 27 panel A).

Although Src inhibition reduced the potentiating effect of Compound 2 on eNOS tyrosine phosphorylation, the effect was not abolished. This result suggested that another factor contributed to the phosphorylation of eNOS on Tyr81, e.g., an unidentified tyrosine kinase. An in silico screening for kinases that could target eNOS Tyr81 using PhosphoNET Kinase Predictor identified a potential role for abelson-tyrosine protein kinase (ABL1). ABL1 is required for vascular homeostasis and can be activated following endothelial cell stimulation of VEGFR2 and Tie-2.

To determine whether ABL1 phosphorylates eNOS, HEK293 endothelial cells were co-transfected with eNOS and either the wild-type ABL1 or a dominant-negative ABL1 mutant. eNOS phosphorylation was also assessed in immunoprecipitates from HEK293 cells expressing eNOS and either a control plasmid (C), the wild-type (WT), or dominant-negative (DN) ABL1 for 48 hours, as shown in FIG. 27 panel B. eNOS activity in the same cells was determined using the NO analyzer. While the wild-type ABL1 elicited a robust phosphorylation of eNOS on Tyr81 and increased NO generation, the dominant-negative ABL1 mutant did not have an effect, as shown in FIG. 27 panel B.

Consistent with these findings, siRNA-mediated downregulation of ABL1 in human endothelial cells significantly attenuated basal and the Yoda1-induced phosphorylation and activation of eNOS. Again, however, downregulation of ABL1 attenuated, but did not completely prevent the phosphorylation of eNOS on Tyr81 elicited by VE-PTP inhibition, as shown in FIG. 27 panels C-D.

FIG. 27 panels C-D illustrate the effects of ABL1 downregulation on eNOS phosphorylation and activity. eNOS phosphorylation on Tyr81 was determined in eNOS immunoprecipitates, as shown in FIG. 27 panel C. The effects on nitrite levels in the supernatant of cells treated with a control siRNA (siCTL) or siRNA targeting ABL1 (siABL1) are shown in FIG. 27 panel D. Forty-eight hours after transfection, cells were treated with Compound 2 (30 μmol/L) or solvent (Sol) for 30 minutes prior to the addition of Yoda1 (1 μmol/L, 30 minutes); n=4-6 independent cell batches. *P<0.05, **P<0.01, ***P<0.001 (A, C-D: 2-way ANOVA and Holm-Sidak; B: 1-way ANOVA and Bonferroni).

VE-PTP Interacts with eNOS and Dephosphorylates Tyr81.

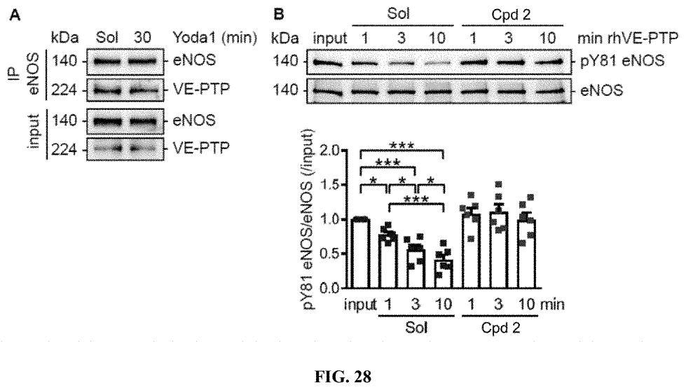

Since the data from the previous studies suggest a direct link between VE-PTP and eNOS, the association of the two proteins was assessed. FIG. 28 panels A-B illustrate assessment results of the association of VE-PTP and eNOS in human endothelial cells. FIG. 28 panel A illustrates the interaction of VE-PTP with eNOS immunoprecipitated (IP) from cells treated with solvent or Yoda1 (1 μmol/L, 30 minutes). Similar results were obtained using 6 independent cell batches. In the co-immunoprecipitation experiments, VE-PTP associated with eNOS under basal (unstimulated) conditions (FIG. 28 panel A). This association was not altered following stimulation with Yoda1.

The ability of VE-PTP to dephosphorylate eNOS Tyr81 was also investigated. In a cell-free assay using eNOS immunoprecipitated from Yoda1-stimulated endothelial cells as a substrate, recombinant VE-PTP induced time-dependent dephosphorylation of eNOS Tyr81. This effect was abolished by pre-incubation with Compound 2 (FIG. 28 panel B). These in vitro data indicate that VE-PTP formed a complex with eNOS in endothelial cells and directly dephosphorylated eNOS on Tyr81. The in vitro phosphatase assay used eNOS immunoprecipitated from Yoda1-stimulated cells and recombinant human VE-PTP (added for 1, 3, or 10 minutes). Assays were performed in the absence and presence of Compound 2 (10 μmol/L); n=6 independent cell batches. *P<0.05, ***P<0.001 (2-way ANOVA and Holm-Sidak).

VE-PTP Inhibition Abrogates Diabetes-Induced Endothelial Dysfunction.

To determine whether VE-PTP inhibition affects endothelial dysfunction that accompanies diabetes, aortic rings were isolated from 12-week old diabetic Ins2^{Akita}(Akita) mice and their non-diabetic littermates.

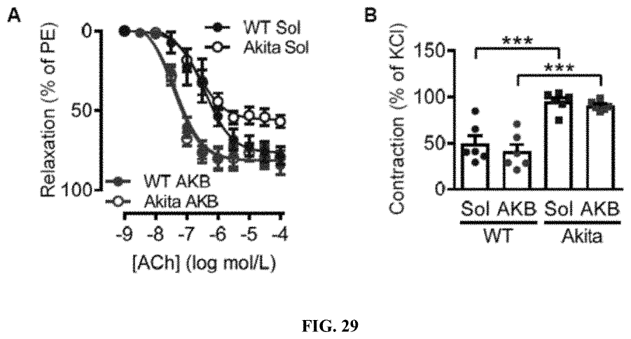

FIG. 29 panels A-B illustrate the effects of Compound 2 on diabetes-induced endothelial dysfunction. FIG. 29 panel A illustrates the effects of Compound 2 on acetylcholine-induced relaxation of endothelium-intact aortic rings from WT and Akita mice. Endothelium-intact aortic rings from diabetic Ins2^{Akita} mice demonstrated pronounced endothelial dysfunction, i.e., impaired responsiveness to acetylcholine. As illustrated in TABLE 29, acetylcholine-induced relaxation of endothelium-intact aortic rings from WT and Ins2^{Akita} mice in the presence of solvent (Sol) or Compound 2 (10 μmol/L). **P<0.01 versus WT; ^{#}P<0.05 versus Ins2^{Akita} (2-way ANOVA and Holm-Sidak). Compound 2 had a profound effect on acetylcholine-induced endothelium-dependent relaxation and potentiated responses in tissues from non-diabetic mice, which was consistent with the earlier observations illustrated in FIG. 24.

TABLE 29

| Groups | pEC50 | Emax (%) |
|---|---|---|
| WT + Sol | 6.42 ± 0.14 | 76.5 ± 3.8 |
| WT + Compound 2 | 7.35 ± 0.11** | 81.3 ± 2.6 |
| Ins2^{Akita} + Sol | 6.66 ± 0.12 | 55.7 ± 2.3 |
| Ins2^{Akita} + Compound 2 | 7.43 ± 0.09^{#} | 81.4 ± 2.2^{#} |

FIG. 29 panel B illustrates the effects of Compound 2 on phenylephrine-induced contraction of aortic rings from WT and Akita mice. Compound 2 restored acetylcholine-induced and NO-mediated relaxation in aortic rings from diabetic mice. While the phenylephrine-induced contraction of aortic rings was elevated in vessels from Ins2^{Akita} mice, this effect was not influenced by VE-PTP inhibition.

Collectively, these studies suggest that VE-PTP inhibition enhances eNOS activity and endothelial function and can be an effective treatment for diabetes-induced endothelial dysfunction and hypertension.

### Example 11. Effects of Compound 1 on Blood Pressure and Heart Rate of Diabetes Patients

Hemodynamic Assessment of Diabetes Subjects Treated with Compound 1.

A Phase 2, randomized, placebo-controlled, double-masked study was conducted to assess the safety and efficacy of subcutaneously administered Compound 1 at doses of 15 mg once daily or 15 mg twice daily for 48 weeks in subjects with moderate to severe non-proliferative diabetic retinopathy (NPDR).

Subject Eligibility and Exclusion Criteria: Eligible subjects were aged 18 to 80 years with moderate to severe NPDR. Relevant systemic exclusion criteria for the study included, resting systolic blood pressure ≥180 mmHg or <100 mmHg, diastolic blood pressure ≥100 mg Hg, and hemoglobin A1c ≥12%.

Study treatments and hemodynamic measurements: Subjects were randomized 1:1:1 to Compound 1 at a dose of 15 mg once daily (QD), Compound 1 at a dose of 115 mg twice daily (BID), or placebo BID treatment groups. Subjects self-administered masked study medication (Compound 1 or placebo) supplied as sterile pre-filled single use syringes. Subjects visited the site monthly during the 48-week treatment period. Resting blood pressure and heart rate was assessed at these monthly visits. Additionally, On Day 1 and Week 24, blood pressure and heart rate were measured prior to dosing and at 30 and 90 minutes post-dose.

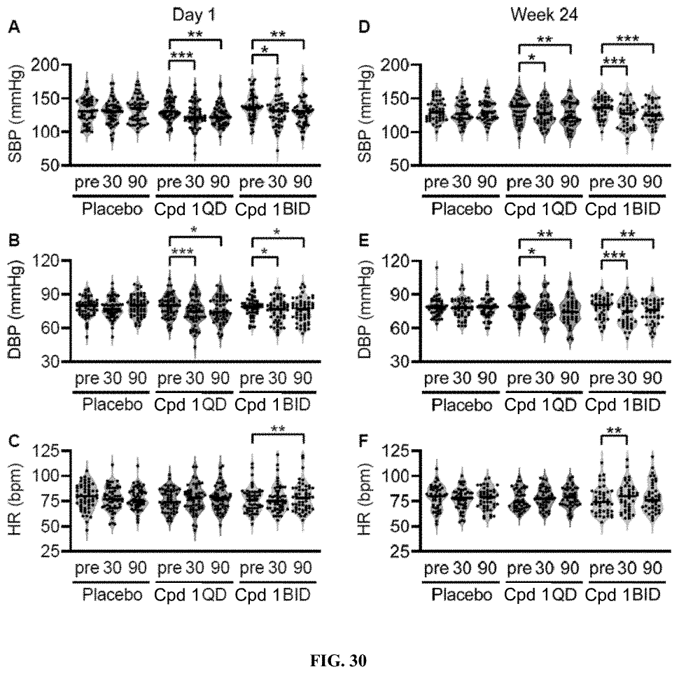

FIG. 30 illustrates changes in systolic blood pressure (FIG. 30 panels A and D), diastolic blood pressure (FIG. 30 panels B and E), and heart rate (FIG. 30 panels C and F) compared to baseline (pre) in diabetic patients administered with Compound 1 (Cpd 1) once (QD) or twice (BID) daily. Measurements were taken on Day 1 (FIG. 30 panels A-C) and at Week 24 after the start of the treatment regimen (FIG. 30 panels D-F). Day 1: n=57 placebo, n=55 Cpd 1 QD, n=55 Cpd 1 BID; Week 24: n=48 placebo, n=44 Cpd 1 QD, n=43 Cpd 1 BID. *P<0.05, **P<0.01, ***P<0.001 versus baseline (repeated measures one-way ANOVA and Bonferroni).

Consistent with the findings in vessels from diabetic mice, daily subcutaneous administration of Compound 1 (15 mg QD or BID) elicited a consistent reduction in systolic blood pressure, as well as diastolic blood pressure in patients with diabetes that was accompanied by a small change in heart rate. The reduction in blood pressure was larger in patients with higher baseline blood pressures, including patients on standard-of-care antihypertensive drugs. Moreover, the blood pressure reduction was similar on Day 1 and Week 24. This observation indicated a lack of tolerance to Compound 1. The decrease in systolic blood pressure was maintained throughout the duration of the study (48 weeks). Thus, chronic VE-PTP inhibition of hypertensive diabetic patients can significantly reduce systolic blood pressure. Given that endothelial dysfunction is the underlying cause of many cardiometabolic diseases in which enhanced NO bioavailability can be beneficial, VE-PTP inhibition can be an effective method of treating hypertension in diabetic subjects.

Data and Statistical Analysis

Results are presented as mean SEM. Differences between three groups or more were compared by one-way ANOVA followed by the Bonferroni posttest. All experiments in which the effects of two variables were tested were analyzed by two-way ANOVA followed by the Holm-Sidak posttest. Differences were considered statistically significant when P<0.05.

Pre-specified descriptive statistics included the number of subjects (n), mean, and standard error (SE). Post-hoc analysis of hemodynamic data included analysis of SBP, DBP, PP, and heart rate (HR) change from pre-dose baseline at 30 and 90 minutes post-dose on Day 1 and Week 24 within groups and difference between active treatment groups (QD and BID Compound 1) and placebo were compared by paired t-test and ANCOVA (Analysis of Covariance), respectively. For post-hoc analysis of monthly hemodynamic data, change from baseline between the active in the active treatment groups and placebo were compared by MMRM analysis (Mixed Effect Model Repeated Measures) across all visits (excluding the 24-week visit).

### Example 12. Assessment of Hypoxia on VE-PTP Expression a Tie-2 Activator for the Treatment of PAH In Vitro

The effects of a Tie-2 activator on maintaining barrier function in the pulmonary vasculature can be assessed by analyzing neutrophil transmigration with cytokine-stimulated human pulmonary artery endothelial cells (HPAECs). TGF-β1 and TNF-α are two pathologically relevant cytokines whose downstream signaling pathways induce pulmonary vascular leakage, a symptom of PAH. Gene-silencing of BMPR2 in HPAECs pre-treated with inflammatory cytokines can serve as a model of PAH in vitro.

HPAECs can be cultured in endothelial cell growth medium (EGM) and supplemented with 2% (v/v) fetal calf serum (FCS), 0.4% (v/v) endothelial cell growth supplement, 0.1 ng/mL epidermal growth factor, 1 ng/mL basic fibroblast growth factor, 22.5 μg/mL heparin and 1 μg/mL hydrocortisone (complete medium) until confluent.

HPAECs can be dissociated from flasks using trypsin/EDTA and seeded onto uncoated, low-density, 3-μm pore polycarbonate Transwell filters, which can be positioned into matching 24- or 6-well plates or Ibidi VI slides that are pre-coated with 0.2 μg/mL fibronectin in PBS overnight. Seeding density can be chosen to yield confluent monolayers in Ibidi slides within 2 hours or filters within 24 hours.

Prior to the assay, HPAECs can be unstimulated or stimulated with 1 ng/mL human TNF-α for 4 hours or 2 ng/mL human TGF-β1 for 24 hours.

For BMPR2 silencing, HPAECs can be transfected using a scrambled nontargeting control small-interfering (si)RNA (NTCsi) or siRNA targeting human BMPR2. The day before transfection, HPAECs can be seeded in 6- or 12-well plates in serum and antibiotic-free EC growth medium. Lipofectamine 2000 (Invitrogen)-RNA complexes can be made, per manufacturer's instructions, in Optimem-1 (Invitrogen) and added dropwise to each well. HPAECs can be incubated with lipid-RNA complexes for approximately 5 hours, after which transfection medium can be replaced with complete medium, and the cells can be cultured for a further 48 hours. HPAECs treated with NTCsi or BMPR2 siRNA can be confirmed by western blotting and qRT-PCR.

Neutrophil transmigration can be assessed 6-well formal Transwell filters in matching plates. Neutrophil samples suspended in PBSA can be added to the upper chamber and allowed to transmigrate for 2 hours in an incubator maintained at 37° C. in 5% CO₂. The number of transmigrated neutrophils can be determined by counting the number of neutrophils in the lower chamber using a Vicell Series cell-viability analyzer. The total percentage of transmigrated neutrophils can be calculated by dividing the number of transmigrated neutrophils by the known number of neutrophils added.

HPAEC permeability can be assessed by FITC albumin leakage. HPAECs can be seeded onto 24-well inserts and cultured untreated or treated with siRNA-targeting BMPR2 or NTC siRNA. After transfection, the HPAEC monolayer can be washed with complete EGM, 1% (w/v) fluorescein isothiocyanate (FITC)-labeled albumin suspended in complete EGM can be added to the upper chamber, and 800 μL of complete EGM containing 1% (w/v) BSA can be added to the lower chamber. Leakage of FITC-labeled albumin into the lower chamber can be assessed by removing a sample from the lower chamber after 0.5, 1, and 2 hours for fluorescence analysis.

A Tie-2 activator disclosed herein can be used to treat HPAECs described above. The treated HPAECs can be assessed for neutrophil transmigration efficiency and permeability.

### Example 13. Assessment of a Tie-2 Activator for Treatment of PAH In Vivo Using the MCT Lung Injury Rat Model

The MCT lung injury rat model was used to assess the effects of a Tie-2 activator disclosed herein, Compound 2, as a vasodilator of the pulmonary vasculature. Sprague-Dawley rats were divided into two groups: 1) monocrotaline to induce PH, or 2) control, as shown in TABLE 30. At day 0, rats were treated with a single dose of 60 mg/kg monocrotaline (N=5) and compared to control rats (N=5), which were treated with vehicle. Animals from both groups underwent normal rodent care until day 21. At day 21, rats underwent simultaneous cannulation of the carotid and pulmonary artery/right ventricle. Arterial pressure was measured before and after subcutaneous injection of vehicle. Following the washout period of time, rats underwent the same measurements before and after subcutaneous injection of Compound 2. Hemodynamic endpoints included systemic pressure (via carotid catheterization) and pulmonary vascular pressure (right ventricular systolic pressure) vs. true pulmonary arterial pressure.

TABLE 30

| Group | Sample Size | Challenge |
|---|---|---|
| 1 | N = 5 | MCT |
| | | 60 mg/kg |
| | | Day 0 |
| 2 | N = 5 | Vehicle |
| | | 60 mg/kg |
| | | Day 0 |

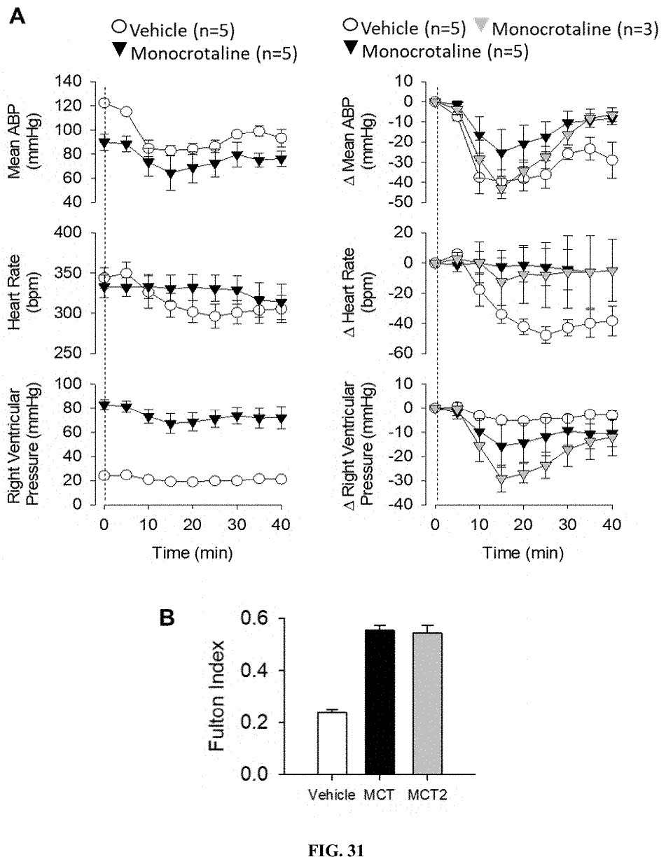

FIG. 31 panel A illustrates the effects of Compound 2 on mean arterial blood pressure (ABP), heart rate, and right ventricular pressure. Each time point represents a beat-to-beat analysis for each variable over a 2-min period, which were then averaged. The change (A) in each variable is divided into 3 groups: control or vehicle group (white), monocrotaline group (black), and a subset (n=3) of the monocrotaline group that showed a significant drop in right ventricular pressure (gray). All animals received Compound 2 at time=0 min.

After obtaining hemodynamic data, the rats were euthanized to assess the extent of RV hypertrophy, a marker of PH. The heart was then dissected to separate the right ventricle (RV) free wall from the left ventricle (LV) and septum (S). RV hypertrophy was calculated based on the Fulton index using the following weight ratio: (RV/(LV+S)). FIG. 31 panel B illustrates assessment of RV hypertrophy of the 3 groups based on the Fulton index: vehicle group (white), monocrotaline group (black; MCT), and a subset (n=3) of the monocrotaline group that showed a significant drop in right ventricular pressure (gray; MCT2)

### Example 14. Assessment of a Tie-2 Activator for Treatment of PAH In Vivo Using the SU5416-Chronic Hypoxia Rat Model

A SU5416-chronic hypoxia rat model of severe PAH can be generated by exposing rats to chronic hypoxia in combination with the VEGF receptor inhibitor, SU5416.

Male Sprague Dawley rats can be given a single intraperitoneal injection of SU5416 (20 mg/kg) in vehicle (0.5% carboxyl methylcellulose sodium, 0.4% polysorbate 80, 0.9% benzyl alcohol) and placed immediately into a 10% O₂ chamber and maintained in hypoxia for 3 weeks, followed by 5 weeks in a normoxic environment to develop pulmonary hypertension. After 5 weeks, the rats develop severe pulmonary hypertension and right ventricular hypertrophy and extensive pulmonary arterial muscularization. At the 8-week time point, rats can be randomized into 3 groups. One group can be assessed for cardiopulmonary function and sacrificed as described for the MCT lung injury model described in Example 3. The other two groups can receive 3 weeks of daily i.p. injections with 600 ng/day BMP9 or saline vehicle as a control prior to cardiopulmonary phenotyping and sacrifice.

A Tie-2 activator disclosed herein can be administered throughout the period of treatment with SU5416 and hypoxia. Rats can be assessed for normalization of RVSP and right ventricular mass size, and abrogation of pulmonary arterial muscularization.

### Example 15. Assessment of a Tie-2 Activator for Treatment of PAH In Vivo Using a Lung Injury Mice Model

A PAH mouse model can be generated by heterozygous knock-in of a human BMPR2 mutation, R899X. A BMPR2 signaling-deficient mouse can be designed to bear the human disease-associated R899X premature stop mutation in exon 12 of the endogenous Bmpr2 locus. A Tie-2 activator disclosed herein can be administered to the Bmpr2^{+/R899X} knock-in mouse. Mice can be assessed for normalization of RSVP and reversal of pulmonary arterial muscularization.

### Embodiments

The following non-limiting embodiments provide illustrative examples of the invention, but do not limit the scope of the invention.

Embodiment A1. A method for modulating a blood pressure in a human in need thereof, the method comprising administering to the human a therapeutically effective amount of a Tie-2 activator, wherein the administration changes the blood pressure in the human by about 0.1 mmHg to about 100 mmHg.

Embodiment A2. The method of Embodiment A1, wherein the modulating the blood pressure is reducing the blood pressure.

Embodiment A3. The method of Embodiment A1 or A2, wherein the blood pressure is systolic blood pressure.

Embodiment A4. The method of Embodiment A1 or A2, wherein the blood pressure is diastolic blood pressure.

Embodiment A5. The method of Embodiment A1 or A2, wherein the blood pressure is a mean arterial blood pressure.

Embodiment A6. The method of Embodiment A1 or A2, wherein the blood pressure is a pulmonary artery blood pressure.

Embodiment A7. The method of Embodiment A1 or A2, wherein the blood pressure is a pulmonary artery systolic blood pressure.

Embodiment A8. The method of any one of Embodiments A1-A7, wherein the administration reduces a pulse pressure in the human.

Embodiment A9. The method of any one of Embodiments A1-A8, wherein the administration increases a level of a signaling molecule in the human.

Embodiment A10. The method of Embodiment A9, wherein the signaling molecule is nitric oxide.

Embodiment A11. The method of Embodiment A9, wherein the signaling molecule is cyclic guanosine monophosphate.

Embodiment A12. The method of any one of Embodiments A9-A11, wherein the level of the signaling molecule is increased by decreasing metabolism of the signaling molecule in a tissue of the human.

Embodiment A13. The method of any one of Embodiments A9-A11, wherein the level of the signaling molecule is increased by decreasing export of the signaling molecule in a tissue of the human.

Embodiment A14. The method of any one of Embodiments A9-A11, wherein the level of the signaling molecule is increased in an endothelial cell in the human.

Embodiment A15. The method of any one of Embodiments A12-A14, wherein the level of the signaling molecule is increased in an endothelial cell in the human, wherein the signaling molecule is nitric oxide.

Embodiment A16. The method of any one of Embodiments A1-A15, wherein the administration increases endothelial function in the human.

Embodiment A17. The method of any one of Embodiments A1-A16, wherein the administration increases phenylephrine-induced contraction in the human.

Embodiment A18. The method of any one of Embodiments A1-A17, wherein the administration increases acetylcholine-induced relaxation in the human.

Embodiment A19. The method of any one of Embodiments A1-A18, wherein the administration activates endothelial nitric oxide synthase (eNOS) in the human.

Embodiment A20. The method of Embodiment A19, wherein the administration activates eNOS in the human by increasing eNOS phosphorylation on Tyr81 and Ser1177.

Embodiment A21. The method of Embodiment A19, wherein the administration activates eNOS in the human via activation of proto-oncogene tyrosine-protein kinase (Src) in the human.

Embodiment A22. The method of Embodiment A19, wherein the administration activates eNOS in the human via activation of Abelson murine leukemia viral oncogene homolog 1 (ABL1) in the human.

Embodiment A23. The method of any one of Embodiments A1-A22, wherein the administration activates Akt in the human.

Embodiment A24. The method of any one of Embodiments A1-A23, wherein the administration increases vascularization in the human.

Embodiment A25. The method of any one of Embodiments A1-A23, wherein the administration increases vascularization in a lung of the human.

Embodiment A26. The method of any one of Embodiments A1-A25, wherein the administration reduces a rate of blood efflux in the human.

Embodiment A27. The method of Embodiment A26, wherein the rate of blood efflux is reduced by reducing vascular permeability.

Embodiment A28. The method of Embodiment A26, wherein the rate of blood efflux is reduced by reducing venous drainage.

Embodiment A29. The method of Embodiment A26, wherein the rate of blood efflux is reduced by reducing venous leak.

Embodiment A30. The method of any one of Embodiments A1-A29, wherein the administration increases a level of dilation of a blood vessel in the human.

Embodiment A31. The method of Embodiment A30, wherein the blood vessel is an artery.

Embodiment A32. The method of Embodiment A30, wherein the blood vessel is a vein.

Embodiment A33. The method of Embodiment A30, wherein the blood vessel is a capillary.

Embodiment A34. The method of any one of Embodiments A1-A33, wherein the human has diabetes.

Embodiment A35. The method of any one of Embodiment A34, wherein the administration treats the diabetes in the human.

Embodiment A36. The method of any one of Embodiments A1-A35, wherein the human has elevated blood pressure.

Embodiment A37. The method of Embodiment A36, wherein the administration treats the elevated blood pressure in the human.

Embodiment A38. The method of any one of Embodiments A1-A37, wherein the human has hypertension.

Embodiment A39. The method of Embodiment A38, wherein the hypertension is stage 1 hypertension.

Embodiment A40. The method of Embodiment A38, wherein the hypertension is stage 2 hypertension.

Embodiment A41. The method of Embodiment A38, wherein the administration treats the hypertension in the human.

Embodiment A42. The method of Embodiment A41, wherein the hypertension is stage 1 hypertension.

Embodiment A43. The method of Embodiment A41, wherein the hypertension is stage 2 hypertension.

Embodiment A44. The method of any one of Embodiments A1-A43, wherein the human has pulmonary hypertension.

Embodiment A45. The method of Embodiment A44, wherein the administration treats the pulmonary hypertension in the human.

Embodiment A46. The method of any one of Embodiments A1-A45, wherein the human has pulmonary arterial hypertension.

Embodiment A47. The method of Embodiment A46, wherein the administration treats the pulmonary arterial hypertension in the human.

Embodiment A48. The method of any one of Embodiments A1-A47, wherein the human is undergoing a hypertensive crisis.

Embodiment A49. The method of Embodiment A48, wherein the administration treats the hypertensive crisis in the human.

Embodiment A50. The method of any one of Embodiments A1-A49, wherein the human has a cardiovascular disorder.

Embodiment A51. The method of Embodiment A50, wherein the cardiovascular disorder is atherosclerosis.

Embodiment A52. The method of Embodiment A50, wherein the cardiovascular disorder is heart failure.

Embodiment A53. The method of Embodiment A50, wherein the cardiovascular disorder is left ventricular hypertrophy.

Embodiment A54. The method of Embodiment A50, wherein the cardiovascular disorder is coronary artery disease.

Embodiment A55. The method of Embodiment A50, wherein the cardiovascular disorder is coronary microvascular disease.

Embodiment A56. The method of Embodiment A50, wherein the cardiovascular disorder is cardiac arrhythmia.

Embodiment A57. The method of Embodiment A50, wherein the administration treats the cardiovascular disorder in the human.

Embodiment A58. The method of any one of Embodiments A1-A57, wherein the administration treats atherosclerosis in the human.

Embodiment A59. The method of any one of Embodiments A1-A57, wherein the administration treats heart failure in the human.

Embodiment A60. The method of any one of Embodiments A1-A57, wherein the administration treats left ventricular hypertrophy in the human.

Embodiment A61. The method of any one of Embodiments A1-A57, wherein the administration treats coronary artery disease in the human.

Embodiment A62. The method of any one of Embodiments A1-A57, wherein the administration treats coronary microvascular disease in the human.

Embodiment A63. The method of any one of Embodiments A1-A57, wherein the administration treats cardiac arrhythmia in the human.

Embodiment A64. The method of any one of Embodiments A1-A63, wherein the human has an ocular condition.

Embodiment A65. The method of Embodiment A64, wherein the ocular condition is glaucoma.

Embodiment A66. The method of Embodiment A64, wherein the ocular condition is diabetic macular edema.

Embodiment A67. The method of Embodiment A64, wherein the ocular condition is diabetic retinopathy.

Embodiment A68. The method of Embodiment A64, wherein the ocular condition is ocular edema.

Embodiment A69. The method of Embodiment A64, wherein the administration treats the ocular condition.

Embodiment A70. The method of any one of Embodiments A1-A69, wherein the therapeutically-effective amount is from about 0.1 mg to about 100 mg.

Embodiment A71. The method of any one of Embodiments A1-A69, wherein the therapeutically-effective amount is from about 5 mg to about 60 mg.

Embodiment A72. The method of any one of Embodiments A1-A69, wherein the therapeutically-effective amount is from about 0.5 mg to about 30 mg.

Embodiment A73. The method of any one of Embodiments A1-A69, wherein the therapeutically-effective amount is about 5 mg.

Embodiment A74. The method of any one of Embodiments A1-A69, wherein the therapeutically-effective amount is about 15 mg.

Embodiment A75. The method of any one of Embodiments A1-A69, wherein the therapeutically-effective amount is about 22.5 mg.

Embodiment A76. The method of any one of Embodiments A1-A69, wherein the therapeutically-effective amount is about 30 mg.

Embodiment A77. The method of any one of Embodiments A1-A76, wherein the administering is by subcutaneous administration.

Embodiment A78. The method of any one of Embodiments A1-A76, wherein the administering is by oral administration.

Embodiment A79. The method of any one of Embodiments A1-A76, wherein the administering is by intravenous administration.

Embodiment A80. The method of any one of Embodiments A1-A76, wherein the administering is by inhalation.

Embodiment A81. The method of any one of Embodiments A1-A76, wherein the administering is by intratracheal administration.

Embodiment A82. The method of any one of Embodiments A1-A76, wherein the administering is by nasal administration.

Embodiment A83. The method of any one of Embodiments A1-A82, wherein the administration changes the blood pressure in the human by about 1 mmHg to about 50 mmHg.

Embodiment A84. The method of any one of Embodiments A1-A83, wherein:
a. the human has a baseline systolic blood pressure below about 140 mmHg; and
b. the administration changes the blood pressure in the human by about 1 mmHg to about 10 mmHg.

Embodiment A85. The method of any one of Embodiments A1-A83, wherein:
a. the human has a baseline systolic blood pressure above about 140 mmHg; and
b. the administration changes the blood pressure in the human by about 10 mmHg to about 100 mmHg.

Embodiment A86. The method of any one of Embodiments A1-A85, wherein the Tie-2 activator binds a phosphatase.

Embodiment A87. The method of any one of Embodiments A1-A86, wherein the Tie-2 activator inhibits a phosphatase.

Embodiment A88. The method of any one of Embodiments A1-A87, wherein the Tie-2 activator binds HPTPβ.

Embodiment A89. The method of any one of Embodiments A1-A88, wherein the Tie-2 activator inhibits HPTPβ.

Embodiment A90. The method of any one of Embodiments A1-A89, wherein the Tie-2 activator is a phosphate mimetic.

Embodiment A91. The method of any one of Embodiments A1-A90, wherein the Tie-2 activator is a compound of the formula:

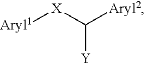

wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

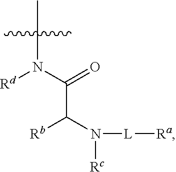

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R⁹ is H, alkyl, alkenyl, alkynyl, aryl, arylalkyl, heterocyclyl, heterocyclylalkyl, heteroaryl, or heteroarylalkyl, any of which is substituted or unsubstituted,

or a pharmaceutically-acceptable salt thereof.

Embodiment A92. The method of Embodiment A91, wherein:
Aryl¹ is substituted or unsubstituted phenyl;
Ary² is substituted or unsubstituted heteroaryl; and
X is alkylene.

Embodiment A93. The method of Embodiment A91 or A92, wherein:
Aryl¹ is substituted phenyl;
Ary² is substituted heteroaryl; and
X is methylene.

Embodiment A94. The method of any one of Embodiments A91-A93, wherein the compound that activates Tie-2 is a compound of the formula:

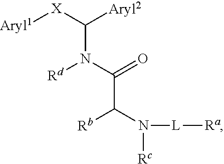

wherein
Aryl¹ is para-substituted phenyl;
Aryl² is substituted heteroaryl;
X is methylene;
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

Embodiment A95. The method of Embodiment A94, wherein:
Aryl¹ is para-substituted phenyl;
Aryl² is a substituted thiazole moiety;
X is methylene;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

Embodiment A96. The method of Embodiment any one of Embodiments A91-A95, wherein Aryl² is:

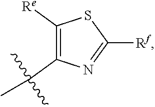

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment A97. The method of Embodiment A96, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment A98. The method of Embodiment A96, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment A99. The method of any one of Embodiments A91-A98, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

Embodiment A100. The method of Embodiment A91, wherein the compound is:

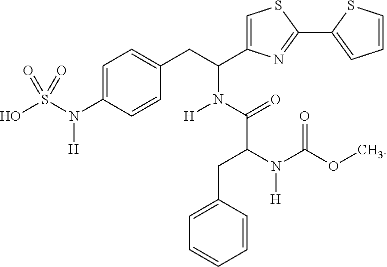 [SMILES: COC(=O)NC(Cc1ccccc1)C(=O)NC(Cc1ccc(NS(=O)(=O)O)cc1)c1csc(-c2cccs2)n1]

Embodiment A101. The method of Embodiment A91, wherein the compound is:

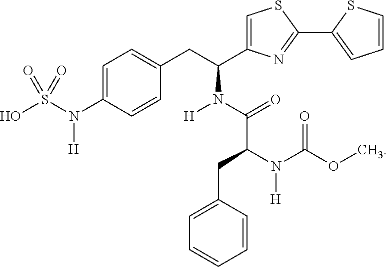 [SMILES: COC(=O)N[C@@H](Cc1ccccc1)C(=O)N[C@@H](Cc1ccc(NS(=O)(=O)O)cc1)c1csc(-c2cccs2)n1]

Embodiment A102. The method of Embodiment A96, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

Embodiment A103. The method of Embodiment A91, wherein the compound is:

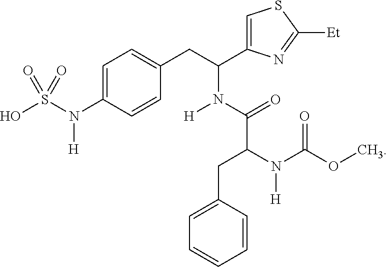 [SMILES: CCc1nc(C(Cc2ccc(NS(=O)(=O)O)cc2)NC(=O)C(Cc2ccccc2)NC(=O)OC)cs1]

Embodiment A104. The method of Embodiment A91, wherein the compound is:

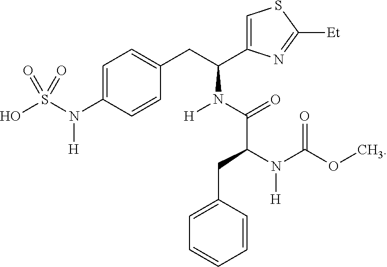 [SMILES: CCc1nc([C@H](Cc2ccc(NS(=O)(=O)O)cc2)NC(=O)[C@H](Cc2ccccc2)NC(=O)OC)cs1]

Embodiment A105. The method of any one of Embodiments A91-A95, wherein Aryl² is:

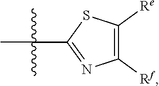

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment A106. The method of Embodiment A105, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment A107. The method of Embodiment A105, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment A108. The method of Embodiment A105, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

Embodiment A109. The method of Embodiment A91, wherein the compound is:

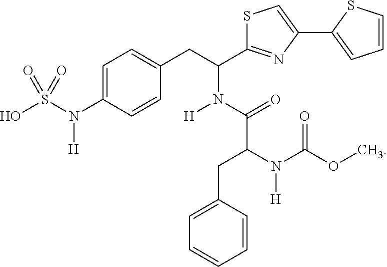 [SMILES: COC(=O)NC(Cc1ccccc1)C(=O)NC(Cc1ccc(NS(=O)(=O)O)cc1)c1nc(-c2cccs2)cs1]

Embodiment A110. The method of Embodiment A91, wherein the compound is:

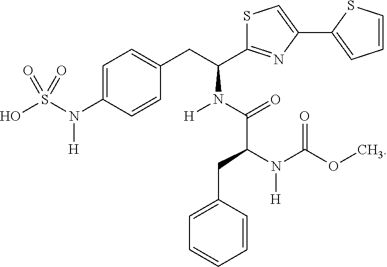 [SMILES: COC(=O)N[C@@H](Cc1ccccc1)C(=O)N[C@@H](Cc1ccc(NS(=O)(=O)O)cc1)c1nc(-c2cccs2)cs1]

Embodiment A111. A method of modulating blood pressure in a subject in need thereof, comprising administering to the subject a therapeutically effective amount of a Tie-2 activator, wherein: in a study of a human with hypertension, modulation in blood pressure in the human 90 minutes after administration of the Tie-2 activator to the human correlates to a baseline sitting blood pressure of the human as illustrated in the bottom panel of FIG. 22, and wherein the modulation in blood pressure in the human versus the baseline sitting blood pressure in the human has at most a 30% deviation from the regression line shown above.

Embodiment A112. The method of Embodiment A111, wherein the Tie-2 activator is a compound of any one of Embodiments A86-A110.

Embodiment A113. A method of modulating blood pressure in a subject in need thereof, comprising administering to the subject a therapeutically effective amount of a HPTPβ inhibitor, wherein: in a study of a human with hypertension, modulation in blood pressure in the human 90 minutes after administration of the HPTPβ inhibitor to the human correlates to a baseline sitting blood pressure of the human as illustrated in the bottom panel of FIG. 22.

Embodiment A114. The method of Embodiment A113, wherein the Tie-2 activator is a compound of any one of Embodiments A86-A110.

Embodiment B1. A method of treating pulmonary hypertension in a subject in need thereof, the method comprising administering to the subject a therapeutically-effective amount of a Tie-activator, wherein the Tie-2 activator is a small organic molecule.

Embodiment B2. The method of Embodiment B1, wherein the pulmonary hypertension is pulmonary arterial hypertension.

Embodiment B3. The method of Embodiment B1 or B2, wherein the Tie-2 activator modulates a blood pressure in the subject.

Embodiment B4. The method of Embodiment B3, wherein the blood pressure is systolic blood pressure.

Embodiment B5. The method of Embodiment B3, wherein the blood pressure is diastolic blood pressure.

Embodiment B6. The method of Embodiment B3, wherein the blood pressure is a mean arterial blood pressure.

Embodiment B7. The method of Embodiment B3, wherein the blood pressure is a pulmonary artery blood pressure.

Embodiment B8. The method of Embodiment B3, wherein the blood pressure is a pulmonary artery systolic blood pressure.

Embodiment B9. The method of any one of Embodiments B1-B8, wherein the Tie-2 activator modulates a blood pressure in the subject by about 0.1 mmHg to about 100 mmHg.

Embodiment B10. The method of any one of Embodiments B1-B8, wherein the Tie-2 activator modulates a blood pressure in the subject by about 1 mmHg to about 50 mmHg.

Embodiment B11. The method of any one of Embodiments B1-B10, wherein the Tie-2 activator reduces a blood pressure in the subject.

Embodiment B12. The method of any one of Embodiments B1-B11, wherein the administration reduces a pulse pressure in the subject.

Embodiment B13. The method of any one of Embodiments B1-B12, wherein the administration increases a level of a signaling molecule in the subject.

Embodiment B14. The method of Embodiment B13, wherein the signaling molecule is nitric oxide.

Embodiment B15. The method of Embodiment B13, wherein the signaling molecule is cyclic guanosine monophosphate.

Embodiment B16. The method of any one of Embodiments B13-B15, wherein the level of the signaling molecule is increased by decreasing metabolism of the signaling molecule in a tissue of the subject.

Embodiment B17. The method of any one of Embodiments B13-B15, wherein the level of the signaling molecule is increased by decreasing export of the signaling molecule in a tissue of the subject.

Embodiment B18. The method of any one of Embodiments B13-B15, wherein the level of the signaling molecule is increased in an endothelial cell in the subject.

Embodiment B19. The method of any one of Embodiments B16-B18, wherein the level of the signaling molecule is increased in an endothelial cell in the subject, wherein the signaling molecule is nitric oxide.

Embodiment B20. The method of any one of Embodiments B1-B19, wherein the administration increases endothelial function in the subject.

Embodiment B21. The method of any one of Embodiments B1-B20, wherein the administration increases phenylephrine-induced contraction in the subject.

Embodiment B22. The method of any one of Embodiments B1-B21, wherein the administration increases acetylcholine-induced relaxation in the subject.

Embodiment B23. The method of any one of Embodiments B1-B22, wherein the administration activates endothelial nitric oxide synthase (eNOS) in the subject.

Embodiment B24. The method of Embodiment B23, wherein the administration activates eNOS in the subject by increasing eNOS phosphorylation on Tyr81 and Ser1177.

Embodiment B25. The method of Embodiment B23, wherein the administration activates eNOS in the subject via activation of proto-oncogene tyrosine-protein kinase (Src) in the subject.

Embodiment B26. The method of Embodiment B23, wherein the administration activates eNOS in the subject via activation of Abelson murine leukemia viral oncogene homolog 1 (ABL1) in the subject.

Embodiment B27. The method of any one of Embodiments B1-B26, wherein the administration activates Akt in the subject.

Embodiment B28. The method of any one of Embodiments B1-B27, wherein the administration increases vascularization in the subject.

Embodiment B29. The method of any one of Embodiments B1-B27, wherein the administration increases vascularization in a lung of the subject.

Embodiment B30. The method of any one of Embodiments B1-B29, wherein the administration reduces a rate of blood efflux in the subject.

Embodiment B31. The method of Embodiment B30, wherein the rate of blood efflux is reduced by reducing vascular permeability.

Embodiment B32. The method of Embodiment B30, wherein the rate of blood efflux is reduced by reducing venous drainage.

Embodiment B33. The method of Embodiment B30, wherein the rate of blood efflux is reduced by reducing venous leak.

Embodiment B34. The method of any one of Embodiments B1-B33, wherein the administration increases a level of dilation of a blood vessel in the subject.

Embodiment B35. The method of Embodiment B34, wherein the blood vessel is an artery.

Embodiment B36. The method of Embodiment B34, wherein the blood vessel is a vein.

Embodiment B37. The method of Embodiment B34, wherein the blood vessel is a capillary.

Embodiment B38. The method of any one of Embodiments B1-B37, wherein the subject has diabetes.

Embodiment B39. The method of Embodiment B38, wherein the administration treats the diabetes in the subject.

Embodiment B40. The method of any one of Embodiments B1-B39, wherein the subject is undergoing a hypertensive crisis.

Embodiment B41. The method of Embodiment B40, wherein the administration treats the hypertensive crisis in the subject.

Embodiment B42. The method of any one of Embodiments B1-B41, wherein the subject has a cardiovascular disorder.

Embodiment B43. The method of Embodiment B42, wherein the cardiovascular disorder is atherosclerosis.

Embodiment B45. The method of Embodiment B42, wherein the cardiovascular disorder is heart failure.

Embodiment B46. The method of Embodiment B42, wherein the cardiovascular disorder is left ventricular hypertrophy.

Embodiment B47. The method of Embodiment B42, wherein the cardiovascular disorder is coronary artery disease.

Embodiment B48. The method of Embodiment B42, wherein the cardiovascular disorder is coronary microvascular disease.

Embodiment B49. The method of Embodiment B42, wherein the cardiovascular disorder is cardiac arrhythmia.

Embodiment B50. The method of Embodiment B42, wherein the administration treats the cardiovascular disorder in the subject.

Embodiment B51. The method of any one of Embodiments B1-B50, wherein the administration treats atherosclerosis in the subject.

Embodiment B52. The method of any one of Embodiments B1-B51, wherein the administration treats heart failure in the subject.

Embodiment B53. The method of any one of Embodiments B1-B52, wherein the administration treats left ventricular hypertrophy in the subject.

Embodiment B54. The method of any one of Embodiments B1-B53, wherein the administration treats coronary artery disease in the subject.

Embodiment B55. The method of any one of Embodiments B1-B54, wherein the administration treats coronary microvascular disease in the subject.

Embodiment B56. The method of any one of Embodiments B1-B55, wherein the administration treats cardiac arrhythmia in the subject.

Embodiment B57. The method of any one of Embodiments B1-B56, wherein the subject has an ocular condition.

Embodiment B58. The method of Embodiment B57, wherein the ocular condition is glaucoma.

Embodiment B59. The method of Embodiment B57, wherein the ocular condition is diabetic macular edema.

Embodiment B60. The method of Embodiment B57, wherein the ocular condition is diabetic retinopathy.

Embodiment B61. The method of Embodiment B57, wherein the ocular condition is ocular edema.

Embodiment B62. The method of Embodiment B57, wherein the administration treats the ocular condition in the subject.

Embodiment B63. The method of any one of Embodiments B1-B62, wherein the therapeutically-effective amount is from about 0.1 mg to about 100 mg.

Embodiment B64. The method of any one of Embodiments B1-B62, wherein the therapeutically-effective amount is from about 5 mg to about 60 mg.

Embodiment B65. The method of any one of Embodiments B1-B62, wherein the therapeutically-effective amount is from about 0.5 mg to about 30 mg.

Embodiment B66. The method of any one of Embodiments B1-B62, wherein the therapeutically-effective amount is about 5 mg.

Embodiment B67. The method of any one of Embodiments B1-B62, wherein the therapeutically-effective amount is about 15 mg.

Embodiment B68. The method of any one of Embodiments B1-B62, wherein the therapeutically-effective amount is about 22.5 mg.

Embodiment B69. The method of any one of Embodiments B1-B62, wherein the therapeutically-effective amount is about 30 mg.

Embodiment B70. The method of any one of Embodiments B1-B69, wherein the administering is by subcutaneous administration.

Embodiment B71. The method of any one of Embodiments B1-B69, wherein the administering is by oral administration.

Embodiment B72. The method of any one of Embodiments B1-B69, wherein the administering is by intravenous administration.

Embodiment B73. The method of any one of Embodiments B1-B69, wherein the administering is by inhalation.

Embodiment B74. The method of any one of Embodiments B1-B69, wherein the administering is by intratracheal administration.

Embodiment B75. The method of any one of Embodiments B1-B69, wherein the administering is by nasal administration.

Embodiment B76. The method of any one of Embodiments B1-B75, wherein:
a. the subject has a baseline systolic blood pressure below about 140 mmHg; and
b. the administration changes the blood pressure in the subject by about 1 mmHg to about 10 mmHg.

Embodiment B77. The method of any one of Embodiments B1-B75, wherein:
a. the subject has a baseline systolic blood pressure above about 140 mmHg; and
b. the administration changes the blood pressure in the subject by about 10 mmHg to about 100 mmHg.

Embodiment B78. The method of any one of Embodiments B1-B77, wherein the Tie-2 activator binds a phosphatase.

Embodiment B79. The method of any one of Embodiments B1-B78, wherein the Tie-2 activator inhibits a phosphatase.

Embodiment B80. The method of any one of Embodiments B1-B79, wherein the Tie-2 activator binds HPTPβ.

Embodiment B81. The method of any one of Embodiments B1-B80, wherein the Tie-2 activator inhibits HPTPβ.

Embodiment B82. The method of any one of Embodiments B1-B81, wherein the Tie-2 activator is a phosphate mimetic.

Embodiment B83. The method of any one of Embodiments B1-B82, wherein the Tie-2 activator is a compound of the formula:

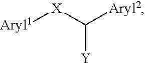

wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

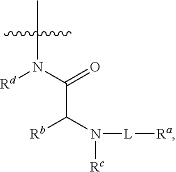

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,

or a pharmaceutically-acceptable salt thereof.

Embodiment B84. The method of Embodiment B83, wherein:
Aryl¹ is substituted or unsubstituted phenyl;
Ary² is substituted or unsubstituted heteroaryl; and
X is alkylene.

Embodiment B85. The method of Embodiment B83 or B84, wherein:
Aryl¹ is substituted phenyl;
Ary² is substituted heteroaryl; and
X is methylene.

Embodiment B86. The method of any one of Embodiments B83-B85, wherein the compound that activates Tie-2 is a compound of the formula:

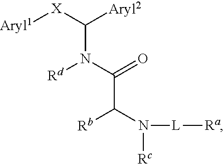

wherein
Aryl¹ is para-substituted phenyl;
Aryl² is substituted heteroaryl;
X is methylene;
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond;
R^{a} is H, alkyl, alkenyl, alkynyl, aryl, arylalkyl, heterocyclyl, heterocyclylalkyl, heteroaryl, or heteroarylalkyl, any of which is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted;
R^{c} is H or alkyl which is substituted or unsubstituted; and
R^{d} is H or alkyl which is substituted or unsubstituted.

Embodiment B87. The method of Embodiment B85, wherein:
Aryl¹ is para-substituted phenyl;
Aryl² is a substituted thiazole moiety;
X is methylene;
L² together with the nitrogen atom to which L² is bound forms a carbamate linkage;
R^{a} is alkyl, which is substituted or unsubstituted;
R^{b} is arylalkyl, which is substituted or unsubstituted;
R^{c} is H; and
R^{d} is H.

Embodiment B88. The method of any one of Embodiments B83-B87, wherein Aryl² is:

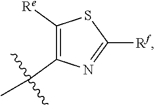

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment B89. The method of Embodiment B88, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment B90. The method of Embodiment B88, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment B91. The method of any one of Embodiments B83-B90, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

Embodiment B92. The method of Embodiment B83, wherein the compound is:

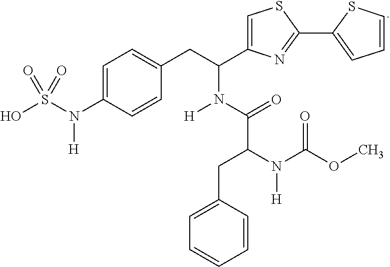 [SMILES: COC(=O)NC(Cc1ccccc1)C(=O)NC(Cc1ccc(NS(=O)(=O)O)cc1)c1csc(-c2cccs2)n1]

Embodiment B93. The method of Embodiment B83, wherein the compound is:

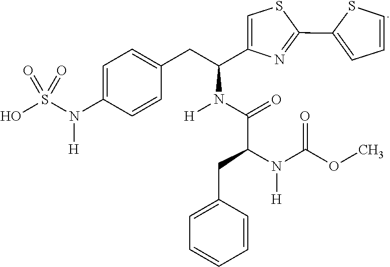 [SMILES: COC(=O)N[C@@H](Cc1ccccc1)C(=O)N[C@@H](Cc1ccc(NS(=O)(=O)O)cc1)c1csc(-c2cccs2)n1]

Embodiment B94. The method of Embodiment B88, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

Embodiment B95. The method of Embodiment B83, wherein the compound is:

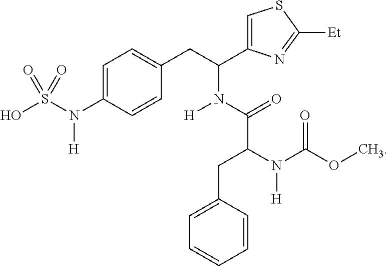 [SMILES: CCc1nc(C(Cc2ccc(NS(=O)(=O)O)cc2)NC(=O)C(Cc2ccccc2)NC(=O)OC)cs1]

Embodiment B96. The method of Embodiment B83, wherein the compound is:

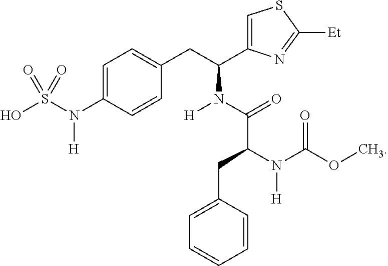 [SMILES: CCc1nc([C@H](Cc2ccc(NS(=O)(=O)O)cc2)NC(=O)[C@H](Cc2ccccc2)NC(=O)OC)cs1]

Embodiment B97. The method of any one of Embodiments B83-B87, wherein Aryl² is:

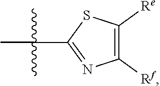

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment B98. The method of Embodiment B97, wherein:
R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and
R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted.

Embodiment B99. The method of Embodiment B97, wherein:
R^{e} is H, OH, F, Cl, Br, I, alkyl, or an alkoxy group, any of which is substituted or unsubstituted; and
R^{f} is alkyl, aryl, heterocyclyl, or heteroaryl, any of which is substituted or unsubstituted.

Embodiment B100. The method of Embodiment B97, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

Embodiment B101. The method of Embodiment B83, wherein the compound is:

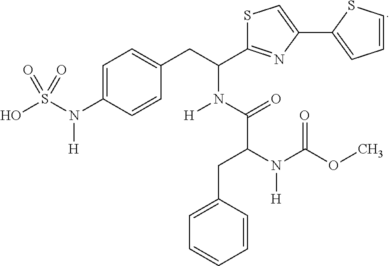 [SMILES: COC(=O)NC(Cc1ccccc1)C(=O)NC(Cc1ccc(NS(=O)(=O)O)cc1)c1nc(-c2cccs2)cs1]

Embodiment B102. The method of Embodiment B83, wherein the compound is:

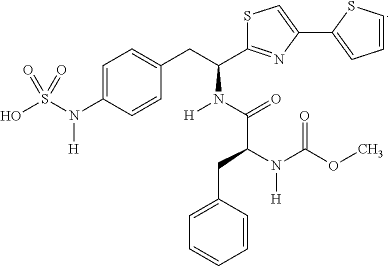 [SMILES: COC(=O)N[C@@H](Cc1ccccc1)C(=O)N[C@@H](Cc1ccc(NS(=O)(=O)O)cc1)c1nc(-c2cccs2)cs1]

Embodiment B103. A method of treating hypertension in a subject in need thereof, comprising administering to the subject a therapeutically effective amount of a Tie-2 activator, wherein: in a study of a human with hypertension, modulation in blood pressure in the human 90 minutes after administration of the Tie-2 activator to the human correlates to a baseline sitting blood pressure of the human as illustrated in the bottom panel of FIG. 22, and wherein the modulation in blood pressure in the human versus the baseline sitting blood pressure in the human has at most a 30% deviation from the regression line shown above.

Embodiment B104. The method of Embodiment B103, wherein the Tie-2 activator is a compound of any one of Embodiments B78-B102.

Embodiment B105. A method of treating hypertension in a subject in need thereof, comprising administering to the subject a therapeutically effective amount of a HPTPβ inhibitor, wherein: in a study of a human with hypertension, modulation in blood pressure in the human 90 minutes after administration of the HPTPβ inhibitor to the human correlates to a baseline sitting blood pressure of the human as illustrated in the bottom panel of FIG. 22.

Embodiment B106. The method of Embodiment B105, wherein the Tie-2 activator is a compound of any one of Embodiments B78-B102.

1-178. (canceled)

179. A method for modulating a blood pressure in a human in need thereof, the method comprising administering to the human a therapeutically effective amount of a Tie-2 activator, wherein the administration changes the blood pressure in the human by about 0.1 mmHg to about 100 mmHg.

180. The method of claim 179, wherein the modulating the blood pressure is reducing the blood pressure.

181. The method of claim 179, wherein the blood pressure is systolic blood pressure.

182. The method of claim 179, wherein the blood pressure is diastolic blood pressure.

183. The method of claim 179, wherein the blood pressure is a mean arterial blood pressure.

184. The method of claim 179, wherein the blood pressure is a pulmonary artery blood pressure.

185. The method of claim 179, wherein the blood pressure is a pulmonary artery systolic blood pressure.

186. The method of claim 179, wherein the administration reduces a pulse pressure in the human.

187. The method of claim 179, wherein the administration increases a level of nitric oxide in the human.

188. The method of claim 179, wherein the administration activates endothelial nitric oxide synthase in the human.

189. The method of claim 179, wherein the human has hypertension.

190. The method of claim 179, wherein the therapeutically-effective amount is from about 0.1 mg to about 100 mg.

191. The method of claim 179, wherein the therapeutically-effective amount is about 15 mg.

192. The method of claim 179, wherein the therapeutically-effective amount is about 40 mg.

193. The method of claim 179, wherein the Tie-2 activator is administered as part of a composition at a concentration of about 0.1 mg/mL to about 100 mg/mL.

194. The method of claim 179, wherein the Tie-2 activator is administered as part of a composition at a concentration of about 40 mg/mL.

195. The method of claim 179, wherein the administering is by subcutaneous administration.

196. The method of claim 179, wherein the administering is by oral administration.

197. The method of claim 179, wherein the administering is by inhalation.

198. The method of claim 179, wherein the administering is by intratracheal administration.

199. The method of claim 179, wherein the administering is by nasal administration.

200. The method of claim 179, wherein the administering changes the blood pressure in the human by about 1 mmHg to about 50 mmHg.

201. The method of claim 179, wherein the Tie-2 activator is a compound of the formula: 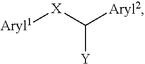
wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or 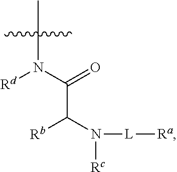
wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{c} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted, or a pharmaceutically-acceptable salt thereof.

202. The method of claim 201, wherein:
Aryl¹ is substituted or unsubstituted phenyl;
Ary² is substituted or unsubstituted heteroaryl; and
X is alkylene.

203. The method of claim 201, wherein Ary² is: 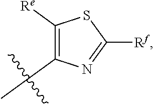
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

204. The method of claim 203, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is alkyl.

205. The method of claim 179, wherein the Tie-2 activator is: 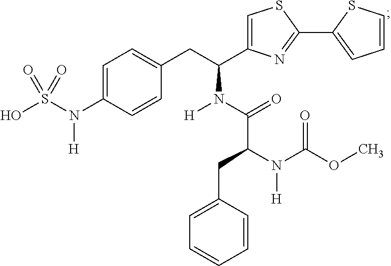 [SMILES: COC(=O)N[C@@H](Cc1ccccc1)C(=O)N[C@@H](Cc1ccc(NS(=O)(=O)O)cc1)c1csc(-c2cccs2)n1]
or a pharmaceutically-acceptable salt thereof.

206. The method of claim 179, wherein the Tie-2 activator is: 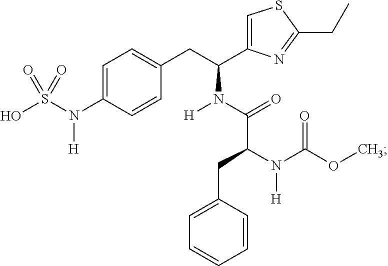 [SMILES: CCc1nc([C@H](Cc2ccc(NS(=O)(=O)O)cc2)NC(=O)[C@H](Cc2ccccc2)NC(=O)OC)cs1]
or a pharmaceutically-acceptable salt thereof.

207. The method of claim 201, wherein Aryl² is: 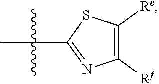
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

208. The method of claim 207, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl.

209. The method of claim 179, wherein the Tie-2 activator is: 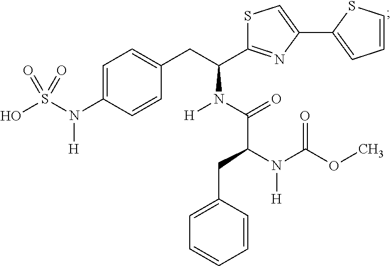 [SMILES: COC(=O)N[C@@H](Cc1ccccc1)C(=O)N[C@@H](Cc1ccc(NS(=O)(=O)O)cc1)c1nc(-c2cccs2)cs1]
or a pharmaceutically-acceptable salt thereof.

210. The method of claim 179, wherein the Tie-2 activator is: 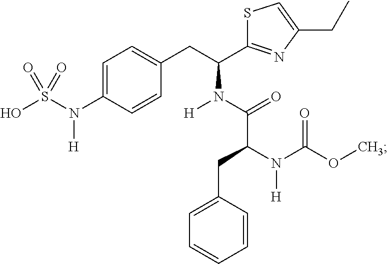 [SMILES: CCc1csc([C@H](Cc2ccc(NS(=O)(=O)O)cc2)NC(=O)[C@H](Cc2ccccc2)NC(=O)OC)n1]
or a pharmaceutically-acceptable salt thereof.
